(19) United States
(12) Patent Application Publication
(10) Pub. No.: US 2026/0355082 A1
(43) Pub. Date: Nov. 12, 2026

(54) Composition with Improved Water Resistance

(71) Applicant: Symrise AG, Holzminden (DE)

(72) Inventors: Jürgen CLAUS, Bevern (DE); Ev SUESS, Bevern (DE); Imke MEYER, Bodenwerder (DE); Nikolas BUGDAHN, Holzminden (DE); Sabine LANGE, Holzminden (DE); Carolin HEIN, Elsenfeld (DE); Daniel Berndt, Halle (DE); Lukas Schaaf, Hameln (DE)

(21) Appl. No.: 19/100,527

(22) PCT Filed: Aug. 7, 2023
(86) PCT No.: PCT/EP2023/071782

§ 371 Date: Jan. 31, 2025

(30) Foreign Application Priority Data
Aug. 5, 2022 (WO) PCT/EP2022/072098
Aug. 4, 2023 (WO) PCT/EP2023/071719

(51) Int. Cl. A61K 8/44; A61K 8/04; A61K 8/46; A61K 8/49; A61Q 17/04; A61Q 19/08; C11D 3/33

(52) U.S. Cl. CPC A61K 8/44; A61K 8/04; A61K 8/466; A61K 8/49; A61K 8/4926; A61Q 17/04; A61Q 19/08; C11D 3/33

(57) ABSTRACT

The present invention relates to sunscreen products, cosmetic or pharmaceutical preparations or homecare products comprising at least one primary organic and/or inorganic UV-filter, a certain mycosporine-like amino acid compound and optionally at least one film forming agent. The products and preparations provide improved water resistance and, thus, increased SPF performance without being unpleasantly sticky. Additionally, the present invention relates to the use of such mycosporine-like amino acid compound for improving water resistance and, thus, SPF performance, of a sunscreen product, cosmetic or pharmaceutical preparation or homecare product comprising at least one primary organic and/or inorganic UV-filter and optionally at least one film forming agent. Finally, the present invention relates to a method of increasing the water resistance and, thus, SPF performance, of a sunscreen product, cosmetic or pharmaceutical preparation or homecare product comprising at least one primary organic and/or inorganic UV-filter and optionally at least one film forming agent by the addition of a certain mycosporine-like amino acid compound.

### TECHNICAL FIELD

The present invention relates to sunscreen products, cosmetic or pharmaceutical preparations or homecare products comprising at least one primary organic and/or inorganic UV-filter, a certain mycosporine-like amino acid compound and optionally at least one film forming agent. The products and preparations provide improved water resistance and, thus, increased SPF performance without being unpleasantly sticky. Additionally, the present invention relates to the use of such mycosporine-like amino acid compound for improving water resistance and, thus, SPF performance, of a sunscreen product, cosmetic or pharmaceutical preparation or homecare product comprising at least one primary organic and/or inorganic UV-filter and optionally at least one film forming agent. Finally, the present invention relates to a method of increasing the water resistance and, thus, SPF performance, of a sunscreen product, cosmetic or pharmaceutical preparation or homecare product comprising at least one primary organic and/or inorganic UV-filter and optionally at least one film forming agent by the addition of a certain mycosporine-like amino acid compound.

### BACKGROUND ART

The negative effects of exposure of UV light are well-known. Prolonged exposure to sunlight causes damage such as sunburn to the skin and dries out hair making it brittle. When skin is exposed to UV light having a wavelength of from 290 nm to 400 nm, long term damage can lead to serious conditions such as skin cancer, the type of damage depending on the wavelength of the radiation.

UV light also contributes to aging by causing free radicals to form in the skin. Free radicals include, for example, singlet oxygen, hydroxyl radical, the superoxide anion, nitric oxide and hydrogen radicals. Free radicals attack DNA, membrane lipids and proteins, generating carbon radicals. These in turn react with oxygen to produce a peroxyl radical that can attack adjacent fatty acids to generate new carbon radicals. This cascade leads to a chain reaction producing lipid peroxidation products. Damage to the cell membrane results in loss of cell permeability, increased intercellular ionic concentration, and decreased ability to excrete or detoxify waste products. The end result is a loss of skin elasticity and the appearance of wrinkles. This process is commonly referred to as photo-aging.

Moreover, UVA-radiation can trigger phototoxic or photo allergic skin reactions.

UV rays are classified according to their wavelength as UVA rays (320 to 400 nm) or UVB rays (280 to 320 nm).

To attenuate these negative effects of UV-radiation, i.e. for the purpose of protecting the skin and keratin materials against UV-radiation, anti-sun/sunscreen compositions comprising screening agents that are active in the UVA-range and in the UVB-range, i.e. within the full range of 280 nm to 400 nm, are generally used.

UV-filters are compounds which have a pronounced absorption capacity for ultraviolet radiation. They are used especially in sunscreen products, cosmetic, dermatological and pharmacological preparations or sunscreen products, but also to improve the light fastness of industrial products, such as paints, varnishes, plastics, textiles, polymers such as, for example, polymers and copolymers of mono- and di-olefins, polystyrenes, polyurethanes, polyamides, polyesters, polyureas and polycarbonates, packaging materials and rubbers.

The organic and inorganic UV-filters are classified as UVA and UVB filters depending on the location of their absorption maxima; if a UV-filter absorbs both UVA and UVB, it is referred to as a UVA/B broadband absorber or UVA/UVB broadband filter.

In order to achieve the desired maximum protection from UV-radiation sunscreen products or cosmetic, especially dermatological, and pharmacological preparations are formulated to contain a mixture of UV-filters with varying concentrations and the choice of UV-filters used is determined by the legislation within the country or economic area. For example, UV-filters which can be used for the protection of skin are regulated in the USA by the America FDA via their OTC monograph system and are regulated in the European Union by the Cosmetic Regulation. Regulations covering the use of UV-filters exist in other countries and regions as well. These regulations not only stipulate the filters which can be used but also fix a maximum usage level for each UV-filter. Thus, there are limited UV-filters available to achieve high efficacy with respect to both SPF and UVA or UVB protection.

When updating a sunscreen formulation to meet today's requirements, formulators must consider improved water-resistance as a key parameter, since sunscreens are often used outdoors near bodies of water. Exposure of a sunscreen product to water, such as typically occurring when bathing, results in a significant removal of the UV-filter from the skin, either by leaching out or by physical removing, which leads to a decrease of protection capacity against the UV light. This can be shown by a significant decrease of the composition's SPF. Reapplication of the product after each water exposure is undesirable for modern consumers who seek both comfort and ease of use from their sun care products. Moreover, the protection provided by a sunscreen can be compromised with sweating; in fact, the reduced efficacy of sun care products due to perspiration has been shown in athletes, where rates of skin cancer have grown.

In general, sufficient or high water-resistance can be accomplished by preparing a fully anhydrous formulation; creating a w/o emulsion; adding alcohol to dissolve water-insoluble substances that deposit onto the skin from an insoluble layer; obtaining a meta-stable o/w emulsion with a low level of emulsifiers; and entrapping filters in skin-substantive liposomes or sponges. One or more of these strategies are used when formulating a water-resistant formulation.

Water resistance of sun care products can further be improved by the addition of film forming polymers. Water-resistant products are more likely to achieve lower transdermal delivery since their components are contained inside the applied film. This factor allows water-resistant sunscreens to provide longer-lasting SPF protection than standard sunscreen products. In addition, the UV-filters entrapped in the film will have low transdermal delivery to the body, reducing the risk of chemicals penetrating the skin and lowering the accumulation of said substances.

These film forming polymers, however, are either not sufficiently effective and/or render the product unattractive for the end consumer due to the resulting sensory properties as such products often exhibit an oily, dull, heavy or sticky feeling on the skin.

Beyond maintaining the efficacy of sunscreens when they come in contact with water, there is also increasing concern related to the accumulation of trace sunscreen ingredients that could harm the resident flora and fauna in lakes, rivers and oceans.

Thus, there is still an ongoing need to provide further substances which improve the water resistance of a sunscreen product, cosmetic or pharmaceutical preparation or homecare product comprising a UV-filter and optionally a film forming agent.

It was therefore the object of the present invention to remedy the disadvantage of the prior art and to provide sunscreen products, cosmetic or pharmaceutical preparations or homecare products which exhibit an improved water resistance and, thus, exhibit high SPF performance and provide excellent UV protection even after bathing. Furthermore, such products or preparations should exhibit excellent sensory properties in particular without being unpleasant sticky.

It is another object of the present invention to provide the use of a substance, for improving water resistance and, thus, SPF performance in a sunscreen product, cosmetic or pharmaceutical preparation or homecare product comprising a UV-filter and a film forming agent.

It is still another object of the present invention to provide a method of improving water resistance and, thus, SPF performance in a sunscreen product, cosmetic or pharmaceutical preparation or homecare product comprising a UV-filter and a film forming agent.

Surprisingly, it has been found that the water resistance and, thus, SPF performance in a sunscreen product, cosmetic or pharmaceutical preparation or homecare product can be significantly improved by the addition of a certain mycosporine-like amino acid compound without negatively affecting the sensory properties.

The problem is solved by the subject matter of the independent patent claims. Further aspects of the present invention are however also apparent from the wording of the dependent patent claims, the following detailed description in conjunction with the accompanying examples and figures.

### SUMMARY OF THE INVENTION

In order to accomplish the above problem, the present invention provides in a first aspect a sunscreen product, a cosmetic or pharmaceutical preparation or homecare product, comprising or consisting of
(a) at least one primary organic and/or inorganic UV-filter; and
(b) at least one mycosporine-like amino acid compound, represented either by the general formula (V)

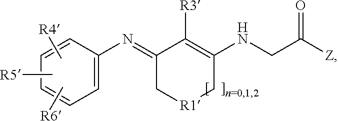

as defined herein, or a tautomer or a stereoisomer or a salt thereof;
or
represented by the general formula (VI)

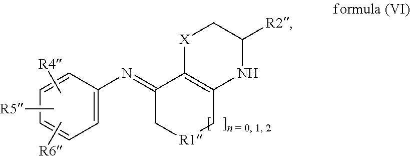

as defined herein, or a tautomer or a stereoisomer or a salt thereof; or any mixture of the afore-mentioned compounds; and
(c) optionally at least one film forming agent.

In a second aspect, the present invention provides for the use of at least one mycosporine-like amino acid compound as defined herein or a mixture thereof for improving the water resistance and SPF performance of a sunscreen product, cosmetic or pharmaceutical preparation or homecare product comprising at least one primary organic and/or inorganic UV-filter and optionally a film forming agent.

Finally, the present invention relates in a further aspect to a method of increasing the water resistance and SPF of a sunscreen product, cosmetic or pharmaceutical preparation or homecare product comprising at least one primary organic and/or inorganic UV-filter and optionally a film forming agent, said method comprising the addition of an effective amount of a mycosporine-like amino acid compound as defined herein or a mixture thereof to said sunscreen product, cosmetic or pharmaceutical preparation or homecare product comprising at least one UV-filter and optionally a film forming agent.

The present invention is specified in the appended claims. The invention itself, and its preferred variants, other objects and advantages, are however also apparent form the following detailed description in conjunction with the accompanying examples.

### DETAILED DESCRIPTION OF THE INVENTION

In the context of the present invention, the following general meanings apply:

As used herein, the singular form “a”, “a” and “the” include plural references unless the context clearly dictates otherwise. Thus, for example reference to “cosmetic preparation”, “the cosmetic preparation” or “a cosmetic preparation” also includes a plurality of cosmetic preparations.

The terms “comprising”, “including”, and “containing” are to be understood as open-ended terms and mean that the named components following said term are essential but not “limited to”, and other components may be added and are still embraced by the present invention.

The term “consisting of” as used according to the present invention means that the total amount of components (a) and (b) adds up to 100% by weight, based on the total weight of the sunscreen product or cosmetic or pharmaceutical preparation, and signifies that the subject matter is closed-ended and can only include the limitations that are expressly recited.

Whenever reference is made to “comprising” it is intended to cover both meanings as alternatives, that is the meaning can be either “comprising” or “consisting of” unless the context dictates otherwise.

The term “optionally” means that the subsequently described compound may but need not to be present in the composition, and that the description includes variants, where the compound is included or variants, where the compound is absent.

The term “or” or “and/or” is used as a function word to indicate that two words or phrases are to be taken together or separately.

The endpoints of all ranges directed to the same component or property are inclusive and independently combinable.

The term “compound(s)” or “compound(s) of the present invention” refers to all compounds encompassed by the structural formulae (V) and/or formula (VI) and/or formula (VII) and/or formula (XIII) and/or formula (IX) and/or formula (X) and/or formula (A) and their sub-formulae disclosed herein and includes each subgenus and all specific compounds within the formula whose structure is disclosed herein.

The compounds may be identified by either their chemical structure and/or chemical name. When the chemical structure and chemical name are in conflict, the chemical structure determines the identity of the compound.

The term “at least one . . . compound” means that the sunscreen product, cosmetic or pharmaceutical preparation or homecare product according to the present invention can comprise either one of said subsequently described individual compound or a mixture of two, three, four, five, six or even more different of said subsequently compounds.

The term “effective amount of a compound” means the amount of compound, that is sufficient to achieve the desired effect or improvement. For example: the “effective amount of an antimicrobial” means the amount or concentration of an antimicrobial, that is sufficient to result in an antimicrobial efficacy.

The term “cosmetic or pharmaceutical preparations” in the context of the present invention are compositions for cosmetic or pharmaceutical purposes which contain a UV absorber in order to protect skin or hair against UV-radiation. In addition to providing photo-protection, the cosmetic or pharmaceutical preparations according to the present invention are useful for providing anti-aging benefits to skin, whitening or preventing darkening of skin, improving appearance of skin, diminishing the visible signs of skin aging and improving skin's radiance and firmness.

The term “sunscreen composition” or “sunscreen” or “skin-care product”, also known as “sunblock”, “sun cream” or “suntan lotion”, refers to any topical product, which reflects and/or absorbs certain parts of UV radiation and thus helps protect against sunburn and most importantly prevent skin cancer. Thus, the term “sunscreen composition” is to be understood as not only including sunscreen compositions, but also any cosmetic compositions that provide UV protection. According to the present invention the sunscreen composition may comprise one or more active agents, e.g., organic UV-filters, as well as other ingredients or additives, e.g., emulsifiers, emollients, viscosity regulators, stabilizers, preservatives, or fragrances. Sunscreens come as lotions, sprays, gels, foams (such as an expanded foam lotion or whipped lotion), sticks, powders and other topical products. UV-absorbing compounds are used not only in sunscreen, but also in other personal care products, such as lipstick, shampoo, hair spray, body wash, toilet soap, and insect repellent.

The term “homecare products” in the context of the present invention are the essentials for daily care and cleaning purpose in households. They are used for maintaining hygiene and a good aura of the homes. The home care products generally include laundry detergents (powder, liquid and tablet), fabric conditioners, dishwashing detergents (liquid and tablet), hard floor and surface cleaners, glass cleaners, carpet cleaners, oven cleaners, air fresheners, disinfectants, stain removers, car wash products. These products are usually manufactured in the form of a liquid, powder, spray, granules and others. The sunscreen containing formulations prevent premature photodamage and photobleaching to surfaces and the homecare formulation itself.

The term “light stabilizers” as used herein are compounds suitable for protecting body-care and household cleaning and treating agents against photolytic degradation.

The term “photostability” refers to the ability of a UV-filter or any other molecule, which is exposed to sunlight, to stay stable upon irradiation. In particular, this means that the compound does not undergo a degradation process upon UV radiation.

The term “sun protection factor (SPF)” as used herein indicates how well the skin is protected by a sunscreen composition. In particular, the factor indicates how much longer the protected skin may be exposed to the sun without getting a sunburn in comparison to untreated skin. For example, if a sunscreen composition with an SPF of 15 is evenly applied to the skin of a person usually getting a sunburn after 10 minutes in the sun, the sunscreen allows the skilled person to stay in the sun 15 times longer. In other words, SPF 15 means that 1/15 of the burning UV radiation will reach the skin, assuming sunscreen is applied evenly at a thick dosage of 2 milligrams per square centimeter (mg/cm²).

The term “critical wavelength” is defined as the wavelength at which the area under the UV protection curve (% protection versus wavelength) represents 90% of the total area under the curve in the UV region (280-400 nm). For example, a critical wavelength of 370 nm indicates that the protection of the sunscreen composition is not limited to the wavelengths of UV-B, i.e. wavelengths from 280-320 nm, but extends to 370 nm in such a way that 90% of the total area under the protective curve in the UV region are reached at 370 nm. The higher the critical wavelength the better the protection against UV radiation.

According to the present invention, the primary object of the present invention is solved by a sunscreen product, cosmetic, especially dermatological, or pharmaceutical preparation or homecare product, comprising or consisting of
(a) at least one primary organic and/or inorganic UV-filter;
(b) at least one mycosporine-like amino acid compound,
   represented either by the general formula (V)

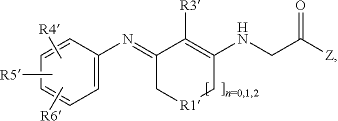

wherein
   Z is selected from the group consisting of O—R2′ or amide′;
   R1′ is selected from the group consisting of CH₂, unsubstituted or substituted CH(alkyl), unsubstituted or substituted C(alkyl)₂, unsubstituted or substituted CH(cycloalkyl), unsubstituted or substituted CH(aryl), unsubstituted or substituted CH(heterocycloalkyl), unsubstituted or substituted CH(heteroaryl), CR7′R8′, C═O, halogen, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), and unsubstituted or substituted N(heteroaryl), and unsubstituted or substituted C-spirocycles;
   R2′ is selected from the group consisting of H, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted cycloalkyl, unsubstituted or substituted aryl, unsubstituted or substituted heterocycloalkyl, unsubstituted or substituted heteroaryl;
   amide′ is selected from the group consisting of NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), and unsubstituted or substituted NR9′R10′, unsubstituted or substituted —N(O-alkyl)(alkyl) (Weinreb amide), —N(OH)(H) (hydroxamate), and unsubstituted or substituted —N(OH)(alkyl) (alkylated hydroxamate);
   R3′ is selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9′R10′, and unsubstituted or substituted C-spirocycles;
   the substituents R4′, R5′ and R6′ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, —S(═O)₂OH, sulfonyl, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), and unsubstituted or substituted NH(heteroaryl), unsubstituted or substituted NR9′R10′, and unsubstituted or substituted C-spirocycles;
   R7′ and R8′ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl) and unsubstituted or substituted NR9′R10′;
   R9′ und R10′ which may be identical or different, are independently from each other selected from the group consisting of H, OH, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, unsubstituted or substituted cycloalkyl, unsubstituted or substituted aryl, unsubstituted or substituted heterocycloalkyl, and unsubstituted or substituted heteroaryl;
   Y is selected from the group consisting of H and unsubstituted or substituted alkyl; and
   R is unsubstituted or substituted alkyl;
or a tautomer or a stereoisomer or a salt of the afore-mentioned compounds;
or represented by the general formula (VI)

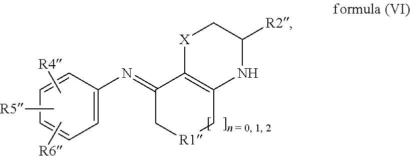

wherein
   R1″ is selected from the group consisting of CH₂, unsubstituted or substituted CH(alkyl), unsubstituted or substituted C(alkyl)₂, unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, halogen, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles;
   R2″ is selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —C(═O)-amide″, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, and unsubstituted or substituted C-spirocycles;
   X is selected from the group consisting of CH₂, unsubstituted or substituted CH(alkyl), unsubstituted or substituted CH(alkenyl), unsubstituted or substituted CH(alkynyl), unsubstituted or substituted CH(alkoxy), unsubstituted or substituted CH(alkylthio), unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles;
   the substituents R4″, R5″ and R6″ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, —S(═O)₂OH, sulfonyl, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), and unsubstituted or substituted NH(heteroaryl), unsubstituted or substituted NR9″R10″, and unsubstituted or substituted C-spirocycles;
   R7″ and R8″ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl) and unsubstituted or substituted NR9″R10″;
   amide″ is selected from the group consisting of NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, unsubstituted or substituted —N(O-alkyl)(alkyl) (Weinreb amide), —N(OH)(H) (hydroxamate), and unsubstituted or substituted —N(OH)(alkyl) (alkylated hydroxamate);
   R9″ und R10″ which may be identical or different, are independently from each other selected from the group consisting of H, OH, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, unsubstituted or substituted cycloalkyl, unsubstituted or substituted aryl, unsubstituted or substituted heterocylcoalkyl, and unsubstituted or substituted heteroaryl;
   Y is selected from the group consisting of H and unsubstituted or substituted alkyl; and
   R is unsubstituted or substituted alkyl;
or a tautomer or a stereoisomer or a salt of the afore-mentioned compounds;
or any mixture of the afore-mentioned compounds; and
(c) optionally at least one film forming agent.

The component (a) of the sunscreen product, cosmetic or pharmaceutical preparation or homecare product according to the first aspect of the present invention relates to at least one primary organic and/or inorganic UV-filter.

UV-filters are well known in the art for their use in stopping radiation. A UV-filter is a compound or a mixture of compounds that block or absorb ultraviolet (UV) light. UV classifications include UVA (320 to 400 nm), UVB (290 to 320 nm) and UVC (200 to 280 nm). UV-absorbing compounds are used not only in sunscreen, but also in other personal care products, such as lipstick, shampoo, hair spray, body wash, toilet soap, and insect repellent. Chemical filters protect against UV-radiation by absorbing, reflecting, or scattering. Reflection and scattering are accomplished by inorganic physical UV-filters, such as titanium dioxide (TiO₂) and zinc oxide (ZnO). Absorption, mainly of UVB, is done by organic UV-filters, which are known as chemical UV-filters.

The sunscreen product, cosmetic or pharmaceutical preparation or homecare product according to the invention advantageously contains at least one UVA filter and/or at least one further UVB filter and/or a broadband filter and/or at least one inorganic pigment, preferably at least one UVA filter and at least one UVB filter for their use in stopping UV radiation.

For example, the at least one primary UV-filter may be one or more organic UV-filters and/or one or more inorganic UV-filters. Non-limiting examples of UV-filters include:
(i) sparingly soluble UV-filters (not appreciably soluble in either water or oil) such as Methylene Bis-benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl-]phenyl];
(ii) oil soluble organic UV-filters (at least partially soluble in oil or organic solvent), such as Bis-Ethylhexyloxyphenol Methoxyphenyl Triazine, Butyl Methoxydibenzoylmethane (BMBM), Oxybenzone, Sulisobenzone, Diethylhexyl Butamido Triazone (DBT), Drometrizole Trisiloxane, Ethylhexyl Methoxycinnamate (EHMC), Ethylhexyl Salicylate (EHS), Ethylhexyl Triazone (EHT), Homosalate, Isoamyl p-Methoxycinnamate, 4-Methylbenzylidene Camphor, Octocrylene (OCR), Polysilicone-15, and Diethylamino Hydroxy Benzoyl Hexyl Benzoate (DHHB);
(iii) inorganic UV-filters such as titanium oxide and zinc oxide, iron oxide, zirconium oxide and cerium oxide; and
(iv) water soluble UV-filters such as Phenylbenzimidazole Sulfonic Acid (PBSA), Sulisobenzone-sodium salt, Benzylidene Camphor Sulfonic Acid, Camphor Benzalkonium Methosulfate, Cinoxate, Disodium Phenyl Dibenzylmidazole Tetrasulfonate, Terephthalylidene Dicamphor Sulfonic Acid, PABA, and PEG-25 PAB; and any mixture thereof.

Many different organic compounds can serve as UV-filters. They fall into several structural classes:
(1) Benzophenones
   a. Benzophenone-3 (BP3)
   b. Benzophenone-4 (BP4)
(2) Salicylates
   a. Homosalate (HMS)
   b. 2-ethylhexyl salicylate (EHS)
(3) p-Aminobenzoic acid and derivatives
   a. Ethylhexyl dimethyl PABA (OD-PABA)
   b. 4-p-aminobenzoic acid (PABA)
(4) Benzimidazole derivatives
   a. Phenylbenzimidazole sulfonic acid (PMDSA)
   b. Disodium phenyl dibenzimidazole tetrasulfonate (bisdisulizole disodium)
(5) Triazines
   a. Ethylhexyltiazone (OT)
   b. Diethylhexyl butamido triazone (DBT)
   c. Bis-ethylhexyloxyphenol methoxyphenyl triazine (EMT)
(6) Benzotriazoles
   a. Drometrizole trisiloxane (DRT)
   b. Methylene bis-benzotriazolyl tetramethylbutylphenol (MBP, bisoctrizole)
(7) Dibenzoylmethane derivatives
   a. 4-tert-Butyl-4′-methoxydibenzoylmethane (BM-DBM, avobenzone)
(8) Cinnamates
   a. Ethylhexyl methoxycinnamate (OMC)
   b. Isoamyl p-methoxycinnamate (IMC, amiloxate)
(9) Camphor derivatives
   a. Terephtalydene dicamphor sulfonic acid (PDSA)
   b. 3-benzylidene camphor (3BC)
   c. Benzylidene camphor sulfonic acid (BCSA)
   d. 4-methylbenzylidene camphor (4-MBC)
   e. Polyacrylamidomethyl benzylidene camphor (PBC)
(10) Camphor benzalkonium methosulfate (CBM)

According to the present invention, the at least one inorganic UV-filter (a) is selected from the group of pigments consisting of titanium dioxide (TiO₂) (amorphous or crystallized in rutile and/or anatase form), zinc oxide (ZnO), iron oxide (Fe₂O₃), zirconium oxide (ZrO₂), silicon dioxide (SiO₂), manganese oxide (e.g. MnO), aluminium oxide (Al₂O₃), cerium oxide (Ce₂O₃), barium carbonate (BaCO₃), calcium carbonate (CaCO₃), and mixtures thereof.

Preferably, the at least one inorganic UV-filter is titanium dioxide, zinc oxide, and mixtures thereof, more preferably the at least one inorganic UV-filter is titanium oxide and/or zinc oxide, and most preferably, the at least one inorganic UV-filter is zinc oxide. ZnO has a broad UVA/UVB absorption curve, while TiO₂ provides better UVB protection.

According to another variant, the at least one inorganic UV-filter is in form of particles having a weight medium particle size d₅₀ from 1 nm to 1000 nm, preferably from 3 nm to 800 nm, more preferably from 5 nm to 600 nm, and most preferably from 10 nm to 400 nm.

The inorganic UV-filters also encompass nano pigments (mean size of the primary particles: generally from 1 nm to 100 nm, preferably from 3 nm to 90 nm, more preferred from 5 nm to 80 nm and most preferred from 10 to 70 nm) of untreated or treated metal oxides such as, for example, nano pigments of titanium dioxide (TiO₂) (amorphous or crystallized in rutile and/or anatase form), zinc oxide (ZnO), iron oxide (Fe₂O₃), zirconium oxide (ZrO₂), silicon dioxide (SiO₂), manganese oxide (e.g. MnO), aluminium oxide (Al₂O₃), cerium oxide (Ce₂O₃), barium carbonate (BaCO₃), calcium carbonate (CaCO₃), or mixtures thereof.

The treated nano pigments and non-nano pigments are pigments that have undergone one or more surface treatments of chemical, electronic, mechanochemical and/or mechanical nature with compounds, such as amino acids, beeswax, fatty acids, fatty acid esters, fatty alcohols, anionic surfactants, lecithins, sodium, potassium, zinc, iron or aluminium salts of fatty acids, metal (titanium or aluminium) alkoxides, polyethylene, silicones, proteins (collagen or elastin), alkanolamines, silicon oxides, metal oxides, sodium hexametaphosphate, alumina or glycerol, hydrated silica, stearic acid, jojoba esters, or glutamic acid derivates,

The treated nano pigments and non-nano pigments may more particularly be titanium oxides treated with silica and alumina, alumina and aluminium stearate, alumina and aluminium laurate, aluminium hydroxide, iron oxides and iron stearate, silica, alumina and silicone, sodium hexametaphosphate, octyltrimethoxysilane, alumina and stearic acid, alumina and glycerol, or alumina and silicone or polyhydroxy stearic acid, hydrated silica, jojoba esters, stearoyl glutamic acid, glutamic acid and derivates, trimethoxycaprylylsilane, glycerin, dimethicone, hydrogen dimethicone, simethicone or combinations thereof. Other titanium oxide nano pigments treated with a silicone are preferably TiO₂ treated with octyltrimethylsilane, preferably for which the mean size of the elementary particles is from 25 to 40 nm; TiO₂ treated with a polydimethylsiloxane, preferably for which the mean size of the elementary particles is 21 nm; or TiO₂ treated with a polydimethylhydrogenosiloxane, preferably for which the mean size of the elementary particles is 25 nm.

The coated zinc oxide nano pigments and zinc oxide non-nano pigments are for example ZnO coated with polymethylhydrogenosiloxane; ZnO dispersions in cyclopolymethylsiloxane/oxyethylenated polydimethylsiloxane, containing 30% or 80% of nano or non-nano zinc oxides coated with silica and polymethylhydrogenosiloxane; ZnO coated with perfluoroalkyl phosphate and copolymer based on perfluoroalkylethyl as a dispersion in cyclopentasiloxane; ZnO coated with dimethoxydiphenylsilanetriethoxycaprylylsilane cross-polymer; ZnO coated with glutamic acid; ZnO coated with octyltriethoxy silane; ZnO coated with dimethicone; ZnO coated with silicone-grafted acrylic polymer, dispersed in cyclodimethylsiloxane; ZnO coated with triethoxycaprylylsilane; ZnO coated with polyhydroxystearic acid; alumina-treated ZnO dispersed in an ethylhexyl methoxycinnamate/PVP-hexadecene/methicone copolymer mixture; ZnO coated with silica and polymethylsilsesquioxane; ZnO dispersed in hydroxystearic acid polycondensate; or ZnO coated with a mixture of stearoyl glutamic acid, jojoba esters, polyhydroxystearic acid, isopropyl titanium triisostearate, hydrogenated olive oil stearyl esters, cetearyl nonanoate, cera alba, dimethicone, dimethoxydiphenylsilanetriethoxycaprylylsilane cross-polymer, octyltriethoxysilane, glutamic acid and sodium stearoyl lactylate.

Also preferred are particulate UV-filters or inorganic pigments, which can optionally be hydrophobed, such as the oxides of iron (Fe₂O₃), zirconium (ZrO₂), silicon (SiO₂), manganese (e.g. MnO), aluminium (Al₂O₃), cerium (e.g. Ce₂O₃), barium carbonate (BaCO₃), calcium carbonate (CaCO₃), or mixtures thereof.

The at least one primary organic and/or inorganic UV-filter is preferably selected from the group consisting of Camphor Benzalkonium Methosulfate, Homosalate, Benzophenone-3, Phenylbenzimidazole Sulfonic Acid, Terephthalylidene Dicamphor Sulfonic Acid, Butyl Methoxydibenzoylmethane, Benzylidene Camphor Sulfonic Acid, Octocrylene, Polyacrylamidomethyl Benzylidene Camphor, Ethylhexyl Methoxycinnamate, PEG-25 PABA, Isoamyl p-Methoxycinnamate, Ethylhexyl Triazone, Drometrizole Trisiloxane, Diethylhexyl Butamido Triazone, 4-Methylbenzylidene Camphor, Ethylhexyl Salicylate, Ethylhexyl Dimethyl PABA, Benzophenone-4, Benzophenone-5, Methylene Bis-Benzotriazolyl Tetramethylbutylphenol (nano), Disodium Phenyl Dibenzimidazole Tetrasulfonate, Bis-Ethylhexyloxyphenol Methoxyphenyl Triazine, Polysilicone-15, Titanium Dioxide, Titanium Dioxide (nano), Diethylamino Hydroxy benzoyl Hexyl Benzoate, Tris-biphenyl triazine (nano), Zinc Oxide, Zinc Oxide (nano), Phenylene Bis-Diphenyltriazine, Methoxypropylamino Cyclohexenylidene Ethoxyethylcyanoacetate, Bis-(Diethylaminohydroxybenzoyl Benzoyl) Piperazine, TEA-Salicylate, Benzophenone-8, PABA, Ethylhexyl Dimethyl PABA, Menthyl Anthranilate, and any mixture thereof.

More preferred, the at least one primary organic and/or inorganic UV-filter is selected from the group consisting of Camphor Benzalkonium Methosulfate, Benzophenone-3, Terephthalylidene Dicamphor Sulfonic Acid, Benzylidene Camphor Sulfonic Acid, Polyacrylamidomethyl Benzylidene Camphor, PEG-25 PABA, Drometrizole Trisiloxane, Ethylhexyl Dimethyl PABA, Benzophenone-4, Benzophenone-5, Methylene Bis-Benzotriazolyl Tetramethylbutylphenol, Polysilicone-15, Titanium Dioxide (nano), Tris-biphenyl triazine (nano), Phenylene Bis-Diphenyltriazine, Methoxypropylamino Cyclohexenylidene Ethoxyethylcyanoacetate, Bis-(Diethylaminohydroxybenzoyl Benzoyl) Piperazine, Benzophenone-8, Ethylhexyl Dimethyl PABA, Homosalate, Phenylbenzimidazole Sulfonic Acid, Butyl Methoxydibenzoylmethane, Octocrylene, Ethylhexyl Methoxycinnamate, Isoamyl p-Methoxycinnamate, Ethylhexyl Triazone, Diethylhexyl Butamido Triazone, 4-Methylbenzylidene Camphor, Ethylhexyl Salicylate, Disodium Phenyl Dibenzimidazole Tetrasulfonate, Bis-Ethylhexyloxyphenol Methoxyphenyl Triazine, Titanium Dioxide, Diethylamino Hydroxy benzoyl Hexyl Benzoate, Zinc Oxide, Zinc Oxide (nano), Menthyl Anthranilate, and any mixture thereof.

Most preferred, the at least one primary organic and/or inorganic UV-filter is selected from the group consisting of Homosalate, Phenylbenzimidazole Sulfonic Acid, Butyl Methoxydibenzoylmethane, Octocrylene, Ethylhexyl Methoxycinnamate, Isoamyl p-Methoxycinnamate, Ethylhexyl Triazone, Diethylhexyl Butamido Triazone, 4-Methylbenzylidene Camphor, Ethylhexyl Salicylate, Disodium Phenyl Dibenzimidazole Tetrasulfonate, Bis-Ethylhexyloxyphenol Methoxyphenyl Triazine, Titanium Dioxide, Diethylamino Hydroxy benzoyl Hexyl Benzoate, Zinc Oxide, Zinc Oxide (nano), Menthyl Anthranilate, and any mixture thereof.

The UV-filters cited before which can be used in the context of the present invention are preferred but naturally are not limiting.

The aforesaid primary organic and/or inorganic UV-filters are used in the sunscreen, cosmetic or pharmaceutical preparation or homecare product either as a single component or preferably in a mixture with two, three, four or more further of said UV-filters as specified above.

In order to optimize the SPF, i.e. to obtain a high SPF in a range of 6 to 100, preferably in a range of 6 to 70, and to cover a broad UVA and UVB range, the sunscreen product, cosmetic or pharmaceutical preparation or homecare product according to the present invention comprises at least two, more preferably at least three, most preferably at least four or even more different primary organic and/or inorganic UV-filters, i.e. in particular organic UV-filters and/or inorganic pigments (UV-filtering pigments). Such compositions according to the invention are particularly suitable for protecting the skin and hair.

Advantageously, the sunscreen product, cosmetic or pharmaceutical preparation or homecare product according to the present invention comprises at least one UVA filter and at least one UVB filter. Such sunscreen products, cosmetic or pharmaceutical preparations or homecare products according to the present invention provide broad spectrum photo protection, i.e. protection from both UVA and UVB.

The combination of effective sun protection UV-filters of different categories such as UVA filter, UVB, broadband filter, inorganic pigments provides reliable protection against the different UV rays in the wavelength range of 290 to 400 nm.

In a further preferred variant the sunscreen product, cosmetic or pharmaceutical preparation or homecare product according to the present invention comprises a combination with one, two, three or more broadband filters which are selected from the group consisting of 2-ethylhexyl-2-cyano-3,3-diphenyl acrylate, ethyl-2-cyano-3,3′-diphenyl acrylate, dihydroxy-4-methoxybenzophenone, 2,4-dihydroxybenzophenone, tetrahydroxy benzophenone, 2,2′-dihydroxy-4,4′-dimethoxybenzophenone, 2-hydroxy-4-n-octoxybenzophenone, 2-hydroxy-4-methoxy-4′-methylbenzophenone, sodium hydroxymethoxy benzophenone sulfonate, disodium-2,2′-dihydroxy-4,4′-dimethoxy-5,5′-disulfobenzophenone, phenol, 2-(2H-benzotriazol-2-yl)-4-methyl-6-(2-methyl-3(1,3,3,3-tetramethyl-1-(trimethylsilyl)oxy)disiloxyanyl) propyl), 2,2′-methylene-bis-(6-(2H-benzotriazol-2-yl)-4-1,1,3,3-tetramethylbutyl)phenol) (methylene-bis-benzotriazolyl tetramethylbutylphenol), 2,4-bis-[4-(2-ethylhexyloxy)-2-hydroxyphenyl]-1,3,5-triazine, 2,4-bis-[{(4-(2-ethylhexyloxy)-2-hydroxy}phenyl]-6-(4-methoxyphenyl)-1,3,5-triazine (INCI: Aniso Triazin), 2,4-bis-[{(4-(3-sulfonato)-2-hydroxypropyloxy)-2-hydroxy}phenyl]-6-(4-methoxyphenyl)-1,3,5-triazine sodium salt, 2,4-bis-[{(3-(2-propyloxy)-2-hydroxypropyloxy)-2-hydroxy}phenyl]-6-(4-methoxyphenyl)-1,3,5-triazine, 2,4-bis-[{(4-(2-ethylhexyloxy)-2-hydroxy}phenyl]-6-[4-(2-methoxyethylcarbonyl) phenylamino]-1,3,5-triazine, 2,4-bis-[{(4-(3-(2-propyloxy)-2-hydroxypropyloxy)-2-hydroxy}phenyl]-6-[4-(2-ethylcarboxyl) phenylamino]-1,3,5-triazine, 2,4-bis-[{(4-(2-ethylhexyloxy)-2-hydroxy}phenyl]-6-(1-methylpyrrol-2-yl)-1,3,5-triazine, 2,4-bis-[{4-tis-(timethylsiloxysilylpropyloxy)-2-hydroxy}phenyl]-6-(4-methoxyphenyl)-1,3,5-trazine, 2,4-bis-[{4-(2″-methylpropenyloxy)-2-hydroxy}phenyl]-6-(4-methoxyphenyl)-1,3,5-triazine, 2,4-bis-[{4-(1′,1′,1′,3′,5′,5′,5′-heptamethylsiloxy-2″-methylpropyloxy)-2-hydroxy}phenyl]-6-(4-methoxyphenyl)-1,3,5-triazine, dioctylbutylamidotriazone (INCI: Dioctylbutamidotriazone), 2,4-bis-[5-1(dimethylpropyl)benzoxazol-2-yl-(4-phenyl)-imino]-6-(2-ethylhexyl)-imino-1,3,5-trazine, 4,4′,4″-(1,3,5-trazine-2,4,6-triyltriimino)tris-benzoic acid-tis(2-ethyl-hexylester) (also: 2,4,6-tris[anilino(p-carbo-2′-ethyl-1′-hexyloxy)]-1,3,5-triazine (INCI: Octyl Triazone), 2,4,6-tribiphenyl-4-yl-1,3,5-triazine, 2-ethylhexyl 4-methoxycinnamate (ethylhexyl methoxycinnamate), benzoic acid, 2-hydroxy-, 3,3,5-trimethylcyclohexyl ester (Homosalate), 2-ethylhexyl salicylate (Ethylhexyl Salicylate), 2-ethylhexyl 4-(dimethylamino)benzoate (Ethylhexyl Dimethyl PABA), N,N,N-Trimethyl-4-(2-oxoborn-3-ylidenemethyl) anilinium methyl sulfate (Camphor Benzalkonium Methosulfate), dimethicodiethylbenzalmalonate (Polysilicone-15), and any mixtures thereof.

The sunscreen product, cosmetic or pharmaceutical preparation or homecare product according to the invention contains the primary sun protection factors, i.e. inorganic and/or organic UV-filters, in a total amount so that the product or preparation according to the invention has a sun protection factor SPF of 6 to 100, preferably 6 to 70. Such compositions according to the invention are particularly suitable for protecting the skin, hair and nails.

The component (b) in the sunscreen product, cosmetic or pharmaceutical preparation or homecare product according to the first aspect of the present invention is at least one mycosporine-like amino acid compound represented either by the general formula (V)

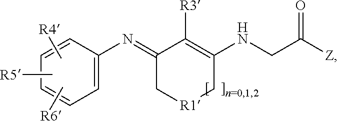

as defined herein;
or
represented by the general formula (VI)

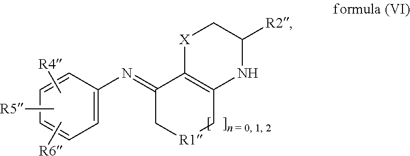

as defined herein.

Mycosporine-like amino acids (MAAs) are small secondary metabolites produced by organisms that live in environments with high volumes of sunlight, usually marine environments. The MAAs are imine derivatives of mycosporines and contain an amino-cyclohexene-imine ring linked to an amino acid, amino alcohol or amino group. The compounds are capable of electron delocalization. In addition, said compounds demonstrate antioxidant qualities.

Unless stated otherwise, in the context of the present invention, especially for the definition of the mycosporine-like amino acid compounds represented by any of the general formulae the following general meanings apply:

The term “halogen” residue/moiety or group alone or as part of another substituent according to the present invention refers to F, Cl, Br or I.

The term “alkyl” alone or as part of another substituent according to the present invention refers to a saturated or mono- or polyunsaturated linear or branched monovalent hydrocarbon radical obtained by removing a hydrogen atom from a single carbon atom of a corresponding parent alkane.

In a preferred variation, the term “alkyl” also includes any alkyl moieties in radicals derived therefrom, such as alkoxy, alkylthio, alkylsulphonyl saturated linear or branched hydrocarbon radicals having 1 to 10, 1 to 8, 1 to 6, or 1 to 4 carbon atoms.

If the alkyl radical is further bonded to another atom, it becomes an alkylene radical or alkylene group. In other words, the term “alkylene” also refers to a divalent linear or branched alkyl. For example, —CH₂CH₃ is an ethyl, while —CH₂CH₂— is an ethylene.

The term “alkylene” alone or as part of another substituent refers to a saturated linear or branched divalent hydrocarbon radical obtained by removing two hydrogen atoms from a single carbon atom or two different carbon atoms of a starting alkane.

In preferred variants according to the present invention, the linear or branched alkyl group or alkylene group comprises 1 to 10 carbon atoms. In other still more preferred variants, the linear or branched alkyl group or alkylene group comprises 1 to 6 carbon atoms.

More preferred according to the invention are saturated linear or branched C1 to C6 alkyl groups or saturated linear or branched C1 to C6 alkylene groups.

Most preferred the linear or branched alkyl groups or alkylene groups with 1 to 4 carbon atoms.

Preferred alkyl radicals/moieties or alkyl groups include, but are not limited to: C1 to C6 alkyl comprising methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

In a preferred variant, the alkyl radical/moiety is selected from the group consisting of methyl, ethyl, propyl, isopropyl, butyl, isobutyl, and tert-butyl, more preferred from the group consisting of methyl and ethyl.

The alkyl group or alkylene group as defined above may further be substituted.

The term “alkyl” or “alkylene” further includes radicals or groups having any degree of saturation, i.e., groups having only single carbon-carbon bonds (“alkyl” or “alkylene”), groups having one or more double carbon-carbon bonds (“alkenyl”), radicals having one or more triple carbon-carbon bonds (“alkynyl”), and groups having a mixture of single, double and/or triple carbon-carbon bonds.

The term “alkenyl” alone or as part of another substituent according to the present invention refers to an unsaturated linear or branched monovalent hydrocarbon radical having at least one carbon-carbon double bond (C═C double bond). The radical may be in either the cis or trans conformation around the double bond(s). So that the term “alkenyl” also includes the corresponding cis/trans isomers.

In preferred variants according to the present invention, the linear or branched alkenyl group comprises 2 to 10 carbon atoms. In other preferred variants, the linear or branched alkenyl group comprises 2 to 6 carbon atoms.

In still further preferred variants, the linear or branched alkenyl group comprises 2 to 4 carbon atoms.

Preferred according to the invention are mono- or di-unsaturated linear or branched C1 to C6 alkenyl groups.

Typical alkenyl radicals or alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl, cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl and the like.

The alkenyl group as defined above may further be substituted.

The term “alkynyl” alone or as part of another substituent according to the present invention refers to an unsaturated linear or branched monovalent hydrocarbon radical having at least one carbon-carbon triple bond (C≡C triple bond).

In preferred variants according to the present invention, the linear or branched alkynyl group comprises 2 to 10 carbon atoms. In other preferred variants, the alkynyl group comprises 2 to 6 carbon atoms. In still further preferred variants, the alkynyl group comprises 2 to 4 carbon atoms.

Most preferred according to the invention are mono- or di-unsaturated linear or branched C1 to C6 alkynyl groups.

Typical alkynyl radicals/moieties or alkynyl groups include, but are not limited to, ethynyl; propynyls such as prop-1-yn-1-yl, prop-2-in-1-yl, etc.; butynyls such as but-1-in-1-yl, but-1-in-3-yl, but-3-in-1-yl, and the like.

The alkynyl group as defined above may further be substituted. The alkyl group or alkylene group as defined above may further be substituted.

The term “alkoxy” alone or as part of another substituent according to the present invention refers to a linear or branched radical of the formula —O—R, where R is alkyl or substituted alkyl, as defined herein.

In preferred variants according to the present invention, the linear or branched alkoxy group comprises 2 to 10 carbon atoms. In other preferred variants, the linear or branched alkoxy group comprises 2 to 6 carbon atoms. In still further preferred variants, the linear or branched alkoxy group comprises 2 to 4 carbon atoms.

Most preferred according to the invention are linear or branched C1 to C6 alkoxy groups.

Typical alkoxy radicals/moieties or alkoxy groups include C1 to C6 alkoxy comprising C1 to C4 alkoxy such as. methoxy, ethoxy, n-propoxy, 1-methylethoxy, butoxy, 1-methylpropoxy, 2-methylpropoxy or 1,1-dimethylethoxy; as well as pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

In a preferred variant, the alkoxy radical or alkoxy group is selected from the group consisting of methoxy (—O-methyl), ethoxy (—O-ethyl), propoxy (—O-propoxy) and butoxy (—O-buty), more preferred from the group consisting of methoxy (—O-methyl) and ethoxy (—O-ethyl).

The alkoxy group or alkylene group as defined above may further be substituted.

The term “alkylthio” or “thioalkoxy” alone or as part of another substituent according to the present invention refers to a radical of the formula —S—R, wherein R is alkyl or substituted alkyl, as defined herein.

According to the invention, the term “alkyl” or “alkylene” also includes heteroalkyl radicals or heteroalkyl groups. The term “heteroalkyl” by itself or as part of other substituents refers to alkyl groups in which one or more of the carbon atom(s) is/are independently replaced by the same or another heteroatom or by the same or another heteroatomic group(s). Typical heteroatoms or heteroatomic groups that may replace the carbon atoms include, but are not limited to, —O—, —S—, —N—, —Si—, —NH—, —S(O)—, —S(O)₂—, —S(O)NH—, —S(O)₂NH—, and the like, and combinations thereof. The heteroatoms or heteroatomic groups may be located at any internal position of the alkyl group.

Typical heteroatomic groups that may be included in these groups include, but are not limited to, —O—, —S—, —O—O—, —S—S—, —O—S—, —NRR—, ═NN═, —N═N—, —N═N—NRR, —PR—, —P(O)₂—, —POR—, —O—P(O)₂—, —SO—, —SO₂—, —SR₂OR—, and the like, wherein R is independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl as defined herein.

The alkyl group or alkylene group as defined above may further be substituted.

The term “acyl” or “alkanoyl” alone or as part of another substituent according to the present invention refers to a radical R—C(═O)—, wherein R is hydrogen, unsubstituted or substituted alkyl, unsubstituted substituted cycloalkyl, unsubstituted or substituted aryl, arylalkyl, unsubstituted or substituted arylalkyl, unsubstituted or substituted heteroalkyl, unsubstituted or substituted heteroarylalkyl, as defined herein.

Representative examples include, but are not limited to, formyl, acetyl, propionyl, butyryl, valeryl, benzoyl, cyclohexylcarbonyl, cyclohexylmethylcarbonyl, benzylcarbonyl, and the like.

The term “cycloalkyl” alone or as part of another substituent according to the present invention refers to a saturated or mono- or polyunsaturated, non-aromatic, cyclic monovalent hydrocarbon radical in which the carbon atoms are ring-linked and which has no heteroatom.

The carbon ring can occur as a monocyclic compound, which has only a single ring, or as a polycyclic compound, which has two or more rings.

In one preferred variation, the term “cycloalkyl” includes a three- to ten-membered monocyclic cycloalkyl radical or cycloalkyl group or a nine- to twelve-membered polycyclic cycloalkyl radical or cycloalkyl group. In other still more preferred variants, the cycloalkyl moiety comprises a five-, six- or seven-membered monocyclic cycloalkyl moiety or a nine- to twelve-membered bicyclic cycloalkyl moiety.

In a preferred embodiment according to the present invention, a cycloalkyl radical or group comprises 3 to 20 carbon atoms. In an even more preferred embodiment, a cycloalkyl radical comprises 6 to 15 carbon atoms. In a most preferred embodiment, a cycloalkyl radical comprises 6 to 10 carbon atoms. Most preferred are monocyclic C3 to C7 cycloalkyl groups.

Typical cycloalkyl radicals or cycloalkyl groups include, but are not limited to, saturated carbocyclic radicals having 3 to 20 carbon atoms, such as C3 to C12 carbocyclyl, comprising cyclopropyl, cyclobutyl, cyclopentyl, cyclohexyl, cycloheptyl, cyclooctyl, cyclononyl, cyclodecyl, cycloundecyl, and cyclododecyl; cyclopentyl, cyclohexyl, cycloheptyl, as well as cyclopropyl-methyl, cyclopropyl-ethyl, cyclobutyl-methyl, cyclobutyl-ethyl, cyclopentyl-methyl, cyclopentyl-ethyl, cyclohexyl-methyl, or C3- to C7-carbocyclyl comprising cyclopropyl, cyclobutyl, cyclopentyl, cyclohexyl, cycloheptyl, cyclopropyl-methyl, cyclopropyl-ethyl, cyclobutyl-methyl, cyclopentyl-ethyl, cyclohexyl-methyl, cyclobut-1-en-1-yl, cyclobut-1-en-3-yl, 3-dien-1-yl and the like.

Polycyclic cycloalkyl radicals or cycloalkyl groups preferred according to the invention include but are not limited to naphthyl, indenyl groups and the like.

According to the invention, the term “cycloalkyl” also includes cycloalkenyls, i.e. unsaturated cyclic hydrocarbon radicals containing C═C double bonds between two carbon atoms of the ring molecule. In a broader sense, cycloalkenyls are compounds with one, two or more double bond(s), where the number of possible, mostly conjugated double bonds in the molecule depends on the ring size.

Typical cycloalkenyls include, but are not limited to, cyclopropenyl, cyclopentenyl, cyclohexenyl, cyclopentadienyl, and the like.

According to the invention, the term “cycloalkyl” further includes cycloalkynyls, i.e. unsaturated, —C≡C-triple bonds, containing cyclic hydrocarbon radicals between two carbon atoms of the ring molecule, the triple bond depending on the ring size for reasons of ring tension.

Typical cycloalkynyles include cyclooctin.

The cycloalkyl, cycloalkenyl or cycloalkynyl moieties or groups, as defined above, may further be substituted.

The term “aryl” alone or as part of another substituent according to the present invention refers to a monovalent aromatic hydrocarbon radical derived by removing a hydrogen atom from a single carbon atom of an aromatic ring SY-stem.

In one preferred variation, the term “aryl” includes a three- to ten-membered monocyclic aryl radical or aryl group or a nine- to twelve-membered polycyclic aryl radical or aryl group. In other still more preferred variants, the carboaryl radical comprises a five-, six-, or seven-membered monocyclic carboaryl radical or a nine- to twelve-membered bicyclic carboaryl radical.

In a preferred embodiment according to the present invention, the aryl radical comprises 3 to 20 carbon atoms. In a preferred variation, the aryl moiety comprises 6 to 15 ring atoms. In an even more preferred embodiment, an aryl radical comprises 6 to 10 carbon atoms.

Most preferred according to the invention are monocyclic C3 to C10 aryl groups.

Most preferred are monocyclic C3 to C7 aryl groups.

Typical aryl radicals include, without being limited thereto, benzene, phenyl, biphenyl, naphthyl such as 1- or 2-naphthyl, tetrahydronaphthyl, fluorenyl, indenyl, and phenanthrenyl. Typical carboaryl moieties further include, but are not limited to, groups derived from aceanthrylene, acenaphthylene, acephenanthrylene, anthracene, azulene, benzene, chrysene, corone, fluoranthene, fluorene, hexacene, hexaphene, hexalene, as-indacene, S-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

Aromatic polycyclic aryl radicals or aryl groups preferred according to the invention include, but are not limited to, naphthalene, biphenyl and the like.

The aryl moiety or group, as defined above, may further be substituted.

The term “arylalkyl” alone or as part of another substituent according to the present invention refers to an acyclic alkyl group in which one of the hydrogen atoms attached to a carbon atom, typically a terminal or sp carbon atom, is replaced by an aryl group as defined herein. In other words, arylalkyl may also be considered as alkyl substituted by aryl. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like.

The term “heteroarylalkyl” alone or as part of another substituent refers to a cyclic alkyl group in which one of the hydrogen atoms attached to a carbon atom is replaced by a heteroaryl group. In a preferred embodiment according to the present invention, the heteroarylalkyl group is a 6- to 20-membered heteroarylalkyl, e.g., the alkyl, alkenyl, or alkynyl group of the heteroarylalkyl is a C1- to C6-alkyl and the heteroaryl group is a 5- to 15-membered heteroaryl group. In other embodiments, the heteroarylalkyl is a 6- to 13-membered heteroarylalkyl, e.g., the alkyl, alkenyl, or alkynyl group is a C1- to C3-alkyl and the heteroaryl group is a 5 to 10-membered heteroaryl.

The term “heterocycloalkyl” alone or as part of another substituent according to the present invention refers to a saturated or mono- or polyunsaturated, non-aromatic, cyclic monovalent hydrocarbon radical in which one or more carbon atom(s) is/are independently replaced by the same or a different heteroatom. Typical heteroatoms to replace the carbon atom(s) include, but are not limited to, N, P, O, S, Si, etc. Typical heterocycloalkyl groups include, without being limited thereto, groups derived from epoxides, azirines, thiiranes, imidazolidine, morpholine, piperazine, piperidine, pyrazolidine, pyrrolidone, quinuclidine and the like.

In a preferred embodiment according to the present invention, the heterocycloalkyl moiety or group comprises 3 to 20 ring atoms. In a preferred variation, the heterocycloalkyl moiety comprises 6 to 15 ring atoms. In an even more preferred embodiment, the heterocycloalkyl moiety comprises 6 to 10 carbon atoms.

The heterocycloalkyl moiety can occur as a monocyclic compound, which has only a single ring, or as a polycyclic compound, which has two or more rings, such as bicyclic, tricyclic or spirocyclic.

Preferably, the term “heterocycloalkyl” includes three- to seven-membered, saturated or mono- or polyunsaturated heterocycloalkyl moieties comprising one, two, three or four heteroatoms selected from the group consisting of O, N and S. The heteroatom or heteroatoms may occupy any position in the heterocycloalkyl ring.

In one preferred variation, the term “heterocycloalkyl” includes a three- to ten-membered monocyclic heterocycloalkyl radical or a nine- to twelve-membered polycyclic heterocycloalkyl radical. In other still more preferred variants, the heterocycloalkyl moiety comprises a five-, six- or seven-membered monocyclic heterocycloalkyl moiety or a nine- to twelve-membered bicyclic heterocycloalkyl moiety.

Most preferred according to the invention are monocyclic heterocycloalkyl radicals comprising 3 to 12 carbon atoms. Most preferred are monocyclic heterocycloalkyl radicals having 5 to 7 ring atoms.

Typical heterocycloalkyl moieties include, but are not limited to: Five- or six-membered, saturated or monounsaturated heterocycloalkyl containing one or two nitrogen atoms and/or one oxygen or sulphur atom or one or two oxygen and/or sulphur atoms as ring members comprising 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-Isoxazolidinyl, 4-Isoxazolidinyl, 5-Isoxazolidinyl, 3-Isothiazolidinyl, 4-Isothiazolidinyl, 5-Isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-Imidazolidinyl, 4-Imidazolidinyl, 2-pyrrolin-2-yl, 2-pyrrolin-3-yl, 3-pyrrolin-2-yl, 3-pyrrolin-3-yl, 1-piperidinyl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl and the like.

The heterocycloalkyl moiety or group, as defined above, may further be substituted.

The term “heteroaryl” by itself or as part of another substituent according to the present invention refers to a monovalent heteroaromatic radical obtained by removing a hydrogen atom from a single atom of a heteroaromatic ring SY-stem. Typical heteroaryl radicals, or. Heteroaryl groups include, but are not limited to, those derived from acridine, P-carboline, chroman, chromium, cinnoline, furan, imidazole, indazole, indole, indoline, indolizine, isobenzofuran, isochromium, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, thiazole, thiophene, triazole, xanthene and the like are derived.

The heteroaryl moiety can occur as a monocyclic compound having only a single ring or as a polycyclic compound having two or more rings.

In one preferred variation, the term “heteroaryl” includes a three- to ten-membered monocyclic heteroaryl radical or a nine- to twelve-membered polycyclic heteroaryl radical. In other still more preferred variants, the heteroaryl moiety comprises a five-, six- or seven-membered monocyclic heteroaryl moiety or a nine- to twelve-membered bicyclic heteroaryl moiety.

Preferably, the term “heteroaryl” includes three- to seven-membered monocyclic heteroaryl radicals comprising one, two, three or four heteroatoms selected from the group consisting of O, N and S. The heteroatom or heteroatoms may occupy any position in the heteroaryl ring.

In a preferred embodiment according to the present invention, the heteroaryl moiety or group comprises 3 to 20 ring atoms. In an even more preferred variant, the heteroaryl moiety comprises 6 to 15 ring atoms. In a most preferred embodiment, the heteroaryl group comprises 6 to 10 ring atoms. Most preferred according to the invention are monocyclic C3 to C7 heteroaryl groups.

Particularly preferred heteroaryl moieties or heteroaryl groups include, but are not limited to, those derived from furan, thiophene, pyrrole, benzothiophene, benzofuran, benzimidazole, indole, pyridine, pyrazole, quinoline, imidazole, oxazole, isoxazole, and pyrazine.

Five-membered aromatic heteroaryl radicals containing, in addition to carbon atoms, one, two or three nitrogen atoms or one or two nitrogen atoms and one sulfur or oxygen atom as ring atoms include 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-Imidazolyl, 4-Imidazolyl, and 1,3,4-triazol-2-yl.

Five-membered aromatic heteroaryl radicals containing one, two, three or four nitrogen atoms as ring atoms include 1-, 2- or 3-pyrrolyl, 1-, 3- or 4-pyrazolyl, 1-, 2- or 4-Imidazolyl, 1,2,3-[1H]-triazol-1-yl, 1,2,3-[2H]-triazol-2-yl, 1,2,3-[1H]-triazol-4-yl, 1,2,3-[1H]-triazol-5-yl, 1,2,3-[2H]-triazol-4-yl, 1,2,4-[1H]-triazol-1-yl, 1,2,4-[1H]-triazol-3-yl, 1,2,4-[1H]-triazol-5-yl, 1,2,4-[4H]-triazol-4-yl, 1,2,4-[4H]-triazol-3-yl, [1H]-tetrazol-1-yl, [1H]-tetrazol-5-yl, [2H]-tetrazol-2-yl, [2H]-tetrazol-5-yl and the like.

Five-membered aromatic heteroaryl radicals containing a heteroatom selected from oxygen or sulphur and optionally one, two or three nitrogen atoms as ring atoms include 2-furyl, 3-furyl, 2-thienyl, 3-thienyl, 3- or 4-Isoxazolyl, 3- or 4-isothiazolyl, 2-, 4- or 5-oxazolyl, 2-, 4- or 5-thiazolyl, 1,2,4-thiadiazol-3-yl, 1,2,4-thiadiazol-5-yl, 1,3,4-thiadiazol-2-yl, 1,2,4-oxadiazol-3-yl, 1,2,4-oxadiazol-5-yl and 1,3,4-oxadiazol-2-yl.

Six-membered heteroaryl radicals containing, in addition to carbon atoms, one or two or one, two or three nitrogen atoms as ring atoms, and comprising, for example. 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,2,4-triazin-3-yl; 1,2,4-triazin-5-yl, 1,2,4-triazin-6-yl and 1,3,5-triazin-2-yl.

The heteroaryl moiety or group, as defined above, may further be substituted.

The term “C-spirocycles” in the context of the present application means compounds that have at least two molecular rings with only one common atom. The simplest spiro compounds are bicyclic (having just two rings) or have a bicyclic portion as part of the larger ring system, in either case with the two rings connected through the defining single common atom. The one common atom connecting the participating rings distinguishes spiro compounds from other bicyclic structures: from isolated ring compounds like biphenyl that have no connecting atoms, from fused ring compounds like decalin having two rings linked by two adjacent atoms, and from bridged ring compounds like norbornane with two rings linked by two non-adjacent atoms. Spiro compounds may be fully carbocyclic (all carbon) or heterocyclic (having one or more non-carbon atom, such as N, O and S). The common atom that connects the two (or sometimes three) rings is called the spiro atom. Preferably, the spiro atom is a carbon atom. More preferably, the C-spirocycles compound means compounds that are fully carbocyclic (all carbon).

The term “substituted” in the context of the present invention means that one or more hydrogen atoms of the indicated radical or group is/are independently replaced by the same or a different substituent(s). Additionally, the term “substituted” specifically provides for one or more, i.e., two, three, four, five, six or more, substitutions commonly used in the art. However, it is generally known that the substituents should be selected so that they do not adversely affect the useful properties of the compound or its function.

Suitable substituents in the context of the present invention preferably include halogen, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy groups or heteroaryloxy groups, arylalkyl or heteroarylalkyl groups, arylalkoxy or heteroarylalkoxy groups, amino groups, alkyl and dialkylamino groups, carbamoyl groups, alkylcarbonyl groups, carboxyl groups, alkoxycarbonyl groups, alkylaminocarbonyl groups, dialkylaminocarbonyl groups, arylcarbonyl groups, aryloxycarbonyl groups, alkylsulfonyl groups, arylsulfonyl groups, cycloalkyl groups, cyano groups, C1 to C6 alkylthio groups, arylthio groups, nitro groups, keto groups, acyl groups, boronate or boranyl groups, phosphate or phosphonyl groups, sulfamyl groups, sulfonyl groups, sulfinyl groups, and combinations thereof.

Substituents or substituent groups useful for substituting saturated carbon atoms in the indicated group or radical more preferably include, but are not limited to, halogen, hydroxyl, alkyl, alkenyl, alkynyl, alkoxyl, —NH₂, amino (primary, secondary, or tertiary), nitro, thiol, thioether, imine, cyano, amido, phosphonato, phosphine, carboxyl, thiocarbonyl, sulfonyl, sulfonamide, ketone, aldehyde, ester, acetyl, acetoxy, carbamoyl, oxygen (O); haloalkyl (e.g., trifluoromethyl); aminoacyl and aminoalkyl, carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g. pyrrolidinyl, piperidinyl, piperazinyl, morpholinyl, or thiazinyl), carbocyclic or heterocyclic, monocyclic or fused or non-fused polycyclic aryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl), —CO₂CH₃, —CONH₂, —OCH₂CONH₂; —SO₂NH₂, —OCHF₂, —CF₃, —OCF₃.

According to the invention, the substituents used to replace a particular radical or radical may in turn be further substituted, typically with one or more of the same or different radicals selected from the various groups indicated above and as defined in detail above. In the case of substituted combinations such as substituted arylalkyl, either the aryl or the alkyl group may be substituted, or both the aryl and the alkyl group may be substituted with one or more substituents.

In a preferred variant, Z in the general formula (V) is either —O—R2′ or amide′.

In a further preferred variant, R1′ in the general formula (V) is CH₂.

In a further preferred variant, R1′ in the general formula (V) is C(alkyl)₂.

In a further preferred variant, R1′ in the general formula (V) is O.

In a further preferred variant, R1′ in the general formula (V) is S.

In a further preferred variant, R1′ in the general formula (V) is SO.

In a further preferred variant, R1′ in the general formula (V) is SO₂.

In a further preferred variant, R1′ in the general formula (V) is NH.

In a still further preferred variant, R1′ in the general formula (V) is N(alkyl).

Most preferred the R1′ in the general formula (V) is either CH₂ or C(alkyl)₂.

In a preferred variant, R2′ in the general formula (V) is H.

In a further preferred variant, R2′ in the general formula (V) is methyl.

In a further preferred variant, R2′ in the general formula (V) is ethyl.

In a further preferred variant, R2′ in the general formula (V) is propyl.

In a further preferred variant, R2′ in the general formula (V) is isopropyl.

In a further preferred variant, R2′ in the general formula (V) is butyl.

In a further preferred variant, R2′ in the general formula (V) is isobutyl.

In a further preferred variant, R2′ in the general formula (V) is tert-butyl.

In a further preferred variant, R2′ in the general formula (V) is

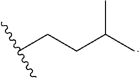

In a further preferred variant, R2′ in the general formula (V) is

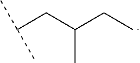

In a preferred variant, R2′ in the general formula (V) is

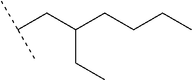

(2-ethyl-hexyl).

In a still further preferred variant, R2′ in the general formula (V) is phenyl.

Most preferred the R2′ in the general formula (V) is either H or ethyl or isopropyl or 2-ethylhexyl or phenyl.

In a preferred variant, amide′ in the general formula (V) is NH₂.

In a further preferred variant, amide′ in the general formula (V) is N(Halkyl), wherein alkyl is selected from the group consisting of methyl, ethyl, propyl, isopropyl, butyl, isobutyl, and tert-butyl.

In a further preferred variant, amide′ in the general formula (V) is N(alkyl)₂, wherein alkyl is selected from the group consisting of methyl, ethyl, propyl, isopropyl, butyl, isobutyl, and tert-butyl.

In a further preferred variant, amide′ in the general formula (V) is —N(O-alkyl)(alkyl) (Weinreb amide), wherein alkyl is selected from the group consisting of methyl, ethyl, propyl, isopropyl, butyl, isobutyl, and tert-butyl.

In a still further preferred variant, amide′ in the general formula (V) is —N(OH)(H) (hydroxamate).

Most preferred the amide′ in the general formula (V) is NH₂, N(methyl)₂ or N(ethyl)₂ or —N(O-alkyl)(alkyl) (Weinreb amide), wherein alkyl is selected from the group consisting of methyl, ethyl, propyl, isopropyl, butyl, isobutyl, and tert-butyl, or —N(OH)(H) (hydroxamate).

In a preferred variant, R3′ in the general formula (V) is H.

In a further preferred variant, R3′ in the general formula (V) is methyl.

In a further preferred variant, R3′ in the general formula (V) is ethyl.

In a further preferred variant, R3′ in the general formula (V) is —O-methyl.

In a further preferred variant, R3′ in the general formula (V) is —C(═O)—OH.

In a further preferred variant, R3′ in the general formula (V) is —C(═O)—CH₃.

In a further preferred variant, R3′ in the general formula (V) is —C(═O)—C₃H₇.

In a still further preferred variant, R3′ in the general formula (V) is —C(═O)—O-methyl.

Most preferred the R3′ in the general formula (V) is either H or methyl or —O-methyl.

In a preferred variant according to the first aspect of the present invention, the mycosporine-like amino acid compound (b) of the general formula (V) is either an acid compound or an ester compound represented by the general formula (VII)

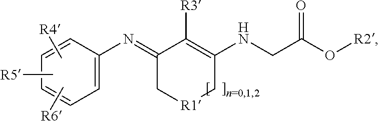

wherein R1′, R2′, R3′, R4′, R5′ and R6′ have the same meaning as defined herein for the general formula (V).

In a preferred variant according to the first aspect of the present invention, the mycosporine-like amino acid compound (b) of the general formula (V) is an acid compound represented by the general formula (VII-acid)

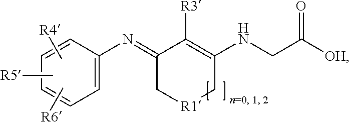

wherein R1′, R3′, R4′, R5′ and R6′ have the same meaning as defined herein for the general formula (V).

The mycosporine-like amino acid compounds are particularly photo-stable and temperature-stable compounds and show stability in pH solution or in different emulsion types and do not degrade.

Especially, the mycosporine-like amino acid compounds having an acid group have a better pH stability in a broader pH-range compared to their respective ester compounds which hydrolize, and, thus, are not stable in alkaline solutions having a pH higher than 9.

In addition, said mycosporine-like amino acid compounds have also a better solubility in water compared to their corresponding ester compounds. Due to their better solubility in water, said mycosporine-like amino acid compounds can be incorporated into the water-phase of emulsions in higher concentrations, resulting in an improved boosting effect for film forming agents.

In a preferred variant according to the first aspect of the present invention, the mycosporine-like amino acid compound (b) of the general formula (V) is an ester compound represented by the general formula (VII-ester)

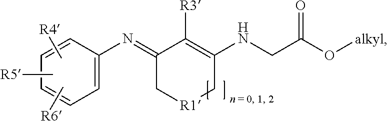

wherein R1′, R3′, R4′, R5′ and R6′ have the same meaning as defined herein for the general formula (V).

Preferably, the alkyl residue in the general formula (VII-ester) is methyl or ethyl or propyl or isopropyl or butyl or isobutyl or tert-butyl. Most preferred the alkyl residue in the general formula (VII-ester) is methyl. Such ester compounds are particularly stable.

In an alternative preferred variant according to the first aspect of the present invention, the mycosporine-like amino acid compound (b) of the general formula (V) is an amide compound represented by the general formula (VIII)

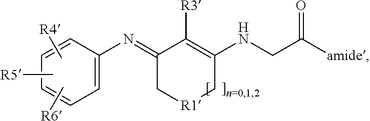

wherein R1′, amide′, R3′, R4′, R5′ and R6′ have the same meaning as defined herein for the general formula (V).

In a more preferred variant according to the first aspect of the present invention, the mycosporine-like amino acid compound (b) of the general formula (V) is an amide compound represented by the general formula (VIII-amide)

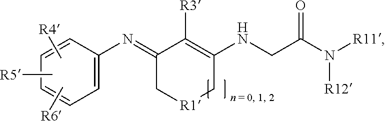

wherein R1′, R3′, R4′, R5′ and R6′ have the same meaning as defined herein for the general formula (V) and R11′ and R12′ are independently from each other selected from the group consisting of H, OH and alkyl.

In a preferred variant according to the first aspect of the present invention, in the general formula (VIII-amide), R11′ and/or R12′ are H.

In a further preferred variant, the R11′ alkyl and R12′ alkyl in the general formula (VIII-amide) are independently from each other selected from the group consisting of methyl, ethyl, propyl, isopropyl, butyl, isobutyl, and tert-butyl.

Preferably, R11′ and/or R12′ in the general formula (VIII-amide) are each H or each methyl.

In a still more preferred variant, the mycosporine-like amino acid compound (b) of the general formula (V) is a Weinreb amide compound represented by the general formula (VIII-Weinreb amide)

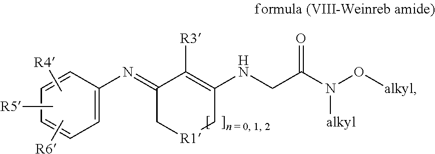

wherein R1′, R3′, R4′, R5′ and R6′ have the same meaning as defined herein for the general formula (V).

In a preferred variant, the alkyl residue in the general formula (VIII-Weinreb amide) is selected from the group consisting of methyl, ethyl, propyl, isopropyl, butyl, isobutyl, and tert-butyl, more preferred the alkyl residue is methyl.

Alternatively, in a still more preferred variant, the mycosporine-like amino acid compound (b) of the general formula (V) is a hydroxamate compound represented by the general formula (VIII-hydroxamate derivative)

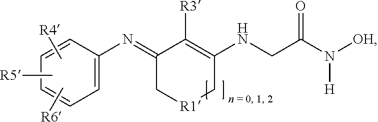

wherein R1′, R3′, R4′, R5′ and R6′ have the same meaning as defined herein for the general formula (V).

Alternatively, in a still more preferred variant, the mycosporine-like amino acid compound (b) of the general formula (V) is an alkylated hydroxamate compound represented by the general formula (VIII-alkylated hydroxamate derivative)

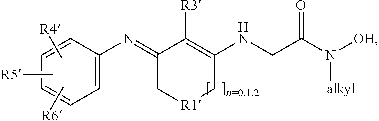

wherein R1′, R3′, R4′, R5′ and R6′ have the same meaning as defined herein for the general formula (V).

In a preferred variant, the alkyl residue in the general formula (VIII-alkylated hydroxamate derivative) is selected from the group consisting of methyl, ethyl, propyl, isopropyl, butyl, isobutyl, and tert-butyl, more preferred the alkyl is methyl.

In a preferred alternatively variant the mycosporine-like amino acid compound (b) is represented by the general formula (A)

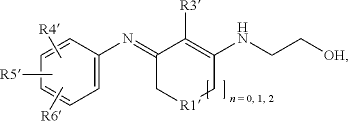

wherein R1′, R3′, R4′, R5′ and R6′ have the same meaning as defined herein for the general formula (V).

Within the present invention, the same preferred variants as defined herein for the mycosporine-like amino acid compounds according to the general formula (V) apply analogously for the mycosporine-like amino acid compounds according to the general formulae (VII) or (Vill) and their variants or general formula (A).

In a further preferred variant, R1″ in the general formula (VI) is CH₂.

In a further preferred variant, R1″ in the general formula (VI) is C(alkyl)₂.

In a further preferred variant, R1″ in the general formula (VI) is O.

In a further preferred variant, R1″ in the general formula (VI) is S.

In a further preferred variant, R1″ in the general formula (VI) is SO.

In a further preferred variant, R1″ in the general formula (VI) is SO₂.

In a further preferred variant, R1″ in the general formula (VI) is NH.

In a still further preferred variant, R1″ in the general formula (VI) is N(alkyl).

Most preferred the R1″ in the general formula (VI) is either CH₂ or C(alkyl)₂.

In a preferred variant, R2″ in the general formula (VI) is —CH₂—OH.

In a further preferred variant, R2″ in the general formula (VI) is —C(═O)—O—Y.

In a further preferred variant, R2″ in the general formula (VI) is —C(═O)-amide″.

Most preferred the R2″ in the general formula (VI) is —C(═O)—O—Y or —C(═O)-amide″.

In a further preferred variant, X in the general formula (VI) is CH₂.

In a further preferred variant, X in the general formula (VI) is C═O.

In a further preferred variant, X in the general formula (VI) is O.

In a further preferred variant, X in the general formula (VI) is S.

In a further preferred variant, X in the general formula (VI) is SO.

In a further preferred variant, X in the general formula (VI) is SO₂.

In a further preferred variant, X in the general formula (VI) is NH.

In a still further preferred variant, X in the general formula (VI) is N(alkyl), wherein alkyl is selected from the group consisting of methyl, ethyl, propyl, isopropyl, butyl, isobutyl, and tert-butyl.

Most preferred the X in the general formula (VI) is either S, SO or SO₂.

In a preferred variant, Y in the general formula (VI) is H.

In a further preferred variant, Y in the general formula (VI) is methyl.

In a further preferred variant, Y in the general formula (VI) is ethyl.

In a further preferred variant, Y in the general formula (VI) is propyl.

In a further preferred variant, Y in the general formula (VI) is isopropyl.

In a further preferred variant, Y in the general formula (VI) is butyl.

In a further preferred variant, Y in the general formula (VI) is isobutyl.

In a further preferred variant, Y in the general formula (VI) is tert-butyl.

In a further preferred variant, Y in the general formula (VI) is

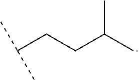

In a further preferred variant, Y in the general formula (VI) is

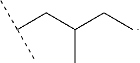

In a preferred variant, Y in the general formula (VI) is

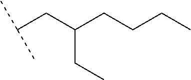

(2-ethyl-hexyl).

In a still further preferred variant, Y in the general formula (VI) is phenyl.

Most preferred the Y in the general formula (VI) is H or ethyl or isopropyl or 2-ethylhexyl or phenyl.

In a preferred variant, amide″ in the general formula (VI) is NH₂.

In a further preferred variant, amide″ in the general formula (VI) is N(Halkyl), wherein alkyl is selected from the group consisting of methyl, ethyl, propyl, isopropyl, butyl, isobutyl, and tert-butyl.

In a further preferred variant, amide″ in the general formula (VI) is N(alkyl)₂, wherein alkyl is selected from the group consisting of methyl, ethyl, propyl, isopropyl, butyl, isobutyl, and tert-butyl.

In a further preferred variant, amide″ in the general formula (VI) is —N(O-alkyl)(alkyl) (Weinreb amide), wherein alkyl is selected from the group consisting of methyl, ethyl, propyl, isopropyl, butyl, isobutyl, and tert-butyl.

In a still further preferred variant, amide″ in the general formula (V) is —N(OH)(H) (hydroxamate).

Most preferred the amide″ in the general formula (V) is NH₂, N(methyl)₂ or N(ethyl)₂ or —N(O-alkyl)(alkyl) (Weinreb amide), wherein alkyl is selected from the group consisting of methyl, ethyl, propyl, isopropyl, butyl, isobutyl, and tert-butyl, or —N(OH)(H) (hydroxamate).

In a more preferred variant according to the first aspect of the present invention, the mycosporine-like amino acid compound (b) is an alcohol compound represented by the general formula (IX-alcohol)

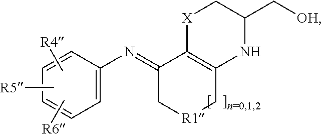

wherein R1″, X, R4″, R5″ and R6″ have the same meaning as defined herein for the general formula (VI).

Alternatively, in a more preferred variant according to the first aspect of the present invention, the mycosporine-like amino acid compound (b) of the general formula (VI) is an acid compound represented by the general formula (IX-acid)

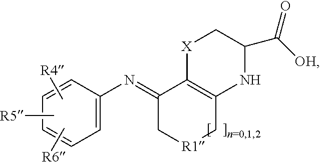

wherein R1″, X, R4″, R5″ and R6″ have the same meaning as defined herein for the general formula (VI).

Alternatively, in a more preferred variant according to the first aspect of the present invention, the mycosporine-like amino acid compound (b) of the general formula (VI) is an ester compound represented by the general formula (VII-ester)

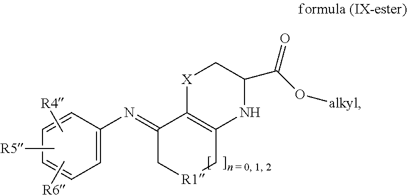

wherein R1″, X, R4″, R5″ and R6″ have the same meaning as defined herein for the general formula (VI).

Preferably, the alkyl residue in the general formula (VII-ester) is methyl or ethyl or propyl or isobutyl or butyl or isobutyl or tert-butyl, more preferred the alkyl residue is methyl.

In an alternative more preferred variant according to the first aspect of the present invention, the mycosporine-like amino acid compound (b) of the general formula VI) is an amide compound represented by the general formula (X)

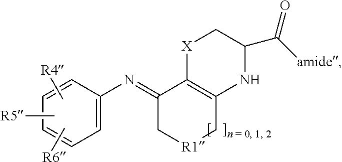

wherein R1″, amide″, X, R4″, R5″ and R6″ have the same meaning as defined herein for the general formula (VI).

In a more preferred alternatively variant according to the first aspect of the present invention, the mycosporine-like amino acid compound (b) of the general formula (VI) is an amide compound represented by the general formula (X-amide),

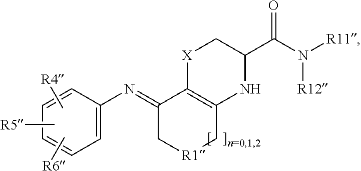

wherein R1″, X, R4″, R5″ and R6″ have the same meaning as defined herein for the general formula (VI) and R11″ and R12″ are independently from each other selected from the group consisting of H, OH and alkyl.

In a preferred variant according to the first aspect of the present invention, in the general formula (X-amide), R11″ and/or R12″ are H.

Preferably, the R11″ alkyl and R12″ alkyl in the general formula (X-amide) is methyl or ethyl or propyl or isopropyl or butyl or isobutyl or tert-butyl.

Preferably, R11″ and R12″ in the general formula (X-amide) are each H or each methyl.

In a still more preferred variant, the mycosporine-like amino acid compound (b) of the general formula (VI) is a Weinreb amide compound represented by the general formula (X-Weinreb amide)

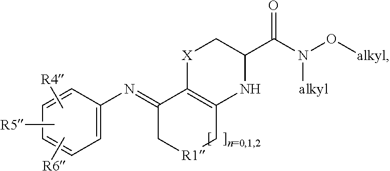

wherein R1″, X, R4″, R5″ and R6″ have the same meaning as defined herein for the general formula (VI).

In a preferred variant, the alkyl residue in the general formula (X-Weinreb amide) is selected from the group consisting of methyl, ethyl, propyl, isopropyl, butyl, isobutyl, and tert-butyl, more preferred the alkyl residue is methyl.

Alternatively, in a still more preferred variant, the mycosporine-like amino acid compound (b) of the general formula (VI) is a hydroxamate compound represented by the general formula (X-hydroxamate derivative)

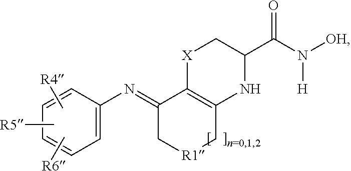

wherein R1″, X, R4″, R5″ and R6″ have the same meaning as defined above for the general formula (VI).

Alternatively, in a still further preferred variant, the mycosporine-like amino acid compound (b) of the general formula (VI) is an alkylated hydroxamate compound represented by the general formula (VIII-alkylated hydroxamate derivative)

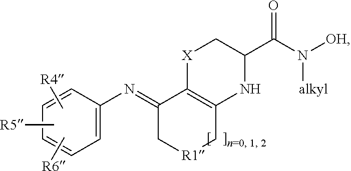

wherein R1″, X, R4″, R5″ and R6″ have the same meaning as defined herein for the general formula (VI).

In a preferred variant, the alkyl residue in the general formula (X-alkylated hydroxamate derivative) is selected from the group consisting of methyl, ethyl, propyl, isopropyl, butyl, isobutyl, and tert-butyl, more preferred the alkyl residue is methyl.

Within the present invention, the same preferred variants as defined herein for the mycosporine-like amino acid compounds according to general formula (VI) apply analogously for the mycosporine-like amino acid compounds according to the general formulae (IX) or (X) and their variants.

In the following, the term “according to the general formulae YZ and their variants” includes any sub-formula(e) of said general formulae as disclosed herein. Accordingly, variants of the general formulae (V) and (VI) as defined herein, include formula (VII), formula (VIII), formula (IX) and formula (X) as defined herein. Variants of the general formulae (VII) and (VIII) as defined herein, include sub-formula (VII-acid), sub-formula (VII-ester), sub-formula (VIII-amide), sub-formula (VIII-Weinreb amide), sub-formula (VIII-hydroxamate derivative) and sub-formula (VIII-alkylated hydroxamate derivative) as defined herein. Variants of the general formulae (IX) and (X) as defined herein, include formula (IX-acid), formula (IX-ester), formula (X-amide), formula (X-Weinreb amide), formula (X-hydroxamate derivative) and formula (X-alkylated hydroxamate derivative) as defined herein.

In a further preferred variant, in the general formulae (VI), (IX), (X) and their variants, X is selected from the group consisting of selected from the group consisting of CH₂, unsubstituted or substituted CH(alkyl), unsubstituted or substituted C(alkyl)₂, unsubstituted or substituted CH(alkenyl), unsubstituted or substituted CH(alkynyl), unsubstituted or substituted CH(alkoxy), unsubstituted or substituted CH(alkylthio), unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles.

In a further preferred variant according to the first aspect of the present invention, the mycosporine-like amino acid compound is a compound according to general formulae (V), wherein
Z is —O—R2′ or amide′; and/or
R1′ is selected from the group consisting of CH₂, C(alkyl)₂, O, S, SO, SO₂, NH and N(alkyl); and/or
R2′ is selected from the group consisting of H, methyl, ethyl, propyl, isopropyl, butyl, isobutyl, tert-butyl,

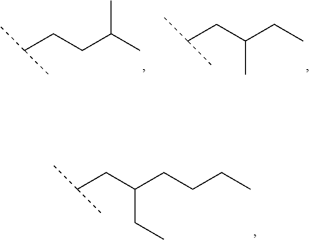

(2-ethyl-hexyl) and phenyl; or
amide′ is selected from the group consisting of NH₂, NH(alkyl), N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In another preferred variant according to the first aspect of the present invention, the mycosporine-like amino acid compound is a compound according to general formulae (VII) or (VIII), wherein
R1′ is selected from the group consisting of CH₂, C(alkyl)₂, O, S, SO, SO₂, NH and N(alkyl); and/or
R2′ is selected from the group consisting of H, methyl, ethyl, propyl, isopropyl, butyl, isobutyl, tert-butyl,

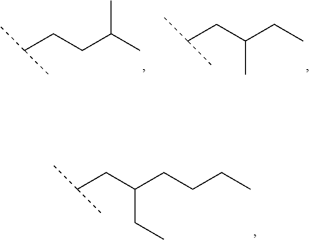

(2-ethyl-hexyl) and phenyl; or
amide′ is selected from the group consisting of NH₂, N(Halkyl), N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In another preferred variant according to the first aspect of the present invention, the mycosporine-like amino acid compound is a compound according to general formula (VI), wherein
R1″ is selected from the group consisting of CH₂, C(alkyl)₂, O, S, SO, SO₂, NH and N(alkyl); and/or
R2″ is selected from the group consisting of —CH₂—OH, —C(═O)—O—Y, and —C(═O)-amide″; and/or
X is selected from the group consisting of CH₂, C═O, O, S, SO, SO₂, NH, and N(alkyl); and/or
Y is selected from the group consisting of H, methyl, ethyl, propyl, isopropyl, butyl, isobutyl, tert-butyl,

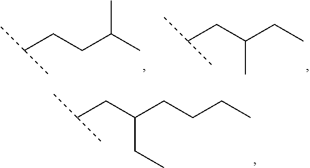

(2-ethyl-hexyl) and phenyl; and/or
amide″ is selected from the group consisting of NH₂, NH(alkyl), N(alkyl)₂, —N(O-alkyl)(alkyl) (Weinreb amide) and —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and tert-butyl.

In a further preferred variant according to the first aspect of the present invention, the mycosporine-like amino acid compound is a compound according to general formulae (V), wherein
Z is —O—R2′ or amide′; and/or
R1′ is selected from the group consisting of CH₂ and C(methyl)₂; and/or
R2′ is selected from the group consisting of H, methyl, ethyl, propyl, isopropyl, butyl, isobutyl, tert-butyl,

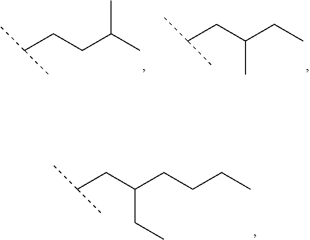

(2-ethyl-hexyl) and phenyl; or
amide′ is selected from the group consisting of NH₂, NH(alkyl), N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In another preferred variant according to the first aspect of the present invention, the mycosporine-like amino acid compound is a compound according to general formulae (VII) or (VIII), wherein
R1′ is selected from the group consisting of CH₂ and C(methyl)₂; and/or
R2′ is selected from the group consisting of H, methyl, ethyl, propyl, isopropyl, butyl, isobutyl, tert-butyl,

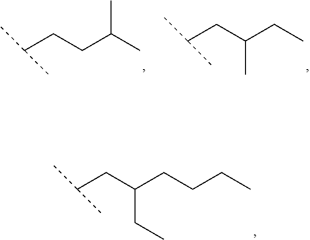

(2-ethyl-hexyl) and phenyl; or
amide′ is selected from the group consisting of NH₂, NH(alkyl), N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In another preferred variant according to the first aspect of the present invention, the mycosporine-like amino acid compound is a compound according to general formula (VI), wherein
R1″ is selected from the group consisting of CH₂ and C(methyl)₂; and/or
R2″ is selected from the group consisting of —CH₂—OH, —C(═O)—O—Y, and —C(═O)-amide″; and/or
X is selected from the group consisting of CH₂, C═O, O, S, SO, SO₂, NH, and N(alkyl); and/or
Y is selected from the group consisting of H, methyl, ethyl, propyl, isopropyl, butyl, isobutyl, tert-butyl,

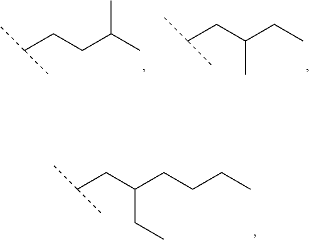

(2-ethyl-hexyl) and phenyl; and/or
amide″ is selected from the group consisting of NH₂, NH(alkyl), N(alkyl)₂, —N(O-alkyl)(alkyl) (Weinreb amide) and —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and tert-butyl.

In a still more preferred variant according to the present invention, the sunscreen product, cosmetic or pharmaceutical preparation or homecare product comprises at least one mycosporine-like amino acid compound selected from the group consisting of the following compounds I-1 to I-128 according to the general formula (V) and II-1 to II-192 according to the general formula (VI):

TABLE 1

| 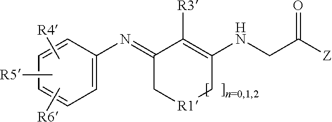 | R1′ | R3′ | Z |
|---|---|---|---|
| I-1 | CH2 | H | —O—R2′ |
| I-2 | CH2 | methyl | —O—R2′ |
| I-3 | CH2 | ethyl | —O—R2′ |
| I-4 | CH2 | —O-methyl | —O—R2′ |
| I-5 | CH2 | —C(═O)—OH | —O—R2′ |
| I-6 | CH2 | —C(═O)—CH3 | —O—R2′ |
| I-7 | CH2 | —C(═O)—C3H7 | —O—R2′ |
| I-8 | CH2 | —C(═O)—O-methyl | —O—R2′ |
| I-9 | CH2 | H | amide′ |
| I-10 | CH2 | methyl | amide′ |
| I-11 | CH2 | ethyl | amide′ |
| I-12 | CH2 | —O-methyl | amide′ |
| I-13 | CH2 | —C(═O)—OH | amide′ |
| I-14 | CH2 | —C(═O)—CH3 | amide′ |
| I-15 | CH2 | —C(═O)—C3H7 | amide′ |
| I-16 | CH2 | —C(═O)—O-methyl | amide′ |
| I-17 | C(methyl)2 | H | —O—R2′ |
| I-18 | C(methyl)2 | methyl | —O—R2′ |
| I-19 | C(methyl)2 | ethyl | —O—R2′ |
| I-20 | C(methyl)2 | —O-methyl | —O—R2′ |
| I-21 | C(methyl)2 | —C(═O)—OH | —O—R2′ |
| I-22 | C(methyl)2 | —C(═O)—CH3 | —O—R2′ |
| I-23 | C(methyl)2 | —C(═O)—C3H7 | —O—R2′ |
| I-24 | C(methyl)2 | —C(═O)—O-methyl | —O—R2′ |
| I-25 | C(methyl)2 | H | amide′ |
| I-26 | C(methyl)2 | methyl | amide′ |
| I-27 | C(methyl)2 | ethyl | amide′ |
| I-28 | C(methyl)2 | —O-methyl | amide′ |
| I-29 | C(methyl)2 | —C(═O)—OH | amide′ |
| I-30 | C(methyl)2 | —C(═O)—CH3 | amide′ |
| I-31 | C(methyl)2 | —C(═O)—C3H7 | amide′ |
| I-32 | C(methyl)2 | —C(═O)—O-methyl | amide′ |
| I-33 | C | H | —O—R2′ |
| I-34 | O | methyl | —O—R2′ |
| I-35 | O | ethyl | —O—R2′ |
| I-36 | O | —O-methyl | —O—R2′ |
| I-37 | O | —C(═O)—OH | —O—R2′ |
| I-38 | O | —C(═O)—CH3 | —O—R2′ |
| I-39 | O | —C(═O)—C3H7 | —O—R2′ |
| I-40 | O | —C(═O)—O-methyl | —O—R2′ |
| I-41 | O | H | amide′ |
| I-42 | O | methyl | amide′ |
| I-43 | O | ethyl | amide′ |
| I-44 | O | —O-methyl | amide′ |
| I-45 | O | —C(═O)—OH | amide′ |
| I-46 | O | —C(═O)—CH3 | amide′ |
| I-47 | O | —C(═O)—C3H7 | amide′ |
| I-48 | O | —C(═O)—O-methyl | amide′ |
| I-49 | S | H | —O—R2′ |
| I-50 | S | methyl | —O—R2′ |
| I-51 | S | ethyl | —O—R2′ |
| I-52 | S | —O-methyl | —O—R2′ |
| I-53 | S | —C(═O)—OH | —O—R2′ |
| I-54 | S | —C(═O)—CH3 | —O—R2′ |
| I-55 | S | —C(═O)—C3H7 | —O—R2′ |
| I-56 | S | —C(═O)—O-methyl | —O—R2′ |
| I-57 | S | H | amide′ |
| I-58 | S | methyl | amide′ |
| I-59 | S | ethyl | amide′ |
| I-60 | S | —O-methyl | amide′ |
| I-61 | S | —C(═O)—OH | amide′ |
| I-62 | S | —C(═O)—CH3 | amide′ |
| I-63 | S | —C(═O)—C3H7 | amide′ |
| I-64 | S | —C(═O)—O-methyl | amide′ |
| I-65 | SC | H | —O—R2′ |
| I-66 | SO | methyl | —O—R2′ |
| I-67 | SO | ethyl | —O—R2′ |
| I-68 | SO | —O-methyl | —O—R2′ |
| I-69 | SO | —C(═O)—OH | —O—R2′ |
| I-70 | SO | —C(═O)—CH3 | —O—R2′ |
| I-71 | SO | —C(═O)—C3H7 | —O—R2′ |
| I-72 | SO | —C(═O)—O-methyl | —O—R2′ |
| I-73 | SC | H | amide′ |
| I-74 | SC | methyl | amide′ |
| I-75 | SC | ethyl | amide′ |
| I-76 | SC | —O-methyl | amide′ |
| I-77 | SC | —C(═O)—OH | amide′ |
| I-78 | SC | —C(═O)—CH3 | amide′ |
| I-79 | SO | —C(═O)—C3H7 | amide′ |
| I-80 | SO | —C(═O)—O-methyl | amide′ |
| I-81 | SO2 | H | —O—R2′ |
| I-82 | SO2 | methyl | —O—R2′ |
| I-83 | SO2 | ethyl | —O—R2′ |
| I-84 | SO2 | —O-methyl | —O—R2′ |
| I-85 | SO2 | —C(═O)—OH | —O—R2′ |
| I-86 | SO2 | —C(═O)—CH3 | —O—R2′ |
| I-87 | SO2 | —C(═O)—C3H7 | —O—R2′ |
| I-88 | SO2 | —C(═O)—O-methyl | —O—R2′ |
| I-89 | SO2 | H | amide′ |
| I-90 | SO2 | methyl | amide′ |
| I-91 | SO2 | ethyl | amide′ |
| I-92 | SO2 | —O-methyl | amide′ |
| I-93 | SO2 | —C(═O)—OH | amide′ |
| I-94 | SO2 | —C(═O)—CH3 | amide′ |
| I-95 | SO2 | —C(═O)—C3H7 | amide′ |
| I-96 | SO2 | —C(═O)—O-methyl | amide′ |
| I-97 | NH | H | —O—R2′ |
| I-98 | NH | methyl | —O—R2′ |
| I-99 | NH | ethyl | —O—R2′ |
| I-100 | NH | —O-methyl | —O—R2′ |
| I-101 | NH | —C(═O)—OH | —O—R2′ |
| I-102 | NH | —C(═O)—CH3 | —O—R2′ |
| I-103 | NH | —C(═O)—C3H7 | —O—R2′ |
| I-104 | NH | —C(═O)—O-methyl | —O—R2′ |
| I-105 | NH | H | amide′ |
| I-106 | NH | methyl | amide′ |
| I-107 | NH | ethyl | amide′ |
| I-108 | NH | —O-methyl | amide′ |
| I-109 | NH | —C(═O)—OH | amide′ |
| I-110 | NH | —C(═O)—CH3 | amide′ |
| I-111 | NH | —C(═O)—C3H7 | amide′ |
| I-112 | NH | —C(═O)—O-methyl | amide′ |
| I-113 | N(alkyl) | H | —O—R2′ |
| I-114 | N(alkyl) | methyl | —O—R2′ |
| I-115 | N(alkyl) | ethyl | —O—R2′ |
| I-116 | N(alkyl) | —O-methyl | —O—R2′ |
| I-117 | N(alkyl) | —C(═O)—OH | —O—R2′ |
| I-118 | N(alkyl) | —C(═O)—CH3 | —O—R2′ |
| I-119 | N(alkyl) | —C(═O)—C3H7 | —O—R2′ |
| I-120 | N(alkyl) | —C(═O)—O-methyl | —O—R2′ |
| I-121 | N(alkyl) | H | amide′ |
| I-122 | N(alkyl) | methyl | amide′ |
| I-123 | N(alkyl) | ethyl | amide′ |
| I-124 | N(alkyl) | —O-methyl | amide′ |
| I-125 | N(alkyl) | —C(═O)—OH | amide′ |
| I-126 | N(alkyl) | —C(═O)—CH3 | amide′ |
| I-127 | N(alkyl) | —C(═O)—C3H7 | amide′ |
| I-128 | N(alkyl) | —C(═O)—O-methyl | amide′ |
| 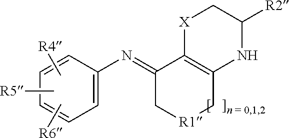 | R1″ | X | R2″ |
| II-1 | CH2 | CH2 | —CH2—OH |
| II-2 | CH2 | C═O | —CH2—OH |
| II-3 | CH2 | O | —CH2—OH |
| II-4 | CH2 | S | —CH2—OH |
| II-5 | CH2 | SO | —CH2—OH |
| II-6 | CH2 | SO2 | —CH2—OH |
| II-7 | CH2 | NH | —CH2—OH |
| II-8 | CH2 | N(alkyl) | —CH2—OH |
| II-9 | CH2 | CH2 | —C(═O)—O—Y |
| II-10 | CH2 | C═O | —C(═O)—O—Y |
| II-11 | CH2 | O | —C(═O)—O—Y |
| II-12 | CH2 | S | —C(═O)—O—Y |
| II-13 | CH2 | SO | —C(═O)—O—Y |
| II-14 | CH2 | SO2 | —C(═O)—O—Y |
| II-15 | CH2 | NH | —C(═O)—O—Y |
| II-16 | CH2 | N(alkyl) | —C(═O)—O—Y |
| II-17 | CH2 | CH2 | —C(═O)-amide″ |
| II-18 | CH2 | C═O | —C(═O)-amide″ |
| II-19 | CH2 | O | —C(═O)-amide″ |
| II-20 | CH2 | S | —C(═O)-amide″ |
| II-21 | CH2 | SO | —C(═O)-amide″ |
| II-22 | CH2 | SO2 | —C(═O)-amide″ |
| II-23 | CH2 | NH | —C(═O)-amide″ |
| II-24 | CH2 | N(alkyl) | —C(═O)-amide″ |
| II-25 | C(methyl)2 | CH2 | —CH2—OH |
| II-26 | C(methyl)2 | C═O | —CH2—OH |
| II-27 | C(methyl)2 | O | —CH2—OH |
| II-28 | C(methyl)2 | S | —CH2—OH |
| II-29 | C(methyl)2 | SO | —CH2—OH |
| II-30 | C(methyl)2 | SO2 | —CH2—OH |
| II-31 | C(methyl)2 | NH | —CH2—OH |
| II-32 | C(methyl)2 | N(alkyl) | —CH2—OH |
| II-33 | C(methyl)2 | CH2 | —C(═O)—O—Y |
| II-34 | C(methyl)2 | C═O | —C(═O)—O—Y |
| II-35 | C(methyl)2 | O | —C(═O)—O—Y |
| II-36 | C(methyl)2 | S | —C(═O)—O—Y |
| II-37 | C(methyl)2 | SO | —C(═O)—O—Y |
| II-38 | C(methyl)2 | SO2 | —C(═O)—O—Y |
| II-39 | C(methyl)2 | NH | —C(═O)—O—Y |
| II-40 | C(methyl)2 | N(alkyl) | —C(═O)—O—Y |
| II-41 | C(methyl)2 | CH2 | —C(═O)-amide″ |
| II-42 | C(methyl)2 | C═O | —C(═O)-amide″ |
| II-43 | C(methyl)2 | O | —C(═O)-amide″ |
| II-44 | C(methyl)2 | S | —C(═O)-amide″ |
| II-45 | C(methyl)2 | SO | —C(═O)-amide″ |
| II-46 | C(methyl)2 | SO2 | —C(═O)-amide″ |
| II-47 | C(methyl)2 | NH | —C(═O)-amide″ |
| II-48 | C(methyl)2 | N(alkyl) | —C(═O)-amide″ |
| II-49 | O | CH2 | —CH2—OH |
| II-50 | O | C═O | —CH2—OH |
| II-51 | O | C | —CH2—OH |
| II-52 | O | S | —CH2—OH |
| II-53 | O | SO | —CH2—OH |
| II-54 | O | SO2 | —CH2—OH |
| II-55 | O | NH | —CH2—OH |
| II-56 | O | N(alkyl) | —CH2—OH |
| II-57 | O | CH2 | —C(═O)—O—Y |
| II-58 | O | C═O | —C(═O)—O—Y |
| II-59 | O | O | —C(═O)—O—Y |
| II-60 | O | S | —C(═O)—O—Y |
| II-61 | O | SO | —C(═O)—O—Y |
| II-62 | O | SO2 | —C(═O)—O—Y |
| II-63 | O | NH | —C(═O)—O—Y |
| II-64 | O | N(alkyl) | —C(═O)—O—Y |
| II-65 | O | CH2 | —C(═O)-amide″ |
| II-66 | O | C═O | —C(═O)-amide″ |
| II-67 | O | O | —C(═O)-amide″ |
| II-68 | O | S | —C(═O)-amide″ |
| II-69 | O | SO | —C(═O)-amide″ |
| II-70 | O | SO2 | —C(═O)-amide″ |
| II-71 | O | NH | —C(═O)-amide″ |
| II-72 | O | N(alkyl) | —C(═O)-amide″ |
| II-73 | S | CH2 | —CH2—OH |
| II-74 | S | C═O | —CH2—OH |
| II-75 | S | O | —CH2—OH |
| II-76 | S | S | —CH2—OH |
| II-77 | S | SO | —CH2—OH |
| II-78 | S | SO2 | —CH2—OH |
| II-79 | S | NH | —CH2—OH |
| II-80 | S | N(alkyl) | —CH2—OH |
| II-81 | S | CH2 | —C(═O)—O—Y |
| II-82 | S | C═O | —C(═O)—O—Y |
| II-83 | S | O | —C(═O)—O—Y |
| II-84 | S | S | —C(═O)—O—Y |
| II-85 | S | SO | —C(═O)—O—Y |
| II-86 | S | SO2 | —C(═O)—O—Y |
| II-87 | S | NH | —C(═O)—O—Y |
| II-88 | S | N(alkyl) | —C(═O)—O—Y |
| II-89 | S | CH2 | —C(═O)-amide″ |
| II-90 | S | C═O | —C(═O)-amide″ |
| II-91 | S | O | —C(═O)-amide″ |
| II-92 | S | S | —C(═O)-amide″ |
| II-93 | S | SO | —C(═O)-amide″ |
| II-94 | S | SO2 | —C(═O)-amide″ |
| II-95 | S | NH | —C(═O)-amide″ |
| II-96 | S | N(alkyl) | —C(═O)-amide″ |
| II-97 | SO | CH2 | —CH2—OH |
| II-98 | SO | C═O | —CH2—OH |
| II-99 | SO | O | —CH2—OH |
| II-100 | SO | S | —CH2—OH |
| II-101 | SO | SO | —CH2—OH |
| II-102 | SO | SO2 | —CH2—OH |
| II-103 | SO | NH | —CH2—OH |
| II-104 | SO | N(alkyl) | —CH2—OH |
| II-105 | SO | CH2 | —C(═O)—O—Y |
| II-106 | SO | C═O | —C(═O)—O—Y |
| II-107 | SO | O | —C(═O)—O—Y |
| II-108 | SO | S | —C(═O)—O—Y |
| II-109 | SO | SO | —C(═O)—O—Y |
| II-110 | SO | SO2 | —C(═O)—O—Y |
| II-111 | SO | NH | —C(═O)—O—Y |
| II-112 | SO | N(alkyl) | —C(═O)—O—Y |
| II-113 | SO | CH2 | —C(═O)-amide″ |
| II-114 | SO | C═O | —C(═O)-amide″ |
| II-115 | SO | O | —C(═O)-amide″ |
| II-116 | SO | S | —C(═O)-amide″ |
| II-117 | SO | SO | —C(═O)-amide″ |
| II-118 | SO | SO2 | —C(═O)-amide″ |
| II-119 | SO | NH | —C(═O)-amide″ |
| II-120 | SO | N(alkyl) | —C(═O)-amide″ |
| II-121 | SO2 | CH2 | —CH2—OH |
| II-122 | SO2 | C═O | —CH2—OH |
| II-123 | SO2 | O | —CH2—OH |
| II-124 | SO2 | S | —CH2—OH |
| II-125 | SO2 | SO | —CH2—OH |
| II-126 | SO2 | SO2 | —CH2—OH |
| II-127 | SO2 | NH | —CH2—OH |
| II-128 | SO2 | N(alkyl) | —CH2—OH |
| II-129 | SO2 | CH2 | —C(═O)—O—Y |
| II-130 | SO2 | C═O | —C(═O)—O—Y |
| II-131 | SO2 | O | —C(═O)—O—Y |
| II-132 | SO2 | S | —C(═O)—O—Y |
| II-133 | SO2 | SO | —C(═O)—O—Y |
| II-134 | SO2 | SO2 | —C(═O)—O—Y |
| II-135 | SO2 | NH | —C(═O)—O—Y |
| II-136 | SO2 | N(alkyl) | —C(═O)—O—Y |
| II-137 | SO2 | CH2 | —C(═O)-amide″ |
| II-138 | SO2 | C═O | —C(═O)-amide″ |
| II-139 | SO2 | O | —C(═O)-amide″ |
| II-140 | SO2 | S | —C(═O)-amide″ |
| II-141 | SO2 | SO | —C(═O)-amide″ |
| II-142 | SO2 | SO2 | —C(═O)-amide″ |
| II-143 | SO2 | NH | —C(═O)-amide″ |
| II-144 | SO2 | N(alkyl) | —C(═O)-amide″ |
| II-145 | NH | CH2 | —CH2—OH |
| II-146 | NH | C═O | —CH2—OH |
| II-147 | NH | O | —CH2—OH |
| II-148 | NH | S | —CH2—OH |
| II-149 | NH | SO | —CH2—OH |
| II-150 | NH | SO2 | —CH2—OH |
| II-151 | NH | NH | —CH2—OH |
| II-152 | NH | N(alkyl) | —CH2—OH |
| II-153 | NH | CH2 | —C(═O)—O—Y |
| II-154 | NH | C═O | —C(═O)—O—Y |
| II-155 | NH | O | —C(═O)—O—Y |
| II-156 | NH | S | —C(═O)—O—Y |
| II-157 | NH | SO | —C(═O)—O—Y |
| II-158 | NH | SO2 | —C(═O)—O—Y |
| II-159 | NH | NH | —C(═O)—O—Y |
| II-160 | NH | N(alkyl) | —C(═O)—O—Y |
| II-161 | NH | CH2 | —C(═O)-amide″ |
| II-162 | NH | C═O | —C(═O)-amide″ |
| II-163 | NH | O | —C(═O)-amide″ |
| II-164 | NH | S | —C(═O)-amide' |
| II-165 | NH | SO | —C(═O)-amide″ |
| II-166 | NH | SO2 | —C(═O)-amide″ |
| II-167 | NH | NH | —C(═O)-amide″ |
| II-168 | NH | N(alkyl) | —C(═O)-amide″ |
| II-169 | N(alkyl) | CH2 | —CH2—OH |
| II-170 | N(alkyl) | C═O | —CH2—OH |
| II-171 | N(alkyl) | O | —CH2—OH |
| II-172 | N(alkyl) | S | —CH2—OH |
| II-173 | N(alkyl) | SO | —CH2—OH |
| II-174 | N(alkyl) | SO2 | —CH2—OH |
| II-175 | N(alkyl) | NH | —CH2—OH |
| II-176 | N(alkyl) | N(alkyl) | —CH2—OH |
| II-177 | N(alkyl) | CH2 | —C(═O)—O—Y |
| II-178 | N(alkyl) | C═O | —C(═O)—O—Y |
| II-179 | N(alkyl) | O | —C(═O)—O—Y |
| II-180 | N(alkyl) | S | —C(═O)—O—Y |
| II-181 | N(alkyl) | SO | —C(═O)—O—Y |
| II-182 | N(alkyl) | SO2 | —C(═O)—O—Y |
| II-183 | N(alkyl) | NH | —C(═O)—O—Y |
| II-184 | N(alkyl) | N(alkyl) | —C(═O)—O—Y |
| II-185 | N(alkyl) | CH2 | —C(═O)-amide″ |
| II-186 | N(alkyl) | C═O | —C(═O)-amide″ |
| II-187 | N(alkyl) | O | —C(═O)-amide″ |
| II-188 | N(alkyl) | S | —C(═O)-amide″ |
| II-189 | N(alkyl) | SO | —C(═O)-amide″ |
| II-190 | N(alkyl) | SO2 | —C(═O)-amide″ |
| II-191 | N(alkyl) | NH | —C(═O)-amide' |
| II-192 | N(alkyl) | N(alkyl) | —C(═O)-amide″ |

or a tautomer or a stereoisomer a salt of the afore-mentioned compounds, or any mixture of the afore-mentioned compounds.

In the mycosporine-like amino acid compounds of Table 1, the phenyl ring may be unsubstituted or substituted. The substituents R4′, R5′, R6′, R4″, R5″, R6″, amide′, Y, amide″ and alkyl have the same meaning as defined for the mycosporine-like amino acid compounds according to general formula (V) or (VI) before.

Particular preferred are those mycosporine-like amino acid compounds, wherein in the general formula (V) R1′ is CH₂ or C(methyl)₂ or wherein in the general formula (VI) R1″ is CH₂ or C(methyl)₂:

TABLE 2

| 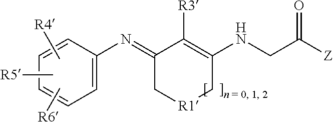 | R1′ | R3′ | R1′ |
|---|---|---|---|
| I-1 | CH2 | H | —O—R2′ |
| I-2 | CH2 | methyl | —O—R2′ |
| I-3 | CH2 | ethyl | —O—R2′ |
| I-4 | CH2 | —O-methyl | —O—R2′ |
| I-5 | CH2 | —C(═O)—OH | —O—R2′ |
| I-6 | CH2 | —C(═O)—CH3 | —O—R2′ |
| I-7 | CH2 | —C(═O)—C3H7 | —O—R2′ |
| I-8 | CH2 | —C(═O)—O-methyl | —O—R2′ |
| I-9 | CH2 | H | amide′ |
| I-10 | CH2 | methyl | amide′ |
| I-11 | CH2 | ethyl | amide′ |
| I-12 | CH2 | —O-methyl | amide′ |
| I-13 | CH2 | —C(═O)—OH | amide′ |
| I-14 | CH2 | —C(═O)—CH3 | amide′ |
| I-15 | CH2 | —C(═O)—C3H7 | amide′ |
| I-16 | CH2 | —C(═O)—O-methyl | amide′ |
| I-17 | C(methyl)2 | H | —O—R2′ |
| I-18 | C(methyl)2 | methyl | —O—R2′ |
| I-19 | C(methyl)2 | ethyl | —O—R2′ |
| I-20 | C(methyl)2 | —O-methyl | —O—R2′ |
| I-21 | C(methyl)2 | —C(═O)—OH | —O—R2′ |
| I-22 | C(methyl)2 | —C(═O)—CH3 | —O—R2′ |
| I-23 | C(methyl)2 | —C(═O)—C3H7 | —O—R2′ |
| I-24 | C(methyl)2 | —C(═O)—O-methyl | —O—R2′ |
| I-25 | C(methyl)2 | H | amide′ |
| I-26 | C(methyl)2 | methyl | amide′ |
| I-27 | C(methyl)2 | ethyl | amide′ |
| I-28 | C(methyl)2 | —O-methyl | amide′ |
| I-29 | C(methyl)2 | —C(═O)—OH | amide′ |
| I-30 | C(methyl)2 | —C(═O)—CH3 | amide′ |
| I-31 | C(methyl)2 | —C(═O)—C3H7 | amide′ |
| I-32 | C(methyl)2 | —C(═O)—O-methyl | amide′ |
| 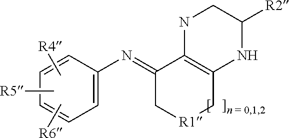 | R1″ | X | R2″ |
| II-1 | CH2 | CH2 | —CH2—OH |
| II-2 | CH2 | C═O | —CH2—OH |
| II-3 | CH2 | O | —CH2—OH |
| II-4 | CH2 | S | —CH2—OH |
| II-5 | CH2 | SC | —CH2—OH |
| II-6 | CH2 | SO2 | —CH2—OH |
| II-7 | CH2 | NH | —CH2—OH |
| II-8 | CH2 | N(alkyl) | —CH2—OH |
| II-9 | CH2 | CH2 | —C(═O)—O—Y |
| II-10 | CH2 | C═O | —C(═O)—O—Y |
| II-11 | CH2 | O | —C(═O)—O—Y |
| II-12 | CH2 | S | —C(═O)—O—Y |
| II-13 | CH2 | SC | —C(═O)—O—Y |
| II-14 | CH2 | SO2 | —C(═O)—O—Y |
| II-15 | CH2 | NH | —C(═O)—O—Y |
| II-16 | CH2 | N(alkyl) | —C(═O)—O—Y |
| II-17 | CH2 | CH2 | —C(═O)-amide″ |
| II-18 | CH2 | C═O | —C(═O)-amide″ |
| II-19 | CH2 | O | —C(═O)-amide″ |
| II-20 | CH2 | S | —C(═O)-amide″ |
| II-21 | CH2 | SO | —C(═O)-amide″ |
| II-22 | CH2 | SO2 | —C(═O)-amide″ |
| II-23 | CH2 | NH | —C(═O)-amide″ |
| II-24 | CH2 | N(alkyl) | —C(═O)-amide″ |
| II-25 | C(methyl)2 | CH2 | —CH2—OH |
| II-26 | C(methyl)2 | C═O | —CH2—OH |
| II-27 | C(methyl)2 | C | —CH2—OH |
| II-28 | C(methyl)2 | S | —CH2—OH |
| II-29 | C(methyl)2 | SO | —CH2—OH |
| II-30 | C(methyl)2 | SO2 | —CH2—OH |
| II-31 | C(methyl)2 | NH | —CH2—OH |
| II-32 | C(methyl)2 | N(alkyl) | —CH2—OH |
| II-33 | C(methyl)2 | CH2 | —C(═O)—O—Y |
| II-34 | C(methyl)2 | C═O | —C(═O)—O—Y |
| II-35 | C(methyl)2 | C | —C(═O)—O—Y |
| II-36 | C(methyl)2 | S | —C(═O)—O—Y |
| II-37 | C(methyl)2 | SO | —C(═O)—O—Y |
| II-38 | C(methyl)2 | SO2 | —C(═O)—O—Y |
| II-39 | C(methyl)2 | NH | —C(═O)—O—Y |
| II-40 | C(methyl)2 | N(alkyl) | —C(═O)—O—Y |
| II-41 | C(methyl)2 | CH2 | —C(═O)-amide″ |
| II-42 | C(methyl)2 | C═O | —C(═O)-amide″ |
| II-43 | C(methyl)2 | O | —C(═O)-amide″ |
| II-44 | C(methyl)2 | S | —C(═O)-amide″ |
| II-45 | C(methyl)2 | SO | —C(═O)-amide″ |
| II-46 | C(methyl)2 | SO2 | —C(═O)-amide″ |
| II-47 | C(methyl)2 | NH | —C(═O)-amide″ |
| II-48 | C(methyl)2 | N(alkyl) | —C(═O)-amide″ |

or a tautomer or a stereoisomer or a salt of the afore-mentioned compounds, or any mixture of the afore-mentioned compounds.

In the mycosporine-like amino acid compounds of Table 2, the phenyl ring may be unsubstituted or substituted. The substituents R4′, R5′, R6′, R4″, R5″, R6″, amide′, Y, amide″ and alkyl have the same meaning as defined for the mycosporine-like amino acid compounds according to general formula (V) or (VI) before.

Preferably, in the mycosporine-like amino acid compounds according to the general formula (V) and formula (VI) specified in Table 1 and Table 2
R2′ is selected from the group consisting of H, methyl, ethyl, propyl, isopropyl, butyl, isobutyl, tert-butyl,

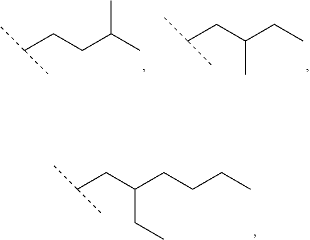

(2-ethylhexyl) and phenyl;
amide′ is selected from the group consisting of NH₂, NH(alkyl), N(alkyl)₂, —N(O-alkyl)(alkyl) (Weinreb amide) and —N(OH)(H) (hydroxamate);
Y is selected from the group consisting of H, methyl, ethyl, propyl, isopropyl, butyl, isobutyl, tert-butyl,

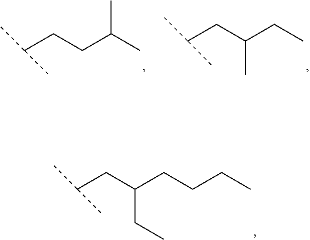

(2-ethylhexyl) and phenyl;
amide″ is selected from the group consisting of NH₂, NH(alkyl), N(alkyl)₂, —N(O-alkyl)(alkyl) (Weinreb amide) and —N(OH)(H) (hydroxamate); and
alkyl is selected from the group consisting of methyl, ethyl, propyl, isopropyl, butyl, isobutyl, and tert-butyl.

In a most preferred variant according to the first aspect of the present invention, the mycosporine-like amino acid compound is a compound according to general formula (V) and its variants or formula (VI) and its variants, wherein R1′ or R1″ are CH₂. Such mycosporine-like amino acid compounds have a particular good water solubility.

In a still further preferred variant according to the first aspect of the present invention, the mycosporine-like amino acid compound is a compound according to general formulae (VI), (IX), (X) and their variants, wherein X is selected from the group consisting of S, SO, and SO₂.

Such mycosporine-like amino acid compounds alter the UV absorption profile of the sunscreen product or cosmetic or pharmaceutical preparation and broaden the UV absorption spectrum of the formulation across the UV radiation spectrum.

In a still more preferred variant according to the first aspect of the present invention, the mycosporine-like amino acid compound is a compound according to general formulae (VI), (IX), (X) and their variants, wherein X is S. However, the S atom in said compounds is prone to oxidation.

In a most preferred variant according to the first aspect of the present invention, the mycosporine-like amino acid compound is a compound according to general formulae (VI), (IX), (X) and their variants, wherein X is either SO or SO₂. Such mycosporine-like amino acid compounds are particular stable, due to their oxidation state.

In a still further preferred variant according to the present invention, in the general formulae (V) to (X) and their variants, n is 1.

In a more preferred variant according to the first aspect of the present invention, the mycosporine-like amino acid compound is a compound according to general formula (V), wherein
Z is —O—R2′ or amide′; and/or
R1′ is either CH₂ or C(methyl)₂; and/or
R2′ is either H or ethyl or isopropyl or 2-ethylhexyl or phenyl; or
amide′ is either NH₂ or N(methyl)₂ or N(ethyl)₂ or —N(O-alkyl)(alkyl) (Weinreb amide) or —N(OH)(H) (hydroxamate); and/or
R3′ is either H or —O-methyl or —C(═O)—OH or —C(═O)—CH₃ or —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In a more preferred variant according to the first aspect of the present invention, the mycosporine-like amino acid compound is a compound according to general formulae (VII) or (VIII), wherein
R1′ is either CH₂ or C(methyl)₂; and/or
R2′ is either H or ethyl or isopropyl or 2-ethylhexyl or phenyl; or
amide′ is either NH₂ or N(methyl)₂ or N(ethyl)₂ or —N(O-alkyl)(alkyl) (Weinreb amide) or —N(OH)(H) (hydroxamate); and/or
R3′ is either H or —O-methyl or —C(═O)—OH or —C(═O)—CH₃ or —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In a more preferred variant according to the first aspect of the present invention, the mycosporine-like amino acid compound is a compound according to general formula (VI), wherein
R1″ is either CH₂ or C(methyl)₂; and/or
R2″ is either —C(═O)—O—Y, or —C(═O)-amide″; and/or
X is S, SO or SO₂; and/or
Y is either H or ethyl or isopropyl or 2-ethylhexyl or phenyl; or
amide″ is either NH₂ or N(methyl)₂ or N(ethyl)₂ or —N(O-alkyl)(alkyl) (Weinreb amide) or —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and butyl.

The phenyl ring in the mycosporine-like amino acid compounds according to the general formulae (V) to (X) and their variants can be unsubstituted or substituted.

In a preferred variant, the phenyl ring in the mycosporine-like amino acid compounds according to the general formulae (V) to (X) is unsubstituted, i.e. the substituents R4′, R5′ and R6′ on the phenyl ring in the general formula (V) are H; or the substituents R4″, R5″ and R6″ on the phenyl ring in the general formula (VI) are H.

In a further preferred variant, the phenyl ring in the mycosporine-like amino acid compounds according to the general formulae (V) to (X) and their variants is substituted.

Most preferred, the phenyl ring in the mycosporine-like amino acid compounds according to the general formula (V), (VII), (Vill) and their variants is unsubstituted, i.e. the substituents R4′, R5′ and R6′ are each H. Alternatively, most preferred, the phenyl ring in the mycosporine-like amino acid compounds according to the general formula (VI), (IX), (X) and their variants is unsubstituted, i.e. the substituents R4″, R5″ and R6″ are each H. Said compounds have an improved water solubility.

Preferably, the phenyl ring in the mycosporine-like amino acid compounds according to the general formulae (V) to (X) and their variants is monosubstituted, disubstituted or trisubstituted.

In a still more preferred variant according to the first aspect of the present invention, the mycosporine-like amino acid is a compound according to general formulae (V), (VII), (VIII) and their variants, wherein the substituents R4′, R5′ and R6′ which may be identical or different, are independently from each other selected from the group consisting of H, OH, alkyl, preferably methyl, ethyl, propyl, iso-propyl, butyl, isobutyl or tert.-butyl, alkoxy, preferably, O-methyl, O-ethyl, or O-butyl, —S(═O)₂OH, and sulfonyl.

Still more preferred, the substituents R4′, R5′ and R6′ in the general formulae (V), (VII), (Vill) and their variants, which may be identical or different, are independently from each other selected from the group consisting of H, OH, methyl, O-methyl, —S(═O)₂OH, and sulfonyl.

Alternatively, the mycosporine-like amino acid compound is a compound according to general formulae (VI), (IX), (X) and their variants, wherein the substituents R4″, R5″ and R6″, which may be identical or different, are independently from each other selected from the group consisting of H, OH, alkyl, preferably methyl, ethyl, propyl, iso-propyl, butyl, isobutyl, tert-butyl, alkoxy, preferably O-methyl, O-ethyl, or O-butyl, —S(═O)₂OH, and sulfonyl.

Still more preferred, the substituents R4″, R5″ and R6″ in the general formula (VI), (IX), (X) and their variants, which may be identical or different, are independently from each other selected from the group consisting of H, OH, methyl, O-methyl, —S(═O)₂OH, and sulfonyl.

In the general formulae (V) to (X) and their variants, the following substitution patterns are particularly preferred:
R4′=H and R5′=H and R6′=H; or
R4′=H and R5′=H and R6′=OH; or
R4′=H and R5′=H and R6′=methyl; or
R4′=H and R5′=H and R6′=O-methyl; or
R4′=H and R5′=H and R6′=—S(═O)₂OH; or
R4′=H and R5′=H and R6′=sulfonyl; or
R4′=H and R5′=OH and R6′=H; or
R4′=H and R5′=OH and R6′=OH; or
R4′=H and R5′=OH and R6′=methyl; or
R4′=H and R5′=OH and R6′=O-methyl; or
R4′=H and R5′=OH and R6′=—S(═O)₂OH; or
R4′=H and R5′=OH and R6′=sulfonyl; or
R4′=H and R5′=methyl and R6′=H; or
R4′=H and R5′=methyl and R6′=OH; or
R4′=H and R5′=methyl and R6′=methyl; or
R4′=H and R5′=methyl and R6′=O-methyl; or
R4′=H and R5′=methyl and R6′=—S(═O)₂OH; or
R4′=H and R5′=methyl and R6′=sulfonyl; or
R4′=H and R5′=O-methyl and R6′=H; or
R4′=H and R5′=O-methyl and R6′=OH; or
R4′=H and R5′=O-methyl and R6′=methyl; or
R4′=H and R5′=O-methyl and R6′=O-methyl; or
R4′=H and R5′=O-methyl and R6′=—S(═O)₂OH; or
R4′=H and R5′=O-methyl and R6′=sulfonyl; or
R4′=H and R5′=—S(═O)₂OH and R6′=H; or
R4′=H and R5′=—S(═O)₂OH and R6′=OH; or
R4′=H and R5′=—S(═O)₂OH and R6′=methyl; or
R4′=H and R5′=—S(═O)₂OH and R6′=O-methyl; or
R4′=H and R5′=—S(═O)₂OH and R6′=—S(═O)₂OH; or
R4′=H and R5′=—S(═O)₂OH and R6′=sulfonyl; or
R4′=H and R5′=sulfonyl and R6′=H; or
R4′=H and R5′=sulfonyl and R6′=OH; or
R4′=H and R5′=sulfonyl and R6′=methyl; or
R4′=H and R5′=sulfonyl and R6′=O-methyl; or
R4′=H and R5′=sulfonyl and R6′=—S(═O)₂OH; or
R4′=H and R5′=sulfonyl and R6′=sulfonyl; or
R4′=OH and R5′=H and R6′=H; or
R4′=OH and R5′=H and R6′=OH; or
R4′=OH and R5′=H and R6′=methyl; or
R4′=OH and R5′=H and R6′=O-methyl; or
R4′=OH and R5′=H and R6′=—S(═O)₂OH; or
R4′=OH and R5′=H and R6′=sulfonyl; or
R4′=OH and R5′=OH and R6′=H; or
R4′=OH and R5′=OH and R6′=OH; or
R4′=OH and R5′=OH and R6′=methyl; or
R4′=OH and R5′=OH and R6′=O-methyl; or
R4′=OH and R5′=OH and R6′=—S(═O)₂OH; or
R4′=OH and R5′=OH and R6′=sulfonyl; or
R4′=OH and R5′=methyl and R6′=H; or
R4′=OH and R5′=methyl and R6′=OH; or
R4′=OH and R5′=methyl and R6′=methyl; or
R4′=OH and R5′=methyl and R6′=O-methyl; or
R4′=OH and R5′=methyl and R6′=—S(═O)₂OH; or
R4′=OH and R5′=methyl and R6′=sulfonyl; or
R4′=OH and R5′=O-methyl and R6′=H; or
R4′=OH and R5′=O-methyl and R6′=OH; or
R4′=OH and R5′=O-methyl and R6′=methyl; or
R4′=OH and R5′=O-methyl and R6′=O-methyl; or
R4′=OH and R5′=O-methyl and R6′=—S(═O)₂OH; or
R4′=OH and R5′=O-methyl and R6′=sulfonyl; or
R4′=OH and R5′=—S(═O)₂OH and R6′=H; or
R4′=OH and R5′=—S(═O)₂OH and R6′=OH; or
R4′=OH and R5′=—S(═O)₂OH and R6′=methyl; or
R4′=OH and R5′=—S(═O)₂OH and R6′=O-methyl; or
R4′=OH and R5′=—S(═O)₂OH and R6′=—S(═O)₂OH; or
R4′=OH and R5′=—S(═O)₂OH and R6′=sulfonyl; or
R4′=OH and R5′=sulfonyl and R6′=H; or
R4′=OH and R5′=sulfonyl and R6′=OH; or
R4′=OH and R5′=sulfonyl and R6′=methyl; or
R4′=OH and R5′=sulfonyl and R6′=O-methyl; or
R4′=OH and R5′=sulfonyl and R6′=—S(═O)₂OH; or
R4′=OH and R5′=sulfonyl and R6′=sulfonyl; or
R4′=methyl and R5′=H and R6′=H; or
R4′=methyl and R5′=H and R6′=OH; or
R4′=methyl and R5′=H and R6′=methyl; or
R4′=methyl and R5′=H and R6′=O-methyl; or
R4′=methyl and R5′=H and R6′=—S(═O)₂OH; or
R4′=methyl and R5′=H and R6′=sulfonyl; or
R4′=methyl and R5′=OH and R6′=H; or
R4′=methyl and R5′=OH and R6′=OH; or
R4′=methyl and R5′=OH and R6′=methyl; or
R4′=methyl and R5′=OH and R6′=O-methyl; or
R4′=methyl and R5′=OH and R6′=—S(═O)₂OH; or
R4′=methyl and R5′=OH and R6′=sulfonyl; or
R4′=methyl and R5′=methyl and R6′=H; or
R4′=methyl and R5′=methyl and R6′=OH; or
R4′=methyl and R5′=methyl and R6′=methyl; or
R4′=methyl and R5′=methyl and R6′=O-methyl; or
R4′=methyl and R5′=methyl and R6′=—S(═O)₂OH; or
R4′=methyl and R5′=methyl and R6′=sulfonyl; or
R4′=methyl and R5′=O-methyl and R6′=H; or
R4′=methyl and R5′=O-methyl and R6′=OH; or
R4′=methyl and R5′=O-methyl and R6′=methyl; or
R4′=methyl and R5′=O-methyl and R6′=O-methyl; or
R4′=methyl and R5′=O-methyl and R6′=—S(═O)₂OH; or
R4′=methyl and R5′=O-methyl and R6′=sulfonyl; or
R4′=methyl and R5′=—S(═O)₂OH and R6′=H; or
R4′=methyl and R5′=—S(═O)₂OH and R6′=OH; or
R4′=methyl and R5′=—S(═O)₂OH and R6′=methyl; or
R4′=methyl and R5′=—S(═O)₂OH and R6′=O-methyl; or
R4′=methyl and R5′=—S(═O)₂OH and R6′=—S(═O)₂OH; or
R4′=methyl and R5′=—S(═O)₂OH and R6′=sulfonyl; or
R4′=methyl and R5′=sulfonyl and R6′=H; or
R4′=methyl and R5′=sulfonyl and R6′=OH; or
R4′=methyl and R5′=sulfonyl and R6′=methyl; or
R4′=methyl and R5′=sulfonyl and R6′=O-methyl; or
R4′=methyl and R5′=sulfonyl and R6′=—S(═O)₂OH; or
R4′=methyl and R5′=sulfonyl and R6′=sulfonyl; or
R4′=O-methyl and R5′=H and R6′=H; or
R4′=O-methyl and R5′=H and R6′=OH; or
R4′=O-methyl and R5′=H and R6′=methyl; or
R4′=O-methyl and R5′=H and R6′=O-methyl; or
R4′=O-methyl and R5′=H and R6′=—S(═O)₂OH; or
R4′=O-methyl and R5′=H and R6′=sulfonyl; or
R4′=O-methyl and R5′=OH and R6′=H; or
R4′=O-methyl and R5′=OH and R6′=OH; or
R4′=O-methyl and R5′=OH and R6′=methyl; or
R4′=O-methyl and R5′=OH and R6′=O-methyl; or
R4′=O-methyl and R5′=OH and R6′=—S(═O)₂OH; or
R4′=O-methyl and R5′=OH and R6′=sulfonyl; or
R4′=O-methyl and R5′=methyl and R6′=H; or
R4′=O-methyl and R5′=methyl and R6′=OH; or
R4′=O-methyl and R5′=methyl and R6′=methyl; or
R4′=O-methyl and R5′=methyl and R6′=O-methyl; or
R4′=O-methyl and R5′=methyl and R6′=—S(═O)₂OH; or
R4′=O-methyl and R5′=methyl and R6′=sulfonyl; or
R4′=O-methyl and R5′=O-methyl and R6′=H; or
R4′=O-methyl and R5′=O-methyl and R6′=OH; or
R4′=O-methyl and R5′=O-methyl and R6′=methyl; or
R4′=O-methyl and R5′=O-methyl and R6′=O-methyl; or
R4′=O-methyl and R5′=O-methyl and R6′=—S(═O)₂OH; or
R4′=O-methyl and R5′=O-methyl and R6′=sulfonyl; or
R4′=O-methyl and R5′=—S(═O)₂OH and R6′=H; or
R4′=O-methyl and R5′=—S(═O)₂OH and R6′=OH; or
R4′=O-methyl and R5′=—S(═O)₂OH and R6′=methyl; or
R4′=O-methyl and R5′=—S(═O)₂OH and R6′=O-methyl; or
R4′=O-methyl and R5′=—S(═O)₂OH and R6′=—S(═O)₂OH; or
R4′=O-methyl and R5′=—S(═O)₂OH and R6′=sulfonyl; or
R4′=O-methyl and R5′=sulfonyl and R6′=H; or
R4′=O-methyl and R5′=sulfonyl and R6′=OH; or
R4′=O-methyl and R5′=sulfonyl and R6′=methyl; or
R4′=O-methyl and R5′=sulfonyl and R6′=O-methyl; or
R4′=O-methyl and R5′=sulfonyl and R6′=—S(═O)₂OH; or
R4′=O-methyl and R5′=sulfonyl and R6′=sulfonyl; or
R4′=—S(═O)₂OH and R5′=H and R6′=H; or
R4′=—S(═O)₂OH and R5′=H and R6′=OH; or
R4′=—S(═O)₂OH and R5′=H and R6′=methyl; or
R4′=—S(═O)₂OH and R5′=H and R6′=O-methyl; or
R4′=—S(═O)₂OH and R5′=H and R6′=—S(═O)₂OH; or
R4′=—S(═O)₂OH and R5′=H and R6′=sulfonyl; or
R4′=—S(═O)₂OH and R5′=OH and R6′=H; or
R4′=—S(═O)₂OH and R5′=OH and R6′=OH; or
R4′=—S(═O)₂OH and R5′=OH and R6′=methyl; or
R4′=—S(═O)₂OH and R5′=OH and R6′=O-methyl; or
R4′=—S(═O)₂OH and R5′=OH and R6′=—S(═O)₂OH; or
R4′=—S(═O)₂OH and R5′=OH and R6′=sulfonyl; or
R4′=—S(═O)₂OH and R5′=methyl and R6′=H; or
R4′=—S(═O)₂OH and R5′=methyl and R6′=OH; or
R4′=—S(═O)₂OH and R5′=methyl and R6′=methyl; or
R4′=—S(═O)₂OH and R5′=methyl and R6′=O-methyl; or
R4′=—S(═O)₂OH and R5′=methyl and R6′=—S(═O)₂OH; or
R4′=—S(═O)₂OH and R5′=methyl and R6′=sulfonyl; or
R4′=—S(═O)₂OH and R5′=O-methyl and R6′=H; or
R4′=—S(═O)₂OH and R5′=O-methyl and R6′=OH; or
R4′=—S(═O)₂OH and R5′=O-methyl and R6′=methyl; or
R4′=—S(═O)₂OH and R5′=O-methyl and R6′=O-methyl; or
R4′=—S(═O)₂OH and R5′=O-methyl and R6′=—S(═O)₂OH; or
R4′=—S(═O)₂OH and R5′=O-methyl and R6′=sulfonyl; or
R4′=—S(═O)₂OH and R5′=—S(═O)₂OH═ and R6′=H; or
R4′=—S(═O)₂OH and R5′=—S(═O)₂OH and R6′=OH; or
R4′=—S(═O)₂OH and R5′=—S(═O)₂OH and R6′=methyl; or
R4′=—S(═O)₂OH and R5′=—S(═O)₂OH and R6′=O-methyl; or
R4′=—S(═O)₂OH and R5′=sulfonyl=and R6′=H; or
R4′=—S(═O)₂OH and R5′=sulfonyl and R6′=OH; or
R4′=—S(═O)₂OH and R5′=sulfonyl and R6′=methyl; or
R4′=—S(═O)₂OH and R5′=sulfonyl and R6′=O-methyl; or
R4′=sulfonyl and R5′=H and R6′=H; or
R4′=sulfonyl and R5′=H and R6′=OH; or
R4′=sulfonyl and R5′=H and R6′=methyl; or
R4′=-sulfonyl and R5′=H and R6′=O-methyl; or
R4′=sulfonyl and R5′=H and R6′=—S(═O)₂OH; or
R4′=sulfonyl and R5′=H and R6′=sulfonyl; or
R4′=sulfonyl and R5′=OH and R6′=H; or
R4′=sulfonyl and R5′=OH and R6′=OH; or
R4′=sulfonyl and R5′=OH and R6′=methyl; or
R4′=sulfonyl and R5′=OH and R6′=O-methyl; or
R4′=sulfonyl and R5′=OH and R6′=—S(═O)₂OH; or
R4′=sulfonyl and R5′=OH and R6′=sulfonyl; or
R4′=sulfonyl and R5′=methyl and R6′=H; or
R4′=sulfonyl and R5′=methyl and R6′=OH; or
R4′=sulfonyl and R5′=methyl and R6′=methyl; or
R4′=sulfonyl and R5′=methyl and R6′=O-methyl; or
R4′=sulfonyl and R5′=methyl and R6′=—S(═O)₂OH; or
R4′=sulfonyl and R5′=methyl and R6′=sulfonyl; or
R4′=sulfonyl and R5′=O-methyl and R6′=H; or
R4′=sulfonyl and R5′=O-methyl and R6′=OH; or
R4′=sulfonyl and R5′=O-methyl and R6′=methyl; or
R4′=sulfonyl and R5′=O-methyl and R6′=O-methyl; or
R4′=sulfonyl and R5′=O-methyl and R6′=—S(═O)₂OH; or
R4′=sulfonyl and R5′=O-methyl and R6′=sulfonyl; or
R4′=sulfonyl and R5′=—S(═O)₂OH and R6′=H; or
R4′=sulfonyl and R5′=—S(═O)₂OH and R6′=OH; or
R4′=sulfonyl and R5′=—S(═O)₂OH and R6′=methyl; or
R4′=sulfonyl and R5′=—S(═O)₂OH and R6′=O-methyl; or
R4′=sulfonyl and R5′=sulfonyl and R6′=H; or
R4′=sulfonyl and R5′=sulfonyl and R6′=OH; or
R4′=sulfonyl and R5′=sulfonyl and R6′=methyl; or
R4′=sulfonyl and R5′=sulfonyl and R6′=O-methyl; or
R4″=H and R5″=H and R6″=H; or
R4″=H and R5″=H and R6″=OH; or
R4″=H and R5″=H and R6″=methyl; or
R4″=H and R5″=H and R6″=O-methyl; or
R4″=H and R5″=H and R6″=—S(═O)₂OH; or
R4″=H and R5″=H and R6″=sulfonyl; or
R4″=H and R5″=OH and R6″=H; or
R4″=H and R5″=OH and R6″=OH; or
R4″=H and R5″=OH and R6″=methyl; or
R4″=H and R5″=OH and R6″=O-methyl; or
R4″=H and R5″=OH and R6″=—S(═O)₂OH; or
R4″=H and R5″=OH and R6″=sulfonyl; or
R4″=H and R5″=methyl and R6″=H; or
R4″=H and R5″=methyl and R6″=OH; or
R4″=H and R5″=methyl and R6″=methyl; or
R4″=H and R5″=methyl and R6″=O-methyl; or
R4″=H and R5″=methyl and R6″=—S(═O)₂OH; or
R4″=H and R5″=methyl and R6″=sulfonyl; or
R4″=H and R5″=O-methyl and R6″=H; or
R4″=H and R5″=O-methyl and R6″=OH; or
R4″=H and R5″=O-methyl and R6″=methyl; or
R4″=H and R5″=O-methyl and R6″=O-methyl; or
R4″=H and R5″=O-methyl and R6″=—S(═O)₂OH; or
R4″=H and R5″=O-methyl and R6″=sulfonyl; or
R4″=H and R5″=—S(═O)₂OH and R6″=H; or
R4″=H and R5″=—S(═O)₂OH and R6″=OH; or
R4″=H and R5″=—S(═O)₂OH and R6″=methyl; or
R4″=H and R5″=—S(═O)₂OH and R6″=O-methyl; or
R4″=H and R5″=—S(═O)₂OH and R6″=—S(═O)₂OH; or
R4″=H and R5″=—S(═O)₂OH and R6″=sulfonyl; or
R4″=H and R5″=sulfonyl and R6″=H; or
R4″=H and R5″=sulfonyl and R6″=OH; or
R4″=H and R5″=sulfonyl and R6″=methyl; or
R4″=H and R5″=sulfonyl and R6″=O-methyl; or
R4″=H and R5″=sulfonyl and R6″=—S(═O)₂OH; or
R4″=H and R5″=sulfonyl and R6″=sulfonyl; or
R4″=OH and R5″=H and R6″=H; or
R4″=OH and R5″=H and R6″=OH; or
R4″=OH and R5″=H and R6″=methyl; or
R4″=OH and R5″=H and R6″=O-methyl; or
R4″=OH and R5″=H and R6″=—S(═O)₂OH; or
R4″=OH and R5″=H and R6″=sulfonyl; or
R4″=OH and R5″=OH and R6″=H; or
R4″=OH and R5″=OH and R6″=OH; or
R4″=OH and R5″=OH and R6″=methyl; or
R4″=OH and R5″=OH and R6″=O-methyl; or
R4″=OH and R5″=OH and R6″=—S(═O)₂OH; or
R4″=OH and R5″=OH and R6″=sulfonyl; or
R4″=OH and R5″=methyl and R6″=H; or
R4″=OH and R5″=methyl and R6″=OH; or
R4″=OH and R5″=methyl and R6″=methyl; or
R4″=OH and R5″=methyl and R6″=O-methyl; or
R4″=OH and R5″=methyl and R6″=—S(═O)₂OH; or
R4″=OH and R5″=methyl and R6″=sulfonyl; or
R4″=OH and R5″=O-methyl and R6″=H; or
R4″=OH and R5″=O-methyl and R6″=OH; or
R4″=OH and R5″=O-methyl and R6″=methyl; or
R4″=OH and R5″=O-methyl and R6″=O-methyl; or
R4″=OH and R5″=O-methyl and R6″=—S(═O)₂OH; or
R4″=OH and R5″=O-methyl and R6″=sulfonyl; or
R4″=OH and R5″=—S(═O)₂OH and R6″=H; or
R4″=OH and R5″=—S(═O)₂OH and R6″=OH; or
R4″=OH and R5″=—S(═O)₂OH and R6″=methyl; or
R4″=OH and R5″=—S(═O)₂OH and R6″=O-methyl; or
R4″=OH and R5″=—S(═O)₂OH and R6″=—S(═O)₂OH; or
R4″=OH and R5″=—S(═O)₂OH and R6″=sulfonyl; or
R4″=OH and R5″=sulfonyl and R6″=H; or
R4″=OH and R5″=sulfonyl and R6″=OH; or
R4″=OH and R5″=sulfonyl and R6″=methyl; or
R4″=OH and R5″=sulfonyl and R6″=O-methyl; or
R4″=OH and R5″=sulfonyl and R6″=—S(═O)₂OH; or
R4″=OH and R5″=sulfonyl and R6″=sulfonyl; or
R4″=methyl and R5″=H and R6″=H; or
R4″=methyl and R5″=H and R6″=OH; or
R4″=methyl and R5″=H and R6″=methyl; or
R4″=methyl and R5″=H and R6″=O-methyl; or
R4″=methyl and R5″=H and R6″=—S(═O)₂OH; or
R4″=methyl and R5″=H and R6″=sulfonyl; or
R4″=methyl and R5″=OH and R6″=H; or
R4″=methyl and R5″=OH and R6″=OH; or
R4″=methyl and R5″=OH and R6″=methyl; or
R4″=methyl and R5″=OH and R6″=O-methyl; or
R4″=methyl and R5″=OH and R6″=—S(═O)₂OH; or
R4″=methyl and R5″=OH and R6″=sulfonyl; or
R4″=methyl and R5″=methyl and R6″=H; or
R4″=methyl and R5″=methyl and R6″=OH; or
R4″=methyl and R5″=methyl and R6″=methyl; or
R4″=methyl and R5″=methyl and R6″=O-methyl; or
R4″=methyl and R5″=methyl and R6″=—S(═O)₂OH; or
R4″=methyl and R5″=methyl and R6″=sulfonyl; or
R4″=methyl and R5″=O-methyl and R6″=H; or
R4″=methyl and R5″=O-methyl and R6″=OH; or
R4″=methyl and R5″=O-methyl and R6″=methyl; or
R4″=methyl and R5″=O-methyl and R6″=O-methyl; or
R4″=methyl and R5″=O-methyl and R6″=—S(═O)₂OH; or
R4″=methyl and R5″=O-methyl and R6″=sulfonyl; or
R4″=methyl and R5″=—S(═O)₂OH and R6″=H; or
R4″=methyl and R5″=—S(═O)₂OH and R6″=OH; or
R4″=methyl and R5″=—S(═O)₂OH and R6″=methyl; or
R4″=methyl and R5″=—S(═O)₂OH and R6″=O-methyl; or
R4″=methyl and R5″=—S(═O)₂OH and R6″=—S(═O)₂OH; or
R4″=methyl and R5″=—S(═O)₂OH and R6″=sulfonyl; or
R4″=methyl and R5″=sulfonyl and R6″=H; or
R4″=methyl and R5″=sulfonyl and R6″=OH; or
R4″=methyl and R5″=sulfonyl and R6″=methyl; or
R4″=methyl and R5″=sulfonyl and R6″=O-methyl; or
R4″=methyl and R5″=sulfonyl and R6″=—S(═O)₂OH; or
R4″=methyl and R5″=sulfonyl and R6″=sulfonyl; or
R4″=O-methyl and R5″=H and R6″=H; or
R4″=O-methyl and R5″=H and R6″=OH; or
R4″=O-methyl and R5″=H and R6″=methyl; or
R4″=O-methyl and R5″=H and R6″=O-methyl; or
R4″=O-methyl and R5″=H and R6″=—S(═O)₂OH; or
R4″=O-methyl and R5″=H and R6″=sulfonyl; or
R4″=O-methyl and R5″=OH and R6″=H; or
R4″=O-methyl and R5″=OH and R6″=OH; or
R4″=O-methyl and R5″=OH and R6″=methyl; or
R4″=O-methyl and R5″=OH and R6″=O-methyl; or
R4″=O-methyl and R5″=OH and R6″=—S(═O)₂OH; or
R4″=O-methyl and R5″=OH and R6″=sulfonyl; or
R4″=O-methyl and R5″=methyl and R6″=H; or
R4″=O-methyl and R5″=methyl and R6″=OH; or
R4″=O-methyl and R5″=methyl and R6″=methyl; or
R4″=O-methyl and R5″=methyl and R6″=O-methyl; or
R4″=O-methyl and R5″=methyl and R6″=—S(═O)₂OH; or
R4″=O-methyl and R5″=methyl and R6″=sulfonyl; or
R4″=O-methyl and R5″=O-methyl and R6″=H; or
R4″=O-methyl and R5″=O-methyl and R6″=OH; or
R4″=O-methyl and R5″=O-methyl and R6″=methyl; or
R4″=O-methyl and R5″=O-methyl and R6″=O-methyl; or
R4″=O-methyl and R5″=O-methyl and R6″=—S(═O)₂OH; or
R4″=O-methyl and R5″=O-methyl and R6″=sulfonyl; or
R4″=O-methyl and R5″=—S(═O)₂OH and R6″=H; or
R4″=O-methyl and R5″=—S(═O)₂OH and R6″=OH; or
R4″=O-methyl and R5″=—S(═O)₂OH and R6″=methyl; or
R4″=O-methyl and R5″=—S(═O)₂OH and R6″=O-methyl; or
R4″=O-methyl and R5″=—S(═O)₂OH and R6″=—S(═O)₂OH; or
R4″=O-methyl and R5″=—S(═O)₂OH and R6″=sulfonyl; or
R4″=O-methyl and R5″=sulfonyl and R6″=H; or
R4″=O-methyl and R5″=sulfonyl and R6″=OH; or
R4″=O-methyl and R5″=sulfonyl and R6″=methyl; or
R4″=O-methyl and R5″=sulfonyl and R6″=O-methyl; or
R4″=O-methyl and R5″=sulfonyl and R6″=—S(═O)₂OH; or
R4″=O-methyl and R5″=sulfonyl and R6″=sulfonyl; or
R4″=—S(═O)₂OH and R5″=H and R6″=H; or
R4″=—S(═O)₂OH and R5″=H and R6″=OH; or
R4″=—S(═O)₂OH and R5″=H and R6″=methyl; or
R4″=—S(═O)₂OH and R5″=H and R6″=O-methyl; or
R4″=—S(═O)₂OH and R5″=H and R6″=—S(═O)₂OH; or
R4″=—S(═O)₂OH and R5″=H and R6″=sulfonyl; or
R4″=—S(═O)₂OH and R5″=OH and R6″=H; or
R4″=—S(═O)₂OH and R5″=OH and R6″=OH; or
R4″=—S(═O)₂OH and R5″=OH and R6″=methyl; or
R4″=—S(═O)₂OH and R5″=OH and R6″=O-methyl; or
R4″=—S(═O)₂OH and R5″=OH and R6″=—S(═O)₂OH; or
R4″=—S(═O)₂OH and R5″=OH and R6″=sulfonyl; or
R4″=—S(═O)₂OH and R5″=methyl and R6″=H; or
R4″=—S(═O)₂OH and R5″=methyl and R6″=OH; or
R4″=—S(═O)₂OH and R5″=methyl and R6″=methyl; or
R4″=—S(═O)₂OH and R5″=methyl and R6″=O-methyl; or
R4″=—S(═O)₂OH and R5″=methyl and R6″=—S(═O)₂OH; or
R4″=—S(═O)₂OH and R5″=methyl and R6″=sulfonyl; or
R4″=—S(═O)₂OH and R5″=O-methyl and R6″=H; or
R4″=—S(═O)₂OH and R5″=O-methyl and R6″=OH; or
R4″=—S(═O)₂OH and R5″=O-methyl and R6″=methyl; or
R4″=—S(═O)₂OH and R5″=O-methyl and R6″=O-methyl; or
R4″=—S(═O)₂OH and R5″=O-methyl and R6″=—S(═O)₂OH; or
R4″=—S(═O)₂OH and R5″=O-methyl and R6″=sulfonyl; or
R4″=—S(═O)₂OH and R5″=—S(═O)₂OH═ and R6″=H; or
R4″=—S(═O)₂OH and R5″=—S(═O)₂OH and R6″=OH; or
R4″=—S(═O)₂OH and R5″=—S(═O)₂OH and R6″=methyl; or
R4″=—S(═O)₂OH and R5″=—S(═O)₂OH and R6″=O-methyl; or
R4″=—S(═O)₂OH and R5″=sulfonyl=and R6″=H; or
R4″=—S(═O)₂OH and R5″=sulfonyl and R6″=OH; or
R4″=—S(═O)₂OH and R5″=sulfonyl and R6″=methyl; or
R4″=—S(═O)₂OH and R5″=sulfonyl and R6″=O-methyl; or
R4″=sulfonyl and R5″=H and R6″=H; or
R4″=sulfonyl and R5″=H and R6″=OH; or
R4″=sulfonyl and R5″=H and R6″=methyl; or
R4″=-sulfonyl and R5″=H and R6″=O-methyl; or
R4″=sulfonyl and R5″=H and R6″=—S(═O)₂OH; or
R4″=sulfonyl and R5″=H and R6″=sulfonyl; or
R4″=sulfonyl and R5″=OH and R6″=H; or
R4″=sulfonyl and R5″=OH and R6″=OH; or
R4″=sulfonyl and R5″=OH and R6″=methyl; or
R4″=sulfonyl and R5″=OH and R6″=O-methyl; or
R4″=sulfonyl and R5″=OH and R6″=—S(═O)₂OH; or
R4″=sulfonyl and R5″=OH and R6″=sulfonyl; or
R4″=sulfonyl and R5″=methyl and R6″=H; or
R4″=sulfonyl and R5″=methyl and R6″=OH; or
R4″=sulfonyl and R5″=methyl and R6″=methyl; or
R4″=sulfonyl and R5″=methyl and R6″=O-methyl; or
R4″=sulfonyl and R5″=methyl and R6″=—S(═O)₂OH; or
R4″=sulfonyl and R5″=methyl and R6″=sulfonyl; or
R4″=sulfonyl and R5″=O-methyl and R6″=H; or
R4″=sulfonyl and R5″=O-methyl and R6″=OH; or
R4″=sulfonyl and R5″=O-methyl and R6″=methyl; or
R4″=sulfonyl and R5″=O-methyl and R6″=O-methyl; or
R4″=sulfonyl and R5″=O-methyl and R6″=—S(═O)₂OH; or
R4″=sulfonyl and R5″=O-methyl and R6″=sulfonyl; or
R4″=sulfonyl and R5″=—S(═O)₂OH and R6″=H; or
R4″=sulfonyl and R5″=—S(═O)₂OH and R6″=OH; or
R4″=sulfonyl and R5″=—S(═O)₂OH and R6″=methyl; or
R4″=sulfonyl and R5″=—S(═O)₂OH and R6″=O-methyl; or
R4″=sulfonyl and R5″=sulfonyl and R6″=H; or
R4″=sulfonyl and R5″=sulfonyl and R6″=OH; or
R4″=sulfonyl and R5″=sulfonyl and R6″=methyl; or
R4″=sulfonyl and R5″=sulfonyl and R6″=O-methyl.

In the general formula (V), formula (VI), formula (VII), formula (Vill), formula (IX) and formula (X) and their variants, the substituted phenyl ring bonded to the imino functionality is preferably selected from the group consisting of

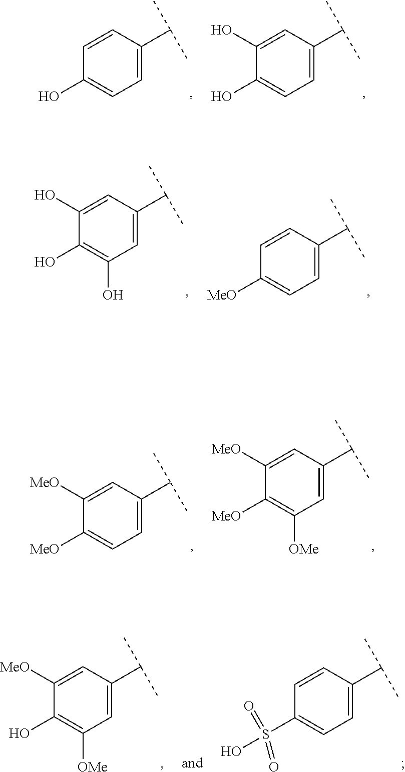

wherein the dotted line designates the binding site to the cyclohexene imine ring.

In a still more preferred variant according to the first aspect of the present invention, in the general formulae (V), (VII), (VIII) and their variants, the substituents R4′, R5′ and R6′, which may be identical or different, are independently from each other selected from the group consisting of H, OH, alkyl, preferably methyl, ethyl, propyl, iso-propyl, butyl, isobutyl or tert.-butyl, alkoxy, preferably, O-methyl, O-ethyl or O-butyl, —S(═O)₂OH, and sulfonyl; with the proviso, that if the substituted phenyl ring is monosubstituted, the substituent is either in the ortho or meta-position to the imino-functionality of the general formulae (V), (VI) and (VIII), but not in the para position to the imino-functionality.

In a still more preferred variant according to the first aspect of the present invention, in the general formulae (VI), (IX), (X) and their variants, the substituents R4″, R5″ and R6″, which may be identical or different, are independently from each other selected from the group consisting of H, OH, alkyl, preferably methyl, ethyl, propyl, iso-propyl, butyl, isobutyl or tert.-butyl, alkoxy, preferably, O-methyl, O-ethyl or O-butyl, —S(═O)₂OH, and sulfonyl; with the proviso, that if the substituted phenyl ring is monosubstituted, the substituent is either in the ortho or meta-position to the imino-functionality of the general formulae (VI), (IX), (X), but not in the para position to the imino-functionality.

Still more preferred according to the first aspect of the present invention, the sunscreen product, cosmetic or pharmaceutical preparation or homecare product comprises at least one mycosporine-like amino acid compound selected from the group consisting of:

TABLE 3

| Com- | | |
|---|---|---|
| pound | Structure | INCI/chemical name |
| MAA-1 | 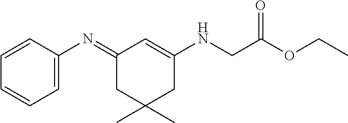 [SMILES: CCOC(=O)CNC1=C/C(=N/c2ccccc2)CC(C)(C)C1] | ethyl 2-[5,5-dimethyl-3- phenylimino-cyclohexen-1- yl]amino]acetate |
| MAA-2 | 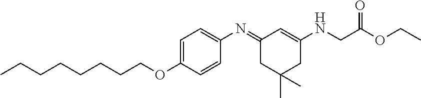 [SMILES: CCCCCCCCOc1ccc(/N=C2/C=C(NCC(=O)OCC)CC(C)(C)C2)cc1] | ethyl 2-((5,5-dimethyl-3-((4- octyoxy)phenyl)imino)cyclohex-1- en-1-yl)amino)acetate |
| MAA-3 | 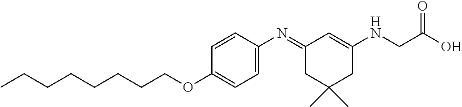 [SMILES: CCCCCCCCOc1ccc(/N=C2/C=C(NCC(=O)O)CC(C)(C)C2)cc1] | 5,5-dimethyl-3-((4- (octyloxy)phenyl)imino)cyclohex- 1-en-1-yl-glycine |
| MAA-4 | 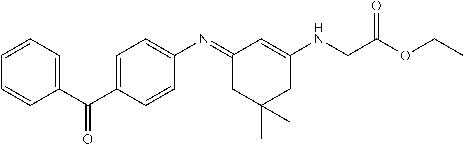 [SMILES: CCOC(=O)CNC1=C/C(=N/c2ccc(C(=O)c3ccccc3)cc2)CC(C)(C)C1] | ethyl 2-((3-((4- benzoylphenyl)imino)-5,5- dimethylcyclohex-1-en-1- yl)amino)acetate |
| MAA-5 | 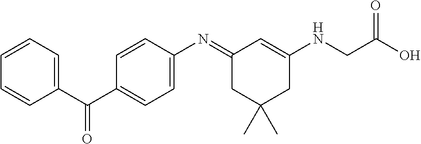 [SMILES: CC1(C)CC(NCC(=O)O)=C/C(=N/c2ccc(C(=O)c3ccccc3)cc2)C1] | 3-((4-benzoylphenyl)imino)-5,5- dimethylcyclohex-1-en-1-yl- glycine |
| MAA-6 | 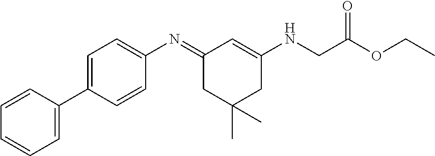 [SMILES: CCOC(=O)CNC1=C/C(=N/c2ccc(-c3ccccc3)cc2)CC(C)(C)C1] | ethyl 2-((3-([1,1′-biphenyl]-4- ylimino)-5,5-dimethylcyclohex-1- en-1.yl)amino)acetate |
| MAA-7 | 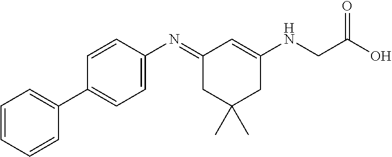 [SMILES: CC1(C)CC(NCC(=O)O)=C/C(=N/c2ccc(-c3ccccc3)cc2)C1] | 3-([1,1′-biphenyl]-4-ylimino)-5,5- dimethylcyclohex-1-en-1-yl- glycine |
| MAA-8 | 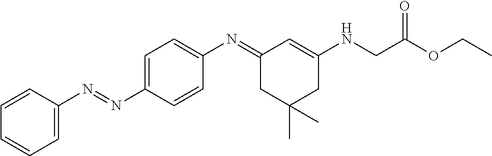 [SMILES: CCOC(=O)CNC1=C/C(=N/c2ccc(/N=N/c3ccccc3)cc2)CC(C)(C)C1] | ethyl 2-((5,5-dimethyl-3-((4- (phenyldiazenyl)phenyl)inino(cyclo- hex-1-en-1-yl)amino)acetate |
| MAA-9 | 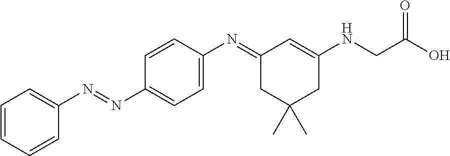 [SMILES: CC1(C)CC(NCC(=O)O)=C/C(=N/c2ccc(/N=N/c3ccccc3)cc2)C1] | 5,5-dimethyl-3-((4-((E)- phenyldiazenyl)phenyl)imino)cyclo- hex-1-en-1-yl-glycine |
| MAA-11 | 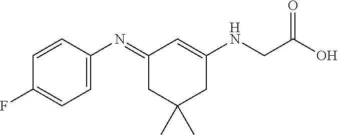 [SMILES: CC1(C)CC(NCC(=O)O)=C/C(=N/c2ccc(F)cc2)C1] | 3-((4-fluorophenyl)imino)-5,5- dimethylcyclohex-1-en-1-yl- glycine |
| MAA-12 | 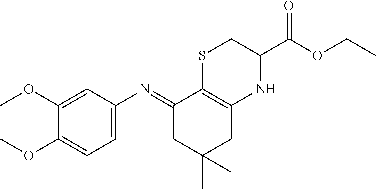 [SMILES: CCOC(=O)C1CSC2=C(CC(C)(C)C/C2=N\c2ccc(OC)c(OC)c2)N1] | ethyl 8-((3,4- dimethoyxphenyl)imino)-6,6- dimethyl-3,4,5,6,7,8-hexahydro-2- H-benzol[b][1,4]thiazine-3- carboxylate |
| MAA-13 | 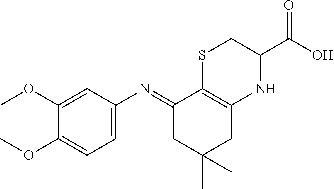 [SMILES: COc1ccc(/N=C2\CC(C)(C)CC3=C2SCC(C(=O)O)N3)cc1OC] | 8-((3,4-dimethoyxphenyl)imino)- 6,6-dimethyl-3,4,5,6,7,8- hexahydro-2-H- benzol[b][1,4]thiazine-3- carboxylic acid |
| MAA-14 | 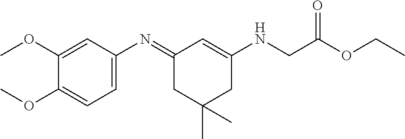 [SMILES: CCOC(=O)CNC1=C/C(=N/c2ccc(OC)c(OC)c2)CC(C)(C)C1] | ethyl 2-((3-((3,4- dimethoxyphenyl)imino)-5,5- dimethylcyclohex-1-en-1- yl)amino)acetate |
| MAA-16 | 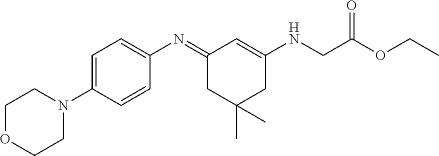 [SMILES: CCOC(=O)CNC1=C/C(=N/c2ccc(N3CCOCC3)cc2)CC(C)(C)C1] | ethyl 2-((5,5-dimethyl-3-((4- morpholinophenyl)imino)cyclohex- 1-en-1-yl)amino)acetate |
| MAA-17 | 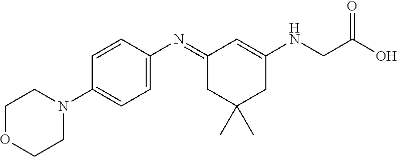 [SMILES: CC1(C)CC(NCC(=O)O)=C/C(=N/c2ccc(N3CCOCC3)cc2)C1] | 5,5-dimethyl-3-((4- morpholinophenyl)imino)cyclohex- 1-en-1-yl-glycine |
| MAA-20 | 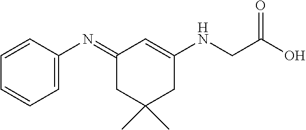 [SMILES: CC1(C)CC(NCC(=O)O)=C/C(=N/c2ccccc2)C1] | 2-[(5,5-dimethyl-3-phenylimino- cyclohexen-1-yl)amino]acetic acid |
| MAA-21 | 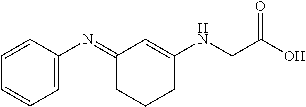 [SMILES: O=C(O)CNC1=C/C(=N/c2ccccc2)CCC1] | 2-[(3-phenyliminocyclohexen-1- yl)amino]acetic acid |
| MAA-22 | 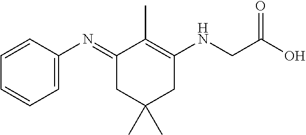 [SMILES: CC1=C(NCC(=O)O)CC(C)(C)C/C1=N\c1ccccc1] | 2,5,5-trimethyl-3- (phenylimino)cyclohex-1-en-1-yl- glycine |
| MAA-23 | 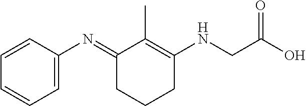 [SMILES: CC1=C(NCC(=O)O)CCC/C1=N\c1ccccc1] | 2-[(2-methyl-3-phenylimino- cyclohexen-1-yl)amino]acetic acid |
| MAA-24 | 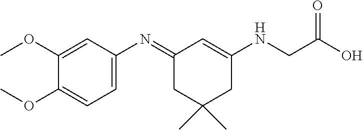 [SMILES: COc1ccc(/N=C2/C=C(NCC(=O)O)CC(C)(C)C2)cc1OC] | 3-((3,4-dimethoxyphenyl)imino)- 5,5-dimethylcyclohex-1-en-1-yl- glycine |
| MAA-25 | 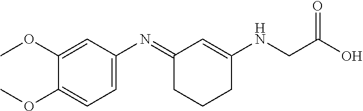 [SMILES: COc1ccc(/N=C2/C=C(NCC(=O)O)CCC2)cc1OC] | 3-((3,4- dimethoxyphenyl)imino)cyclohex- 1-en-1-yl-glycine |
| MAA-26 | 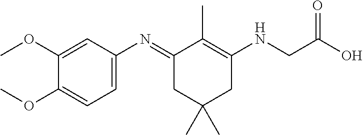 [SMILES: COc1ccc(/N=C2\CC(C)(C)CC(NCC(=O)O)=C2C)cc1OC] | 3-((3,4-dimethoxyphenyl)imino)- 2,5,5-trimethylcyclohex-1-en-1-yl- glycine |
| MAA-27 | 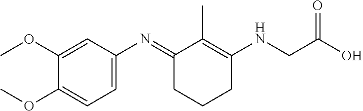 [SMILES: COc1ccc(/N=C2\CCCC(NCC(=O)O)=C2C)cc1OC] | 2-[(3-(3,4-dimethoxyphenyl)imino- 2-methyl-cyclohexen-1- yl)amino]acetic acid |
| MAA-28 | 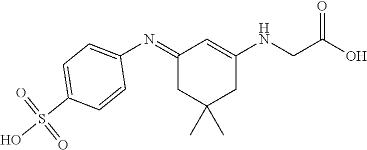 [SMILES: CC1(C)CC(NCC(=O)O)=C/C(=N/c2ccc(S(=O)(=O)O)cc2)C1] | 2-[(5,5-dimethyl-3-(4- sulfophenyl)imino-cyclohexen-1- yl)amino]acetic acid |
| MAA-29 | 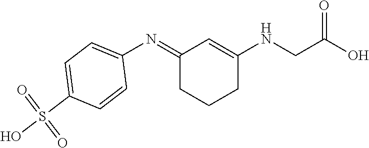 [SMILES: O=C(O)CNC1=C/C(=N/c2ccc(S(=O)(=O)O)cc2)CCC1] | 2-[(3-(4- sulfophenyl)iminocyclohexen-1- yl)amino]acetic acid |
| MAA-30 | 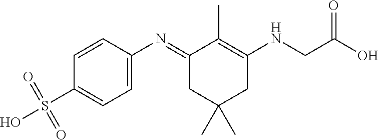 [SMILES: CC1=C(NCC(=O)O)CC(C)(C)C/C1=N\c1ccc(S(=O)(=O)O)cc1] | 2,5,5-trimethyl-3-((4- sulfophenyl)imino)cyclohex-1-en- 1-yl-glycine |
| MAA-31 | 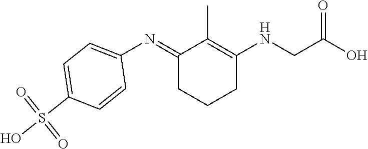 [SMILES: CC1=C(NCC(=O)O)CCC/C1=N\c1ccc(S(=O)(=O)O)cc1] | 2-methyl-3-((4- sulfophenyl)imino)cyclohex-1-en- 1-yl-glycine |
| MAA-32 | 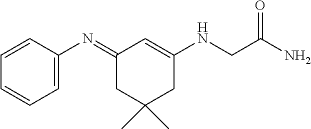 [SMILES: CC1(C)CC(NCC(N)=O)=C/C(=N/c2ccccc2)C1] | 2-[(5,5-dimethyl-3-phenylimino- cyclohexen-1-yl)amino]acetamide |
| MAA-33 | 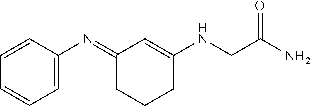 [SMILES: NC(=O)CNC1=C/C(=N/c2ccccc2)CCC1] | 2-[(3-phenyliminocyclohexen-1- yl)amino]acetamide |
| MAA-34 | 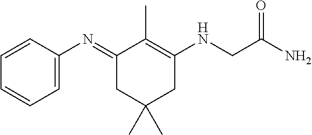 [SMILES: CC1=C(NCC(N)=O)CC(C)(C)C/C1=N\c1ccccc1] | 2-((2,5,5-trimethyl-3- (phenylimino)cyclohex-1-en-1- yl)amino)acetamide |
| MAA-35 | 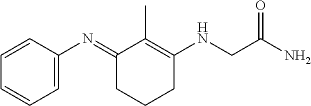 [SMILES: CC1=C(NCC(N)=O)CCC/C1=N\c1ccccc1] | 2-((2-methyl-3- (phenylimino)cyclohex-1-en-1- yl)amino)acetamide |
| MAA-36 | 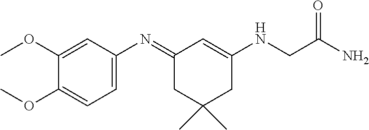 [SMILES: COc1ccc(/N=C2/C=C(NCC(N)=O)CC(C)(C)C2)cc1OC] | 2-((3-((3,4- dimethoxyphenyl)imino)-5,5- dimethylcyclohex-1-en-1- yl)amino)acetamide |
| MAA-37 | 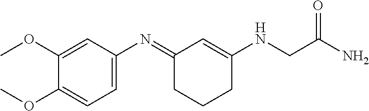 [SMILES: COc1ccc(/N=C2/C=C(NCC(N)=O)CCC2)cc1OC] | 2-[(3-(3,4- dimethoxyphenyl)iminocyclohexen- 1-yl)amino]acetamide |
| MAA-38 | 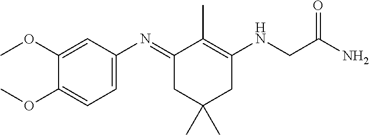 [SMILES: COc1ccc(/N=C2\CC(C)(C)CC(NCC(N)=O)=C2C)cc1OC] | 2-((3-((3,4- dimethoxyphenyl)imino)-2,5,5- trimethylcyclohex-1-en-1- yl)amino)acetamide |
| MAA-39 | 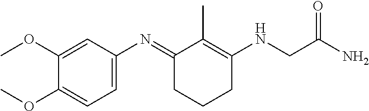 [SMILES: COc1ccc(/N=C2\CCCC(NCC(N)=O)=C2C)cc1OC] | 2-((3-((3,4- dimethoxyphenyl)imino)-2- methylcyclohex-1-en-1- yl)amino)acetamide |

or a tautomer or a stereoisomer or a salt of the afore-mentioned compounds, or any mixture of the afore-mentioned compounds.

Among the above specified mycosporine-like amino acid compounds the compounds MAA-1 and MAA-13 are particularly preferred, since they have a pronounced water resistance improving effect. Most preferred are the acid compounds MAA-20, MAA-21, MAA-22, MAA-23, MAA-24, MAA-24, MAA-26, MAA-27, MAA-28, MAA-29, MAA-30, MAA-31, and the amide compounds MAA-32, MAA-33, MAA-34, MAA-35, MAA-36, MAA-37, MAA-38, and MAA-39. Said compound having an acid functionality or an amide functionality are photo-stable and temperature-stable and show stability in pH solution or in oil-in-water emulsions and do not degrade.

Especially, the mycosporine-like amino acid compounds having an acid functionality have a better pH stability in a broader pH-range compared to their respective ester compounds which hydrolize, and, thus, are not stable in alkaline solutions having a pH higher than 9.

Due to their better solubility in water, said mycosporine-like amino acid compounds can be incorporated into the water-phase of emulsions in higher concentrations, resulting in a better boosting effect for film forming agents, and, thus and improved sunscreen efficacy in sunscreen products, cosmetic or pharmaceutical preparations or homecare products, compared to their respective ester compounds.

Still more preferred according to the first aspect of the present invention, the sunscreen product, cosmetic or pharmaceutical preparation or homecare product comprises at least one mycosporine-like amino acid compound selected from the group consisting of:

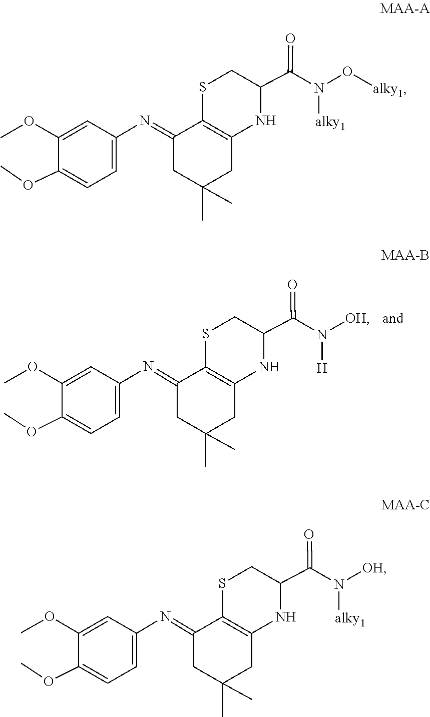

or a tautomer or a stereoisomer or a salt of the afore-mentioned compounds, or any mixture of the afore-mentioned compounds, wherein alkyl is preferably either methyl or ethyl or propyl or isopropyl or butyl or isobutyl or tert-butyl, most preferred methyl.

The mycosporine-like amino acid compounds according to the present invention and defined herein are used either as single substance or in a mixture with one, two or more different mycosporine-like amino acid compounds as defined herein.

Particular preferred are mixtures of two mycosporine-like amino acid compounds according to the present invention and defined herein. Such mixtures show a particular enhanced boosting effect, as it is demonstrated by the following examples.

The sunscreen product, cosmetic or pharmaceutical composition or homecare product according to the first aspect of the present invention is further characterized in that it does not contain one or more of the following mycosporine-like amino acid compounds:

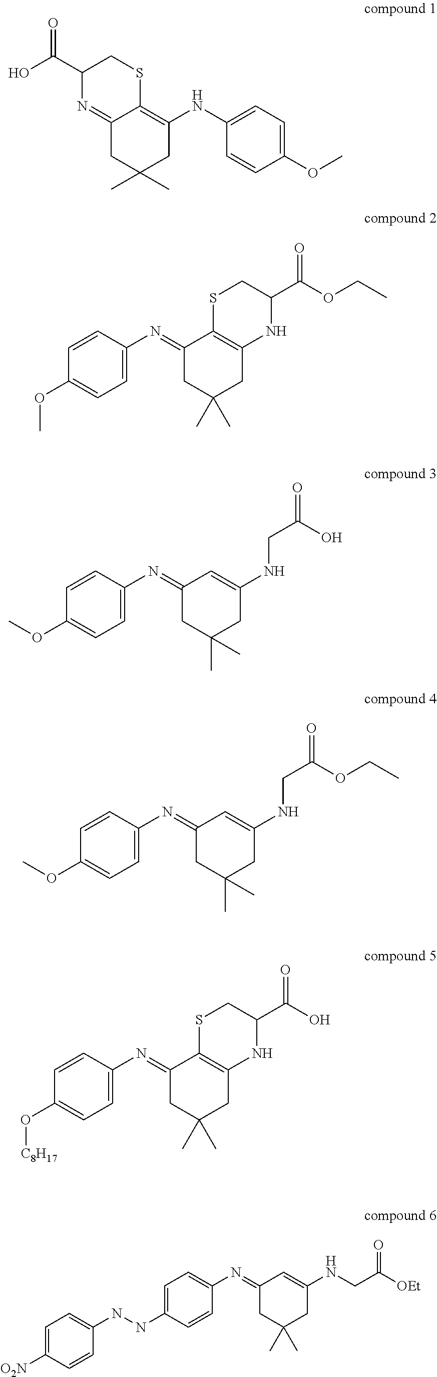

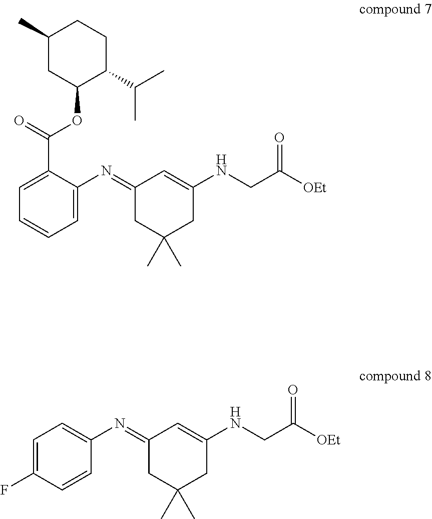 [SMILES: CCOC(=O)CNC1=C/C(=N/c2ccccc2C(=O)O[C@H]2C[C@@H](C)CC[C@@H]2C(C)C)CC(C)(C)C1 ; CCOC(=O)CNC1=C/C(=N/c2ccc(F)cc2)CC(C)(C)C1]

Various mycosporine-like amino acid compounds (b) as described herein contain one or more chiral centers, and, thus, can exist as racemic mixtures of enantiomers, mixtures of diastereomers or enantiomerically or optically pure compounds. It should also be noted the compounds of the invention include E and Z isomers, or a mixture thereof, and cis and trans isomers or a mixture thereof.

Accordingly, the chemical structures of the general formulae (V) to (X) and their variants and compounds explicitly disclosed herein include all possible enantiomers and diastereomers or stereoisomers.

In certain embodiments, the mycosporine-like amino acid compounds (b) of the invention are isolated as either the E or Z isomer. In other embodiments, the compounds of the invention are a mixture of the E and Z isomers.

Alternatively, stereomerically pure compounds are used in the sunscreen product, cosmetic or pharmaceutical preparation or homecare product according to the present invention. As used herein and unless otherwise indicated, the term “stereomerically pure” means that one stereoisomer of a compound is substantially free of other stereoisomers of that compound or one geometric isomer (e.g., about a double bond) is substantially free of the other geometric isomer. For example, a stereomerically pure compound of the invention having one chiral centre, will be substantially free of the opposite enantiomer of the compound. A stereomerically pure compound of the invention having two chiral centres, or a composition thereof, will be substantially free of other diastereomers of the compound. A stereomerically pure compound of the invention having a double bond capable of E/Z isomerism, or a composition thereof, will be substantially free of one of the E/Z isomers.

A typical stereomerically pure compound comprises greater than about 80% by weight of one stereoisomer or E/Z isomer of the compound and less than about 20% by weight of other stereoisomers or E/Z isomer of the compound, more preferably greater than about 90% by weight of one stereoisomer or E/Z isomer of the compound and less than about 10% by weight of the other stereoisomers or E/Z isomer of the compound, even more preferably greater than about 95% by weight of one stereoisomer or E/Z isomer of the compound and less than about 5% by weight of the other stereoisomers or E/Z isomer of the compound, and most preferably greater than about 97% by weight of one stereoisomer or E/Z isomer of the compound and less than about 3% by weight of the other stereoisomers or E/Z isomer of the compound.

As used herein and unless otherwise indicated, the term “stereomerically enriched” means a compound of the invention, or a composition thereof, that comprises greater than about 60% by weight of one stereoisomer or E/Z isomer of a compound of the invention, preferably greater than about 70% by weight, more preferably greater than about 80% by weight of one stereoisomer or E/Z isomer of a compound of the invention.

Thus, it should be noted that unless stated otherwise, the term mycosporine-like amino acid compound according to the general formulae (V) to (X) and their variants as well as individual mycosporine-like amino acid compounds specified herein, are to be interpreted as encompassing racemic mixtures of enantiomers, mixtures of diastereomers or enantiomerically or optically pure compounds.

Due to their amino-cyclohexene-imine structure and their capability of electron delocalization, the compounds according to general formulae (I) to (X) are in equilibrium with their tautomer structures, in which the hydrogen of the amino group changes its places with the double bond of the cyclohexene ring.

Tautomerism is defined as each of two or more isomers of a compound which exist together in equilibrium and are readily interchanged by migration of an atom or group within the molecule. Tautomeric pure or enriched systems will readily interchange into the equilibrium state over time.

The tautomers of general formula (V) are represented by general formulae (V-tau):

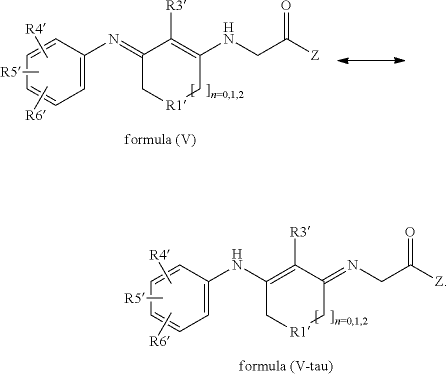

The tautomers of general formula (VI) are represented by general formula (VI-tau):

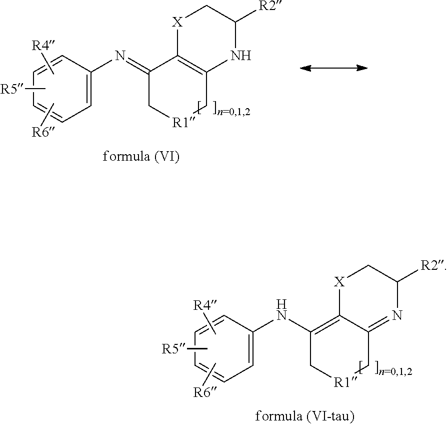

The above principles of tautomerism and redeposition explained on the basis of the mycosporine-like amino acid compounds according to the general formula (V) and (VI) are likewise applicable with respect to any of the mycosporine-like amino acid compounds defined by the general formula (VII), (Vill), (IX), (X) and their variants or with respect to any individual mycosporine-like amino acid compounds specified herein.

Thus, in the following and throughout the description, the reference to a mycosporine-like amino acid compound represented by any of the general formulae (V) to (X) and their variants or to an individual mycosporine-like amino acid compound specified herein encompasses likewise its tautomer isomer(s).

The mycosporine-like amino acid compounds according to the general formulae (V) to (X) and their variants as well as individual mycosporine-like amino acid compounds specified herein are used according to the first aspect of the present invention either in neutral, i.e. uncharged form, or in the form of their salts, such as an acid addition salt, with inorganic or organic acids.

The term “salt” in the context of the present invention refers to a salt of a compound that possesses the desired effect or pharmacological activity of the parent compound. Such salts include:
(1) acid addition salts formed with inorganic acids, or formed with organic acids, preferably monovalent or polyvalent carboxylic acids; or
(2) salts formed when an acidic proton present in the starting compound is replaced by a metal ion, e.g., an alkali metal ion, an alkaline earth ion, or an aluminium ion; or coordinated with an organic base.

Among the salts, acid addition salts are again particularly preferred, since the mycosporine-like amino acid compounds according to the general formulae (V) to (X) and their variants or the mycosporine-like amino acid compounds specified herein comprise a protonable N atom.

The inorganic acids that form acid addition salts with the mycosporine-line amino acid compound are preferably selected from the group consisting of hydrochloric acid, hydrobromic acid, sulfuric acid, nitric acid, phosphoric acid and the like. Particularly preferred is the hydrochloride salt or the sulphate salt or the phosphate salt.

Even more preferred are acid addition salts with organic mono- or polycarboxylic acids. Further preferred are acid addition salts with organic mono- or polycarboxylic acids, wherein the carboxylic acid is selected from saturated or mono- or polyunsaturated C1 to C30 monocarboxylic acids, saturated or mono- or polyunsaturated C3 to 10 di- or tricarboxylic acids. The carboxylic acid may be mono- or poly-substituted with hydroxy groups, preferably α-hydroxycarboxylic acids in which the hydroxy group is located on the carbon atom adjacent to the carboxy group. Many representatives occur naturally as so-called fruit acids. Preferred α-hydroxycarboxylic acids are malic acid, citric acid, 2-hydroxy-4-methylmercaptobutyric acid, glycolic acid, isocitric acid, mandelic acid, lactic acid, tartronic acid, or tartaric acid.

The organic acids that form acid addition salts with the mycosporine-like amino acid compound are preferably selected from the group consisting of amino acids, acetic acid, trifluoroacetic acid, propionic acid, hexanoic acid, cyclopentane propionic acid, glycolic acid, pyruvic acid, lactic acid, malonic acid, oxalic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2. 2.2]-oct-2-ene-1-carboxylic acid, glucoheptonic acid, 3-phenylpropionic acid, trimethylacetic acid, tert. Butylacetic acid, laurylsulfuric acid, gluconic acid, glutamic acid, hydroxynaphthoic acid, salicylic acid, stearic acid, muconic acid, 4-hydroxybutanoic acid, and the like.

Suitable anionic counterions are in particular chloride, bromide, hydrogensulfate, sulfate, dihydrogenphosphate, hydrogenphosphate, phosphate, nitrate, bicarbonate, carbonate, hexafluorosilicate, hexafluorophosphate, benzoate, and the anions of C1- to C4-alkanoic acids, preferably formate, acetate, trifluoroacetate, propionate and butyrate, furthermore lactate, gluconate, and the anions of poly acids such as succinate, oxalate, maleate, fumarate, malate, tartrate and citrate, furthermore sulfonate anions such as besylate (benzenesulfonate), tosylate (p-toluenesulfonate), napsylate (naphthalene-2-sulfonate), mesylate (methanesulfonate), esylate (ethanesulfonate), and ethanedisulfonate. They can be formed by reacting compounds according to the invention that have a basic functionality with an acid of the corresponding anion. Preferred salts of the compounds of formulae (I) to (III) as well as of the individual mycosporine-like amino acid compounds specified herein are chloride salts.

The metal ions for salt formation that replace an acidic proton present in the starting compound are selected from the group consisting of alkali metal ions, preferably Na+, K+, or Li+, alkaline earth metal ions, preferably Ca++ or Mg++, Al+++, or NH₄+.

The coordinating organic base for salt formation is selected from the group consisting of ethanolamine, diethanolamine, triethanolamine, N-methylglucamine, triethylamine, and the like.

In the following description and claims, and unless stated otherwise, the term mycosporine-like amino acid compound includes both the neutral, uncharged form of the compound/molecule and equally the salt form of the compound.

The salt form of the mycosporine-like amino acid compounds leads to a lower log P_{OW} and, thus, making the compounds more hydrophilic, which results in a better water solubility. In particular, the cationic salts of the mycosporine-like amino acid compounds support emulsification processes in emulsions, due to their surface active, i.e. co-emulsifying, properties. In addition, the cationic salt form of the mycosporine-like compounds shows excellent substantivity behaviour on skin and hair most conditioning actives are cationic; the conditioning effect leads to a softer skin feel, which makes the end products more accepted by the consumer. In addition, the mycosporine-like amino acid compounds in the cationic salt form exhibit antimicrobial activity.

The sunscreen product, cosmetic or pharmaceutical preparation or homecare product according to the present invention optionally contains at least one film forming agent (c). Optionally means that a film forming agent is not mandatory in the sunscreen product, cosmetic or pharmaceutical preparation or homecare product according to the present invention but can be advantageously incorporated. Film forming agents are well known in the art from their use as agents for improving the water resistance of sunscreen products.

Improving the water resistance is the process of making an object or structure more water-resistant so that it remains relatively unaffected by water or resisting the ingress of water under specified conditions.

A film forming agent is a hydrophobic material that imparts water resistance and film forming characteristics to the sunscreen products, cosmetic or pharmaceutical preparations or homecare products. Typical suitable film forming agents include copolymers derived from polymerization of octadecene-1 and maleic anhydride. A preferred film forming agent is a polyanhydride resin. Another preferred film forming agent is a copolymer of vinyl pyrrolidone and eicosene monomers.

Standard film forming agents are chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds. Preferably, the film forming agents are selected from the group consisting of chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds. Preferred film formers for improving water resistance of the composition are selected from the group consisting of Polyamide-8 (such as Oleocraft LP PA (MV)), Polyamide-3 (such as Oleocraft MP 32 PA (MV)), Trimethylpentanediol/Adipic Acid/Glycerin Crosspolymer (such as WetFilm), Octyldodecyl Citrate Crosspolymer (such as CosmoSurf CE 100), Polyglyceryl-3 Methyl Glucose Distearate (such as TegoCare 450), C28-52 Olefin/Undecylenic Acid Copolymer (such as Performa V6112), Hydrolyzed Jojoba esters, Jojoba Esters, Aqua (such as Floraester Jojoba K100), Cyclopentasiloxane Acrylates/Dimethicone copolymer (such as KP545), Acrylates/Beheneth-25 Methacylate Copolymer (such as Volarest), Styrene/Acrylates Copolymer (such as Dermacryl E Polymer), Polyurethane (such as Baycusan C1001), Polyurethane-2 and Polymethyl Methacrylate (such as Hybridur 875 Polymer), Acrylates/Octylacrylamide Copolymer (such as Dermacryl 2.0 Polymer), Beeswax (Cera Alba) (and) Sodium Stearoyl Lactylate (such as SymEffekt Sun), Hydrogenated Palm Oil, Saccharum officinarum Extract (SymEffect™ UV), Microcrystalline cellulose (and) Sphingomonas ferment extract (and) Cellulose gum (such as PemuPur™ START Polymer), Acacia senegal Gum and Xanthan Gum (such as SOLAGUM™ AX), Microcrystalline Cellulose (and) Cellulose Gum (such as Natpure Cellgum Plus), Sodium Stearoyl Glutamate (such as AMISOFT® HS-11P), Hydrogenated Polycyclopentadiene (and) Caprylic/Capric Triglyceride (such as Koboguard® 5400 CCT), Glyceryl Hydrogenated Rosinate (and) Caprylic/Capric Triglyceride (and) Tocopherol (such as KOBOGUARD® NATURAL 2063-CCT), Galactoarabinan (such as LaraCare® A 200), C26-28 Alkyl Dimethicone (such as Wacker BELSIL® CDM 3526 VP), Caprylyl Methicone (such as Silsoft 034), Stearyl Dimethicone (and) Octadecene (such as Dowsil 2503 Cosmetic Wax), VP/Hexadecene Copolymer (such as Antaron™ V 216), Triacontanyl PVP (such as Antaron™ WP-660), Capryloyl Glycerin/Sebacic Acid Copolymer (such as LexFilm™ Sun Natural MB), Poly C10-30 Alkyl Acrylate (such as Tego SP 13 Sun Up), Microcrystalline Cellulose (such as Sunspheres™ BIO SPF Booster), and any mixture thereof.

According to the present invention, the film forming agents are preferably selected from the group consisting of Polyamide-8 (such as Oleocraft LP 20 PA (MV)), Polyamide-3 (such as Oleocraft MP 32 PA (MV)), Trimethylpentanediol/Adipic Acid/Glycerin Crosspolymer (such as WetFilm), Octyldodecyl Citrate Crosspolymer (such as CosmoSurf CE 100), Polyglyceryl-3 Methyl Glucose Distearate (such as TegoCare 450), C28-52 Olefin/Undecylenic Acid Copolymer (such as Performa V6112), Hydrolyzed Jojoba esters, Jojoba Esters, Aqua (such as Floraester Jojoba K100), Cyclopentasiloxane Acrylates/Dimethicone copolymer (such as KP545), Acrylates/Beheneth-25 Methacylate Copolymer (such as Volarest), Styrene/Acrylates Copolymer (such as Dermacryl E Polymer), Polyurethane (such as Baycusan C1001), Polyurethane-2 and Polymethyl Methacrylate (such as Hybridur 875 Polymer), Acrylates/Octylacrylamide Copolymer (such as Dermacryl 2.0 Polymer), Beeswax (Cera Alba) (and) Sodium Stearoyl Lactylate (such as SymEffekt Sun), Pullulan, Xanthan Gum, Hydrogenated Palm Oil, Saccharum officinarum Extract (SymEffect™ UV), Microcrystalline cellulose (and) Sphingomonas ferment extract (and) Cellulose gum (such as PemuPur™ START Polymer), Acacia senegal Gum and Xanthan Gum (such as SOLAGUM™ AX), Microcrystalline Cellulose (and) Cellulose Gum (such as Natpure Cellgum Plus), Sodium Stearoyl Glutamate (such as AMISOFT® HS-11P), Hydrogenated Polycyclopentadiene (and) Caprylic/Capric Triglyceride (such as Koboguard® 5400 CCT), Glyceryl Hydrogenated Rosinate (and) Caprylic/Capric Triglyceride (and) Tocopherol (such as KOBOGUARD® NATURAL 2063-CCT), Galactoarabinan (such as LaraCare® A 200), C26-28 Alkyl Dimethicone (such as Wacker BELSIL® CDM 3526 VP), Caprylyl Methicone (such as Silsoft 034), Stearyl Dimethicone (and) Octadecene (such as Dowsil 2503 Cosmetic Wax), VP/Hexadecene Copolymer (such as Antaron™ V 216), Triacontanyl PVP (such as Antaron™ WP-660), Capryloyl Glycerin/Sebacic Acid Copolymer (such as LexFilm™ Sun Natural MB), Poly C10-30 Alkyl Acrylate (such as Tego SP 13 Sun Up), Microcrystalline Cellulose (such as Sunspheres™ BIO SPF Booster), and any mixture thereof.

More preferred, the film forming agents are selected from the group consisting of Trimethylpentanediol/Adipic Acid/Glycerin Crosspolymer (such as WetFilm), Octyldodecyl Citrate Crosspolymer (such as CosmoSurf CE 100), Polyglyceryl-3 Methyl Glucose Distearate (such as TegoCare 450), C28-52 Olefin/Undecylenic Acid Copolymer (such as Performa V6112), Hydrolyzed Jojoba esters, Jojoba Esters, Aqua (such as Floraester Jojoba K100), Cyclopentasiloxane Acrylates/Dimethicone copolymer (such as KP545), Styrene/Acrylates Copolymer (such as Dermacryl E Polymer), Acrylates/Octylacrylamide Copolymer (such as Dermacryl 2.0 Polymer), Beeswax (Cera Alba) (and) Sodium Stearoyl Lactylate (such as SymEffect™ Sun), Pullulan, Xanthan Gum, Hydrogenated Palm Oil, Saccharum officinarum Extract (SymEffect™ UV), Microcrystalline cellulose (and) Sphingomonas ferment extract (and) Cellulose gum (such as PemuPur™ START Polymer), Acacia senegal Gum and Xanthan Gum (such as SOLAGUM™ AX), Microcrystalline Cellulose (and) Cellulose Gum (such as Natpure Cellgum Plus), Sodium Stearoyl Glutamate (such as AMISOFT® HS-11P), Hydrogenated Polycyclopentadiene (and) Caprylic/Capric Triglyceride (such as Koboguard® 5400 CCT), Glyceryl Hydrogenated Rosinate (and) Caprylic/Capric Triglyceride (and) Tocopherol (such as KOBOGUARD® NATURAL 2063-CCT), Galactoarabinan (such as LaraCare® A 200), C26-28 Alkyl Dimethicone (such as Wacker BELSIL® CDM 3526 VP), Caprylyl Methicone (such as Silsoft 034), Stearyl Dimethicone (and) Octadecene (such as Dowsil 2503 Cosmetic Wax), VP/Hexadecene Copolymer (such as Antaron™ V 216), Triacontanyl PVP (such as Antaron™ WP-660), Capryloyl Glycerin/Sebacic Acid Copolymer (such as LexFilm™ Sun Natural MB), Poly C₁₀₋₃₀ Alkyl Acrylate (such as Tego SP 13 Sun Up), Microcrystalline Cellulose (such as Sunspheres™ BIO SPF Booster), and any mixture thereof.

Most preferred, the film forming agents are selected from the group consisting of Hydrolyzed Jojoba esters, Jojoba Esters, Aqua (such as Floraester Jojoba K100), Cyclopentasiloxane Acrylates/Dimethicone copolymer (such as KP545), Styrene/Acrylates Copolymer (such as Dermacryl E Polymer), Acrylates/Octylacrylamide Copolymer (such as Dermacryl 2.0 Polymer), Beeswax (Cera Alba) (and) Sodium Stearoyl Lactylate (such as SymEffect Sun), Pullulan, Xanthan Gum, Hydrogenated Palm Oil, Saccharum officinarum Extract (SymEffect™ UV), Microcrystalline cellulose (and) Sphingomonas ferment extract (and) Cellulose gum (such as PemuPur™ START Polymer), Microcrystalline Cellulose (and) Cellulose Gum (such as Natpure Cellgum Plus), Hydrogenated Polycyclopentadiene (and) Caprylic/Capric Triglyceride (such as Koboguard® 5400 CCT), Glyceryl Hydrogenated Rosinate (and) Caprylic/Capric Triglyceride (and) Tocopherol (such as KOBOGUARD® NATURAL 2063-CCT), Galactoarabinan (such as LaraCare® A 200), C26-28 Alkyl Dimethicone (such as Wacker BELSIL® CDM 3526 VP), Stearyl Dimethicone (and) Octadecene (such as Dowsil 2503 Cosmetic Wax), VP/Hexadecene Copolymer (such as Antaron™ V 216), Triacontanyl PVP (such as Antaron™ WP-660), Capryloyl Glycerin/Sebacic Acid Copolymer (such as LexFilm™ Sun Natural MB), Poly C10-30 Alkyl Acrylate (such as Tego SP 13 Sun Up), Microcrystalline Cellulose (such as Sunspheres™ BIO SPF Booster), and any mixture thereof.

The aforesaid film forming agents are used in the sunscreen, cosmetic or pharmaceutical preparation or homecare product either as a single component or preferably in a mixture with two or more further of said film forming agents as specified above.

The film forming agent (c) is used in amounts effective to allow the final formulation to remain on the skin after exposure to circulating water for at least 40 to 80 minutes.

With the addition of a mycosporine-like amino acid compound (b) as defined herein to a sunscreen product or cosmetic or pharmaceutical preparation or homecare product according to the present invention, the adhesion of the film forming agent (c) on the skin or on surfaces can significantly be improved. Preferably, the combination of a mycosporine-like amino acid compound (b) and a film forming agent (c) result in a synergistic adherence effect of the sunscreen product or cosmetic or pharmaceutical preparation or homecare product on the skin or on a surface, to which the formulation is applied. The improved adherence of the film forming agent in turn results in an improved water resistance, and, thus, provides excellent UV-protection even after bathing compared to products or preparations without the addition of the specified mycosporine-like amino acid compounds.

In a particular advantageously variant, the sunscreen product, cosmetic or pharmaceutical preparation or homecare product according to the first aspect of the present invention comprises thus one of the following combinations of components (b) and (c):
at least one MAA compound according to the general formula (V) as defined herein plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
at least one MAA compound according to the general formula (VI) as defined herein plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
at least one compound according to the general formula (VII) as defined herein plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
at least one compound according to the general formula (Vill) as defined herein plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster).

More preferred combinations of component (b) and (c) in the sunscreen product or cosmetic or pharmaceutical preparation according to the first aspect of the present invention are:
MAA-1 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-2 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-3 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-4 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-5 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-6 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-7 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-8 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-9 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-11 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-12 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-13 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-14 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-16 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-17 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-20 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-21 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-22 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-23 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-24 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-25 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-26 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-27 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-28 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-29 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-30 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-31 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-32 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-33 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-34 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-35 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-36 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-37 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-38 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster); or
MAA-39 plus at least one film forming agent: Hydrolyzed Jojoba esters and/or Jojoba Esters and/or Aqua (such as Floraester Jojoba K100) and/or Cyclopentasiloxane Acrylates/Dimethicone copolymer (such as KP545) and/or Styrene/Acrylates Copolymer (such as Dermacryl E Polymer) and/or Acrylates/Octylacrylamide Copolymer (such as Dermacryl 2.0 Polymer) and/or Beeswax (Cera Alba) (and) Sodium Stearoyl Lactylate (such as SymEffect Sun) and/or Pullulan and/or Xanthan Gum and/or Hydrogenated Palm Oil, Saccharum officinarum Extract (SymEffect™ UV) and/or Microcrystalline cellulose (and) Sphingomonas ferment extract (and) Cellulose gum (such as PemuPur™ START Polymer) and/or Microcrystalline Cellulose (and) Cellulose Gum (such as Natpure Cellgum Plus) and/or Hydrogenated Polycyclopentadiene (and) Caprylic/Capric Triglyceride (such as Koboguard® 5400 CCT) and/or Glyceryl Hydrogenated Rosinate (and) Caprylic/Capric Triglyceride (and) Tocopherol (such as KOBOGUARD® NATURAL 2063-CCT) and/or Galactoarabinan (such as LaraCare® A 200) and/or C26-28 Alkyl Dimethicone (such as Wacker BELSIL® CDM 3526 VP) and/or Stearyl Dimethicone (and) Octadecene (such as Dowsil 2503 Cosmetic Wax) and/or VP/Hexadecene Copolymer (such as Antaron™ V 216) and/or Triacontanyl PVP (such as Antaron™ WP-660) and/or Capryloyl Glycerin/Sebacic Acid Copolymer (such as LexFilm™ Sun Natural MB) and/or Poly C10-30 Alkyl Acrylate (such as Tego SP 13 Sun Up) and/or Microcrystalline Cellulose (such as Sunspheres™ BIO SPF Booster).

A synergistic water resistance improving effect can be shown for a combination of a mycosporine-like amino acid compound such as MAA-1, MAA-2, MAA-3, MAA-4, MAA-5, MAA-6, MAA-7, MAA-8, MAA-9, MAA-11, MAA-13, MAA-14, MAA-16, MAA-17, MAA-20, MAA-21, MAA-22, MAA-23, MAA-24, MAA-25, MAA-26, MAA-27, MAA-28, MAA-29, MAA-30, MAA-31. MAA-32, MAA-33, MAA-34, MAA-35, MAA-36, MAA-37, MAA-38, MAA-39 and any of the film forming agent. This tenacity effect can especially be observed when the mycosporine-like amino acid is in its salt form, i.e. in its cationic form. A beneficial tenacity improving effect can preferably be obtained for MAA-1 and MAA-13. The combination of the film building agents with said mycosporine-like amino acid compounds show a distinctive adhesion of the sunscreen products, cosmetic or pharmaceutical preparation or homecare product on the skin or on surfaces, resulting in an improvement of the water resistance.

The adhesion improving effect is especially pronounced for the mycosporine-like amino acid compounds MAA-20 to MAA-39, when combined with any of the film forming agent.

A synergistic adhesion improving effect is observed for the mycosporine-like amino acid compounds MAA-A, MAA-B and MAA-C, when combined with any of the film forming agent.

Besides the group of primary organic and/or inorganic sun protection UV-filters mentioned above, secondary sun protection ingredients of the antioxidant type may also be advantageously used in the sunscreen product, cosmetic or pharmaceutical preparation or homecare product according to the present invention in order to further optimize UV protection.

Secondary sun protection ingredients of the antioxidant type interrupt the photochemical reaction chain which is initiated when UV rays penetrate into the skin.

Typical examples of secondary sun protection ingredients are amino acids (for example arginine, lysine, glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example alpha-carotene, beta-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, alpha-linoleyl, cholesteryl and glyceryl esters thereof) and their salts, dilaurylthiodipropionate, distearyl-thiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages, also (metal) chelators (for example alpha-hydroxy fatty acids, palmitic acid, phytic acid, lactoferrine), alpha-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, sinapic acid, sinapoyl malate, sinapate ester derivatives, titanium dioxide (for example dispersions in ethanol), zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

The group of secondary sun protection ingredients also encompasses plant-based extract(s). Said plant-based extracts have antioxidative and in general photoprotective properties and interrupt the photochemical reaction chain which is initiated when UV rays penetrate into the skin, thus, are effective in preventing skin aging. The plant extracts with UV protection properties are selected from the group consisting of Lignin/Cellulose, Lignin, Lignin Hydrolyzed, Propolis and green propolis, Galanga Extract, macro and micro algae (Porphyra, red algae (Porphyra umbilicalis), Palmaria palmata, Saccharina latissimi, Corallina pilulifera, Eckloina cava, Sargassum sagamianum, Porphyra rosengurttii, Sargassum siliquastrum, Thalassiosira weissflogii, Green Microalgae (e.g. Interfilum and Klebsormidium), Seaweed (e.g. Padina australis, Euchema, cottonii), Pongamia glabra Seed Oil, Karanja oil (Millettia pinnata), Rhatania Extrakt (Krameria lappacea), Lichens, Soybean, Caper (Capparis spinosa), Bamboo, Green Coffee oil, Buriti Oil (Mauritia flexuosa), Black Sea Cucumber (Holothuria atra), Cyanobacteria, Femblock (Polypodium leucotomos), Green Tee, Black Tea (Camellia sinensis), Aloe vera, Walnut, Borage oil, Evening primrose oil, Avocado oil, Tea tree oil, Red Clover (Trifolium pratense L.), Soybean (Glycine max L.), Caper (Capparis spinosa), Almonds, Flame of the forest (Spathodea 145amascene145145), Milk thistle (Silybum marianum L.), Cashew nut shell, grape, Red Orange, Pomegranate (Punica granatu), Bilberry (Vaccinium myrtillus L.), Bog blueberry (Vaccinium uliginosum L.), Strawberry (Fragaria ananassa), Foti (Polygonum multiflorum Thunb), Turmeric (Curcuma longa L.), Ginseng (Panax ginseng), English ivy (Hedera helix), Broccoli (Brassica oleracea var italica), Tamarind (Tamarindus indica), Opuntia ficus-indica, Coffee, Basil (Ocimum basilicum, Linn.), pomegranate seed oil (Punica granatum), wheat germ oil (Triticum vulgare), blackcurrant seed oil (Ribes nigrum), sesame oil (Sesamum indicum), carrot root (Daucuscarota sativa), raspberry seed oil (Rubus idaeus), traditional medicinal plants from Sri Lanka (Aporosa lindleyana (Euphorbiaceae), Atalantia ceylanica (Rutaceae), Hibiscus furcatus (Malvaceae), Olaxzeylanica (Olacaceae), Ophiorrhiza mungos (Rubiaceae), Argyreia populifolia (Convolvulaceae), Ipomoea mauritiana (Convolvulaceae), Lasia spinosa (araceae), Leucas zeylanica (Lamiaceae), Plectranthus zeylanicus (Lamiaceae) and Brazil (D. gardneriana seeds, L. microphylla leafs, Amburana cearensis, Aspidosperma cuspa, Aspidosperma pyrifolium, Croton sonderianus, Curatella americana, Dimophandra gardneriana, Lippia microphylla, Luehea 146amascene146, Sida galheirensis and any mixture thereof.

Preferably, the plant extracts with UV protection properties are selected from the group consisting of Lignin/Cellulose, Lignin, Lignin Hydrolyzed, Galanga Extract, Porphyra, red algae (Porphyra Umbilicalis), Green Microalgae (e.g. Interfilum and Klebsormidium), Seaweed (e.g. Padina australis, Euchema, cottonii), Pongamia glabra Seed Oil, Karanja oil (Millettia pinnata), Rhatania Extrakt (Krameria lappacea), Soybean, Bamboo, Buriti Oil (Mauritia flexuosa), Cyanobacteria, Femblock (Polypodium leucotomos), Green Tee, Black Tea (Camellia sinensis), Evening primrose oil, Cashew nut shell, Pomegranate (Punica granatu), and any mixture thereof.

Advantageous the amount of secondary sun protection substances in the sunscreen product, cosmetic or pharmaceutical preparation or homecare product according to the present invention is advantageously from 0.005 to 5.0% by weight, preferably from 0.01 to 4.0% by weight, most preferred from 0.05 to 3.0% by weight, based on the total weight of the ready-to-use formulation.

The UV-filters are incorporated either in the aqueous or in the lipophilic part of the sunscreen product, cosmetic or pharmaceutical preparation or homecare product, depending if they are water or oil (fat) soluble/miscible UV-filters or may even be added to the final product by standard methods known to a person skilled in the art.

The at least one primary organic and/or inorganic UV-filter (a) according to the first aspect of the present invention is present in the sunscreen product, cosmetic or pharmaceutical preparation or homecare product in an amount of 0.01 to 80.0% by weight, based on the total weight of the final formulation. In a preferred variant, the sunscreen product, cosmetic or pharmaceutical preparation or homecare product comprises the primary organic and/or inorganic UV-filter (a) in an amount of 0.1 to 75.0% by weight, based on the total weight of the final formulation. In a more preferred variant, the UV-filter is advantageously used in the sunscreen product, cosmetic or pharmaceutical preparation or homecare product in an amount of 0.5 to 70.0% by weight, based on the total weight of the final formulation. In a most preferred variant, the UV-filter is advantageously used in the sunscreen product, cosmetic or pharmaceutical preparation or homecare product in an amount of 1.0 to 60.0% by weight, based on the total weight of the final formulation.

For a mixture of primary organic and/or inorganic UV-filters, the above amounts relate to the total content of the UV-filters in the mixture, i.e. the amount is the sum of the content of all primary UV-filters in the mixture.

The primary organic and/or inorganic UV-filter (a) and further UV-protection components, such as secondary sun protection ingredients or secondary sun protection UV-filters, as defined herein, i.e. the total amount of UV-filters, are present in the sunscreen product, cosmetic or pharmaceutical preparation or homecare product according to the present invention, in an amount of 0.1 to 85.0% by weight, based on the total weight of the final formulation. In a preferred variant, the sunscreen product, cosmetic or pharmaceutical preparation comprises the primary organic and/or inorganic UV-filter (a) and the further UV-protection components as defined herein in an amount of 0.3 to 80.0% by weight, based on the total weight of the final formulation. In a more preferred variant, the UV-filter (a) and the further UV-protection components as defined herein are advantageously used in the sunscreen product, cosmetic or pharmaceutical preparation or homecare product in an amount of 0.5 to 75.0% by weight, based on the total weight of the final formulation. Thus, the above amounts relate to the sum of the content of all UV-filters/UV protection components present in the sunscreen or cosmetic or pharmaceutical preparation.

The at least one mycosporine-like amino acid compound (b) according to the first aspect of the present invention is present in the sunscreen product, cosmetic or pharmaceutical preparation or homecare product in an amount of 0.001 to 15.0% by weight, based on the total weight of the final formulation. In a preferred variant, the sunscreen product, cosmetic or pharmaceutical preparation or homecare product comprises the at least one mycosporine-like amino acid compound (b) in an amount of 0.01 to 10.0% by weight, based on the total weight of the final formulation. In a more preferred variant, the at least one mycosporine-like amino acid compound (b) is advantageously used in the sunscreen product, cosmetic or pharmaceutical preparation or homecare product in an amount of at 0.05 to 7.5% by weight, based on the total weight of the final formulation. In a most preferred variant, the at least one mycosporine-like amino acid compound is advantageously used in the sunscreen product, cosmetic or pharmaceutical preparation or homecare product in an amount of at 0.1 to 5.0% by weight, based on the total weight of the final formulation.

For a mixture of mycosporine-like amino acid compounds I, the above amounts relate to the total content of the mycosporine-like amino acid compounds in the mixture, i.e. the amount is the sum of the content of all mycosporine-like amino acid compounds (b) in the mixture.

The optionally at least one film forming agent (c) according to the first aspect of the present invention is present in the sunscreen product, cosmetic or pharmaceutical preparation or homecare product in an amount of 0.01 to 10.0% by weight, based on the total weight of the final formulation. In a preferred variant, the sunscreen product, cosmetic or pharmaceutical preparation or homecare product comprises the film forming agent I in an amount of 0.05 to 8.0% by weight, based on the total weight of the final formulation. In a most preferred variant, the film forming agent I is advantageously used in the sunscreen product, cosmetic or pharmaceutical preparation or homecare product in an amount of 0.1 to 5.0% by weight, based on the total weight of the final formulation.

For a mixture of film forming agents, the above amounts relate to the total content of the film forming agents in the mixture, i.e. the amount is the sum of the content of all film forming agents in the mixture.

Advantageously, the sunscreen product according to the first aspect of the present invention comprises

(i) 0.01 to 80.0% by weight of primary organic and/or inorganic UV-filter;
(ii) 0.001 to 15.0% by weight of at least one mycosporine-like amino acid compound as defined herein; and
(iii) optionally 0.01 to 10.0% by weight of at least one film forming agent; based on the based on the total weight of the final formulation.

In a preferred variant, the cosmetic or pharmaceutical preparation according to the first aspect of the present invention comprises
(iv) 1.0 to 60.0% by weight of at least one primary organic and/or inorganic UV-filter;
(v) 0.1 to 5.0% by weight of at least one mycosporine-like amino acid compound as defined herein; and
(vi) optionally 0.1 to 5.0% by weight of at least one film forming agent;
based on the based on the total weight of the final formulation.

The sunscreen products, cosmetic or pharmaceutical preparations or homecare products according to the present invention provide broad spectrum photo-protection und therefore provide protection from both UVA and UVB radiation. Sun protection factor (SPF) is a measure of UVB protection. Typically, the compositions of the present invention have an SPF of at least 6. The SPF however, can vary as needed. The type and amount of UVA and UVB filters can be varied to obtain a desired level of SPF. Preferably, the SPF of the sunscreen product, cosmetic or pharmaceutical preparation or homecare product according to the present invention is at least 6, 10, 15, 20, 25, 30, 35, 40, 45, 50, 55, 60, 65, 70, 75, 80 or even more.

Within the context of the present invention, it is also possible and in some cases advantageous to combine the sunscreen product, cosmetic or pharmaceutical preparation or homecare product according to the first aspect of the present invention with other customary active substances, adjuvants or additives, customarily used for sunscreen products, cosmetic or pharmaceutical compositions or homecare products, as further described below, in order to obtain a ready-for-use preparation or formulation.

The other active agents and/or adjuvants and/or additives or auxiliaries are for example abrasives, anti-acne agents, agents against ageing of the skin, anti-cellulitis agents, anti-dandruff agents, anti-inflammatory agents, irritation-preventing agents, irritation-inhibiting agents, antioxidants, alkanediols, astringents, odour absorbers, perspiration-inhibiting agents, antiseptic agents, anti-statics, antimicrobial agents, binders, buffers, carrier materials, oil components, chelating agents, cell stimulants, cleansing agents, depilatory agents, surface-active substances, deodorizing agents, antiperspirants, softeners, emulsifiers, enzymes, enzyme inhibitors, essential oils, fibres, film-forming agents, water resistance improving agents fixatives, foam-forming agents, foam stabilizers, substances for preventing foaming, foam boosters, gelling agents, gel-forming agents, hair care agents, hair-setting agents, hair-straightening agents, moisture-donating agents, moisturizing substances, moisture-retaining substances, bleaching agents, strengthening agents, stain-removing agents, optically brightening agents, impregnating agents, dirt-repellent agents, dyes, friction-reducing agents, lubricants, moisturizing creams, ointments, opacifying agents, plasticizing agents, covering agents, polish, preservatives, gloss agents, green and synthetic polymers, powders, proteins, re-oiling agents, abrading agents, silicones, skin-soothing agents, skin-cleansing agents, skin care agents, skin-healing agents, skin-lightening agents, skin-protecting agents, skin-softening agents, hair promotion agents, cooling agents, skin-cooling agents, warming agents, skin-warming agents, stabilizers, surfactants, antimicrobial agents, detergents/surfactants, fabric conditioning agents, suspending agents, skin-tanning agents, actives modulating skin or hair pigmentation, matrix-metalloproteinase inhibitors, skin moisturizing agents, glycosaminoglycan stimulators, TRPV1 antagonists, desquamating agents, anti-cellulite agents or fat enhancing agents, hair growth activators or inhibitors, thickeners, rheology additives, vitamins, oils, waxes, pearlizing waxes, fats, phospholipids, saturated fatty acids, mono- or polyunsaturated fatty acids, α-hydroxy acids, polyhydroxy fatty acids, liquefiers, dyestuffs, colour-protecting agents, pigments, anti-corrosives, fragrances or perfume oils, aromas, flavouring substances, odoriferous substances, polyols, electrolytes, organic solvents, and mixtures of two or more of the aforementioned substances, as further described below.

Of the above cosmetically or pharmaceutically excipients and/or active substances and/or additives and/or auxiliaries carriers, oil components, rheology additives, hydrotropes, solubilizing agents, powders, film formers, water resistance improving agents, skin moisturizing and/or moisture-retaining substances, lenitive substances, physiological cooling agents, alkanediols, antioxidants, agents against ageing of the skin, matrix-metalloproteinase inhibitors (MMPI), anti-inflammatory agents, TRPV1 antagonists, emulsifiers, antimicrobial agents, preservatives, antibacterial or antimycotic active substances, chelating agents, surfactants, fragrances/aroma or perfume oils, and any mixture of two or more of the afore-mentioned substances are particularly preferred in the preparation of sunscreen products, cosmetic and pharmaceutical preparations or homecare products.

Carriers: As the sunscreen product, cosmetic or pharmaceutical, in particular dermatological, preparation or homecare product as defined herein are intended for topical application, such as in the form of ointments, pastes, creams and gels, etc. they are preferably based on a carrier. Most common acceptable carrier is water. Acceptable carries other than water include glycerin, C₁₋₄ alcohols, organic solvents, fatty alcohols, fatty ethers, fatty esters, polyols, glycols, vegetable oils, mineral oils, liposomes, laminar lipid materials, water, or a mixture thereof. Non-limiting examples of organic solvents include mono alcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds. The total amount of carrier in the compositions can vary but is typically 40 to 90% by weight, based on the total weight of the composition.

The products or preparations of the present invention may include at least one water-soluble or organic solvent. Non-limiting examples of organic solvents include monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, 1,2-pentanediol, 1,2-hexanediol, 1,2-heptanediol, 1,2-octanediol, 1,2-decanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and any mixture thereof. The total amount water-soluble or organic solvent(s) in the composition according to the present invention may vary but is typically 0.1 to 50% by weight, based on the total weight of the composition.

Solubilizing agents: The products or preparations of the present invention may include at least one solubilizing agent. Solubilizing agents are compounds that help solubilize the UV-filter(s) and/or other components in the compositions. A particularly useful but non limiting example of a solubilizing agent is a hydrotrope. Hydrotropes (or hydrotropic agents) are a diverse class of typically water-soluble compounds that may be characterized by an amphiphilic molecular structure and an ability to dramatically increase the solubility of poorly soluble organic molecules in water. Non-limiting examples of hydrotropes include sodium 1,3-benzenedisulfonate, sodium benzoate, sodium 4-pyridinecarboxylate, sodium salicylate, sodium benzene sulfonate, caffeine, sodium p-toluene sulfonate, sodium butyl monoglycolsulfate, 4-aminobenzoic acid HCl, sodium cumene sulfonate, N,N-diethylnicotinamide, N-picolylnicotinamide, N-allylnicotinamide, 2-methacryloyloxyethyl phosphorylcholine, resorcinol, butylurea, pyrogallol, N-picolylacetamide 3.5, procaine HCl, proline HCl, nicotinamide, pyridine, 3-picolylamine, sodium ibuprofen, sodium xylenesulfonate, ethyl carbamate, pyridoxal hydrochloride, sodium benzoate, 2-pyrrolidone, ethylurea, N,N-dimethylacetamide, N-methylacetamide, and isoniazid and any mixture thereof. In some cases, particularly useful hydrotropes include nicotinamide (niacinamide), caffeine, sodium PCA, sodium salicylate, urea, and dihydroxyethyl urea, in particular, nicotinamide (niacinamide) and/or caffeine. A combination of two or more, three or more, or four or more hydrotropes may also be used in the compositions according to the present invention. The total amount of solubilizing agent(s) in the compositions of the present invention may vary but are typically in an amount of 0.01 to 20% by weight, based on the total weight of the composition.

Oil components: The sunscreen product, cosmetic or pharmaceutical preparation or homecare product may include at least one oil phase or at least one oil component, waxes, starch, tragacanth, cellulose derivatives, polyethylene glycols, silicones, bentonites, silica, talc and zinc oxide or any mixture thereof. Powders include carriers such as lactose, talc, silica, aluminium hydroxide, calcium silicate and polyamide powder or any mixture thereof. Solutions and emulsions include carriers such as solvents such as water, ethanol, isopropanol, ethyl carbonate, ethyl acetate, benzyl alcohol, propylene glycol, etc. or any mixture thereof. However, preparations solely based on water are also possible. Oils, oil-in-water and water-in-oil emulsions, etc. are preferred.

The oil phase or the oil component in the sunscreen product, cosmetic or pharmaceutical preparation or homecare product according to the present invention which may be suitable are for example plant oils, hydrocarbons, fatty alcohols, fatty acid esters, or mixtures of two or more of the aforesaid oil components.

The oil phase or oil component in the sunscreen product, cosmetic or pharmaceutical preparation or homecare product is preferably a plant oil and even more preferably a liquid plant oil. It can also advantageously be a mixture of two or more plant oils components, especially liquid plant oil mixtures.

Plant oils or vegetable oils are oils extracted from seeds, or less often, from other parts of fruits. Like animal fats, plant oils are mixtures of triglycerides. Soybean oil, rapeseed oil and cocoa butter are examples of plant oils from seeds. Olive oil, palm oil and rice bran oil are examples of oils from other parts of fruits. In common usage, plant oil or vegetable oil may refer exclusively to vegetable fats which are liquid at room temperature or at 35 to 37° C. skin temperature. Vegetable oils are usually edible.

The term “plant oils” also includes unsaturated plant oils. Unsaturated oils or vegetable oils can be transformed through partial or complete “hydrogenation” into oils of higher melting point. The hydrogenation process involves “sparging” the oil at high temperature and pressure with hydrogen in the presence of a catalyst, typically a powdered nickel compound. As each carbon-carbon double-bond is chemically reduced to a single bond, two hydrogen atoms each form single bonds with the two carbon atoms.

The elimination of double bonds by adding hydrogen atoms is called saturation; as the degree of saturation increases, the oil progresses toward being fully hydrogenated. An oil may be hydrogenated to increase resistance to rancidity (oxidation) or to change its physical characteristics. As the degree of saturation increases, the oil's viscosity and melting point increase.

In a preferred variant, the plant oil is selected from the group consisting of Persea gratissima (Avocado Oil), Abies alba Seed Oil, Acacia victoriae Seed Oil, Actinidia chinensis (Kiwi) Seed Oil, Amaranthus hypochondriacus Seed Oil, Arachis hypogaea (Peanut) Oil, Astrocaryum murumuru Seed Butter, Astrocaryum tucuma Seed Butter, Astrocaryum tucuma Seed Oil, Astrocaryum vulgare Fruit Oil, Astrocaryum vulgare Kernel Oil, Avena sativa (Oat) Kernel Oil, Brassica alba Seed Oil, Brassica campestris (Rapeseed) Seed Oil, Butyrospermum parkii (Shea) Butter, Butyrospermum parkii (Shea) Oil, Calendula officinalis Seed Oil, Calophyllum inophyllum Seed Oil, Calophyllum tacamahaca Seed Oil, Camellia oleifera Seed Oil, Camellia reticulata Seed Oil, Camellia sinensis Seed Oil, Cannabis sativa Seed Oil, Cannabis sativa Seed/Stem Oil, Canola Oil, Carthamus tinctorius (Safflower) Seed Oil, Chlorella vulgaris Oil, Citrullus lanatus (Watermelon) Seed Oil, Citrus aurantifolia (Lime) Seed Oil, Citrus aurantium dulcis (Orange) Seed Oil, Citrus grandis (Grapefruit) Seed Oil, Cocos nucifera (Coconut) Oil, Cocos nucifera (Coconut) Seed Butter, Coffea arabica (Coffee) Seed Oil, Chlorella Oil (biotech), Corylus americana (Hazelnut) Seed Oil, Corylus avellana (Hazelnut) Seed Oil, Cucumis melo (Melon) Seed Oil, Cucumis sativus (Cucumber) Seed Oil, Cucurbita pepo (Pumpkin) Seed Oil, Elaeis guineensis (Palm) Oil, Elaeis (Palm) Fruit Oil, Glycine soja (Soybean) Oil, GosSY-pium herbaceum (Cotton) Seed Oil, GosSY-pium hirsutum (Cotton) Seed Oil, Helianthus annuus (Sunflower) Seed Oil, Macadamia integrifolia Seed Oil, Macadamia ternifolia Seed Oil, Mangifera indica (Mango) Seed Butter, Mangifera indica (Mango) Seed Oil, Melissa officinalis Seed Oil, Microalgae Oil, Moringa oleifera Seed Oil, Moringa peregrina Seed Oil, Oenothera biennis (Evening Primrose) Oil, Olea europaea (Olive) Fruit Oil, Olus Oil, Orbignya oleifera Seed Oil, Orbignya speciosa Kernel Oil, Oryza sativa (Rice) Bran/Germ Oil, Oryza sativa (Rice) Bran Oil, Oryza sativa (Rice) Germ Oil, Oryza sativa (Rice) Lipids, Oryza sativa (Rice) Seed Oil, Papaver somniferum Seed Oil, Passiflora edulis Seed Oil, Persea gratissima (Avocado) Butter, Persea gratissima (Avocado) Oil, Prunus amygdalus dulcis (Sweet Almond) Oil, Prunus armeniaca (Apricot) Kernel Oil, Prunus persica (Peach) Kernel Oil, Punica granatum Seed Oil, Pyrus malus (Apple) Seed Oil, Ricinoleic/Caproic/Caprylic/Capric Triglyceride(s), Ricinus communis (Castor) Seed Oil, Rosa canina Fruit Oil, Rosa moschata Seed Oil, Rubus idaeus (Raspberry) Seed Oil, Sesamum indicum (Sesame) Seed Butter, Soybean Glycerides, Theobroma cacao (Cocoa) Seed Butter, Theobroma grandiflorum Seed Butter, Triticum vulgare (Wheat) Bran Lipids, Triticum vulgare (Wheat) Germ Oil, Vitis vinifera (Grape) Seed Oil, Zea mays (Corn) Germ Oil, and Zea mays (Corn) Oil.

The above specified plant oils are the most common natural lipid components used as basic substances for the manufacture of cosmetics or pharmaceutical formulations.

Hydrocarbons (mineral oils) are in general organic compounds consisting entirely of hydrogen and carbon. As defined by IUPAC nomenclature or organic chemistry, the classifications for hydrocarbons are:
1. Saturated hydrocarbons are the simplest of the hydrocarbon species. They are composed entirely of single bonds and are saturated with hydrogen. The formula for acyclic saturated hydrocarbons (i.e., alkanes) is CₙH₂ₙ₊₂. The most general form of saturated hydrocarbons is CₙH₂ₙ₊₂₍₁₋ᵣ₎, where r is the number of rings. Those with exactly one ring are the cycloalkanes. Saturated hydrocarbons are the basis of petroleum fuels and are found as either linear or branched species.
2. Unsaturated hydrocarbons have one or more double or triple bonds between carbon atoms. Those with double bond are called alkenes. Those with one double bond have the formula CₙH₂ₙ (assuming non-cyclic structures). Those containing triple bonds are called alkynes. Those with one triple bond have the formula CₙH₂ₙ₋₂.
3. Aromatic hydrocarbons, also known as arenes, are hydrocarbons that have at least one aromatic ring.

Hydrocarbons can be inter alia liquids (e.g. hexane and benzene), waxes or low melting solids (e.g. paraffin wax and naphthalene). The term ‘aliphatic’ refers to non-aromatic hydrocarbons. Saturated aliphatic hydrocarbons are sometimes referred to as “paraffins”. Mineral oils and waxes are mixtures of predominantly saturated hydrocarbons consisting of straight-chain, branched and ring structures with carbon chain lengths greater than C14. Mineral oils and waxes are chemical substances prepared from naturally occurring crude petroleum oil. They mainly consist of mineral oil saturated hydrocarbons (MOSH) and mineral oil aromatic hydrocarbons (MOAH). Hydrocarbons have been used for many decades in skin and lip care cosmetic products due to their excellent skin tolerance as well as their high protecting and cleansing performance and broad viscosity options. In contrast to vegetable oils, mineral oils are non-allergenic since they are highly stable and not susceptible to oxidation or rancidity.

A fatty alcohol (or long-chain alcohol) is usually a high-molecular-weight, straight-chain primary alcohol, but can also range from as few as 4 to 6 carbons to as many as 22 to 26, derived from natural fats and oils. The precise chain length varies with the source. Some commercially important fatty alcohols are lauryl, stearyl and oleyl alcohols. They are colourless oily liquids (for smaller carbon numbers) or waxy solids, although impure samples may appear yellow. Fatty alcohols usually have an even number of carbon atoms and a single alcohol group (—OH) attached to the terminal carbon. Some are unsaturated and some are branched. Most fatty alcohols in nature are found as waxes which are esters with fatty acids and fatty alcohols. The traditional sources of fatty alcohols have largely been various vegetable oils and these remain a large-scale feedstock. The alcohols are obtained from the triglycerides (fatty acid triesters), which form the bulk of the oil. The process involves the transesterification of the triglycerides to give methyl esters which are then hydrogenated to give the fatty alcohols. Fatty alcohols are also prepared from petrochemical sources. In the Ziegler process, ethylene is oligomerized using triethylaluminium followed by air oxidation. Alternatively, ethylene can be oligomerized to give mixtures of alkenes, which are subjected to hydroformylation, this process affording odd-numbered aldehyde, which is subsequently hydrogenated. Fatty alcohols are mainly used in the production of detergents and surfactants. They are components also of cosmetic solvents. They find use as co-emulsifiers, emollients and thickeners in cosmetics.

In a preferred variant, the fatty alcohol is selected from the group consisting of phenyl propanol, dimethyl phenylbutanol, hexyldecanol, octyldodecanol, octyldecanol, tridecylalcohol, isostearyl alcohol, phenylisohexanol, phenylpropanol, trimethylbenzenepropanol, isoamylalcohol, isostearyl alcohol, and isotridecyl alcohol. In a more preferred variant, the fatty alcohol is selected from the group consisting of hexyldecanol, octyldodecanol, phenylpropanol, isoamylalcohol, and mixtures of two or more of the aforesaid fatty alcohols. The fatty alcohol can be used either as a single component or in a mixture with one or more further different fatty alcohol(s) as specified above.

A fatty acid ester is a type of ester that results from the combination of a fatty acid with an alcohol. When the alcohol component is glycerol, the fatty acid esters produced can be monoglycerides, diglycerides or triglycerides. Fatty acid esters have a conditioning effect of softening the skin to create a smoothing sensation. They are also added to cosmetics to dissolve high-polarity active ingredients and UV absorbers. Esters of straight-chain fatty acids and lower alcohols are effective for dissolving slightly soluble ingredients for oils with a light touch during application. Isostearic acids and other liquid oils with branched fatty acids and unsaturated fatty acids are commonly used as emollients. Higher fatty acid esters and esters of higher alcohols with relatively high melting points are added to skin creams to adjust the application touch.

Oil bodies and emollients: In addition to the oil phase or oil component as defined above, the sunscreen product, cosmetic or pharmaceutical preparation or homecare product according to the present invention preferably includes one or more oil bodies or emollients. An emollient is an oleaginous or oily substance which helps to smooth and soften the skin, and may also reduce its roughness, cracking or irritation. Especially, in sunscreen products emollients counteract the known stickiness of some UV-filters and keep the crystalline and oil-soluble organic and inorganic UV-filters solubilized and prevent them from recrystallisation. In addition, emollients serve as wetting agents for inorganic UV-filters for a homogeneous dispersion in the formulations. Suitable oil bodies, which form constituents of the O/W emulsions, are, for example, Guerbet alcohols based on fatty alcohols having 6 to 18, preferably 8 to 10, carbon atoms, esters of linear C5-C22 fatty acids with linear or branched C5-C22 fatty alcohols or esters of branched C1-C13 carboxylic acids with linear or branched C6-C22 fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C6-C22 fatty acids with branched alcohols, in particular 2-ethylhexanol, esters of C18-C38 alkylhydroxy carboxylic acids with linear or branched C6-C22 fatty alcohols, in particular Dioctyl Malate, esters of linear and/or branched fatty acids with polyhydric alcohols (such as, for example, propylene glycol, dimerdiol or trimertriol) and/or Guerbet alcohols, triglycerides based on C6-C10 fatty acids, liquid mono-/di-/triglyceride mixtures based on C6-C18 fatty acids, esters of C6-C22 fatty alcohols and/or Guerbet alcohols with aromatic carboxylic acids, in particular benzoic acid, esters of C2-C12 dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched CC-C22 fatty alcohol carbonates, such as, for example, Dicaprylyl Carbonate (Cetiol® CC), Guerbet carbonates, based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or asymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicone methicone grades, etc.) and/or aliphatic or naphthenic hydrocarbons, such as, for example, 159amascen, squalene or dialkylcyclohexanes, and any mixture thereof.

Powders: The compositions as defined herein may optionally include powders. The optional powders provide formulas that are smoother and softer on the skin. Representative powders include talc, mica, magnesium carbonate, calcium carbonate, magnesium silicate, aluminium magnesium silicate, silica, titanium dioxide, zinc oxide, red iron oxide, yellow iron oxide, black iron oxide, polyethylene powder, methacrylate powder, polystyrene powder, silk powder, Preferred solid powder materials, which may be a component of the composition according to the invention are hydrocolloids, such as starches, degraded starches, chemically or physically modified starches, dextrins, (powdery) maltodextrins (preferably with a dextrose equivalent value of 5 to 25, preferably of 10-20), lactose, silicon dioxide, glucose, modified celluloses, gum arabic, ghatti gum, traganth, karaya, carrageenan, pullulan, curdlan, xanthan gum, gellan gum, guar flour, carob bean flour, alginates, agar, pectin, inulin, and mixtures of two or more of these solids, in particular maltodextrins (preferably with a dextrose equivalent value of 15-20), lactose, silicon dioxide and/or glucose.

Rheology modifiers: The sunscreen product, cosmetic or pharmaceutical preparation or homecare product according to the present invention advantageously includes one or more thickening agents and/or rheology modifier. The rheology modifier is present in an amount that prevents significant dripping or pooling of the composition after application to the skin. Preferably, the rheology modifier is a carbomer. In some embodiments, the rheology modifier is selected from stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

Additional example of rheology modifiers include thickener or gelling agents, including substances which can increase the viscosity of a composition. Thickening agents include those that can increase the viscosity of a composition without substantially modifying the efficacy of the active ingredient within the formulation. Thickening agents can also increase the stability of the formulations of the present application. In certain aspects of the present application, thickening agents include hydrogenated polyisobutene or trihydroxy stearin, or a mixture of both. Additional non-limiting examples of additional thickening agents that can be used in the context of the present application include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see CTFA International Cosmetic Ingredient Dictionary, Fourth Edition, 1991, pp. 12 and 80). Examples of commercially available carboxylic acid polymers include carbomers, which are homopolymers of acrylic acid crosslinked with allyl ethers of sucrose or pentaerythritol (e.g., Carbopol™ 900 series from B. F. Goodrich). Non-limiting examples of crosslinked polyacrylate polymers include cationic and nonionic polymers.

Non-limiting examples of polyacrylamide polymers (including nonionic polyacrylamide polymers including substituted branched or unbranched polymers) include polyacrylamide, isoparaffin and Laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropyl cellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer are hydroxyalkylated (preferably hydroxy ethylated or hydroxypropylated) to form a hydroxyalkylated cellulose which is then further modified with a C10-C30 straight chain or branched chain alkyl group through an ether linkage. Typically, these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present compositions include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof. In one embodiment, the thickening agent is Chondrus crispus (carrageenan) extract.

Water resistance improving agents: The sunscreen product, cosmetic or pharmaceutical preparation or homecare product according to the present invention may advantageously include one or more agents for improving water resistance. An agent for improving water resistance is a hydrophobic material that imparts film forming and water resistance characteristics to an emulsion. Typical suitable water resistance improving agents include copolymers derived from polymerization of octadecene-1 and maleic anhydride. A preferred water resistance improving agent is a polyanhydride resin. Another preferred water resistance improving agent is a copolymer of vinyl pyrrolidone and 162amascen monomers. The water resistance improving agent(s) is/are used in amounts effective to allow the final formulation to remain on the skin or surface after exposure to circulating water for at least 40 minutes, preferably 80 minutes. One or more water resistance improving agents can optionally be included in the final formulation in an amount ranging from 0.01 to 10.0% by weight, preferably 0.05 to 8.0% by weight, more preferably 0.1 to 5.0% by weight, based on the total weight of the final formulation.

Silicones: In order to impart a silky, spreadable, and luxurious texture and to make skin look and feel smoother, and additionally to improve processability (antifoaming) the sunscreen product, cosmetic or pharmaceutical preparation or homecare product according to the present invention preferably includes one or more silicones or silicone derivatives. The total amount of silicone compound(s) in the compositions according to the present invention can vary but is typically 0.1 to 35.0% by weight, based on the total weight of the composition.

Dry-feel modifiers: The sunscreen product or cosmetic or pharmaceutical preparation according to the present invention is preferably combined with dry-feel modifiers. A dry-feel modifier is an agent which, when incorporated in an emulsion, imparts a “dry feel” to the skin when the emulsion dries. Dry-feel modifiers may also reduce sunscreen migration on the skin. Dry feel modifiers can include starches, talc, kaolin, chalk, zinc oxide, Hydroxyapatite (such as Tinomax CC), silicone fluids, inorganic salts such as barium sulfate and sodium chloride, C6 to C12 alcohols such as octanol; sulfonated oils; surface treated silica, precipitated silica, fumed silica such as Aerosil® available from the Degussa Inc. of New York, N.Y. U.S.A. or mixtures thereof; dimethicone, a mixture of mixture of methylated linear siloxane polymers, available as DC200 fluid, tradename of Dow Corning, Midland, Mich. U.S.A. One or more dry-feel modifiers can optionally be included in the sunscreen in amounts ranging from 0.01 to 20% by weight, more preferably from 0.1 to 10.0% by weight.

Skin moisturizing and/or moisture-retaining substances: The sunscreen product or cosmetic or pharmaceutical preparation according to the present invention comprises advantageously one or more skin-moisturizing and/or moisture-retaining substances. Preferred skin moisturizing and/or moisture-retaining substances are selected from the group consisting of alkane diols or alkane triols comprising 3 to 12 carbon atoms, preferably C₃-C₁₀-alkane diols and C₃-C₁₀-alkane triols. More preferably the skin moisturizing agents are selected from the group consisting of glycerol, 1,2-propylene glycol, 1,2-butylene glycol, 1,3-butylene glycol, 1,2-pentanediol, 1,2-hexanediol, 1,2-octanediol and 1,2-decanediol. Further skin moisturizing and/or moisture-retaining substances include sodium lactate, urea, urea derivatives, alcohols, glycerol, diols such as propylene glycol, hexylene glycol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, 163amascenetin sulphate, lanolin, lanolin esters, amino acids, alpha-hydroxy acids (such as citric acid, lactic acid, malic acid) and their derivatives, mono-, di- and oligosaccharides such as glucose, galactose, fructose, mannose, fructose and lactose, polysugars such as R-glucans, in particular 1,3-1,4-β-glucan from oats, alpha-hydroxy fatty acids, triterpene acids such as betulinic acid or ursolic acid, and algae extracts.

Lenitive substances: The sunscreen product or cosmetic or pharmaceutical preparation according to the present invention can also contain advantageously one or more lenitive substances, wherein any lenitive substances can be used which are suitable or customary in cosmetic or pharmaceutical applications such as alpha-bisabolol, azulene, guaiazulene, 18-beta-glycyrrhetinic acid, allantoin, Aloe vera juice or gel, extracts of Hamamelis virginiana (witch hazel), Echinacea species, Centella asiatica, chamomile, Amici montana, Glycyrrhiza species, algae, seaweed and Calendula officinalis, and vegetable oils such as sweet almond oil, baobab oil, olive oil and panthenol, Laureth-9, Trideceth-9 and 4-t-butylcyclohexanol.

Physiological cooling agents: The sunscreen product or cosmetic or pharmaceutical preparation according to the present invention can be particularly advantageously combined with one or more physiological cooling agent(s). The use of cooling agents can alleviate itching. Preferred individual cooling agents for use within the framework of the present invention are listed below. The person skilled in the art can add many other cooling agents to this list; the cooling agents listed can also be used in combination with one another which are preferably selected here from the following list: menthol and menthol derivatives (for example L-menthol, D-menthol, racemic menthol, isomenthol, neoisomenthol, neomenthol) menthylethers (for example (1-menthoxy)-1,2-propanediol, (1-menthoxy)-2-methyl-1,2-propanediol, 1-menthyl-methylether), menthone glyceryl acetal, menthone glyceryl ketal or mixtures of both, menthylesters (for example menthylformiate, menthylacetate, menthylisobutyrate, menthyhydroxyisobutyrat, menthyllactates, L-menthyl-L-lactate, L-menthyl-D-lactate, menthyl-(2-methoxy)acetate, menthyl-(2-methoxyethoxy)acetate, menthylpyroglutamate), menthylcarbonates (for example menthylpropyleneglycolcarbonate, menthylethyleneglycolcarbonate, menthylglycerolcarbonate or mixtures thereof), the semi-esters of menthols with a dicarboxylic acid or derivatives thereof (for example mono-menthylsuccinate, mono-menthylglutarate, mono-menthylmalonate, O-menthyl succinic acid ester-N,N-(dimethyl)amide, O-menthyl succinic acid ester amide), menthanecarboxylic acid amides (in this case preferably menthanecarboxylic acid-N-ethylamide [WS3] or N^{α}-(menthanecarbonyl)glycinethylester [WS5], menthanecarboxylic acid-N-(4-cyanophenyl)amide or menthanecarboxylic acid-N-(4-cyanomethylphenyl)amide, menthanecarboxylic acid-N-(alkoxyalkyl)amides), menthone and menthone derivatives (for example L-menthone glycerol ketal), 2,3-dimethyl-2-(2-propyl)-butyric acid derivatives (for example 2,3-dimethyl-2-(2-propyl)-butyric acid-N-methylamide [WS23]), isopulegol or its esters (l-(−)-isopulegol, l-(−)-isopulegolacetate), menthane derivatives (for example p-menthane-3,8-diol), cubebol or synthetic or natural mixtures, containing cubebol, pyrrolidone derivatives of cycloalkyldione derivatives (for example 3-methyl-2(1-pyrrolidinyl)-2-cyclopentene-1-one) or tetrahydropyimidine-2-one (for example icilin or related compounds), further carboxamides (for example N-(2-(165amascen-2-yl)ethyl)-3-p-menthanecarboxamide or related compounds), (1R,2S,5R)—N-(4-Methoxyphenyl)-5-methyl-2-(1-isopropyl)cyclohexane-carboxamide [WS12], oxamates and [(1R,2S,5R)-2-isopropyl-5-methyl-cyclohexyl]2-(ethylamino)-2-oxo-acetate (X Cool). Cooling agents which are preferred due to their particular synergistic effect are l-menthol, d-menthol, racemic menthol, menthone glycerol acetal (trade name: Frescolate MGA), menthyl lactate (preferably l-menthyl lactate, in particular l-menthyl l-lactate (trade name: Frescolat® ML)), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate and isopulegol. Particularly preferred cooling agents are l-menthol, racemic menthol, menthone glycerol acetal (trade name: Frescolate MGA), menthyl lactate (preferably 1-menthyl lactate, in particular l-menthyl l-lactate (trade name: Frescolat® ML)), 3-menthoxypropane-1,2-diol, 2-hydroxyethyl menthyl carbonate and 2-hydroxypropyl menthyl carbonate. Very particularly preferred cooling agents are l-menthol, menthone glycerol acetal (trade name: Frescolat® MGA) and menthyl lactate (preferably l-menthyl lactate, in particular l-menthyl l-lactate (trade name: Frescolat® ML).

Alkanediols: In a further preferred variant, the sunscreen product, cosmetic or pharmaceutical preparation or homecare product according to the invention comprises one or more linear alkanediol(s) having a carbon chain of 5 to 12 carbon atoms. Preferably, said one or more linear alkanediol(s) is/are selected from the group consisting of 1,2-alkanediols, 2,3-alkanediols, 3,4-alkanediols and 1,3-alkanediols, more preferably of 2,3-alkanediols and 1,3-alkanediols. Most preferably, said linear alkanediol(s) is/are selected from the group consisting of 1,2-pentanediol, 1,2-hexanediol, 1,2-heptanediol, 1,2-octanediol, 1,2-nonanediol, 1,2-decanediol, 1,2-undecanediol, 1,2-dodecanediol, 2,3-pentanediol, 2,3-hexanediol, 2,3-heptanediol, 2,3-octanediol, 2,3-nonanediol, 2,3-decanediol, 2,3-undecanediol and 2,3-dodecanediol.

Initial observations of the inventors surprisingly indicated that compositions according to the invention comprising one or more linear alkanediol(s) having a carbon chain of 5 to 12 carbon atoms, preferably as defined herein, display particularly advantageous emulsifying properties. In particular, it was surprisingly found that the one or more of said linear alkanediol(s) (if not emulsions themselves) support(s) the formation of water or oil droplets with a particularly small average size in emulsions or (if emulsions themselves) contain water or oil droplets with a particularly small average size.

Additionally, it was found that the presence of an alkanediol, in particular a 1,2-alkanediol or 2,3-alkanediol, improves the solubility of oil components, such as one or more oil body/bodies or wax(es) in the sunscreen product, cosmetic or pharmaceutical preparation or homecare product according to the present invention. Preferably the oil body/bodies or wax(es) are selected from the group consisting of natural fats and oils, fatty alcohols and alcohols, glyceryl esters and derivatives thereof, esters, ethers, siloxanes and silanes, waxes, and naphthenic hydrocarbons, preferably selected from the group consisting of squalene, dialkylcyclohexanes, tetradecane, isohexadecane, dodecane, docosane, paraffin, and mineral oils, and further described below.

A combination of one or more of said alkanediol(s) with 4-hydroxyacetophenon, typically in amounts from 0.1 to 2% by weight, more preferably from 0.1 to 1% by weight, improves the solubility of the oil components even in a synergistic manner.

Preferably, the composition comprises from 0.01 to 10% by weight, more preferably from 0.1 to 5% by weight, still more preferably from 0.3 to 3% by weight, most preferably from 0.5 to 1% by weight, of said linear alkanediol(s) having a carbon chain of 5 to 12 carbon atoms and defined herein, based on the total weight of the composition.

Antioxidants: The sunscreen product, cosmetic or pharmaceutical preparation or homecare product according to the present invention comprises advantageously one or more antioxidants. Suitable antioxidants encompass amino acids (preferably glycine, histidine, tyrosine, tryptophan) and derivatives thereof, imidazoles (preferably urocanic acid) and derivatives thereof, peptides, preferably D,L-carnosine, D-carnosine, L-carnosine and derivatives thereof (preferably anserine), carnitine, creatine, matrikine peptides (preferably lysyl-threonyl-threonyl-lysyl-serine) and palmitoylated pentapeptides, carotenoids, carotenes (preferably alpha-carotene, beta-carotene, lycopene) and derivatives thereof, lipoic acid and derivatives thereof (preferably dihydrolipoic acid), aurothioglucose, propyl thiouracil and other thiols (preferably thioredoxine, glutathione, cysteine, cystine, cystamine and glycosyl, N-acetyl, methyl, ethyl, propyl, amyl, butyl and lauryl, palmitoyl, oleyl, gamma-linoleyl, cholesteryl, glyceryl and oligoglycerol esters thereof) and salts thereof, dilauryl thiodipropionate, distearyl thiodipropionate, thiodipropionic acid and derivatives thereof (preferably esters, ethers, peptides, lipids, nucleotides, nucleosides and salts) and sulfoximine compounds (preferably buthionine sulfoximines, homocysteine sulfoximine, buthionine sulfones, penta-, hexa-, heptathionine sulfoximine) in very small tolerated doses (e.g. pmol to μmol/kg), also (metal) chelators (preferably alpha-hydroxy fatty acids, palmitic acid, phytic acid, lactoferrin, alpha-hydroxy acids (preferably citric acid, lactic acid, malic acid), humic acid, bile acid, bile extracts, tannins, bilirubin, biliverdin, EDTA, EGTA and derivatives thereof), unsaturated fatty acids and derivatives thereof (preferably gamma-linolenic acid, linoleic acid, oleic acid), folic acid and derivatives thereof, ubiquinone and derivatives thereof, ubiquinol and derivatives thereof, vitamin C and derivatives (preferably ascorbyl palmitate, Mg ascorbyl phosphate, ascorbyl acetate, ascorbyl glucoside), tocopherols and derivatives (preferably vitamin E acetate), vitamin A and derivatives (vitamin A palmitate) and coniferyl benzoate of benzoic resin, rutinic acid and derivatives thereof, flavonoids and glycosylated precursors thereof, in particular quercetin and derivatives thereof, preferably alpha-glucosyl rutin, rosmarinic acid, carnosol, carnosolic acid, resveratrol, caffeic acid and derivatives thereof, sinapic acid and derivatives thereof, ferulic acid and derivatives thereof, curcuminoids, chlorogenic acid and derivatives thereof, retinoids, preferably retinyl palmitate, retinol or tretinoin, ursolic acid, levulinic acid, butyl hydroxytoluene, butyl hydroxyanisole, nordihydroguaiac acid, nordihydroguaiaretic acid, trihydroxybutyrophenone, uric acid and derivatives thereof, mannose and derivatives thereof, zinc and derivatives thereof (preferably ZnO, ZnSO₄), selenium and derivatives thereof (preferably selenium methionine), superoxide dismutase, stilbenes and derivatives thereof (preferably stilbene oxide, trans-stilbene oxide) and the derivatives (salts, esters, ethers, sugars, nucleotides, nucleosides, peptides and lipids) of these cited active ingredients which are suitable according to the invention or extracts or fractions of plants having an antioxidant effect, preferably green tea, rooibos, honeybush, grape, rosemary, sage, melissa, thyme, lavender, olive, oats, cocoa, ginkgo, ginseng, liquorice, honeysuckle, sophora, 168amascen, pinus, citrus, Phyllanthus emblica or St. John's wort, grape seeds, wheat germ, Phyllanthus emblica, coenzymes, preferably coenzyme Q10, plastoquinone and menaquinone. Preferred antioxidants are selected from the group consisting of vitamin A and derivatives, vitamin C and derivatives, tocopherol and derivatives, preferably tocopheryl acetate, and ubiquinone.

Emulsifiers: In addition, the sunscreen product, cosmetic or pharmaceutical preparation or homecare product according to the present invention can also advantageously contain one or more emulsifiers in order to keep the sunscreen product, cosmetic or pharmaceutical preparation or homecare product stable. Emulsifiers include amphoteric, anionic, cationic or nonionic emulsifiers, used alone or as a mixture. The emulsifiers are chosen In an appropriate manner according to the emulsion to be obtained. Preferred examples include:
products of the addition of 2 to 30 mol ethylene oxide and/or 0 to 5 mol propylene oxide onto linear C₈₋₂₂ fatty alcohols, onto C₁₂₋₂₂ fatty acids and onto alkyl phenols containing 8 to 15 carbon atoms in the alkyl group;
C_{12/18} fatty acid monoesters and diesters of addition products of 1 to 30 mol ethylene oxide onto glycerol;
glycerol mono- and diesters and sorbitan mono- and diesters of saturated and unsaturated fatty acids containing 6 to 22 carbon atoms and ethylene oxide addition products thereof;
addition products of 15 to 60 mol ethylene oxide onto castor oil and/or hydrogenated castor oil;
polyol esters and, in particular, polyglycerol esters such as, for example, polyglycerol polyricinoleate, polyglycerol poly-12-hydroxystearate or polyglycerol dimerate isostearate. Mixtures of compounds from several of these classes are also suitable;
addition products of 2 to 15 mol ethylene oxide onto castor oil and/or hydrogenated castor oil;
partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose);
mono-, di and trialkyl phosphates and mono-, di- and/or tri-PEG-alkyl phosphates and salts thereof;
wool wax alcohols;
polysiloxane/polyalkyl polyether copolymers and corresponding derivatives;
mixed esters of pentaerythritol, fatty acids, citric acid and fatty alcohol and/or mixed esters of C₆₋₂₂ fatty acids, methyl glucose and polyols, preferably glycerol or polyglycerol,
polyalkylene glycols and
glycerol carbonate.

The addition products of ethylene oxide and/or propylene oxide onto fatty alcohols, fatty acids, alkylphenols, glycerol mono- and diesters and sorbitan mono- and diesters of fatty acids or onto castor oil are known commercially available products. They are homologue mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations. The preferred emulsifiers are described in more detail as follows:

Partial glycerides: Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan esters: Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan 170amascene170, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol esters: Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 to 30 mol ethylene oxide.

Anionic emulsifiers: Typical anionic emulsifiers are aliphatic C12 to C 22 fatty acids, such as palmitic acid, stearic acid or behenic acid for example, and C12 to C22 dicarboxylic acids, such as azelaic acid or sebacic acid for example, potassium cetyl phosphate, and hydrogenated palm glycerides (Emulsiphos/Emulsiphos F).

Amphoteric emulsifiers: Other suitable emulsifiers are amphoteric or zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine.

Anti-ageing actives: A composition according to the present invention is preferably combined with one or more anti-ageing actives. In the context of the invention, anti-ageing or biogenic agents are, for example antioxidants, matrix-metalloproteinase inhibitors (MMPI), skin moisturizing agents, glycosaminoglycan stimulators, anti-inflammatory agents, TRPV1 antagonists and plant extracts.

Matrix-Metalloproteinase inhibitors (MMPI): The compositions according to the present invention are preferably combined with one or more matrix-metalloproteinase inhibitors, especially those inhibiting matrix-metalloproteinases enzymatically cleaving collagen, selected from the group consisting of ursolic acid, retinyl palmitate, propyl gallate, precocenes, 6-hydroxy-7-methoxy-2,2-dimethyl-1(2H)-benzopyran, 3,4-dihydro-6-hydroxy-7-methoxy-2,2-dimethyl-1(2H)-benzopyran, benzamidine hydrochloride, the cysteine proteinase inhibitors N-ethylmalemide and epsilon-amino-n-caproic acid of the serinprotease inhibitors: phenylmethylsufonylfluoride, collhibin (company Pentapharm; INCI: hydrolysed rice protein), oenotherol (company Soliance; INCI: propylene glycol, aqua, Oenothera biennis root extract, ellagic acid and ellagitannins, for example from pomegranate), phosphoramidone hinokitiol, EDTA, MGDA (Trisodiumdicarboxymethyl alaninante), galardin, EquiStat (company Collaborative Group; apple fruit extract, soya seed extract, ursolic acid, soya isoflavones and soya proteins), sage extracts, MDI (company Atrium; INCI: glycosaminoglycans), fermiskin (company Silab/Mawi; INCI: water and Lentinus edodes extract), actimp 1.9.3 (company Expanscience/Rahn; INCI: hydrolysed lupine protein), lipobelle soyaglycone (company Mibelle; INCI: alcohol, polysorbate 80, lecithin and soy isoflavones), extracts from green and black tea and further plant extracts, proteins or glycoproteins from soya, hydrolysed proteins from rice, pea or lupine, plant extracts which inhibit MMPs, preferably extracts from shitake mushrooms, extracts from the leaves of the Rosaceae family, sub-family Rosoideae, quite particularly extracts of blackberry leaf, as e.g. SymMatrix (company Symrise, INCI: Maltodextrin, Rubus fruticosus (Blackberry) Leaf Extract). Preferred actives of are selected from the group consisting of retinyl palmitate, ursolic acid, extracts from the leaves of the Rosaceae family, sub-family Rosoideae, genistein and daidzein.

Skin moisturizing and/or moisture-retaining substances: The sunscreen product or cosmetic or pharmaceutical preparation according to the present invention comprises advantageously one or more skin-moisturizing and/or moisture-retaining substances. Preferred skin moisturizing and/or moisture-retaining substances are selected from the group consisting of alkane diols or alkane triols comprising 3 to 12 carbon atoms, preferably C₃-C₁₀-alkane diols and C₃-C₁₀-alkane triols. More preferably the skin moisturizing agents are selected from the group consisting of glycerol, 1,2-propylene glycol, 1,2-butylene glycol, 1,3-butylene glycol, 1,2-pentanediol, 1,2-hexanediol, 1,2-heptanediol, 1,2-octanediol, and 1,2-decanediol. Further skin moisturizing and/or moisture-retaining substances include sodium lactate, urea, urea derivatives, alcohols, glycerol, diols such as propylene glycol, hexylene glycol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, chondroitin sulfate, lanolin, lanolin esters, amino acids, alpha-hydroxy acids (such as citric acid, lactic acid, malic acid) and their derivatives, mono-, di- and oligosaccharides such as glucose, galactose, fructose, mannose, fructose and lactose, polysugars such as R-glucans, in particular 1,3-1,4-β-glucan from oats, alpha-hydroxy fatty acids, triterpene acids such as betulinic acid or ursolic acid, and algae extracts.

Glycosaminoglycan stimulators: Preferred compositions according to the present invention comprise one or more substances stimulating the synthesis of glycosaminoglycans which are selected from the group consisting of hyaluronic acid and derivatives or salts, Subliskin (Sederma, INCI: Sinorhizobium meliloti Ferment Filtrate, Cetyl Hydroxyethylcellulose, Lecithin), Hyalufix (BASF, INCI: Water, Butylene Glycol, Alpinia galanga leaf extract, Xanthan Gum, Caprylic/Capric Triglyceride), Stimulhyal (Soliance, INCI: Calcium ketogluconate), Syn-Glycan (DSM, INCI: Tetradecyl Aminobutyroylvalylaminobutyric Urea Trifluoroacetate, Glycerin, Magnesium chloride), Kalpariane (Biotech Marine), DC Upregulex (Distinctive Cosmetic Ingredients, INCI: Water, Butylene Glycol, Phospholipids, Hydrolyzed Sericin), glucosamine, N-acetyl glucosamine, retinoids, preferably retinol and vitamin A, Arctium lappa fruit extract, Eriobotrya japonica extract, Genkwanin, N-Methyl-L-serine, (−)-alpha-bisabolol or synthetic alpha-bisabolol such as e.g. Dragosantol and Dragosantol 100 from Symrise, oat glucan, Echinacea purpurea extract and soy protein hydrolysate. Preferred actives are selected from the group consisting of hyaluronic acid and derivatives or salts, retinol and derivatives, (−)-alpha-bisabolol or synthetic alpha-bisabolol such as e.g. Dragosantol and Dragosantol 100 from Symrise, oat glucan, Echinacea purpurea extract, Sinorhizobium meliloti Ferment Filtrate, Calcium ketogluconate, Alpinia galanga leaf extract and tetradecyl aminobutyroylvalylaminobutyric urea trifluoroacetate.

Anti-inflammatory agents: The compositions according to the present invention are preferably combined with anti-inflammatory and/or redness and/or itch ameliorating ingredients, in particular steroidal substances of the corticosteroid type selected from the group consisting of hydrocortisone, dexamethasone, dexamethasone phosphate, methyl prednisolone or cortisone, are advantageously used as anti-inflammatory active ingredients or active ingredients to relieve reddening and itching, the list of which can be extended by the addition of other steroidal anti-inflammatories. Non-steroidal anti-inflammatories can also be used. More particularly:
(i) steroidal anti-inflammatory substances of the corticosteroid type, in particular hydrocortisone, hydrocortisone derivatives such as hydrocortisone 17-butyrate, dexamethasone, dexamethasone phosphate, methylprednisolone or cortisone,
(ii) non-steroidal anti-inflammatory substances, in particular oxicams such as piroxicam or tenoxicam, salicylates such as aspirin, disalcid, solprin or fendosal, acetic acid derivatives such as diclofenac, fenclofenac, indomethacin, sulindac, tolmetin or clindanac, fenamates such as mefenamic, meclofenamic, flufenamic or niflumic, propionic acid derivatives such as ibuprofen, naproxen or benoxaprofen, pyrazoles such as phenylbutazone, oxyphenylbutazone, febrazone or azapropazone,
(iii) natural or naturally occurring anti-inflammatory substances or substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea, or single active compounds thereof,
(iv) histamine receptor antagonists, serine protease inhibitors (e.g. of Soy extracts), TRPV1 antagonists (e.g. 4-t-Butylcyclohexanol), NK1 antagonists (e.g. Aprepitant, Hydroxyphenyl Propamidobenzoic Acid), cannabinoid receptor agonists (e.g. Palmitoyl Ethanolamine) and TRPV3 antagonists.

Examples which can be cited here are oxicams such as piroxicam or tenoxicam; salicylates such as aspirin, disalcid, solprin or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen, benoxaprofen or pyrazoles such as phenylbutazone, oxyphenylbutazone, febrazone or azapropazone. Anthranilic acid derivatives are preferred anti-itch ingredients in a composition according to the present invention.

Also useful are natural or naturally occurring anti-inflammatory mixtures of substances or mixtures of substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea; preferably selected from the group consisting of extracts or fractions from camomile, Aloe vera, oats, calendula, arnica, honeysuckle, rosemary, witch hazel, ginger or Echinacea, and/or pure substances, preferably alpha-bisabolol, apigenin, apigenin-7-glucoside, gingerols, shogaols, gingerdiols, dehydrogingerdiones, paradols, natural or naturally occurring avenanthramides, preferably tranilast, avenanthramide A, avenanthramide B, avenanthramide C, non-natural or non-naturally occurring avenanthramides, preferably dihydroavenanthramide D, dihydroavenanthramide E, avenanthramide D, avenanthramide E, avenanthramide F, boswellic acid, phytosterols, glycyrrhizin, glabridin and licochalcone A; preferably selected from the group consisting of alpha-bisabolol, natural avenanthramides, non-natural avenanthramides, preferably dihydroavenanthramide D (as described in WO 2004 047833 A1), boswellic acid, phytosterols, glycyrrhizin, and licochalcone A, and/or allantoin, panthenol, lanolin, (pseudo-)ceramides [preferably Ceramide 2, hydroxypropyl bispalmitamide MEA, cetyloxypropyl glyceryl methoxypropyl myristamide, N-(1-hexadecanoyl)-4-hydroxy-L-proline (1-hexadecyl) ester, hydroxyethyl palmityl oxyhydroxypropyl palmitamide], glycosphingolipids, phytosterols, chitosan, mannose, lactose and ß-glucans, in particular 1,3->1,4-ß-glucan from oats.

TRPV1 antagonists: Preferred compositions according to the present invention comprise one or more TRPV1 antagonists. Suitable compounds which reduce the hypersensitivity of skin nerves based on their action as TRPV1 antagonists, encompass e.g., trans-4-tert-butyl cyclohexanol, or indirect modulators of TRPV1 by an activation of the p-receptor, e.g., acetyl tetrapeptide-15, are preferred.

Antimicrobial agents: The sunscreen product, cosmetic or pharmaceutical preparation or homecare product according to the present invention can advantageously combined with an antimicrobial agent which act primarily against microorganisms, in particular bacteria, yeast and/or fungi and includes one or more antimicrobial agents such as Caprylhydroxamic Acid, o-Cymen-5-ol, Isopropylparaben, Capryloyl Glycine, Phenylpropanol, Tropolone, PCA Ethyl Cocoyl Arginate, 2-Methyl 5-Cyclohexylpentanol, Phenoxyethanol, Disodium EDTA, Methylparaben and its salts, Sodium Benzoate, Benzyl Alcohol, Potassium Sorbate, Benzyl Salicylate, Propylparaben, Sodium Methylparaben, Methylchloroisothiazolinone, Methylisothiazolinone, Ethylhexylglycerin, Butylparaben, Ethylparaben, Sodium Propylparaben, DMDM Hydantoin, Dehydroacetic Acid, Salicylic Acid, Chlorphenesin, Isobutylparaben, Sodium Ethylparaben, Diazolidinyl Urea, Diazolidinyl Urea, Farnesol, Bisabolol, Glyceryl Caprylate, Sodium Phytate or Phytic Acid, Sodium Levulinate or Levulinic Acid and esters and/or ketals thereof, Chlorhexidine, Glyceryl Laurate, Anisic Acid and its salts, Chlorhexidine Digluconate, TEA-salicylate, Phenethyl Alcohol, Glyceryl Caprate, Sorbitan Caprylate, Tetrasodium Glutamate Diacetate, Trisodium Ethylenediamine Disuccinate, Hydroxyethoxyphenyl Butanone, Hydroxyethoxyphenyl Butanol, Itaconic Acid, Octopirox, Propanediol Caprylate, Climbazole, Undecylenoyl Glycine, Thymol, 4-Hydroxyacetophenone, Lactic Acid, Sodium Lactate, Trisodium Dicarboxymethyl Alaninate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 1,2-undecanediol, 1,2-dodecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, and any mixture thereof.

Preferably, the sunscreen product, cosmetic or pharmaceutical preparation or homecare product according to the present invention comprises one or more antimicrobial agent selected from the group consisting of 1,2-pentanediol, 1,2-hexanediol, 1,2-heptanediol, 1,2-octanediol, 1,2-nonanediol, 1,2-decanediol, 1,2-undecanediol, 1,2-dodecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2-benzylheptanol, 2-hydroxyacetophenone, 2-methyl 5-cyclohexylpentanol, 3-hydroxypropyl 2-hydroxybenzoate, 4-hydroxyacetophenone, and any mixture thereof. More preferably, the sunscreen product, cosmetic or pharmaceutical preparation or homecare product according to the present invention comprises one or more antimicrobial agent selected from the group consisting of 1,2-pentanediol, 1,2-hexanediol, 1,2-heptanediol, 1,2-octanediol, 1,2-nonanediol, 1,2-decanediol, 1,2-undecanediol, 1,2-dodecanediol, 1,2-tridecanediol, 4-hydroxyacetophenone, and any mixture thereof. In order to further enhance antimicrobial efficacy, the sunscreen product, cosmetic or pharmaceutical preparation or homecare product as defined herein, is advantageously combined with at least one further antimicrobial agent which is different from the antimicrobial agents specified above. The combination with a further antimicrobial agent provides reliable protection against microbial degradation and deterioration of the preparation, in particular during storage. Additionally, the further different antimicrobial agent provides reliable protection against other microorganisms as described above, for example Corynebacterium, Anaerococcus, Finegoldia, Moraxella, Porphyromonas, Fusobacterium, Malassezia, Peptoniphilus, Streptococcus, Lactobacillus, Gardnerella, Fannyhessea, Epidermophyton, Trichophyton, Cutibacterium. Hence, the combination with a further different antimicrobial agent allows antimicrobial protection against different groups of microorganisms, and, thus, a broader spectrum of microorganism. The further different antimicrobial agent in the context of the present invention is preferably selected from the group consisting of 2-benzylheptanol, alkyl (C 12-22) trimethyl ammonium bromide and chloride, ascorbic acid and salts thereof, benzalkonium bromide, benzalkonium chloride, benzalkonium saccharinate, benzethonium chloride, Benzoic Acid, camphor, cetylpyridinium chloride, chlorhexidine diacetate, chlorhexidine dihydrochloride, chlorocresol, chloroxylenol, Cyclohexylglycerin, Dimethyl phenylbutanol, Dimethyl phenylpropanol, ethanol, ethyl lauroyl arginate HCl, Ethyl Lauroyl Arginate Laurate, eucalyptol, gluconic acid and salts thereof, glycerin, hexamidine, hexamidine diisethionate, Hexylglycerin, Iodopropynyl Butylcarbamate, jasmol, lauryl alcohol, Levulinic Acid and esters and/or ketals thereof, mannitol, menthol, methyl salicylate, Octenidine HCl, polyarginine, Polyglyceryl-10 Laurate, polyglyceryl-2 caprate, Polyglyceryl-3 caprylate, polylysine, Salvia officinalis (Sage) Oil, silver chloride, silver citrate, sodium caproyl lactylate, Sodium Caproyl/Lauroyl Lactylate, sodium lauroyl lactylate, Sorbic Acid, sorbitol, Tetraselmis extract, triclocarban, triclosan, Triethyl citrate, Xylityl Sesquicaprylate, zinc citrate, zinc pyrithione, Zinc ricinoleate, and any mixture thereof.

The aforesaid antimicrobial agents are used in the sunscreen, cosmetic or pharmaceutical preparation or homecare product either as a single component or preferably in a mixture with two or more further of said antimicrobial agents as specified above.

The antimicrobial agent is present in the sunscreen product, cosmetic or pharmaceutical preparation or homecare product in an amount of 0.01 to 25.0% by weight, preferably 0.02 to 15.0% by weight, most preferred in an amount of 0.05 to 6.0% by weight, based on the total weight of the final formulation.

Advantageously, the at least one antimicrobial alkanediol is present in the sunscreen product, cosmetic or pharmaceutical preparation or homecare product in an amount of 0.1 to 5% by weight, based on the total weight of the final formulation and/or the 4-hydroxyacetophenone in an amount of 0.01 to 2% by weight, based on the total weight of the final formulation.

Preservatives: For preservative purposes, the sunscreen product, cosmetic or pharmaceutical preparation or homecare product according to the present invention preferably includes one or more preservatives which are suitable or customary in sunscreen products, cosmetic or pharmaceutical preparations or homecare products. Suitable and advantageously preservatives are, for example, benzoic acid, sodium benzoate, ammonium benzoate, butyl benzoate, calcium benzoate, ethyl benzoate, isobutyl benzoate, isopropyl benzoate, magnesium benzoate, meta-benzoate, methyl benzoate, phenyl benzoate, potassium benzoate, propyl benzoate, propionic acid, ammonium propionate, calcium propionate, magnesium propionate, potassium propionate, sodium propionate, salicylic acid, calcium salicylate, magnesium salicylate, measalicylate, sodium salicylate, potassium salicylate, teasalicylate, sorbic acid, calcium sorbate, sodium sorbate, potassium sorbate, o-phenylphenol, sodium sulfite, ammonium bisulfite, ammonium sulfite, potassium sulfite, potassium hydrogen sulfite, sodium bisulfite, sodium metabisulfite, potassium metabisulfite, chlorobutanol, 4-hydroxybenzoic acid, methylparaben, potassium ethylparaben, potassium paraben. Sodium methylparaben, sodium ethylparaben, ethylparaben, sodium paraben, potassium methylparaben, calcium paraben, butylparaben, propylparaben, sodium propoylparaben, sodium butylparaben, potassium butylparaben, potassium propylparaben, dehydroacetic acid, sodium dehydroacetate, formic acid, sodium formate, dibromohexamidine isethionate, thimerosal, phenyl mercuric acetate, phenyl mercuric benzoate, undecylenic acid, potassium undecylenate, sodium undecylenate, calcium undecylenate, mea-undecylenate, tea-undecylenate, hexetidine, 5-bromo-5-nitro-1,3-dioxane, 2-bromo-2-nitropropane-1,3-diol, dichlorobenzyl alcohol, triclocarban, p-chloro-m-cresol, triclosan, chloroxylenol, imidazolidinyl urea, polyaminopropyl; biguanide, phenoxyethanol, methenamine, climbazole, DMDM hydantoin, benzyl alcohol, 1-hydroxy-4-methyl-6-(2,4,4-trimethylpentyl)-2 pyridon, piroctone olamine, bromochlorophene, o-cymen-5-ol, methylchloroisothiazolinone and methylisothiazolinone, chlorhexidine, chlorhexidine diacetate, chlorhexidine digluconate, chlorhexidine dihydrochloride, phenoxyisopropanol, behentrimonium chloride(1), cetrimonium bromide, cetrimonium chloride(2), laurtrimoniumbromide, laurtrimonium chloride, steartrimonium bromide, steartrimonium chloride (2), dimethyl oxazolidine, diazolidinyl urea, hexamidine, hexamidine diisethionate, hexamidine diparaben, hexamidine paraben, glutaral, 7-ethylbicyclooxazolidine, chlorphenesin, sodium hydroxymethylglycinate, silver chloride, benzethonium chloride, benzalkonium chloride, benzalkonium bromide, benzalkonium saccharinate, benzylhemiformal, iodopropynyl butylcarbamate, methylisothiazolinone, citric acid, silver citrate, hydroxyethoxy-phenyl butanone (HEPB) and any mixture thereof.

Drugs: The sunscreen product, cosmetic or pharmaceutical preparation or homecare product according to the present invention is preferably combined with one or more drugs. In certain such embodiments, the pharmaceutically active agent is selected from anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including elfomithine and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

Especially useful are natural or naturally occurring anti-inflammatory mixtures of substances, in particular extracts or fractions from 180amascene, Aloe vera, Commiphora species, Rubia species, willow, willow-herb, oats, calendula, arnica, St John's wort, honeysuckle, rosemary, Passiflora incarnata, witch hazel, ginger or Echinacea; preferably selected from the group consisting of extracts or fractions from 180amascene, Aloe vera, oats, calendula, arnica, honeysuckle, rosemary, witch hazel, ginger or Echinacea, and/or pure substances, preferably alpha-bisabolol, apigenin, apigenin-7-glucoside, gingerols, shogaols, gingerdiols, dehydrogingerdiones, paradols, natural or naturally 180amascene avenanthramides, preferably tranilast, avenanthramide A, avenanthramide B, avenanthramide C, non-natural or non-naturally 180amascene avenanthramides, preferably dihydroavenanthramide D, dihydroavenanthramide E, avenanthramide D, avenan-thramide E, avenanthramide F, boswellic acid, phytosterols, glycyrrhizin, glabridin and licochalcone A; preferably selected from the group consisting of alpha-bisabolol, natural avenanthramides, non-natural avenanthramides, preferably dihydroavenanthramide D (as described in WO 2004 047833 A1), boswellic acid, phytosterols, glycyrrhizin, and licochalcone A, and/or allantoin, panthenol, lanolin, (pseudo-)ceramides [preferably Ceramide 2, hydroxypropyl bispalmitamide MEA, cetyloxypropyl glyceryl methoxypropyl myristamide, N-(1-hexadecanoyl)-4-hydroxy-L-proline (1-hexadecyl) ester, hydroxyethyl palmityl oxyhydroxypropyl palmitamide], glycosphingolipids, phytosterols, chitosan, mannose, lactose and ß-glucans, in particular 1,3->1,4-ß-glucan from oats.

In addition to the above-described substances, further ingredients commonly used in sunscreen products, cosmetic or pharmaceutical preparations or homecare products, which are suitable or customary in the compositions of the present invention, can be used.

The sunscreen product or cosmetic or pharmaceutical, in particular dermatological, preparation according to the first aspect of the present invention is intended for topical applications. The term “topical” is understood to mean external applications on a mammal's skin or mucosa, which are in particular for the protection, treatment, care and cleansing of the skin, scalp, eyelashes, eyebrows, nails, mucous membranes and hair. The mammal is preferably a human.

For topical application, the cosmetic or pharmaceutical preparation is either a rinse off or a leave on preparation.

The sunscreen products according to the present invention may be in the form of an aqueous solution, dispersions, a hydro alcoholic vehicle, a stick, an ointment, a gel, an aerosol (foams, sprays, propellant pump) and the like.

The sunscreen product, cosmetic or pharmaceutical, in particular dermatological, preparation or homecare product according to the first aspect of the present invention can be present in different forms, e.g. in the form of a dispersion, in the form of a water free formulation, or in the form of an aqueous, aqueous/alcoholic, in particular aqueous/ethanolic, aqueous/glycolic or alcoholic/glycolic, in particular ethanolic/glycolic, based solution.

In a preferred variant, the sunscreen product, cosmetic or pharmaceutical preparation or homecare product according to the first aspect of the invention is a dispersion. The term “dispersion” in the context of the present invention means, that the sunscreen product, cosmetic or pharmaceutical preparation or homecare product is a disperse two-phase system consisting of colloidal particles (disperse phase) and a medium in which they are suspended (disperse medium). Both phases are not miscible with each other, only with an emulsifier. Such dispersions, for example emulsions, comprise the at least one oil component (without UV-filters) preferably in an amount of Z 1% by weight, more preferably in an amount of ≥3% by weight.

Preferably, the sunscreen product, cosmetic or pharmaceutical preparation or homecare product according to the first aspect of the present invention takes various forms such as an emulsion, in particular a O/W emulsion, a W/O emulsion, a multiple emulsion, a hydrodispersion gel, a balm, a multiple emulsion of the water-in-oil type (W/O/WO) or of the oil-in-water type (O/W/O), PIT emulsion, Pickering emulsion, a micro-emulsion, a liquid, a lotion, a suspension, a milk, an ointment, a paste, a gel, a cream based, an oil based or a liposome-based formulation or a an aerosol such as foams and sprays, including all types of silicon based emulsions.

In a most preferred variant, the sunscreen product, cosmetic or pharmaceutical preparation or homecare product according to the present invention is in the form of an emulsion as defined herein, advantageously in the form of an oil-in-water (O/W) emulsion comprising an oily phase dispersed in an aqueous phase in the presence of an O/W or polymeric emulsifier.

If the sunscreen product, cosmetic or pharmaceutical preparation or homecare product according to the present invention is a dispersion, preferably an emulsion, the oil component (without UV-filters) is present in the cosmetic or pharmaceutical preparation in an amount of 0.01 to 50.0% by weight, based on the total weight of the composition. In a preferred variant, the cosmetic or pharmaceutical preparation comprises the oil component in an amount of 0.1 to 45.0% by weight, based on the total weight of the composition. In a particular preferred variant, the oil component is advantageously used in the cosmetic or pharmaceutical preparation in an amount of at 1.0 to 40% by weight, based on the total weight of the composition.

In a further variant, the sunscreen product, cosmetic or pharmaceutical, preferably dermatological, preparation or homecare product according to the first of the invention is a water free formulation, i.e. an oil formulation.

If the sunscreen product, cosmetic or pharmaceutical preparation or homecare product according to the present invention is a water free formulation, i.e. an oil formulation, the oil component is present in the cosmetic or pharmaceutical preparation in an amount of ≥60% by weight, preferably in an amount of ≥75% by weight, more preferably in an amount of ≥90% by weight, based on the total weight of the composition.

Such water free formulations include e.g. oils, skin butters, powders, lip stick, antiperspirant/deo sticks, and decorative cosmetics.

In addition, further water free formulations are likewise formulations on the basis of ethanol/diols/triols/glycols such as sprays or gels.

Alternatively, the sunscreen product, cosmetic or pharmaceutical preparation or homecare product as disclosed herein is an aqueous or an aqueous/alcoholic, preferably aqueous/ethanolic, or an aqueous/glycolic, or an alcoholic/glycolic, preferably ethanolic/glycolic, based solution. Typically, this could be glycerin in water or alcohol compositions. Said solutions are homogeneous one phase system of water/alcohol/glycol and additional components. The aqueous/alcoholic or aqueous/glycolic or alcoholic/glycolic based solution comprises an aliphatic alcohol or a glycol in an amount of 0.1 to 70.0% by weight, preferably in an amount of 0.5 to 60.0% by weight, more preferably in an amount of 1.0 to 50.0% by weight, based on the total weight of the solution. The aliphatic alcohol is preferably selected from the group consisting of ethanol, isopropanol, n-propanol. The glycol is preferably selected from the group consisting of glycerin, propylene glycol, 1,3-Propanediol, 1,2-Propanediol, 1,2-C5 to C10-alkanediols, butylene glycol or dipropylene glycol.

Preferably, the overall water content in the final aqueous based solutions can be ≥30% by weight, more preferably ≥40% by weight, most preferably ≥50% by weight.

Such aqueous or aqueous/alcoholic or aqueous/glycolic or alcoholic/glycolic based solutions include for example deo/antiperspirant preparations, after shave, cleansing preparations, or anti-acne preparations.

In a preferred variant, the inventive composition is an impregnation solution in the form of an emulsion spray or ethanol/oil spray for wet wipes.

The above formulations or compositions are prepared according to usual and known methods.

The products and preparations according to the present invention are for cosmetic and/or non-therapeutic use for personal care, skin protection, skin care, sun care, scalp protection, scalp care, hair care, nail care, in particular for the prevention and treatment of skin conditions, intolerant or sensitive skin, skin irritation, skin reddening, wheals, pruritis (itching), skin aging, wrinkle formation, loss of skin volume, loss of skin elasticity, piment spots, pigment abnormalities, skin dryness, flaking, greasiness, hypopigmentation and/or hyperpigmentation of the skin; or a preparation for animal care.

Examples of personal care are preferably anti-ageing preparations, skin care emulsions, body oils, body lotions, cleansing lotions, face or body balms, after shave balms, after sun balms, deo emulsions, cationic emulsions, body gels, treatment creams, skin protection ointments, moisturizing gels, face and/or body moisturizers, light protective preparations (sunscreens), micellar water, hair sprays, color protection hair care products, skin lightning products, anti-dark-spot reparation, etc.

The cosmetic preparations include also make-ups, eye-care preparation, eye shadows, mascara, eyeliner, lip care preparation such as lip stick, lip gloss, mail care preparations, such as nail varnish, nail varnish removers,

Alternatively, the preparation according to the present invention is a preparation of medical use.

The pharmaceutical, in particular dermatological, composition according to the present invention is preferably a preparation for the prevention and treatment of a condition of the skin, mucosa, nails or hair.

In order to be used, the sunscreen product or cosmetic or pharmaceutical, in particular dermatological composition according to the first aspect of the present invention is applied to the skin, hair, scalp and/or nails in an adequate amount in such manner as is customary with sunscreen products or cosmetics and dermatological products.

In addition, the products and preparations according to the present invention are homecare products. The home care products generally include laundry detergents (powder, liquid and tablet), fabric conditioners, dishwashing detergents (liquid and tablet), hard floor and surface cleaners, glass cleaners, carpet cleaners, oven cleaners, air fresheners, disinfectants, stain removers, car wash products. These products are usually manufactured in the form of a liquid, powder, spray, granules and others.

Surprisingly, it was found that the addition of a mycosporine-like amino acid compounds or their salts as defined in the present application has the effect of improving the water resistance of a sunscreen product, cosmetic or pharmaceutical preparation or homecare product.

The increased water resistance makes said formulations more substantive to the skin or surfaces. Hence, important ingredients of said formulations, such as UV-filters, are not leached out when they come in contact with water and make the products more effective.

As it is demonstrated in the following example, the water resistance is highly improved, i.e. increased, by the addition of a mycosporine-like amino acid compound (b) as defined therein. The control samples containing no mycosporine-like amino acid compound (b) exhibited average or low water resistance properties.

An improvement of the water resistance can be observed when a mycosporine-like amino acid compound according to general formulae (V) to (X) or as defined herein is added to a sunscreen product or cosmetic or pharmaceutical preparation. A pronounced water resistance improving effect is especially observed for the mycosporine-like amino acid compounds as specified in Table 3.

With the addition of a mycosporine-like amino acid compound according to general formula (VI), wherein X is S, SO or SO₂, a particular pronounced water resistance improving effect can be reached.

Surprisingly, the mycosporine-like amino acid compounds according to the present invention are particularly effective in improving the water resistance of a sunscreen product or cosmetic or pharmaceutical preparation when they are in the form of an acid or in salt form as described above in detail.

With the addition of a mycosporine-like amino acid compound as defined herein, the water resistance of a sunscreen product, a cosmetic or pharmaceutical preparation or homecare product can be increased by at least 5%, in some instances by even more than 10%, as it is demonstrated in the following examples.

The addition of MAA-1 in an amount of 1.0% by weight however results in a water resistance which is 5.9% higher than the water resistance of the reference sample, i.e. the sample without the addition of the mycosporine-like amino acid compound. The addition of MAA-1 in an amount of 3.0% by weight even results in a water resistance which is 10.1% higher than the water resistance of the control sample, i.e. containing none mycosporine-like amino acid compound. The addition of MAA-13 in an amount of 1.0 results in a water resistance which is 6.8% higher than the water resistance of the reference sample, i.e. the sample without the addition of the mycosporine-like amino acid compound. The addition of MAA-13 in an amount of 3.0% by weight even results in a water resistance which is 10.6% higher compared to the water resistance of the reference sample, i.e. the sample without the addition of the mycosporine-like amino acid compound.

Similar water resistance improving effects can also be shown for the mycosporine-like amino acid compounds MAA-2, MAA-3, MAA-4, MAA-5, MAA-6, MAA-7, MAA-8, MAA-9, MAA-11, MAA-12, MAA-13, MAA-14, MAA-16 and MAA-17 and MAA-20 to MAA-39 as specified above or any mixture thereof. Said compounds show an increase in water resistance by at least 5% compared to the water resistance of a sunscreen product or cosmetic or pharmaceutical preparation without the combination with said mycosporine-like amino acid compounds.

A particular water resistance improving effect can also be demonstrated for the Weinreb-amide derivatives, hydroxamate derivatives or alkylated hydroxamate derivatives of the mycosporine-like amino acid compounds as defined herein. The water resistance effect is especially pronounced for the mycosporine-like amino acid compounds MAA-A, MAA-B and MAA-C as specified above or any mixture thereof.

Furthermore, with the addition of a mycosporine-like amino acid as defined herein to the sunscreen product or cosmetic or pharmaceutical preparation or homecare product the adhesion of the film forming agent on the skin or on surfaces can significantly be improved. Preferably, the combination of a film forming agent and a mycosporine-like amino acid result in a synergistic adherence effect of the sunscreen product or cosmetic or pharmaceutical preparation or homecare product on the skin or on a surface, to which the formulation is applied. The improved adherence of the film forming agent in turn results in an improved water resistance, and, thus, provides excellent UV-protection even after bathing compared to products or preparations without the addition of the specified mycosporine-like amino acid compounds.

Such tenacity improving effect can be shown for the mycosporine-like amino acid compounds MAA-1 to MAA-39 or any mixture thereof. Beneficial tenacity improving effect can be obtained preferably for MAA-1 and MAA-13 or a mixture thereof.

This tenacity effect can especially be observed when the mycosporine-like amino acid is in its salt form, i.e. in its cationic form. The combination of the film building agents with said mycosporine-like amino acid compounds show a distinctive adhesion of the sunscreen products, cosmetic or pharmaceutical preparation or homecare product on the skin or on surfaces, resulting in an improvement of the water resistance.

A particular adhesion improving effect can also be demonstrated for the Weinreb-amide derivatives, hydroxamate derivatives or alkylated hydroxamate derivatives of the mycosporine-like amino acid compounds as defined herein. The adhesion improving effect is especially pronounced for the mycosporine-like amino acid compounds MAA-A, MAA-B and MAA-C as specified above or any mixture thereof.

With the improvement of the water resistance of a sunscreen product, cosmetic or pharmaceutical or homecare product, the SPF can also be improved. With the present invention it is therefore possible to offer sunscreen products, cosmetic or pharmaceutical products or homecare product having a significant increase in water resistance as well as high sun protection factor (SPF). The higher the SPF the more protective the sunscreen is.

On the other hand, the improved SPF allows to reduce the amount of the UV-filter, in particular the primary organic and/or inorganic UV-filter, without affecting the envisaged SPF.

With the admixture of a mycosporine-like amino acid compound (b) to a sunscreen product or cosmetic or pharmaceutical preparation comprising a primary UV-filter, the SPF of the sunscreen product or the cosmetic or pharmaceutical preparation remains almost high and thus provide excellent UV-protection even after bathing compared to products or preparations without the addition of the specified mycosporine-like amino acid compounds.

Hence, the present invention provides for a sunscreen product or cosmetic or pharmaceutical preparation wherein the water resistance is increased by at least 5%, compared to a sunscreen product or cosmetic or pharmaceutical preparation without the mycosporine-like amino acid compound, preferred by at least 7.5%, more preferred by at least 10% and most preferred by at least 12.5%.

In particular, this water resistance improving effect is observed without the use of further film forming agents.

Due to this beneficial effect as described before, the present invention relates in a further aspect to the use of a mycosporine-like amino acid compound as defined herein or a mixture thereof for improving the water resistance of a sunscreen product or cosmetic or pharmaceutical preparation stabilizing the photostability of a UV-filter.

By the admixture of a mycosporine-like amino acid compound as defined herein or a mixture thereof the water resistance can be increased by at least 5%, compared to a sunscreen product or cometic or pharmaceutical preparation without the addition of a mycosporine-like amino acid compound. Preferably, the water resistance is increased by at least 7.5%, more preferred by at least 10% and most preferred by at least 12.5%.

In a further aspect, the present invention relates to a method of increasing the water resistance of a sunscreen product or cosmetic or pharmaceutical preparation. In the method an effective amount of at least one mycosporine-like amino acid compound as defined herein or a mixture thereof is added to said sunscreen product or cosmetic or pharmaceutical preparation.

The present invention shall now be described in detail with reference to the following examples, which are merely illustrative of the present invention, such that the content of the present invention is not limited by or to the following examples.

### EXAMPLES

### Example 1—Water Resistance Test

### The Water Resistance Test is Carried Out as In-Vitro Substantivity Determination of UV-Filters

Principle: The formulation to be tested (emulsion, solution) is weighed on fabric strips with the help of an analytical balance and half of the fabric strips are immersed in water. Subsequently, all fabric strips are individually boiled, the extract solution is transferred to a measuring flask, filled up to 100 ml and measured in a UV-Vis spectrometer. The absorption maxima are used to determine the difference between the immersed/watered and the undiluted/non-watered samples and to calculate the substantivity.

### 1. Sample Preparation:

1.1 Fabric: The fabric used in pre-treated as follows: the fabric is washed twice in a conventional washing machine with unscented mild detergent at 30° C. and then air-dried. Fabric made of 100% pure new wool is suitable for the test.
1.2 Watered sample: 0.100 to 0.150 g of a formulation as specified in the following Table 4 to be tested is weighed on a treated wool fabric strip, as described before, of 5×10 cm in size. The formulation is distributed with the finger. The sample is allowed to dry for 30 minutes. Then the sample is weighed back. The formulation on the wool fabric strip is then immersed/watered in 4.5 l water at 20 to 25° C. while the water was stirred with a magnetic stirrer at 100 r/min for 80 minutes. Afterwards, the fabric strip is dried at room temperature for at least 12 hours.
1.3 Reference sample: 0.100 to 0.150 g of a formulation as specified in the following Table 4 to be tested is weighed on a treated wool fabric strip, as described before, of 5×10 cm in size. The formulation is distributed with the finger. The sample is allowed to dry for 30 minutes. Then the sample is weighed back. Afterwards, the fabric strip is dried at room temperature for at least 12 hours.
1.4 Decoction of the samples: The immersed/watered and undiluted/non-watered samples are successively boiled with 60 ml of a suitable solvent in a 100 ml three-neck flask for 10 minutes under reflux. The solution is then cooled to ambient temperature and transferred to a 100 ml volumetric flask and filled up to the calibration mark.

### 2. Determination of UV Absorption and Calculation:

The absorption curves of both samples as described above are measured with an UV/VIS Lambda 25 spectrometer and the maxima absorption between 190 and 400 nm is determined for each sample.

The measurement is carried out as a double determination and the deviation from the mean shall not exceed ±2%.

The results of absorption measurement are used to calculate the substantivity according to the following formula:

$$Substantivity in \% = \frac{UV maximum ( watered sample ) \times weight ( comparative sample )}{UV maximum ( comparison sample ) \times weight ( watered sample )} \times 100$$

### 3. Tested Samples:

The formulation samples A to E as specified in the following Table 4 were prepared as follows:

Phase A: The ingredients of phase A were heated to 85° C. without Keltrol® and Carbopol®, in order to dissolve the ingredients and homogenized for a short time (Ultra Turrax® T25: 5000 rpm/10 sec).

Phase B: The ingredients of phase B were mixed while stirring, in order to dissolve the ingredients. The water phase will be cloudy in combination with sodium hydroxide solution. Afterwards, the water phase B is heated to 80° C. Afterwards, the hot phase B was added to the hot phase A and homogenized (Ultra Turrax® T25: 13000m/13 min). Finally, the mixture was cooled down to ambient temperature while stirring.

The results of the in vitro determination of the water resistance are presented in Table 4.

TABLE 4

| | | | A % | B % | C % | D % | E % |
|---|---|---|---|---|---|---|---|
| Phase | Ingredient | INCI: | w/w | w/w | w/w | w/w | w/w |
| A | Emulsiphos ® | Potassium Cetyl | 2.0 | 2.0 | 2.0 | 2.0 | 2.0 |
| | | Phosphate, | | | | | |
| | | Hydrogenated Palm | | | | | |
| | | Glycerides | | | | | |
| | Lanette ® O | Cetearyl Alcohol | 2.0 | 2.0 | 2.0 | 2.0 | 2.0 |
| | Neo Heliopan ® | Butyl | 3.0 | 3.0 | 3.0 | 3.0 | 3.0 |
| | 357 | Methoxydibenzoylmethane | | | | | |
| | Neo Heliopan ® | Ethylhexyl Salicylate | 5.0 | 5.0 | 5.0 | 5.0 | 5.0 |
| | OS | | | | | | |
| | Neo Heliopan ® | Octocrylene | 7.0 | 7.0 | 7.0 | 7.0 | 7.0 |
| | 303 | | | | | | |
| | Neo Heliopan ® | Homosalate | 10.0 | 10.0 | 10.0 | 10.0 | 10.0 |
| | HMS | | | | | | |
| | Tocopherylacetat | Tocopheryl Acetate | 0.5 | 0.5 | 0.5 | 0.5 | 0.5 |
| | EDTA ® BD | Disodium EDTA | 0.1 | 0.1 | 0.1 | 0.1 | 0.1 |
| | Carbopol ® ETD | Carbomer | 0.3 | 0.3 | 0.3 | 0.3 | 0.3 |
| | 2050 | | | | | | |
| | Keltrol ® T | Xanthan Gum | 0.3 | 0.3 | 0.3 | 0.3 | 0.3 |
| B | Water dem. | Aqua (Water) | 61.0 | 59.5 | 57.0 | 59.5 | 57.0 |
| | Neo Heliopan ® | Phenylbenzimidazole | 2.0 | 2.0 | 2.0 | 2.0 | 2.0 |
| | Hydro | Sulfonic Acid | | | | | |
| | Neo Heliopan ® | Disodium Phenyl | 1.0 | 1.0 | 1.0 | 1.0 | 1.0 |
| | AP | Dibenzimidazole | | | | | |
| | | Tetrasulfonate | | | | | |
| | MAA-1 | | — | 1.0 | 3.0 | — | — |
| | MAA-13 | | — | — | — | 1.0 | 3.0 |
| | NaOH 10% aq | Aqua, Sodium Hydroxide | 5.0 | 5.5 | 6.0 | 5.5 | 6.0 |
| | Phenonip ® XB | Phenoxyethanol, | 0.8 | 0.8 | 0.8 | 0.8 | 0.8 |
| | | Methylparaben, | | | | | |
| | | Ethylparaben, | | | | | |
| | | Propylparaben | | | | | |
| | Sum | | 100.0 | 100.0 | 100.0 | 100.0 | 100.0 |
| | pH value | | 7.4 | 7.4 | 7.3 | 7.5 | 7.4 |
| | about 7-7.5 | | | | | | |
| | Color | | white | Yellowish | yellow | Yellowish | yellow |
| | in vitro water | | 77.2 | 83.1 | 87.3 | 84.0 | 87.8 |
| | resistance (%) | | | | | | |
| | water resistance | | | +5.9 | +10.1 | +6.8 | +10.6 |
| | versus reference | | | | | | |
| | formulation A (%) | | | | | | |

Results: As can be retrieved from Table 4, the water resistance is highly improved, i.e. increased by the combination of the formulation with a certain mycosporine-like amino acid compound. The control example (A) containing no mycosporine-like amino acid compound exhibited average or lower water resistance properties in comparison to the tested sample containing a mycosporine-like amino acid. The addition of MAA-1 in an amount of 1.0% by weight however results in a water resistance which is 5.9% higher than the water resistance of the reference sample, i.e. the sample without the addition of the mycosporine-like amino acid compound. The addition of MAA-1 in an amount of 3.0% by weight even results in a water resistance which is 10.1% higher than the water resistance of the control sample, i.e. containing none mycosporine-like amino acid compound. The addition of MAA-13 in an amount of 1.0 results in a water resistance which is 6.8% higher than the water resistance of the reference sample, i.e. the sample without the addition of the mycosporine-like amino acid compound. The addition of MAA-13 in an amount of 3.0% by weight even results in a water resistance which is 10.6% higher compared to the water resistance of the reference sample, i.e. the sample without the addition of the mycosporine-like amino acid compound.

With the improvement of the water resistance of a sunscreen product or cosmetic or pharmaceutical, the SPF can conversely be improved. It is therefore possible to offer sunscreen products or cosmetic or pharmaceutical products having a significant increase in water resistance as well as high sun protection factor (SPF).

### FORMULATION EXAMPLES

In the following formulation examples, the following five perfume oils PFO1, PFO2, PFO3, PFO4 or PFO5 were each used as fragrance.

TABLE F1
Composition of perfume oil 1 (PO1; amounts in ‰ by weight)

| Ingredients | Amount |
|---|---|
| Aldehyde C14 | 2.0 |
| Aldehyd C12 | 2.0 |
| Allyl amyl glycolate 10% dpg | 5.0 |
| Anisic aldehyde pure | 5.0 |
| Apple oliffac type | 10.0 |
| Benzylacetate | 50.0 |
| Bergamot identoil ® colourless | 15.0 |
| Canthoxal | 5.0 |
| Cetalox 10% ipm | 3.0 |
| Citronellol 950 | 40.0 |
| Damascenone total 1% dpg | 5.0 |
| Damascone alpha 10% dpg | 5.0 |
| Damascone delta 10% dpg | 2.0 |
| Dimethyl benzyl carbinyl butyrate | 2.0 |
| Dipropylene glycol | 176.0 |
| Ebanol | 2.0 |
| Ethyl decadienoate trans cis-2.4 10% ipm | 2.0 |
| Florosa | 5.0 |
| Frambinon ® 10% dpg | 7.0 |
| Galaxolide 50% in ipm | 100.0 |
| Galbex type base | 1.0 |
| Geranyl acetate pure | 2.0 |
| Hedione | 30.0 |
| Heliotropin | 10.0 |
| Hexenyl acetate cis-3 10% dpg | 1.0 |
| Hexenyl salicylate cis-3 | 5.0 |
| Hexyl cinnamic aldehyde alpha | 70.0 |
| Hexyl salicylate | 50.0 |
| Hydroxy citronellal | 10.0 |
| Iso e super | 15.0 |
| Isoraldeine 70 | 20.0 |
| Leafovert ® | 1.0 |
| Lilial | 60.0 |
| Linalool | 60.0 |
| Linalyl acetate | 20.0 |
| Lyral | 7.0 |
| Manzanate | 2.0 |
| Phenoxanol | 7.0 |
| Phenylethyl alcohol | 120.0 |
| Sandal mysore core | 2.0 |
| Sandranol ® | 7.0 |
| Styralyl acetate | 3.0 |
| Tagetes rco 10% tec | 2.0 |
| Terpineol pure | 20.0 |
| Tetrahydrogeraniol 10% dpg | 5.0 |
| Tonalide | 7.0 |
| Vertocitral 10% dpg | 5.0 |
| Vertofix | 15.0 |
| Total | 1000.0 |

TABLE F2
Composition of perfume oil 2 (PO2; amounts in ‰ by weight)

| Ingredients | Amount |
|---|---|
| Acetophenone. 10% in DPG | 10.0 |
| n-Undecanal | 5.0 |
| Aldehyde C14. So-called (peach aldehyde) | 15.0 |
| Allylamyl glycolate. 10% in DPG | 20.0 |
| Amyl salicylate | 25.0 |
| Benzyl acetate | 60.0 |
| Citronellol | 80.0 |
| d-Limonene | 50.0 |
| Decenol trans-9 | 15.0 |
| Dihydromyrcenol | 50.0 |
| Dimethylbenzylcarbinyl acetate | 30.0 |
| Diphenyloxide | 5.0 |
| Eucalyptol | 10.0 |
| Geraniol | 40.0 |
| Nerol | 20.0 |
| Geranium oil | 15.0 |
| Hexenol cis-3. 10% in DPG | 5.0 |
| Hexenyl salicylate cis-3 | 20.0 |
| Indole 10% in DPG | 10.0 |
| Alpha-ionone | 15.0 |
| Beta-ionone | 5.0 |
| Lilial ® (2-methyl-3-(4-tert-butyl-phenyl)propanal) | 60.0 |
| Linalool | 40.0 |
| Methylphenyl acetate | 10.0 |
| Phenylethyl alcohol | 275.0 |
| Styrolyl acetate | 20.0 |
| Terpineol | 30.0 |
| Tetrahydrolinalool | 50.0 |
| Cinnamyl alcohol | 10.0 |
| Total: | 1000.0 |

TABLE F3
Composition of perfume oil 3 (PO3; amounts in ‰ by weight)

| Ingredients | Amount |
|---|---|
| Benzyl acetate | 60.0 |
| Citronellyl acetate | 60.0 |
| Cyclamenaldehyde (2-methyl-3-(4- | 20.0 |
| isopropylphenyl)propanal | |
| Dipropylene glycol (DPG) | 60.0 |
| Ethyllinalool | 40.0 |
| Florol (2-isobutyl-4-methyltetrahydro-2H-pyran- | 30.0 |
| 4-ol) | |
| Globanone ® [(E/Z)-8-cyclohexadecen-1-one] | 180.0 |
| Hedione ® (methyldihydrojasmonate) | 140.0 |
| Hexenyl salicylate. Cis-3 | 10.0 |
| Vertocitral (2.4-dimethyl-3- | 5.0 |
| cyclohexenecarboxaldehyde) | |
| Hydratropaldehyde. 10% in DPG | 5.0 |
| Isodamascone (1-(2.4.4-trimethyl-2-cyclohexen- | 5.0 |
| 1-yl)-2-buten-1-one. 10% in DPG | |
| Isomuscone (cyclohexadecanone) | 40.0 |
| Jacinthaflor (2-methyl-4-phenyl-1.3-dioxolane) | 10.0 |
| Cis-jasmone. 10% in DPG | 20.0 |
| Linalool | 50.0 |
| Linalyl acetate | 30.0 |
| Methyl benzoate. 10% in DPG | 25.0 |
| para-Methyl cresol. 10% in DPG | 10.0 |
| Nerol | 20.0 |
| Phenylpropylaldehyde | 5.0 |
| 2-Phenylethyl alcohol | 82.0 |
| Tetrahydrogeraniol | 13.0 |
| 2.2-Dimethyl-3-cyclohexyl-1-propanol | 80.0 |
| Total: | 1000.0 |

TABLE F4
Composition of perfume oil 4 (PO4; amounts in ‰ by weight)

| Ingredients | Amount |
|---|---|
| Ambrettolide (macro) | 10.0 |
| Ambroxide 10% in ipm | 10.0 |
| Benzyl acetate | 20.0 |
| Benzyl salicylate | 15.0 |
| Bergamot oil. Bergapten-free | 60.0 |
| Calone ® 1951 10% in dpg | 15.0 |
| Coumarin | 5.0 |
| Cyclogalbanate ® 10% in dpg | 10.0 |
| Alpha -damascone 1% in dpg | 20.0 |
| Dihydromyrcenol | 10.0 |
| Ethyl linalool | 75.0 |
| Ethyl linalylacetate | 50.0 |
| Ethyl maltol 1% in dep | 10.0 |
| Ethylene brassylate (macro) | 80.0 |
| Florosa | 40.0 |
| Geranylacetate | 10.0 |
| Hedione ® hc/30 | 35.0 |
| Hedione ® | 210.0 |
| Helional ® | 15.0 |
| Helvetolide ® (alicyc) | 30.0 |
| Hexenylsalicylate cis-3 | 20.0 |
| Iso e super ® | 40.0 |
| Leafovert ® 10% in dep | 10.0 |
| Lilial ® | 80.0 |
| Lyral ® | 20.0 |
| Mandarin oil | 10.0 |
| Styralyl acetate | 5.0 |
| Symrose ® | 15.0 |
| Vanillin 10% in dep | 20.0 |
| Dipropylene glycol (dpg) | 50.0 |
| Total | 1000.0 |

TABLE F5
Composition of perfume oil 5 (PO5; amounts in ‰ by weight)

| Ingredients | Amount |
|---|---|
| Amarocite ® | 10.0 |
| Ambrocenide ® 10% in dpg | 5.0 |
| Ambroxide | 15.0 |
| Aurelione ® (7/8-cyclohexadecenone) (macro) | 70.0 |
| Bergamot oil. Bergapten-free | 90.0 |
| Calone ® 1951 10% in dpg | 20.0 |
| Caraway oil | 10.0 |
| Citral | 20.0 |
| Coumarin | 10.0 |
| Alpha-damascone 1% in dpg | 15.0 |
| Dihydromyrcenol | 70.0 |
| Estragon oil | 10.0 |
| Ethyl linalool | 100.0 |
| Ethyl linalylacetate | 90.0 |
| Eugenol | 10.0 |
| Evernyl ® | 5.0 |
| Fructate ® | 5.0 |
| Geranium oil | 5.0 |
| Hedione ® hc/30 | 100.0 |
| Helional ® | 10.0 |
| Indole 10% in dpg | 5.0 |
| Iso e super ® | 100.0 |
| Kephalis ® | 5.0 |
| Lavender oil | 40.0 |
| Citrus oil | 80.0 |
| Lilial ® | 30.0 |
| Mandarin oil | 20.0 |
| Muscenone (macro) | 5.0 |
| Sandranol ® | 10.0 |
| Vanillin 10% in dpg | 5.0 |
| Dipropylene glycol | 30.0 |
| Total | 1000.0 |

The above perfume oils PO1, PO2, PO3, PO4 or PO5 were incorporated into the formulations presented below.

Sunscreen products, cosmetic and pharmaceutical preparations (formulations); amounts are indicated as % by weight for all formulations:
Sunscreen Oil Spray with Ethanol, expected SPF 30, UVA/UVB balanced
Urban Sun Spray expected SPF 50+
Sunscreen Spray (O/W), expected SPF 30 (all in one pot/cold manufacturing process)
UV protection water spray SPF 50
Sunscreen Spray, *SPF 50+, Very Water Resistant**
Sunscreen Lotion (O/W), expected SPF 50* (cold/cold manufacturing process)
Beach Time Lotion SPF 30
Anti-Sweat Sunscreen Lotion
Sun protection milk (w/o)
Face Protecting Watery Mousse expected SPF 30*
Full protection drops expected SPF 50
Glow Brightener, expected SPF 50plus*
Sunscreen Fluid for acne-prone Skin, expected SPF 30
No More Shine Fluid expected SPF50
Light Sunscreen Fluid. Expected SPF 50
Refreshing Gel Sunscreen, with expected SPF 30, UVA/UVB balanced
Sun protection balm, SPF 40
Anti-Pollution Hair Care (SPF 15)
Perfect Hand with expected SPF 15, UVA/UVB balanced
Tinted Sunscreen cream against hyperpigmentation expected SPF 50*
Bb Cream Dark Tone Spf-20 (expected)
Daily Well Aging, with SPF 30, UVA/UVB balanced
Anti-Wrinkle Day Emulsion expected SPF 15
Daily Tattoo Care with expected SPF 10, UVA/UVB balanced
Sensitive Day Care Cream expected SPF 15
Cream with expected SPF 15, UVA/UVB
All purpose cleaner
APC alkaline
Detergent liquid light duty
Dishwash liquid, manual
Fabric softener
Fabric softener concentrate, encapsulated
Hand soap, liquid
Scent lotion, encapsulated
Cleaner, liquid, citric acid

TABLE F6
Sunscreen Oil Spray with Ethanol,
expected SPF 30, UVA/UVB balanced

| Raw material | INCI | Amount |
|---|---|---|
| Neo Heliopan ® 357 | Butyl methoxydibenzoylmethane | 5.0 |
| Neo Heliopan ® BMT | Bis-ethylhexyloxyphenol | 3.5 |
| | methoxyphenyl triazine | |
| Neo Heliopan ® OS | Ethylhexyl salicylate | 5.0 |
| Neo Heliopan ® EHT | Ethylhexyl triazone | 5.0 |
| SymMollient ® PDCC | Propanediol dicaprylate/caprate | 10.0 |
| Cetiol ® Sensoft | Propylheptyl caprylate | 6.0 |
| Cetiol ® AB | C12-15 alkyl benzoate | Ad 100 |
| Isopropyl Palmitate | Isopropyl palmitate | 20.7 |
| Sym Decanox ™ HA | Caprylic/capric triglyceride | 1.0 |
| | Hydroxymethoxyphenyl decanone | |
| Ethanol 96.5% | Alcohol, Aqua | 25.0 |
| Hydrolite ® 5 green | Pentylene glycol | 3.0 |
| MAA-1 | | 0.1 |
| MAA-13 | | 0.1 |
| Hydrolite ® 7 green | 1,2-heptanediol | 2.0 |
| Perfume oil PO1; PO2; | Perfume oil PO1; PO2; PO3; PO4; or | 0.3 |
| PO3; PO4; or PO5 | PO5 | |

TABLE F7
Urban Sun Spray, o/w emulsion, expected SPF 50+

| Ingredients | INCI | Amount |
|---|---|---|
| Aqua/Water | Aqua | Ad 100 |
| MAA-1 | | 1.0 |
| MAA-13 | | 0.5 |
| Vivapur ® CS | Microcrystalline Cellulose, | 1.0 |
| Tex sun | Cellulose Gum | |
| Vivapur ® CS | Microcrystalline Cellulose, | 1.0 |
| 032 XV | Xanthan Gum | |
| Sodium Hydroxide | Aqua, Sodium Hydroxide | 0.5 |
| solution 10% aq. | | |
| SymOcide ® PH | Phenoxyethanol, Hydroxyacetophenone, | 1.4 |
| | Caprylyl Glycol, Aqua | |
| Emulsiphos ® | Potassium cetyl phosphate, | 1.5 |
| | Hydrogenatedpalm glycerides | |
| Dragoxat ® 89 | Ethylhexyl Isononanoate | 2.0 |
| SymMollient ® PDCC | Propanediol Dicaprylate/Caprate | 7.0 |
| SymSol ® PF-3 | Aqua, Pentylene Glycol, Sodium Lauryl | 2.0 |
| | Sulfoacetate, Sodium Oleoyl | |
| | Sarcosinate, Sodium Chlrodie, Sodium | |
| | Oleate | |
| Prisorine ® 3505 | Isostearic acid | 1.5 |
| Neo Heliopan ® BMT | Bis-Ethylhexyloxyphenol Methoxypenyl | 5.0 |
| | Triazine | |
| Neo Heliopan ® 357 | Butyl Methoxydibenzoylmethane | 4.5 |
| Neo Heliopan ® 303 | Octocrylene | 10.0 |
| Neo Heliopan ® HMS | Homosalate | 10.0 |
| Neo Heliopan ® OS | Ethylhexyl Salicylate | 5.0 |
| Dragosine ® | Carnosine | 0.2 |
| SymUrban ® | Benzylidene | 0.2 |
| | Dimethoxydimetylindanone | |
| Hydrolite ® 5 green | Pentylene glycol | 0.5 |
| SymVital ® AR 3040 | Zingiber officinale (Ginger) Root | 0.1 |
| (974839) | extract | |
| SymDecanox HA | Caprylic/Capric Triglyceride, | 1.0 |
| (972276) | Hydroxymethoxyphenyl decanone | |
| Floraesters ® K-100 | Hydrolyzed jojoba esters, Jojoba | 0.5 |
| Jojoba | esters, Aqua | |
| EDTA ® BD | Disodium EDTA | 0.1 |
| Keltrol ® CG-SFT | Xanthan Gum | 0.3 |
| Perfume oil PO1; | Perfume oil PO1; PO2; PO3; PO4; | 0.5 |
| PO2; PO3; PO4; or | or PO5 | |
| PO5 | | |
| Farmal MS6135 | Calcium Starch Octenylsuccinate | 2.0 |
| Tapioca Pure | Tapioca starch | 1.5 |

TABLE F8
Sunscreen Spray (O/W), expected SPF
30* (cold manufacturing process)

| Ingredients | INCI | Amount |
|---|---|---|
| Dracorin ® GOC | Glyceryl Oleate Citrate Caprylic/ | 2.0 |
| | Capric Triglyceride | |
| Dragosine ® | Carnosine | 0.2 |
| Neo Heliopan ® Flat | Homosalate, Octocrylene, Bis- | 25.0 |
| | Ethylhexyloxyphenol Methoxyphenyl | |
| | Triazine, Butyl | |
| | Methoxydibenzoylmethane Ethylhexyl | |
| | Salicylate | |
| SymMollient ® PDCC | Propanediol Dicaprylate/Caprate | 7.0 |
| Aristoflex ® Silk | Sodium Polyacryloyldiemthyl taurate | 0.6 |
| Hydrolite ® 5 green | Pentylene glycol | 3.0 |
| Aqua/Water | Aqua | Ad 100 |
| MAA-13 | | 1.00 |
| Sodium Hydroxide | Aqua, Sodium Hydroxide | 0.50 |
| solution 10% aq. | | |
| SymOcide ® PH | Phenoxyethanol, | 1.45 |
| | Hydroxyacetophenone, Caprylyl | |
| | Glycol, Aqua | |
| SymMollient ® PDCC | Propanediol Dicaprylate/Caprate | 3.0 |
| Glycerin 99.5% | Glycerin | 3.0 |
| Dragoxat ® 89 | Ethylhexyl Isononanoate | 2.0 |
| PCL-Liquid ® 100 | Cetearyl Ethylhexanoate | 2.0 |
| EDTA ® BD | Disodium EDTA | 0.1 |
| Farmal MS | Calcium Starch Octenylsuccinate | 2.0 |
| Keltrol ® CG-BT | Xanthan Gum | 0.4 |
| Perfume oil PO1; PO2; | Perfume oil PO1; PO2; PO3; PO4; or | 0.3 |
| PO3; PO4; or PO5 | PO5 | |

TABLE F9
UV protection water spray, SPF 50

| Ingredients | INCI | Amount |
|---|---|---|
| Isolan GPS | Isododecane | 10.0 |
| Dermofeel sensolv MB | Diethylhexyl Carboate | 5.0 |
| Tegosoft ® DC MB | Phenoxyethyl Caprylate | 5.0 |
| Neo Heliopan ® 357 | Butyl Methoxydibenzoylmethane | 5.0 |
| Neo Heliopan ® HMS | Homosalate | 5.0 |
| Neo Heliopan ® 303 | Octocrylene | 5.0 |
| Neo Heliopan ® OS | Ethylhexyl Salicylate | 5.0 |
| Neo Heliopan ® BMT | Bis-Ethylhexyloxyphenol | 4.0 |
| | Methoxypenyl Triazine | |
| Neo Heliopan ® EHT | Ethylhexyl Triazone | 3.0 |
| Dermofeel ® Toco 70 | Tocopherol; Helianthus Annuus | 0.5 |
| non GMO | (Sunflower) Seed oil | |
| Tego ® SP 13 Sun Up | Poly C10-30 Alkyl Acrylate | 0.4 |
| SymEffect ® Sun | Cera Alba. Sodium Stearoyl Lactylate | 1.5 |
| SymRepair ® 100 | Hexyldecanol. Bisabolol. | 1.0 |
| | Cethylhydroxyproline Palmitamide. | |
| | Stearic acid. Brassica campestris | |
| | (Rapeseed) sterols | |
| SymDecanox ® HA | Caprylic/Capric Triglyceride, | 1.0 |
| | Hydroxymethoxyphenyl decanone | |
| PCL Liquid ® 100 | Cetearyl Ethlyhexanoate | 2.0 |
| Hydrolite ® 5 green | Pentylene glycol | 0.5 |
| Perfume oil PO1; PO2; | Perfume oil PO1; PO2; PO3; PO4; or | 0.5 |
| PO3; PO4; or PO5 | PO5 | |
| Dermosoft ® OMP | Methylpropandiol; Capryly Glycol; | 3.0 |
| | Phenylpropanol | |
| Water | Water (Aqua) | Ad 100 |
| Neo Heliopan ® Hydro | Phenylbenzimidazole Sulfonic Acid | 2.0 |
| MAA-1 | | 1.00 |
| MAA-13 | | 1.00 |
| Glycerin | Glycerin, Aqua | 0.5 |
| Sodium hydroxide | Aqua, Sodium hydroxicde | 2.5 |
| 10% sol. | | |
| EDTA ® BD | Disosium EDTA | 0.1 |
| Dragosine ® | Carnosine | 0.1 |
| Triethanolamine | Triethanolamine | 0.1 |

TABLE F10
Sunscreen Spray, *SPF 50+, Very Water Resistant**

| Ingredients | INCI | Amount |
|---|---|---|
| Emulsiphos ® | Potassium Cetyl Phosphate | 2.0 |
| | Hydrogenated Palm Glycerides | |
| SymMollient ® PDCC | Propanediol Dicaprylate/Caprate | 5.0 |
| SymMollient ® S green | Cetearyl Nonanoate | 2.5 |
| Neo Heliopan ® 357 | Butyl Methoxydibenzoylmethane | 5.0 |
| Neo Heliopan ® HMS | Homosalate | 8.0 |
| Neo Heliopan ® 303 | Octocrylene | 8.0 |
| Neo Heliopan ® OS | Ethylhexyl Salicylate | 4.0 |
| Neo Heliopan ® BMT | Bis-Ethylhexyloxyphenol | 4.0 |
| | Methoxypenyl Triazine | |
| Tocopherol Alpha DL | Tocopherol | 0.2 |
| Edeta ® BD | EDTA | 0.1 |
| Sym Effect ® Sun | Cera Alba, Sodium Stearoyl Lactylate | 3.0 |
| Keltrol ® CG-SFT | Xanthan Gum | 0.2 |
| Water/Aqua | Aqua | Ad 100 |
| Neo Heliopan ® Hydro | Phenylbenzimidazole Sulfonic Acid | 1.5 |
| MAA-4 | | 2 |
| Biotive ® L-Arginine | Arginine | 1.5 |
| Dragosine ® | Carnosine | 0.1 |
| SymSave ® H | Hydroxyacetophenone | 0.5 |
| Hydrolite ® 5 green | Pentylene glycol | 1.8 |
| SymLite ® G8 | Glyceryl caprylate | 0.6 |
| Cranberry Oil CC | Vaccinium Macrocappon seed oil | 1.0 |
| Tapioca Pure | Tapioca starch | 2.0 |
| VITACEL ® CS 20 FC | Cellulose | 2.0 |
| Perfume oil PO1; PO2; | Perfume oil PO1; PO2; PO3; PO4; or | 0.3 |
| PO3; PO4; or PO5 | PO5 | |

TABLE F11
Sunscreen Lotion (O/W), expected SPF
50* (cold/cold manufacturing process)

| Ingredients | INCI | Amount |
|---|---|---|
| Dracorin ® GOC | Glyceryl Oleate Citrate Caprylic/ | 2.0 |
| | Capric Triglyceride | |
| KP-545 | Cyclopentasiloxane | 1.0 |
| | Acrylates/Dimethicone copolymer | |
| Neo Heliopan ® Flat | Homosalate, Octocrylene, Bis- | 30.0 |
| | Ethylhexyloxyphenol Methoxyphenyl | |
| | Triazine, Butyl | |
| | Methoxydibenzoylmethane | |
| | Ethylhexyl Salicylate | |
| SymMollient ® PDCC | Propanediol Dicaprylate/Caprate | 7.0 |
| Aristoflex ® Silk | Sodium Polyacryloyldiemthyl taurate | 0.6 |
| Aqua/Water | Aqua | Ad 100 |
| MAA-1 | | 3.00 |
| MAA-13 | | 1.0 |
| Sodium Hydroxide | Aqua, Sodium Hydroxide | 1.25 |
| solution 10% aq. | | |
| SymOcide ® PH | Phenoxyethanol, | 1.5 |
| | Hydroxyacetophenone, Caprylyl | |
| | Glycol, Aqua | |
| Hydrolite ® 5 green | Pentylene glycol | 1.8 |
| Glycerin 99.5% | Glycerin | 2.0 |
| VITACEL ® CS 20 FC | Cellulose | 1.0 |
| Perfume oil PO1; | Perfume oil PO1; PO2; PO3; PO4; or | 0.3 |
| PO2; PO3; PO4; or PO5 | PO5 | |

TABLE F12
Beach Time Lotion, SPF 30

| Ingredients | INCI | Amount |
|---|---|---|
| Isolan GPS | Polyglyceryl-4 Diisostearate/ | 3.5 |
| | Polyhydroxystearate/Sebacate | |
| Dermofeel ® sensolv MB | Isoamyl Laurate | 4.5 |
| TEGOSOFT ® DC MB | Decyl Cocoate | 4.0 |
| Neo Heliopan ® BMT | Bis-Ethylhexyloxyphenol | 3.0 |
| | Methoxypenyl Triazine | |
| Uvinul ® A plus | Diethylamino Hydroxybenzoyl | 3.0 |
| | Hexyl Benzoate | |
| Neo Heliopan ® OS | Ethylhexyl Salicylate | 4.0 |
| Neo Heliopan ® EHT | Ethylhexyl Triazone | 3.0 |
| Neo Heliopan ® HMS | Homosalate | 5.0 |
| Verstatil TBO | Triethylcitrate; Caprylyl | 1.0 |
| | Glycol; benzoic Acid | |
| Zinc Stearate | Zinc Stearate | 0.5 |
| Water | Aqua | Ad 100 |
| MAA-8 | | 1.00 |
| MAA-13 | | 3.0 |
| Sodium Hydroxide | Aqua, Sodium Hydroxide | 0.50 |
| solution 10% aq. | | |
| Glycerin | Glycerin | 3.0 |
| Zinc Sulfate Heptahydrate | Zinc Sulfate Heptahydrate | 1.2 |

TABLE F13
Anti-Sweat Sunscreen Lotion

| Raw material | INCI | Amount |
|---|---|---|
| Aqua/Water | Aqua | Ad 100 |
| MAA-8 | | 2.0 |
| MAA-9 | | 0.7 |
| Sodium Hydroxide | Aqua, Sodium Hydroxide | 1.00 |
| solution 10% aq. | | |
| Hydrolite ® 7 Green | 1,2-Heptanediol | 0.5 |
| Citric Acid 30% Sol. | Aqua, Citric acid | 0.6 |
| Aristoflex ® Velvet | Polyacrylate crosspolymer 11 | 0.6 |
| Glycerin 99.5% | Glycerin, Aqua | 2.0 |
| SymSol ® PF-3 | Aqua, Pentylene glycol, Sodium | 1.0 |
| (358712) | lauryl sulfoacetate, Sodium | |
| | oleoyl sarcosinate, Sodium | |
| | chloride, Sodium oleate | |
| SymSave ® H | Hydroxyacetophenone | 0.3 |
| Neo Heliopan ® 357 | Butyl methoxydibenzoylmethane | 3.0 |
| Neo Heliopan ® 303 | Octocrylene | 10.0 |
| Neo Heliopan ® OS | Ethylhexyl salicylate | 5.0 |
| Neo Heliopan ® HMS | Homosalate | 10.0 |
| Neo Heliopan ® E1000 | Isoamyl p-methoxycinnamate | 2.0 |
| Neo Heliopan ® BMT | Bis-ethylhexyloxyphenol | 3.0 |
| | methoxyphenyl triazine | |
| Floraesters ® K-100 | Hydrolyzed jojoba esters | 2.0 |
| Jojoba | Jojoba esters | |
| | Aqua | |
| Dragoxat ® 89 | Ethylhexyl isononanoate | 6.0 |
| SymMollient ® S | Cetearyl nonanoate | 1.0 |
| EDTA ® BD | Disodium edta | 0.1 |
| SymDecanox ™ HA | Caprylic/capric triglyceride | 1.0 |
| | Hydroxymethoxyphenyl decanone | |
| Frescolat ® Plus | Menthol | 0.3 |
| | Menthyl lactate | |
| SymDeo ® Plus | Lauryl alcohol, Phenoxyethanol, 2- | 1.0 |
| | benzylheptanol, Decylene glycol | |
| Hydroviton ® PLUS | Aqua, Pentylene glycol, Glycerin, | 4.0 |
| 2290 | Fructose, Urea, Citric acid, Sodium | |
| | hydroxide, Maltose, Sodium pca, | |
| | Sodium chloride, Sodium lactate, | |
| | Trehalose, Allantoin, Sodium | |
| | hyaluronate, Glucose | |
| Sensocel ® 10 | Cellulose | 2.0 |

TABLE F14
Sun protection milk (w/o)

| Ingredients | INCI | Amount |
|---|---|---|
| Dehymuls ® PGPH | Polyglyceryl-2 dipolyhydroxystearate | 3.0 |
| Beeswax 8100 | Beeswax | 1.0 |
| Monomuls ® 90-0-18 | Glyceryl oleate | 1.0 |
| Zinc stearate | Zinc stearate | 1.0 |
| Cetiol ® SN | Cetearyl isononanoate | 5.0 |
| Cetiol ® OE | Dicaprylyl ether | 5.0 |
| Tegosoft ® TN | C12-15 alkyl benzoate | 4.0 |
| Vitamin E | Tocopherol | 0.5 |
| Neo Heliopan ® OS | Ethylhexyl salicylate | 5.0 |
| Neo Heliopan ® AV | Ethylhexyl methoxycinnamate | 7.5 |
| Neo Heliopan ® EHT | Ethylhexyl triazone | 1.5 |
| Water, distilled | Water (Aqua) | ad 100 |
| MAA-4 | | 2.5 |
| MAA-13 | | 1.0 |
| Trilon ® BD | Disodium EDTA | 0.1 |
| Glycerol | Glycerol | 5.0 |
| Neo Heliopan ® AP | Disodium phenyl dibenzimidazole | 15.0 |
| 10% solution. | tetrasulfonate | |
| Neutralized with NaOH | | |
| Sodium Hydroxide | Aqua, Sodium Hydroxide | 1.5 |
| solution 10% aq. | | |
| Perfume oil PO1; PO2; | Perfume oil PO1; PO2; PO3; PO4; or | 0.3 |
| PO3; PO4; or PO5 | PO5 | |
| Alpha Bisabolol | Bisabolol | 0.1 |
| SymOcide ® PT | Phenoxyethanol. Tropolone | 0.3 |
| Hydrolite ® 7 Green | 1,2-Heptanediol | 0.8 |

TABLE F15
Protecting Watery Mousse, expected SPF 30*

| Ingredients | INCI | Amount |
|---|---|---|
| Aqua/Water | Aqua | Ad 100 |
| MAA-1 | | 3.00 |
| MAA-9 | | 0.5 |
| Neo Heliopan ® Hydro | Phenylbenzimidazole sulfonic acid | 2.0 |
| Neo Heliopan ® AP | Disodium phenyl dibenzimidazole | 1.0 |
| | tetrasulfonate | |
| Biotive ® L-Arginine | Arginine | 1.8 |
| Sodium Hydroxide | Aqua, Sodium Hydroxide | 1.5 |
| solution 10% aq. | | |
| SymSol ® PF-3 | Aqua, Pentylene glycol, Sodium | 2.5 |
| | lauryl, sulfoacetate, Sodium | |
| | oleoyl sarcosinate, Sodium | |
| | chloride, Sodium oleate | |
| Dragosine ® | Carnosine | 0.2 |
| Aqua Keep 10SH-NFC | Sodium acrylates crosspolymer-2 | 2.5 |
| SymSave ® H | Hydroxyacetophenone | 0.5 |
| Phenoxyethanol | Phenoxyethanol | 0.5 |
| Hydrolite ® 5 Green | Pentylene glycol | 1.5 |
| Neo Heliopan ® 357 | Butyl methoxydibenzoylmethane | 3.0 |
| Neo Heliopan ® OS | Ethylhexyl salicylate | 5.0 |
| Neo Heliopan ® BMT | Bis-ethylhexyloxyphenol | 3.0 |
| | methoxyphenyl triazine | |
| Uvasorb ® HEB | Diethylhexyl butamido triazone | 3.0 |
| SymMollient ® S | Cetearyl nonanoate | 3.0 |
| EDTA ® BD | Disodium edta | 0.1 |
| Isoadipate | Diisopropyl adipate | 4.0 |
| Cetiol ® AB | C12-15 alkyl benzoate | 7.0 |
| Caprylic/Capric | Caprylic/capric triglyceride | 6.0 |
| Triglyceride | | |
| Frescolat ® ML | Menthyl Lactate | 2.0 |
| SymGlucan ® | Aqua, Glycerin, 1,2-Hexanediol, | 2.0 |
| | Caprylyl glycol, Beta-glucan | |
| Hydrolite ®-6 Green | 1,2-Hexanediol | 0.5 |

TABLE F16
Full protection emulsion, expected SPF 50

| Ingredients | INCI | Amount |
|---|---|---|
| Dracorin ® GOC | Glyceryl oleate citrate, Caprylic/ | 2.5 |
| | capric triglyceride | |
| Neo Heliopan ® 303 | Octocrylene | 8.0 |
| Neo Heliopan ® 357 | Butyl methoxydibenzoylmethane | 4.5 |
| Neo Heliopan ® HMS | Homosalate | 10.0 |
| Neo Heliopan ® OS | Ethylhexyl salicylate | 5.0 |
| Neo Heliopan ® BMT | Bis-ethylhexyloxyphenol | 5.0 |
| | methoxyphenyl triazine | |
| SymMollient ® PDCC | Propanediol dicaprylate/caprate | 5.0 |
| Velvesil ® DM | Dimethicone, Cetearyl dimethicone | 3.0 |
| | crosspolymer | |
| Dow Corning ® 9041 | Dimethicone, Dimethicone | 2.0 |
| Silicone Elastomer | crosspolymer | |
| Blend | | |
| EDTA ® BD | Disodium edta | 0.1 |
| Keltrol ® CG-SFT | Xanthan gum | 0.2 |
| Pemulen ™ TR-2 | Acrylates/c10-30 alkyl acrylate | 0.1 |
| Polymeric Emulsifier | crosspolymer | |
| Aqua/Water | Aqua | Ad 100 |
| MAA-20 | | 3.0 |
| Glycerin 99.5% | Glycerin, Aqua | 3.0 |
| Sodium Hydroxide | Aqua, Sodium Hydroxide | 1.3 |
| solution 10% aq. | | |
| Dragosine ® | Carnosine | 0.2 |
| SymSave ® H | Hydroxyacetophenone | 0.5 |
| Hydrolite ® 5 green | Pentylene glycol | 0.8 |
| Hydrolite ® CG | Caprylyl glycol | 0.3 |
| Hydrolite ® 8 Green | Caprylyl Glycol | 0.8 |
| Sym Vital ® AR | Zingiber officinale (ginger) | 0.2 |
| | root extract | |
| Tapioca Pure | Tapioca starch | 2.0 |
| Perfume oil PO1; PO2; | Perfume oil PO1; PO2; PO3; PO4; or | 0.5 |
| PO3; PO4; or PO5 | PO5 | |

TABLE F17
Whitening emulsion, expected SPF 50plus*

| Ingredients | INCI | Amount |
|---|---|---|
| Emulsiphos ® F | Potassium cetyl phosphate | 2.0 |
| | Hydrogenated palm glycerides | |
| Lanette ® 16 | Cetyl alcohol | 0.5 |
| Neo Heliopan ® 357 | Butyl methoxydibenzoylmethane | 4.9 |
| Neo Heliopan ® HMS | Homosalate | 10.0 |
| Neo Heliopan ® 303 | Octocrylene | 10.0 |
| Neo Heliopan ® OS | Ethylhexyl salicylate | 5.0 |
| Neo Heliopan ® BMT | Bis-ethylhexyloxyphenol | 4.0 |
| | methoxyphenyl triazine | |
| SymBright ™ 2036 | Sclareolide | 0.2 |
| SymWhite ® PLUS | Caprylic/capric triglyceride, | 1.0 |
| | Pentylene glycol | |
| | Phenylethyl resorcinol, Bisabolol, | |
| | Butyl methoxydibenzoylmethane | |
| Isoadipate | Diisopropyl adipate | 5.0 |
| KP-545 | Cyclopentasiloxane | 1.0 |
| | Acrylates/dimethicone copolymer | |
| Velvesil ® DM | Dimethicone | 2.0 |
| | Cetearyl dimethicone crosspolymer | |
| Prisorine ® 3505 | Isostearic acid | 1.0 |
| EDTA ® BD | Disodium edta | 0.1 |
| Hydrolite ® 5 green | Pentylene glycol | 3.0 |
| Biotive ® Resveratrol | Resveratrol | 0.1 |
| Keltrol ® CG-BT | Xanthan gum | 0.5 |
| Pemulen ® EZ-4U | Acrylates/c10-30 alkyl acrylate | 0.1 |
| | crosspolymer | |
| Aqua/Water | Aqua | Ad 100 |
| MAA-20 | | 1.0 |
| MAA-13 | | 2.5 |
| Biotive ® L-Arginine | Arginine | 0.6 |
| SymSave ® H | Hydroxyacetophenone | 0.5 |
| VITACEL ® CS 20 FC | Cellulose | 2.0 |
| SymOcide ® C | O-cymen-5-ol | 0.1 |
| Propylene Glycol | Propylene glycol | 2.0 |
| Cosmetic Color | Mica, Titanium dioxide | 3.0 |
| Pearlescent Silver AA | | |
| Perfume oil PO1; PO2; | Perfume oil PO1; PO2; PO3; PO4; or | 0.5 |
| PO3; PO4; or PO5 | PO5 | |

TABLE F18
Sunscreen Fluid for acne-prone Skin, expected SPF 30

| Ingredients | INCI | Amount |
|---|---|---|
| Dracorin ® GOC | Glyceryl oleate citrate | 2.5 |
| | Caprylic/capric triglyceride | |
| Neo Heliopan ® 357 | Butyl methoxydibenzoylmethane | 4.2 |
| Neo Heliopan ® OS | Ethylhexyl salicylate | 5.0 |
| Neo Heliopan ® BMT | Bis-ethylhexyloxyphenol | 3.5 |
| | methoxyphenyl triazine | |
| Uvasorb ® HEB | Diethylhexyl butamido triazone | 7.0 |
| SymMollient ® S | Cetearyl nonanoate | 1.0 |
| Caprylic/Capric | Caprylic/capric triglyceride | 8.0 |
| Triglyceride | | |
| EDTA ® BD | Disodium edta | 0.1 |
| Sym Decanox ™ HA | Caprylic/capric triglyceride | 1.0 |
| | Hydroxymethoxyphenyl decanone | |
| Isoadipate | Diisopropyl adipate | 4.0 |
| SymClariol ® | Decylene glycol | 0.3 |
| Cetiol ® AB | C12-15 alkyl benzoate | 13.5 |
| Keltrol ® CG-SFT | Xanthan gum | 0.2 |
| Keltrol ® CG-BT | Xanthan gum | 0.1 |
| Food Color Brown | Titanium dioxides (ci 77891), Iron | 3.0 |
| E172 + E171 Powder | oxides (ci 77492), Iron oxides (ci | |
| | 77491), Iron oxides (ci. 77499) | |
| Eusolex ® T-AVO | Titanium dioxide, Silica | 2.0 |
| Aqua/Water | Aqua | Ad 100 |
| MAA-20 | | 1.00 |
| MAA-13 | | 1.5 |
| Glycerin 99.5% | Glycerin, Aqua | 3.0 |
| Lanette ® E | Sodium cetearyl sulfate | 0.8 |
| Neo Heliopan ® Hydro | Phenylbenzimidazole sulfonic acid | 2.0 |
| Biotive ® L-Arginine | Arginine | 1.0 |
| Sodium Hydroxide 10% | Aqua | 1.0 |
| solution | Sodium hydroxide | |
| SymSave ® H | Hydroxyacetophenone | 0.5 |
| Hydrolite ® 5 | Pentylene glycol | 1.5 |
| Phenoxyethanol | Phenoxyethanol | 0.5 |
| SymGlucan ® | Aqua, Glycerin, 1,2-hexanediol, | 2.0 |
| | Caprylyl glycol, Beta-glucan | |
| Sensocel ® 10 | Cellulose | 2.0 |

TABLE F19
No More Shine Fluid, expected SPF50

| Ingredients | INCI | Amount |
|---|---|---|
| Dracorin ® GOC | Glyceryl oleate citrate | 2.0 |
| | Caprylic/capric triglyceride | |
| Neo Heliopan ® 357 | Butyl methoxydibenzoylmethane | 4.5 |
| Neo Heliopan ® OS | Ethylhexyl salicylate | 5.0 |
| Neo Heliopan ® HMS | Homosalate | 10.0 |
| Neo Heliopan ® 303 | Octocrylene | 8.0 |
| Neo Heliopan ® BMT | Bis-ethylhexyloxyphenol | 3.0 |
| | methoxyphenyl triazine | |
| SymMollient ® PDCC | Propanediol dicaprylate/caprate | 5.0 |
| KP-545 L | Dimethicone | 1.0 |
| | Acrylates/dimethicone copolymer | |
| EDTA ® BD | Disodium edta | 0.1 |
| Velvesil ® DM | Dimethicone | 2.5 |
| | Cetearyl dimethicone crosspolymer | |
| Silsoft ™ 034 | Caprylyl methicone | 1.0 |
| Keltrol ® CG-BT | Xanthan gum | 0.3 |
| Aristoflex ® Silk | Sodium polyacryloyldimethyl taurate | 0.5 |
| Aqua/Water | Aqua | Ad 100 |
| MAA-12 | | 2.5 |
| MAA-13 | | 1.0 |
| Neo Heliopan ® Hydro | Phenylbenzimidazole sulfonic acid | 1.0 |
| Biotive ® L-Arginine | Arginine | 1.1 |
| Lanette ® E | Sodium cetearyl sulfate | 0.8 |
| Dragosine ® | Carnosine | 0.2 |
| SymSave ® H | Hydroxyacetophenone | 0.5 |
| Hydrolite ® 5 green | Pentylene glycol | 1.0 |
| Hydrolite ® CG | Caprylyl glycol | 0.3 |
| Glycerin 99.5% | Glycerin, Aqua | 3.0 |
| Food color brown | Titanium dioxides (ci 77891), Iron | 2.5 |
| e172 + e171 powder | oxides (ci 77492), Iron oxides (ci | |
| | 77491), Iron oxides (ci. 77499) | |
| Cosmetic color titanium | Titanium dioxide | 4.0 |
| dioxide ci77891 | | |
| Sym Decanox ™ HA | Caprylic/capric triglyceride | 0.5 |
| | Hydroxymethoxyphenyl decanone | |
| Sensocel ® 10 | Cellulose | 1.5 |
| Phytoconcentrole ® | Helianthus annuus seed oil, Beta- | 0.3 |
| Carrot | carotene | |
| | Daucus carota sativa root extract | |
| Perfume oil PO1; PO2; | Perfume oil PO1; PO2; PO3; PO4; or | 0.3 |
| PO3; PO4; or PO5 | PO5 | |
| Citric Acid 10% Sol. | Aqua, Citric acid | 0.2 |

TABLE F20
Light Sunscreen Fluid, expected SPF 50

| Ingredients | INCI | Amount |
|---|---|---|
| Aqua/Water | Aqua | Ad 100 |
| MAA-1 | | 1.00 |
| MAA-13 | | 1.00 |
| Sodium Hydroxide | Aqua, Sodium Hydroxide | 1.00 |
| solution 10% aq. | | |
| Glycerin 99.5% | Glycerin, Aqua | 3.0 |
| Hydrolite ® 5 green | Pentylene glycol | 0.8 |
| SymSave ® H | Hydroxyacetophenone | 0.5 |
| Hydrolite ® CG | Caprylyl glycol | 0.3 |
| Neo Heliopan ® Hydro | Phenylbenzimidazole sulfonic acid | 2.0 |
| Biotive ® L-Arginine | Arginine | 1.4 |
| Emulsiphos ® | Potassium cetyl phosphate | 1.5 |
| | Hydrogenated palm glycerides | |
| Neo Heliopan ® 303 | Octocrylene | 10.0 |
| Neo Heliopan ® 357 | Butyl methoxydibenzoylmethane | 5.0 |
| Neo Heliopan ® HMS | Homosalate | 10.0 |
| Neo Heliopan ® BMT | Bis-ethylhexyloxyphenol | 4.0 |
| | methoxyphenyl triazine | |
| Neo Heliopan ® OS | Ethylhexyl salicylate | 5.0 |
| Neo Heliopan ® E1000 | Isoamyl p-methoxycinnamate | 2.0 |
| Dragoxat ® 89 | Ethylhexyl isononanoate | 3.0 |
| Tocopheryl Acetate | Tocopheryl acetate | 0.5 |
| Keltrol ® CG-BT | Xanthan gum | 0.1 |
| Keltrol ® CG-SFT | Xanthan gum | 0.3 |
| VITACEL ® CS 20 FC | Cellulose | 2.0 |
| Tapioca Pure | Tapioca starch | 2.0 |
| Ethanol 96% | Alcohol denat. | 5.0 |
| Silsoft ™ 034 | Caprylyl methicone | 3.0 |
| Perfume oil PO1; PO2; | Perfume oil PO1; PO2; PO3; PO4; or | 0.3 |
| PO3; PO4; or PO5 | PO5 | |

TABLE F21
Refreshing Gel Sunscreen, expected SPF 30, UVA/UVB balanced

| Raw material | INCI | Amount |
|---|---|---|
| Aqua/Water | Aqua | Ad 100 |
| MAA-20 | | 1.5 |
| MAA-13 | | 0.9 |
| Neo Heliopan ® Hydro | Phenylbenzimidazole sulfonic acid | 2.0 |
| Sodium Hydroxide | Aqua, Sodium Hydroxide | 0.9 |
| solution 10% aq. | | |
| Hydrolite ® 5 green | Pentylene glycol | 2.0 |
| Biotive ® L-Arginine | Arginine | 1.0 |
| Sodium Hydroxide | Aqua, Sodium hydroxide | 0.2 |
| 10% sol. | | |
| Dragosine ® | Carnosine | 0.2 |
| SymGuard ® CD | Phenylpropanol, O-cymen-5-ol, | 0.5 |
| | Decylene glycol | |
| SymDeo ® B125 | 2-methyl 5-cyclohexylpentanol | 0.5 |
| SymSave ® H | Hydroxyacetophenone | 0.5 |
| SymSol ® PF-3 | Aqua, Pentylene glycol, Sodium | 1.5 |
| | lauryl sulfoacetate, Sodium oleoyl | |
| | sarcosinate, Sodium chlorid, Sodium | |
| | oleate | |
| Neo Heliopan ® 357 | Butyl methoxydibenzoylmethane | 3.0 |
| Neo Heliopan ® OS | Ethylhexyl salicylate | 5.0 |
| Neo Heliopan ® BMT | Bis-ethylhexyloxyphenol | 1.5 |
| | methoxyphenyl triazine | |
| Neo Heliopan ® HMS | Homosalate | 2.5 |
| Neo Heliopan ® 303 | Octocrylene | 10.0 |
| EDTA ® BD | Disodium edta | 0.1 |
| SymMollient ® PDCC | Propanediol dicaprylate/caprate | 2.0 |
| Floraesters ® K-100 ® | Hydrolyzed jojoba esters, Jojoba | 1.0 |
| Jojoba | esters, Aqua | |
| Frescolat ® X-Cool | Menthyl ethylamido oxalate | 0.8 |
| Aristoflex ® AVC | Ammonium acryloyol- | 1.4 |
| | dimethyltaurate/vp copolymer | |
| Keltrol ® CG-T | Xanthan gum | 0.1 |
| Sensocel ® 10 | Cellulose | 3.0 |
| Perfume oil PO1; PO2; | Perfume oil PO1; PO2; PO3; PO4; or | 0.4 |
| PO3; PO4; or PO5 | PO5 | |
| Phytoconcentrole ® | Helianthus annuus seed oil, Beta- | 0.3 |
| Carrot | carotene, | |
| | Daucus carota sativa root extract | |

TABLE F22
Sun protection balm, SPF 40

| Raw material | INCI | Amount |
|---|---|---|
| Aqua/Water | Aqua | Ad 100 |
| MAA-6 | | 2.0 |
| MAA-13 | | 1.5 |
| Neo Heliopan ® Hydro | Phenylbenzimidazole sulfonic acid | 1.0 |
| Sodium Hydroxide | Aqua, Sodium Hydroxide | 1.4 |
| solution 10% aq. | | |
| Hydrolite ®-5 | Pentylene glycol | 2.0 |
| Biotive ® L-Arginine | Arginine | 0.6 |
| Hydrolite ® CG | Caprylyl glycol | 0.25 |
| EDTA ® BD | Disodium EDTA | 0.1 |
| SymSave ® H | Hydroxyacetophenone | 0.3 |
| Hyaluronic Acid | Hyaluronic Acid | 0.1 |
| Dragosine ® | Carnosine | 0.2 |
| Aristoflex ® Silk | Sodium Polyacrydimethyl Taurate | 1.1 |
| Neo Heliopan ® 357 | Butyl methoxydibenzoylmethane | 5.0 |
| Neo Heliopan ® 303 | Octocrylene | 8.0 |
| Neo Heliopan ® OS | Ethylhexyl salicylate | 5.0 |
| Neo Heliopan ® HMS | Homosalate | 3.0 |
| Neo Heliopan ® BMT | Bis-ethylhexyloxyphenol | 3.0 |
| | methoxyphenyl triazine | |
| Dragoxat ® 89 | Ethylhexyl isononanoate | 2.0 |
| SymMollient ® PDCC | Cetearyl nonanoate | 3.0 |
| Vitacel ® CS 20 FC | Cellulose | 2.0 |

TABLE F23
Anti-Pollution Hair Care, SPF 15

| Raw material | INCI | Amount |
|---|---|---|
| Caprylic/Capric | Caprylic/capric triglyceride | 18.0 |
| Triglyceride | | |
| SymOleo ® Vita7 | Glycine soja oil, Gossypium | 2.0 |
| | herbaceum seed oil, Mangifera indica | |
| | seed butter, Olea europaea fruit oil, | |
| | Persea gratissima oil, Prunus | |
| | amygdalus dulcis (sweet almond) oil, | |
| | Theobroma cacao seed butter | |
| SymMollient ® PDCC | Propanediol dicaprylate/caprate | 5.0 |
| Neo Heliopan ® BMT | Bis-ethylhexyloxyphenol | 2.0 |
| | methoxyphenyl triazine | |
| Neo Heliopan ® 303 | Octocrylene | 5.0 |
| Neo Heliopan ® OS | Ethylhexyl salicylate | 5.0 |
| Cetiol ® CC | Dicaprylyl carbonate | 5.0 |
| Sweet Almond Oil | Prunus amygdalus dulcis (sweet | 15.0 |
| | almond) oil | |
| Cotton Seed Oil | Gossypium herbaceum seed oil | 15.0 |
| Grape Seed Oil | Vitis vinifera seed oil | 12.0 |
| (Refined) | | |
| Xiameter ® PMX-345 | Cyclopentasiloxane, Cyclohexasiloxane | 10.0 |
| SymHair ® Shape and | Cetearyl nonanoate, Triticum vulgare | 1.0 |
| Color | germ oil, Caprylic/capric triglyceride, | |
| | Linoleic acid, Triticum vulgare bran | |
| | extract, Triticum vulgare germ extract | |
| | Camellia oleifera seed oil | |
| Sym Decanox ™ HA | Caprylic/capric triglyceride, | 2.0 |
| | Hydroxymethoxyphenyl decanone | |
| MAA-20 | | 0.05 |
| Hydrolite ® 5 | Pentylene Glycole | 2.5 |
| Perfume oil PO1; PO2; | Perfume oil PO1; PO2; PO3; PO4; or | 2.4 |
| PO3; PO4; or PO5 | PO5 | |

TABLE F24
Perfect Hand emulsion, expected SPF 15, UVA/UVB balanced

| Raw material | INCI | Amount |
|---|---|---|
| Emulsiphos ® | Potassium cetyl phosphate | 1.5 |
| | Hydrogenated palm glycerides | |
| Lanette ® O | Cetearyl alcohol | 1.0 |
| SymWhite ® 377 | Phenylethyl Resorcinol | 0.4 |
| Tegosoft ® MM | Myristyl myristate | 0.5 |
| Neo Heliopan ® BMT | Bis-ethylhexyloxyphenol | 2.0 |
| | methoxyphenyl triazine | |
| Neo Heliopan ® OS | Ethylhexyl salicylate | 5.0 |
| Edeta ® BD | Disodium EDTA | 0.1 |
| Dragoxat ® 89 | Ethylhexyl isononanoate | 2.0 |
| Isoadipate | Diisopropyl adipate | 6.0 |
| PCL-Liquid ® 100 | Cetearyl ethylhexanoate | 3.0 |
| Corapan ® TQ | Diethylhexyl 2,6-naphthalate | 2.0 |
| Cetiol ® SB 45 | Butyrospermum parkii butter | 1.5 |
| SymBright ™ 2036 | Sclareolide | 0.2 |
| Sym Decanox ™ HA | Caprylic/capric triglyceride, | 1.0 |
| | Hydroxymethoxyphenyl decanone | |
| SymCare ® O | Hexyldecanol, Pentylene glycol, 4-t- | 2.0 |
| | butylcyclohexanol, Bisabolol, | |
| | Cetylhydroxyproline palmitamide, | |
| | Hydroxyphenyl propamidobenzoic | |
| | acid, Stearic acid, Brassica | |
| | campestris (rapeseed) sterols, | |
| | Zingiber officinale (ginger) root | |
| | extract | |
| SymDetox ® | Methylcyclohexyl Succinate | 0.5 |
| | Dimethylamide | |
| Genuvisco ® | Chondrus crispus powder | 0.4 |
| Carrageenan CG-131 | | |
| Keltrol ® CG-SFT | Xanthan gum | 0.2 |
| Aqua/Water | Aqua | Ad 100 |
| MAA-1 | | 2.00 |
| MAA-13 | | 1.00 |
| Neo Heliopan ® Hydro | Phenylbenzimidazole sulfonic acid | 2.5 |
| Biotive ® L-Arginine | Arginine | 1.0 |
| Sodium Hydroxide | Aqua | 2.6 |
| 10% solution | Sodium hydroxide | |
| Glycerin | Glycerin | 2.0 |
| SymSave ® H | Hydroxyacetophenone | 0.5 |
| SymDiol ® 68 | 1,2-hexanediol, Caprylyl glycol | 0.5 |
| RonaFlair ® Flawless | Silica, Ci 77892, Ci 77491 | 1.5 |
| Extrapone ® Moroccan | Aqua, Glycerin, Peg-40 hydrogenated | 1.0 |
| Rose GW | castor oil, Rosa 220amascene flower | |
| | extract | |
| Tego ® Feel C10 | Cellulose | 1.5 |
| Perfume oil PO1; PO2; | Perfume oil PO1; PO2; PO3; PO4; or | 0.5 |
| PO3; PO4; or PO5 | PO5 | |

TABLE F25
Tinted Sunscreen cream against hyperpigmentation

| Raw material | INCI | Amount |
|---|---|---|
| Emulsiphos ® F | Potassium cetyl phosphate | 2.0 |
| | Hydrogenated palm glycerides | |
| Neo Heliopan ® 357 | Butyl methoxydibenzoylmethane | 4.2 |
| Neo Heliopan ® OS | Ethylhexyl salicylate | 5.0 |
| Neo Heliopan ® BMT | Bis-ethylhexyloxyphenol | 3.5 |
| | methoxyphenyl triazine | |
| Uvasorb ® HEB | Diethylhexyl butamido triazone | 7.0 |
| SymMollient ® S | Cetearyl nonanoate | 1.0 |
| Isoadipate | Diisopropyl adipate | 4.0 |
| SymBright ™ 2036 | Sclareolide | 0.2 |
| SymSitive ® 1609 | Pentylene glycol, 4-t- | 1.0 |
| | butylcyclohexanol | |
| Caprylic/Capric | Caprylic/capric triglyceride | 8.0 |
| Triglyceride | | |
| Sym Decanox ™ HA | Caprylic/capric triglyceride | 1.0 |
| | Hydroxymethoxyphenyl decanone | |
| Cetiol ® AB | C12-15 alkyl benzoate | 13.5 |
| EDTA ® BD | Disodium edta | 0.1 |
| Keltrol ® CG-BT | Xanthan gum | 0.3 |
| Genuvisco ® | Chondrus crispus powder | 0.2 |
| Carrageenan CG-131 | | |
| Food Color Brown | Titanium dioxides (ci 77891), Iron | 2.0 |
| E172 + E171 Powder | oxides (ci 77492), Iron oxides (ci | |
| | 77491), Iron oxides (ci. 77499) | |
| Eusolex ® T-AVO | Titanium dioxide, Silica | 2.0 |
| Aqua/Water | Aqua | 31.4 |
| MAA-1 | | 1.00 |
| MAA-13 | | 0.5 |
| Neo Heliopan ® Hydro | Phenylbenzimidazole sulfonic acid | 2.0 |
| Biotive ® L-Arginine | Arginine | 1.0 |
| Sodium Hydroxide | Aqua, Sodium hydroxide | 1.2 |
| 10% solution | | |
| Dragosine ® | Carnosine | 0.2 |
| Glycerin 99.5% | Glycerin, Aqua | 3.0 |
| SymSave ® H | Hydroxyacetophenone | 0.5 |
| Hydrolite ® 5 green | Pentylene glycol | 1.5 |
| Phenoxyethanol | Phenoxyethanol | 0.5 |
| Lanette ® E | Sodium cetearyl sulfate | 0.8 |
| Perfume oil PO1; PO2; | Perfume oil PO1; PO2; PO3; | 0.5 |
| PO3; PO4; or PO5 | PO4; or PO5 | |
| SymOat ® PLUS | Aqua, Glycerin, Maltodextrin, 1,2- | 2.0 |
| | hexanediol, | |
| | Beta-glucan, Caprylyl glycol | |
| Sensocel ® 10 | Cellulose | 1.0 |

TABLE F26
BB Cream Dark Tone, expected SPF-20

| Raw material | INCI | Amount |
|---|---|---|
| Emulsiphos ® | Potassium cetyl phosphate | 2.5 |
| | Hydrogenated palm glycerides | |
| Eumulgin ® SG | Sodium stearoyl glutamate | 0.5 |
| PCL-Liquid ® 100 | Cetearyl ethylhexanoate | 2.0 |
| Lanette ® O | Cetearyl alcohol | 1.0 |
| Dragoxat ® 89 | Ethylhexyl isononanoate | 1.0 |
| Cutina ® PES | Pentaerythrityl distearate | 1.0 |
| SymMollient ® S | Cetearyl nonanoate | 1.0 |
| Xiameter ® PMX-0245 | Cyclopentasiloxane | 2.0 |
| Cyclopentasiloxane | | |
| Xiameter ® PMX-200 Silicone | Dimethicone | 1.0 |
| Fluid 50 cs | | |
| Neo Heliopan ® 357 | Butyl methoxydibenzoylmethane | 3.0 |
| Neo Heliopan ® 303 | Octocrylene | 7.0 |
| Neo Heliopan ® HMS | Homosalate | 7.0 |
| Neo Heliopan ® BMT | Bis-ethylhexyloxyphenol methoxyphenyl | 1.0 |
| | triazine | |
| Neo Heliopan ® OS | Ethylhexyl salicylate | 5.0 |
| SymDecanox ™ HA | Caprylic/capric triglyceride | 2.0 |
| | Hydroxymethoxyphenyl decanone | |
| Talcum | Talc | 2.0 |
| Food Color Titanium Dioxide | Ci 77891 | 0.8 |
| Powder E171 | | |
| Keltrol ® CG-SFT | Xanthan gum | 0.4 |
| Carbopol ® Ultrez 20 Polymer | Acrylates/c10-30 alkyl acrylate | 0.3 |
| | crosspolymer | |
| EDTA ® BD | Disodium EDTA | 0.2 |
| Aqua/Water | Aqua | Ad 100 |
| MAA-1 | | 0.5 |
| MAA-13 | | 2.0 |
| Neo Heliopan ® Hydro | Phenylbenzimidazole sulfonic acid | 1.0 |
| Biotive ® L-Arginine | Arginine | 1.0 |
| Sodium Hydroxide 10% solution | Aqua | 1.5 |
| | Sodium hydroxide | |
| COSMETIC COLOR BROWN | Ci 77891, Ci 77492, Ci 77491, Ci 77499 | 1.5 |
| POWDER | | |
| Food Color Iron Oxide Yellow | Ci 77492 | 0.3 |
| Glycerin | Glycerin | 3.0 |
| Hydrolite ® 5 Green | Pentylene glycol | 1.5 |
| Hydrolite ® CG | Caprylyl glycol | 0.2 |
| SymSave ® H | Hydroxyacetophenone | 0.5 |
| Hydroviton ® PLUS 2290 | Aqua, Pentylene glycol, Glycerin, | 2.0 |
| | Fructose, Urea, Citric acid, Sodium | |
| | hydroxide, Maltose, Sodium pca, Sodium | |
| | chloride, Sodium lactate, Trehalose, | |
| | Allantoin, Sodium hyaluronate, Glucose | |
| Perfume oil PO1; PO2; PO3; | Perfume oil PO1; PO2; PO3; PO4; or | 0.5 |
| PO4; or PO5 | PO5 | |

TABLE F27
Daily Well Aging, SPF 30, UVA/UVB balanced, w/o Octocrylene

| Raw material | INCI | Amount |
|---|---|---|
| Lanette ® | Cetearyl alcohol | 1.5 |
| Emulsiphos ® | Potassium cetyl phosphate | 2.5 |
| | Hydrogenated palm glycerides | |
| SymUrban ® | Benzylidene dimethoxydimethylindanone | 0.3 |
| Neo Heliopan ® 357 | Butyl methoxydibenzoylmethane | 4.0 |
| Neo Heliopan ® OS | Ethylhexyl salicylate | 5.0 |
| Neo Heliopan ® BMT | Bis-ethylhexyloxyphenol methoxyphenyl | 3.0 |
| | triazine | |
| Uvasorb ® HEB | Diethylhexyl butamido triazone | 2.0 |
| SymMollient ® S | Cetearyl nonanoate | 1.0 |
| Cetiol ® Sensoft | Propylheptyl caprylate | 4.0 |
| Tegosoft ® TN | C12-15 alkyl benzoate | 11.0 |
| SymDecanox ™ HA | Caprylic/capric triglyceride | 1.0 |
| | Hydroxymethoxyphenyl decanone | |
| Isoadipate | Diisopropyl adipate | 7.5 |
| EDTA ® BD | Disodium edta | 0.1 |
| Carbopol ® ETD 2050 Polymer | Carbomer | 0.3 |
| Keltrol ® CG-T | Xanthan gum | 0.2 |
| Aqua/Water | Aqua | Ad 100 |
| MAA-20 | | 3.0 |
| Hydrolite ® 5 Green | Pentylene glycol | 2.0 |
| Glycerin 99.5% | Glycerin, Aqua | 1.0 |
| Lanette ® E | Sodium cetearyl sulfate | 0.8 |
| Neo Heliopan ® Hydro | Phenylbenzimidazole sulfonic acid | 3.0 |
| Biotive ® L-Arginine | Arginine | 1.5 |
| Propylene Glycol | Propylene glycol | 3.0 |
| Sodium Hydroxide 10% solution | Aqua, Sodium hydroxide | 3.1 |
| SymSave ® H | Hydroxyacetophenone | 0.6 |
| Phenoxyethanol | Phenoxyethanol | 0.5 |
| Dragosine ® | Carnosine | 0.2 |
| SymVital ® AR | Zingiber officinale (ginger) root extract | 0.2 |
| Frescolat ® ML Cryst | Menthyl lactate | 0.5 |
| Perfume oil PO1; PO2; PO3; | Perfume oil PO1; PO2; PO3; PO4; or | 1.0 |
| PO4; or PO5 | PO5 | |
| Tapioca Pure | Tapioca starch | 2.0 |

TABLE F28
Anti-Wrinkle Day Emulsion, expected SPF 15

| Raw material | INCI | Amount |
|---|---|---|
| Emulsiphos ® | Potassium cetyl phosphate, | 2.5 |
| | Hydrogenated palm glycerides | |
| Neo Heliopan ® BMT | Bis-ethylhexyloxyphenol methoxyphenyl | 2.0 |
| | triazine | |
| Neo Heliopan ® 303 | Octocrylene | 2.0 |
| Neo Heliopan ® OS | Ethylhexyl salicylate | 5.0 |
| Isoadipate | Diisopropyl adipate | 2.0 |
| SymMollient ® S | Cetearyl nonanoate | 2.0 |
| Caprylic/Capric Triglyceride | Caprylic/capric triglyceride | 3.0 |
| Dragoxat ® 89 | Ethylhexyl isononanoate | 1.0 |
| PCL-Liquid ® 100 | Cetearyl ethylhexanoate | 3.0 |
| SymUrban ® | Benzylidene dimethoxydimethylindanone | 0.3 |
| SymDecanox ™ HA | Caprylic/capric triglyceride | 2.0 |
| | Hydroxymethoxyphenyl decanone | |
| Edeta ® BD | Disodium edta | 0.1 |
| Keltrol ® CG-SFT | Xanthan gum | 0.2 |
| Cosmedia ® SP | Sodium Polyacrylate | 0.4 |
| Aqua/Water | Aqua | Ad 100 |
| Hydrolite ® 5 Green | Pentylene glycol | 3.0 |
| Glycerin | Glycerin | 3.0 |
| MAA-28 | | 0.7 |
| MAA-13 | | 1.0 |
| Neo Heliopan ® Hydro | Phenylbenzimidazole sulfonic acid | 2.0 |
| Neo Heliopan ® AP | Disodium Phenyl Dibenzimidazole | 1.0 |
| | Tetrasulfonate | |
| Biotive ® L-Arginine | Arginine | 1.0 |
| Sodium Hydroxide 10% solution | Aqua, Sodium hydroxide | 1.5 |
| Dragosine ® | Carnosine | 0.2 |
| SymSave ® H | Hydroxyacetophenone | 0.5 |
| SymDiol ® 68 | 1,2-hexanediol, Caprylyl glycol | 0.3 |
| Phenoxyethanol | Phenoxyethanol | 0.2 |
| Hydroviton ® PLUS 2290 | Aqua, Pentylene glycol, Glycerin, | 3.0 |
| | Fructose, Urea, Citric acid, Sodium | |
| | hydroxide, Maltose, Sodium pca | |
| | Sodium chloride, Sodium lactate, | |
| | Trehalose, Allantoin, Sodium | |
| | hyaluronate, Glucose | |
| SymLift ® | Aqua, Trehalose, Glycerin, Pentylene | 5.0 |
| | glycol, Beta-glucan, Hordeum vulgare | |
| | seed extract, Sodium hyaluronate, 1,2- | |
| | hexanediol, Caprylyl glycol | |
| Macadamia Oil (refined) | Macadamia integrifolia seed oil | 0.2 |
| SymVital ® AR | Zingiber officinale (ginger) root extract | 0.2 |
| Actipone ® Laminaria Saccharina | Glycerin, Aqua, Laminaria saccharina | 1.0 |
| GW | extract | |
| Ethanol 96% | Alcohol denat. | 3.0 |
| Sensocel ® 10 | Cellulose | 1.0 |
| Sipernat ® 11 PC | Hydrated silica | 1.0 |
| Perfume oil PO1; PO2; PO3; | Perfume oil PO1; PO2; PO3; PO4; or | 0.4 |
| PO4; or PO5 | PO5 | |

TABLE F29
Daily Tattoo Care, expected SPF 10, UVA/UVB balanced

| Raw material | INCI | Amount |
|---|---|---|
| Aqua/Water | Aqua | Ad 100 |
| Hydrolite ® 5 Green | Pentylene glycol | 2.0 |
| Neo Heliopan ® Hydro | Phenylbenzimidazole sulfonic acid | 2.5 |
| Biotive ® L-Arginine | Arginine | 1.0 |
| Sodium Hydroxide 10% solution | Aqua, Sodium hydroxide | 3.5 |
| MAA-1 | | 3.00 |
| MAA-13 | | 0.1 |
| SymSave ® H | Hydroxyacetophenone | 0.5 |
| SymDiol ® 68 | 1,2-hexanediol, Caprylyl glycol | 0.5 |
| Edeta ® BD | Disodium EDTA | 0.1 |
| Emulsiphos ® F | Potassium cetyl phosphate | 2.5 |
| | Hydrogenated palm glycerides | |
| Cetiol ® SB 45 | Butyrospermum parkii butter | 2.0 |
| Lanette ® 16 | Cetyl alcohol | 2.0 |
| Lanette ® O | Cetearyl alcohol | 3.5 |
| SymMollient ® S | Cetearyl nonanoate | 1.0 |
| Caprylic/Capric Triglyceride | Caprylic/capric triglyceride | 6.0 |
| Corapan ® TQ | Diethylhexyl 2,6-naphthalate | 1.0 |
| Dragoxat ® 89 | Ethylhexyl isononanoate | 2.0 |
| Isoadipate | Diisopropyl adipate | 3.0 |
| Neo Heliopan ® BMT | Bis-ethylhexyloxyphenol methoxyphenyl | 2.0 |
| | triazine | |
| Neo Heliopan ® OS | Ethylhexyl salicylate | 5.0 |
| SymCare ® C | Hexyldecanol, Pentylene glycol, 4-t- | 4.0 |
| | butylcyclohexanol, Bisabolol, | |
| | Cetylhydroxyproline palmitamide, | |
| | Hydroxyphenyl propamidobenzoic acid, | |
| | Stearic acid, Brassica campestris | |
| | (rapeseed) sterols, Zingiber officinale | |
| | (ginger) root extract | |
| SymDecanox ™ HA | Caprylic/capric triglyceride | 1.0 |
| | Hydroxymethoxyphenyl decanone | |
| Cetiol ® Ultimate | Tridecane, Undecane | 4.0 |
| Carbopol ® Ultrez 21 Polymer | Acrylates/c10-30 alkyl acrylate | 0.4 |
| | crosspolymer | |
| Keltrol ® CG-T | Xanthan gum | 0.2 |
| SymGlucan | Aqua, Glycerin, 1,2-hexanediol, Caprylyl | 2.0 |
| | glycol, Beta-glucan | |
| Perfume oil PO1; PO2; PO3; | Perfume oil PO1; PO2; PO3; PO4; or | 0.5 |
| PO4; or PO5 | PO5 | |
| Tapioca Pure | Tapioca starch | 2.0 |

TABLE F30
Sensitive Day Care Cream, expected SPF 15

| Raw material | INCI | Amount |
|---|---|---|
| Lanette ® | Cetearyl alcohol | 4.0 |
| Emulsiphos ® | Potassium cetyl phosphate, | 2.0 |
| | Hydrogenated palm glycerides | |
| Cetiol ® AB | C12-15 alkyl benzoate | 1.0 |
| Cetiol ® OE | Dicaprylyl ether | 1.0 |
| SymMollient ® S | Cetearyl nonanoate | 1.0 |
| Neo Heliopan ® BMT | Bis-ethylhexyloxyphenol methoxyphenyl | 2.0 |
| | triazine | |
| Neo Heliopan ® 303 | Octocrylene | 3.0 |
| Grape Seed Oil (Refined) | Vitis vinifera seed oil | 1.0 |
| Corapan ® TQ | Diethylhexyl 2,6-naphthalate | 3.0 |
| Eumulgin ® SG | Sodium stearoyl glutamate | 1.0 |
| SymCare ® O | Hexyldecanol, Pentylene glycol, 4-t- | 3.0 |
| | butylcyclohexanol, Bisabolol, | |
| | Cetylhydroxyproline palmitamide, | |
| | Hydroxyphenyl propamidobenzoic acid, | |
| | Stearic acid, Brassica campestris | |
| | (rapeseed) sterols, Zingiber officinale | |
| | (ginger) root extract | |
| Softisan ® 100 | Hydrogenated coco-glycerides | 1.0 |
| Eutanol ® G | Octyldodecanol | 2.0 |
| Caprylic/Capric Triglyceride | Caprylic/capric triglyceride | 5.0 |
| Carbopol ® Ultrez 10 Polymer | Carbomer | 0.3 |
| Aqua/Water | Aqua | Ad 100 |
| Hydrolite ® 5 Green | Pentylene glycol | 1.0 |
| Hydrolite ® 7 Green | 1,2-heptanediol | 0.5 |
| MAA-8 | | 1.0 |
| MAA-1 | | 1.0 |
| MAA-13 | | 0.2 |
| Neo Heliopan ® Hydro | Phenylbenzimidazole sulfonic acid | 3.0 |
| Biotive ® L-Arginine | Arginine | 1.0 |
| Sodium Hydroxide 10% | Aqua, Sodium hydroxide | 2.8 |
| solution | | |
| Edeta ® BD | Disodium EDTA | 0.1 |
| Glycerin | Glycerin | 5.0 |
| SymSave ® H | Hydroxyacetophenone | 0.5 |
| Phenoxyethanol | Phenoxyethanol | 0.5 |
| Hydroviton ® PLUS 2290 | Aqua, Pentylene glycol, Glycerin, | 3.0 |
| | Fructose, Urea, Citric acid, Sodium | |
| | hydroxide, Maltose, Sodium pca, Sodium | |
| | chloride, Sodium lactate, Trehalose, | |
| | Allantoin, Sodium hyaluronate, Glucose | |
| D-Panthenol 75 W | Panthenol, Aqua | 2.0 |
| Tapioca Pure | Tapioca starch | 4.0 |
| Actipone ® Licorice | Aqua, Butylene glycol, Glycyrrhiza glabra | 2.0 |
| | root extract | |
| Sodium Hydroxide 10% solution | Aqua, Sodium hydroxide | 1.5 |

TABLE F31
Cream, expected SPF 15, UVA/UVB

| Raw material | INCI | Amount |
|---|---|---|
| Aqua/Water | Aqua | Ad 100 |
| MAA-20 | | 1.0 |
| MAA-1 | | 1.5 |
| SymSol ® PF-3 | Aqua, Pentylene glycol, Sodium lauryl | 2.0 |
| | sulfoacetate, Sodium oleoyl sarcosinate, | |
| | Sodium chloride, Sodium oleate | |
| Neo Heliopan ® Hydro | Phenylbenzimidazole sulfonic acid | 2.5 |
| Biotive ® L-Arginine | Arginine | 1.0 |
| Sodium Hydroxide 10% solution | Aqua, Sodium hydroxide | 2.1 |
| Aqua Keep 10SH-NFC | Sodium acrylates crosspolymer-2 | 2.0 |
| SymSave ® H | Hydroxyacetophenone | 0.5 |
| Dragosine ® | Carnosine | 0.1 |
| Hydrolite ® 5 Green | Pentylene glycol | 2.5 |
| Neo Heliopan ® OS | Ethylhexyl salicylate | 3.0 |
| Neo Heliopan ® 303 | Octocrylene | 3.0 |
| Neo Heliopan ® BMT | Bis-ethylhexyloxyphenol methoxyphenyl | 1.5 |
| | triazine | |
| Neo Heliopan ® HMS | Homosalate | 3.0 |
| SymMollient ® S | Cetearyl nonanoate | 1.0 |
| Isoadipate | Diisopropyl adipate | 2.5 |
| SymRelief ® 100 | Bisabolol | 0.2 |
| | Zingiber officinale root extract | |
| SymDecanox ™ HA | Caprylic/capric triglyceride | 1.5 |
| | Hydroxymethoxyphenyl decanone | |
| SymDetox ® | 3,3,5-trimethylcyclohexyl succinate | 0.5 |
| | dimethylamide | |
| SymUrban | Benzylidene dimethoxydimethylindanone | 0.5 |
| EDETA ® BD | Disodium edta | 0.1 |
| Frescolat ® X-Cool | Menthyl ethylamido oxalate | 0.5 |
| SymBright ™ 2036 | Sclareolide | 0.1 |
| SymWhite ® PLUS | Caprylic/capric triglyceride, Pentylene | 1.3 |
| | glycol | |
| | Phenylethyl resorcinol, Bisabolol, Butyl | |
| | methoxydibenzoylmethane | |
| Keltrol ® CG-BT | Xanthan gum | 0.2 |
| FOOD COLOR BROWN | Titanium dioxides (ci 77891), Iron oxides | 2.0 |
| E172 + E171 POWDER | (ci 77492), Iron oxides (ci 77491), Iron | |
| | oxides (ci. 77499) | |
| Perfume oil PO1; PO2; PO3; | Perfume oil PO1; PO2; PO3; PO4; or | 0.5 |
| PO4; or PO5 | PO5 | |
| SymGlucan | Aqua, Glycerin, 1,2-hexanediol, Caprylyl | 5.0 |
| | glycol, Beta-glucan | |

TABLE F32
All purpose cleaner

| No | Ingredients | Amount |
|---|---|---|
| 1 | Fettalkoholethoxylat | 4 |
| 2 | Sulfonic acid, C 13-17-sec-Alkyl-, Sodium salt | 6.2 |
| 3 | 2-octyl-2H-isothiazol-3-one | 0.1 |
| 4 | Fragrance | 3.5 |
| 5 | MAA-28 | 1.0 |
| 6 | 1,2-heptanediol | 0.9 |
| 7 | Aqua | add 100 |

Colorless liquid, pH 7

TABLE F33
APC alkaline

| No | Ingredients | Amount |
|---|---|---|
| 1 | Coco-Glycoside | 1 |
| 2 | Benzalkonium Chloride | 0.2 |
| 3 | Sodiumbicarbonate | 0.1 |
| 4 | Tri-sodiumcitrate-dihydrate | 0.1 |
| 5 | Methylglycinediacetic acid | 0.1 |
| 6 | 1,2-Benzisothiazol-3(2H)-on | 0.1 |
| 7 | Fragrance | 0.1 |
| 8 | 1,2-heptanediol | 0.5 |
| 9 | MAA-2 | 0.5 |
| 10 | Hydroxyacetophenone | 0.3 |
| 11 | Aqua | add 100 |

Colorless liquid, pH 9

TABLE F34
Detergent liquid light duty

| No | Ingredients | Amount |
|---|---|---|
| 1 | Cocos fatty acid | 0.85 |
| 2 | potassium hydroxide | 0.45 |
| 3 | 2-octyl-2H-isothiazol-3-one | 0.2 |
| 4 | Sulfonic acid, C 13-17-sec-Alkyl-, Sodium salt | 20 |
| 5 | Sodium Laureth Sulfate | 3 |
| 6 | Trideceth-9 | 5 |
| 7 | Sodium Chloride | 1.3 |
| 8 | MAA-26 | 0.1 |
| 9 | 1,2-heptanediol | 0.9 |
| 10 | Fragrance | 0.5 |
| 11 | Aqua | add 100 |
| 12 | Hydroxyacetophenone | 0.2 |

Liquid, pH 7.3

TABLE F35
Dishwash liquid, manual

| No | Ingredients | Amount |
|---|---|---|
| 1 | Sodium Laureth Sulfate | add 100 |
| 2 | Ethanol | 2.9 |
| 3 | lauramine oxide | 7.7 |
| 4 | propylene glycol | 1.9 |
| 5 | Phenoxyethanol | 0.1 |
| 6 | Benzisothiazolinone, Methylisothiazolinone, Laurylamine | 0.1 |
| | Dipropylenediamine | |
| 7 | 1,2-heptanediol | 0.5 |
| 8 | sodium chloride | 3.85 |
| 9 | Fragrance | 0.3 |
| 10 | Benzyl Alcohol | 0.2 |
| 11 | MAA-28 | 1.5 |

Colorless liquid, pH 8.5

TABLE F36
Fabric softener

| No | Ingredients | Amount |
|---|---|---|
| 1 | Di-(Talg Carboxyethyl) Hydroxyethyl methylammonium- | 5.5 |
| | methosulfat | |
| 2 | Simethicone | 0.3 |
| 3 | 2-octyl-2H-isothiazol-3-one | 0.1 |
| 4 | 1,2-heptanediol | 0.5 |
| 5 | Fragrance | 0.2 |
| 6 | Aqua | add 100 |
| 7 | Phenoxyethanol | 0.2 |
| 8 | MAA-20 | 0.6 |

Liquid, pH 3

TABLE F37
Fabric softener concentrate, encapsulated

| No | Ingredients | Amount |
|---|---|---|
| 1 | Di-(Talg Carboxyethyl) Hydroxyethyl methylammonium- | 16.6 |
| | methosulfat | |
| 2 | 2-octyl-2H-isothiazol-3-one | 0.1 |
| 3 | Simethicone | 0.3 |
| 4 | magnesium chloride | 0.8 |
| 5 | encapsulated perfume oil | 0.3 |
| 6 | crosslinked cationic polymer | 0.15 |
| 7 | MAA-1 | 0.9 |
| 8 | 1,2-heptanediol | 0.5 |
| 9 | Fragrance | 0.6 |
| 10 | Aqua | add 100 |
| 11 | Glyceryl Caprylate | 0.1 |

White liquid, pH 2.5

TABLE F38
Hand soap, liquid

| No | Ingredients | Amount |
|---|---|---|
| 1 | Sodium Laureth Sulfate | 18 |
| 2 | Cocoamidopropylbetaine | 6 |
| 3 | Cocamide DEA | 3 |
| 4 | citric acid | 0.5 |
| 5 | sodium chloride | 1.9 |
| 6 | Glycerol | 2 |
| 7 | Fragrance | 0.2 |
| 8 | MAA-20 | 0.25 |
| 9 | Aqua | add 100 |
| 10 | Propanediol Caprylate | 0.3 |

Slightly colored liquid, pH 6

TABLE F39
Scent Lotion, encapsulated

| No | Ingredients | Amount |
|---|---|---|
| 1 | microcrystalline cellulose/cellulose gum | 1 |
| 2 | PEG-40 hydrogenated Castor oil/Trideceth-9 | 0.5 |
| 3 | encapsulated perfume oil | 1 |
| 4 | 2-octyl-2H-isothiazol-3-one | 0.1 |
| 5 | Fragrance | 5 |
| 6 | 1,2-heptanediol | 1 |
| 7 | Aqua | add 100 |
| 8 | MAA-12 | 0.4 |

White emulsion, pH neutral

TABLE F40
Cleaner, liquid, citric acid

| No | Ingredients | Amount |
|---|---|---|
| 1 | Sulfonic acid, C 13-17-sec-Alkyl-, Sodium salt | 1 |
| 2 | Xanthan gum | 0-3 |
| 3 | citric acid | 5 |
| 4 | 1,2heptanediol | 0.25 |
| 5 | Fragrance | 0.3 |
| 6 | Aqua | add 100 |
| 7 | Anisic Acid | 0.1 |
| 8 | MAA-1 | 0.3 |

Liquid, pH 2

1. A sunscreen product, cosmetic or pharmaceutical preparation or homecare product, comprising
(a) at least one primary organic and/or inorganic UV-filter;
(b) at least one mycosporine-like amino acid compound,
represented either by the general formula (V) 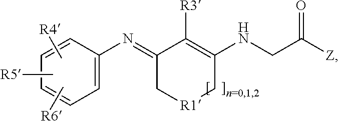
wherein
Z is selected from the group consisting of O—R2′ or amide′;
R1′ is selected from the group consisting of CH₂, unsubstituted or substituted CH(alkyl), unsubstituted or substituted C(alkyl)₂, unsubstituted or substituted CH(cycloalkyl), unsubstituted or substituted CH(aryl), unsubstituted or substituted CH(heterocycloalkyl), unsubstituted or substituted CH(heteroaryl), CR7′R8′, C═O, halogen, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), and unsubstituted or substituted N(heteroaryl), and unsubstituted or substituted C-spirocycles;
R2′ is selected from the group consisting of H, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted cycloalkyl, unsubstituted or substituted aryl, unsubstituted or substituted heterocycloalkyl, and unsubstituted or substituted heteroaryl;
amide′ is selected from the group consisting of NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9′R10′, unsubstituted or substituted —N(O-alkyl)(alkyl) (Weinreb amide), —N(OH)(H) (hydroxamate), and unsubstituted or substituted —N(OH)(alkyl) (alkylated hydroxamate);
R3′ is selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9′R10′, and unsubstituted or substituted C-spirocycles;
the substituents R4′, R5′ and R6′ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, —S(═O)₂OH, sulfonyl, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9′R10′, and unsubstituted or substituted C-spirocycles;
R7′ and R8′ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl) and unsubstituted or substituted NR9′R10′;
R9′ und R10′ which may be identical or different, are independently from each other selected from the group consisting of H, OH, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, unsubstituted or substituted cycloalkyl, unsubstituted or substituted aryl, unsubstituted or substituted heterocycloalkyl, and unsubstituted or substituted heteroaryl;
Y is selected from the group consisting of H and unsubstituted or substituted alkyl; and
R is unsubstituted or substituted alkyl;
or a tautomer or a stereoisomer or a salt of the afore-mentioned compounds;
or represented by the general formula (VI) 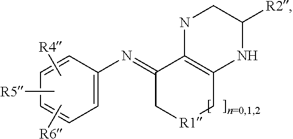
wherein
R1″ is selected from the group consisting of CH₂, unsubstituted or substituted CH(alkyl), unsubstituted or substituted C(alkyl)₂, unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, halogen, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles;
R2″ is selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH—C(═O)—O—Y, —C(═O)-amide″, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, and unsubstituted or substituted C-spirocycles;
X is selected from the group consisting of CH₂, unsubstituted or substituted CH(alkyl), unsubstituted or substituted CH(alkenyl), unsubstituted or substituted CH(alkynyl), unsubstituted or substituted CH(alkoxy), unsubstituted or substituted CH(alkylthio), unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles;
the substituents R4″, R5″ and R6″ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, —S(═O)₂OH, sulfonyl, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, and unsubstituted or substituted C-spirocycles;
R7″ and R8″ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl) and unsubstituted or substituted NR9″R10″;
amide″ is selected from the group consisting of NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, unsubstituted or substituted —N(O-alkyl)(alkyl) (Weinreb amide), —N(OH)(H) (hydroxamate), and unsubstituted or substituted —N(OH)(alkyl) (alkylated hydroxamate);
R9″ und R10″ which may be identical or different, are independently from each other selected from the group consisting of H, OH, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, unsubstituted or substituted cycloalkyl, unsubstituted or substituted aryl, unsubstituted or substituted heterocylcoalkyl, and unsubstituted or substituted heteroaryl;
Y is selected from the group consisting of H and unsubstituted or substituted alkyl; and
R is unsubstituted or substituted alkyl;
or a tautomer or a stereoisomer or a salt of the afore-mentioned compounds;
or any mixture of the afore-mentioned compounds; and
(c) optionally at least one film forming agent;
with the proviso, that the following compounds are disclaimed/excluded:

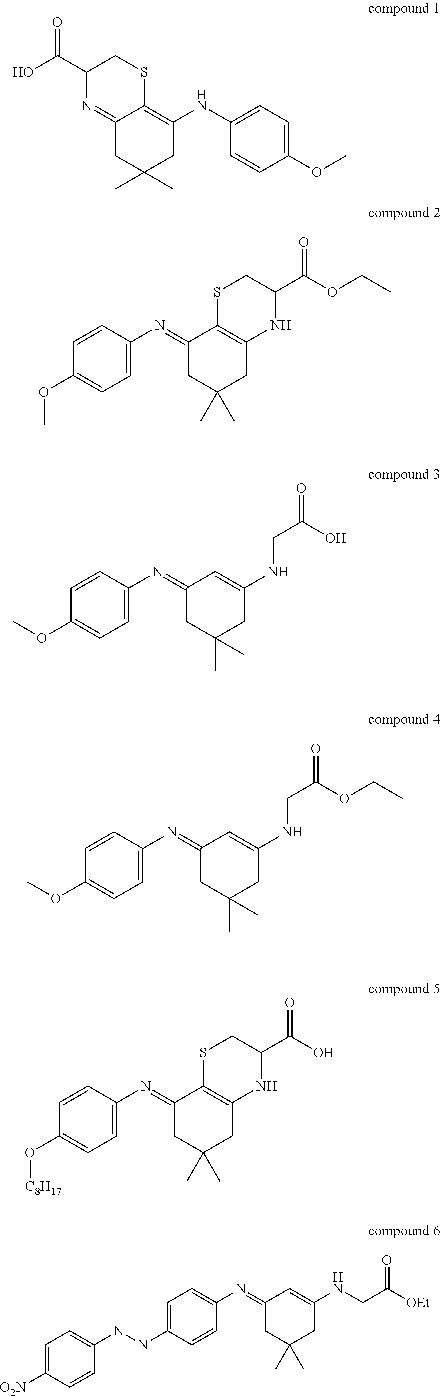

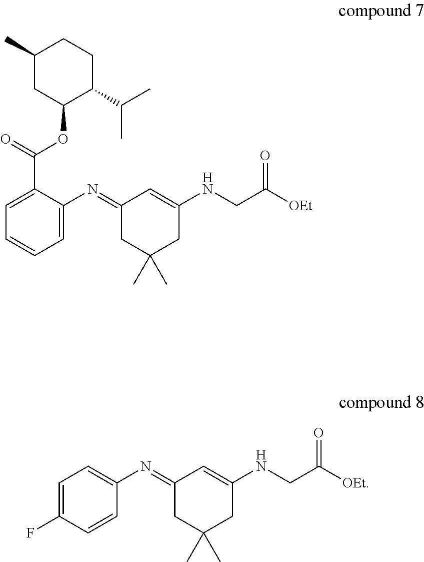 [SMILES: CCOC(=O)CNC1=C/C(=N/c2ccccc2C(=O)O[C@H]2C[C@@H](C)CC[C@@H]2C(C)C)CC(C)(C)C1 ; CCOC(=O)CNC1=C/C(=N/c2ccc(F)cc2)CC(C)(C)C1]

2. The sunscreen product, cosmetic or pharmaceutical preparation or homecare product according to claim 1, wherein the mycosporine-like amino acid compound is represented either by the general formula (VII) 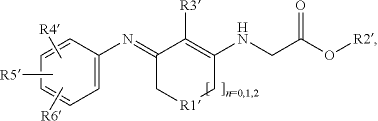
wherein R1′, R2′, R3′, R4′, R5′ and R6′ have the same meaning as defined for formula (V);
or by the general formula (VIII) 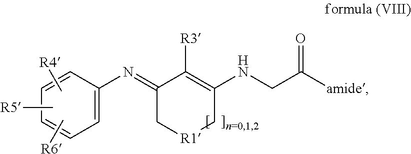
wherein R1′, amide′, R3′, R4′, R5′ and R6′ have the same meaning as defined for formula (V).

3. The sunscreen product, cosmetic or pharmaceutical preparation or homecare product according to claim 1, wherein in the general formula (VI) X is selected from the group consisting of CH₂, unsubstituted or substituted CH(alkyl), unsubstituted or substituted C(alkyl)₂, unsubstituted or substituted CH(alkenyl), unsubstituted or substituted CH(alkynyl), unsubstituted or substituted CH(alkoxy), unsubstituted or substituted CH(alkylthio), unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles.

4. The sunscreen product, cosmetic or pharmaceutical preparation or homecare product according to claim 1, wherein in the general formula (V)
Z is —O—R2′ or amide′; and/or
R1′ is selected from the group consisting of CH₂, C(alkyl)₂, O, S, SO, SO₂, NH and N(alkyl); and/or
R2′ is selected from the group consisting of H, methyl, ethyl, propyl, isopropyl, butyl, isobutyl, tert-butyl, 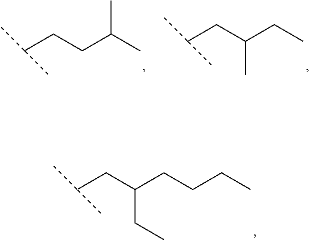
(2-ethylhexyl) and phenyl; or
amide′ is selected from the group consisting of NH₂, NH(alkyl) and N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl;
or wherein in the general formulae (VII) or (VIII)
R1′ is selected from the group consisting of CH₂, C(alkyl)₂, O, S, SO, SO₂, NH and N(alkyl); and/or
R2′ is selected from the group consisting of H, methyl, ethyl, propyl, isopropyl, butyl, isobutyl, tert-butyl, 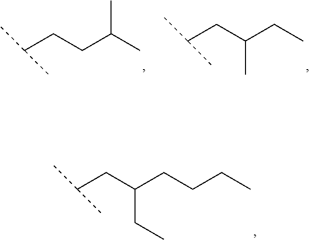
(2-ethylhexyl) and phenyl; or
amide′ is selected from the group consisting of NH₂, NH(alkyl) and N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formulae (VII) and (VIII);
wherein alkyl is selected from the group consisting of methyl, ethyl, propyl, isopropyl, butyl, isobutyl, and tert-butyl;
or wherein in the general formula (VI)
R1″ is selected from the group consisting of CH₂, C(alkyl)₂, O, S, SO, SO₂, NH and N(alkyl); and/or
R2″ is selected from the group consisting of —CH₂—OH, —C(═O)—O—Y, and —C(═O)-amide″; and/or
X is selected from the group consisting of CH₂, C═O, O, S, SO, SO₂, NH, and N(alkyl); and/or
Y is selected from the group consisting of H, methyl, ethyl, propyl, isopropyl, butyl, isobutyl, tert-butyl, 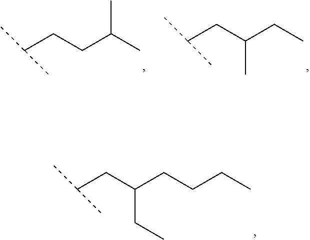
(2-ethylhexyl) and phenyl; and/or
amide″ is selected from the group consisting of NH₂, NH(alkyl) and N(alkyl)₂, —N(O-alkyl)(alkyl) (Weinreb amide) and —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and tert-butyl.

5. The sunscreen product, cosmetic or pharmaceutical preparation or homecare product according to claim 1, wherein in the general formula (V)
Z is —O—R2′ or amide′; and/or
R1′ is selected from the group consisting of CH₂ and C(methyl)₂; and/or
R2′ is selected from the group consisting of H, methyl, ethyl, propyl, isopropyl, butyl, isobutyl, tert-butyl, 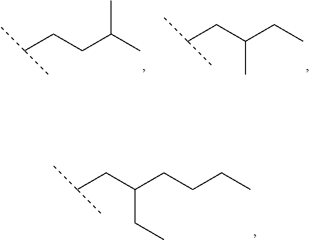
(2-ethylhexyl) and phenyl; or
amide′ is selected from the group consisting of NH₂, NH(alkyl) and N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl;
or wherein in the general formulae (VII) or (VIII)
R1′ is selected from the group consisting of CH₂, and C(methyl)₂;
R2′ is selected from the group consisting of H, methyl, ethyl, propyl, isopropyl, butyl, isobutyl, tert-butyl, 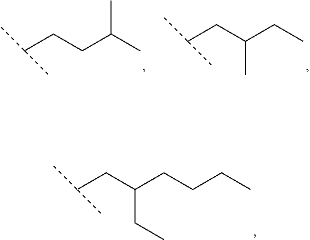
(2-ethylhexyl) and phenyl; or
amide′ is selected from the group consisting of NH₂, NH(alkyl) and N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formulae (VII) and (VIII);
wherein alkyl is selected from the group consisting of methyl, ethyl, propyl, isopropyl, butyl, isobutyl, and tert-butyl; and/or
or wherein in the general formula (VI)
R1″ is selected from the group consisting of CH₂ and C(methyl)₂; and/or
R2″ is selected from the group consisting of —CH₂—OH, —C(═O)—O—Y, and —C(═O)-amide″; and/or
X is selected from the group consisting of CH₂, C═O, O, S, SO, SO₂, NH, and N(alkyl); and/or
Y is selected from the group consisting of H, methyl, ethyl, propyl, isopropyl, butyl, isobutyl, tert-butyl, 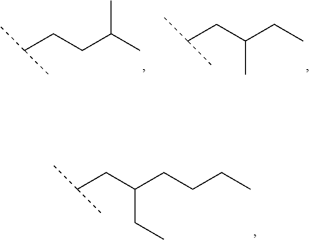
(2-ethylhexyl) and phenyl; and/or
amide″ is selected from the group consisting of NHL, NH(alkyl) and N(alkyl)₂, —N(O-alkyl)(alkyl) (Weinreb amide) and —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and tert-butyl.

6. The sunscreen product, cosmetic or pharmaceutical preparation or homecare product according to claim 1, wherein the mycosporine-like amino acid compound is selected from the group consisting of the following compounds I-1 to I-128 according to the general formula (V) and II-1 to II-192 according to the general formula (VI):

| 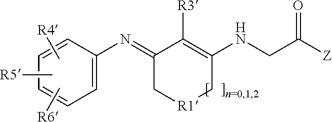 | R1′ | R3′ | Z |
|---|---|---|---|
| I-1 | CH2 | H | —O-R2′ |
| I-2 | CH2 | methyl | —O-R2′ |
| I-3 | CH2 | ethyl | —O-R2′ |
| I-4 | CH2 | —O-methyl | —O-R2′ |
| I-5 | CH2 | —C(═O)—OH | —O-R2′ |
| I-6 | CH2 | —C(═O)—CH3 | —O-R2′ |
| I-7 | CH2 | —C(═O)—C3H7 | —O-R2′ |
| I-8 | CH2 | —C(═O)—O-methyl | —O-R2′ |
| I-9 | CH2 | H | amide′ |
| I-10 | CH2 | methyl | amide′ |
| I-11 | CH2 | ethyl | amide′ |
| I-12 | CH2 | —O-methyl | amide′ |
| I-13 | CH2 | —C(═O)—OH | amide′ |
| I-14 | CH2 | —C(═O)—CH3 | amide′ |
| I-15 | CH2 | —C(═O)—C3H7 | amide′ |
| I-16 | CH2 | —C(═O)—O-methyl | amide′ |
| I-17 | C(methyl)2 | H | —O-R2′ |
| I-18 | C(methyl)2 | methyl | —O-R2′ |
| I-19 | C(methyl)2 | ethyl | —O-R2′ |
| I-20 | C(methyl)2 | —O-methyl | —O-R2′ |
| I-21 | C(methyl)2 | —C(═O)—OH | —O-R2′ |
| I-22 | C(methyl)2 | —C(═O)—CH3 | —O-R2′ |
| I-23 | C(methyl)2 | —C(═O)—C3H7 | —O-R2′ |
| I-24 | C(methyl)2 | —C(═O)—O-methyl | —O-R2′ |
| I-25 | C(methyl)2 | H | amide′ |
| I-26 | C(methyl)2 | methyl | amide′ |
| I-27 | C(methyl)2 | ethyl | amide′ |
| I-28 | C(methyl)2 | —O-methyl | amide′ |
| I-29 | C(methyl)2 | —C(═O)—OH | amide′ |
| I-30 | C(methyl)2 | —C(═O)—CH3 | amide′ |
| I-31 | C(methyl)2 | —C(═O)—C3H7 | amide′ |
| I-32 | C(methyl)2 | —C(═O)—O-methyl | amide′ |
| I-33 | O | H | —O-R2′ |
| I-34 | O | methyl | —O-R2′ |
| I-35 | O | ethyl | —O-R2′ |
| I-36 | O | —O-methyl | —O-R2′ |
| I-37 | O | —C(═O)—OH | —O-R2′ |
| I-38 | O | —C(═O)—CH3 | —O-R2′ |
| I-39 | O | —C(═O)—C3H7 | —O-R2′ |
| I-40 | O | —C(═O)—O-methyl | —O-R2′ |
| I-41 | O | H | amide′ |
| I-42 | O | methyl | amide′ |
| I-43 | O | ethyl | amide′ |
| I-44 | O | —O-methyl | amide′ |
| I-45 | O | —C(═O)—OH | amide′ |
| I-46 | O | —C(═O)—CH3 | amide′ |
| I-47 | O | —C(═O)—C3H7 | amide′ |
| I-48 | O | —C(═O)—O-methyl | amide′ |
| I-49 | S | H | —O-R2′ |
| I-50 | S | methyl | —O-R2′ |
| I-51 | S | ethyl | —O-R2′ |
| I-52 | S | —O-methyl | —O-R2′ |
| I-53 | S | —C(═O)—OH | —O-R2′ |
| I-54 | S | —C(═O)—CH3 | —O-R2′ |
| I-55 | S | —C(═O)—C3H7 | —O-R2′ |
| I-56 | S | —C(═O)—O-methyl | —O-R2′ |
| I-57 | S | H | amide′ |
| I-58 | S | methyl | amide′ |
| I-59 | S | ethyl | amide′ |
| I-60 | S | —O-methyl | amide′ |
| I-61 | S | —C(═O)—OH | amide′ |
| I-62 | S | —C(═O)—CH3 | amide′ |
| I-63 | S | —C(═O)—C3H7 | amide′ |
| I-64 | S | —C(═O)—O-methyl | amide′ |
| I-65 | SO | H | —O-R2′ |
| I-66 | SO | methyl | —O-R2′ |
| I-67 | SO | ethyl | —O-R2′ |
| I-68 | SO | —O-methyl | —O-R2′ |
| I-69 | SO | —C(═O)—OH | —O-R2′ |
| I-70 | SO | —C(═O)—CH3 | —O-R2′ |
| I-71 | SO | —C(═O)—C3H7 | —O-R2′ |
| I-72 | SO | —C(═O)—O-methyl | —O-R2′ |
| I-73 | SO | H | amide′ |
| I-74 | SO | methyl | amide′ |
| I-75 | SO | ethyl | amide′ |
| I-76 | SO | —O-methyl | amide′ |
| I-77 | SO | —C(═O)—OH | amide′ |
| I-78 | SO | —C(═O)—CH3 | amide′ |
| I-79 | SO | —C(═O)—C3H7 | amide′ |
| I-80 | SO | —C(═O)—O-methyl | amide′ |
| I-81 | SO2 | H | —O-R2′ |
| I-82 | SO2 | methyl | —O-R2′ |
| I-83 | SO2 | ethyl | —O-R2′ |
| I-84 | SO2 | —O-methyl | —O-R2′ |
| I-85 | SO2 | —C(═O)—OH | —O-R2′ |
| I-86 | SO2 | —C(═O)—CH3 | —O-R2′ |
| I-87 | SO2 | —C(═O)—C3H7 | —O-R2′ |
| I-88 | SO2 | —C(═O)—O-methyl | —O-R2′ |
| I-89 | SO2 | H | amide′ |
| I-90 | SO2 | methyl | amide′ |
| I-91 | SO2 | ethyl | amide′ |
| I-92 | SO2 | —O-methyl | amide′ |
| I-93 | SO2 | —C(═O)—OH | amide′ |
| I-94 | SO2 | —C(═O)—CH3 | amide′ |
| I-95 | SO2 | —C(═O)—C3H7 | amide′ |
| I-96 | SO2 | —C(═O)—O-methyl | amide′ |
| I-97 | NH | H | —O-R2′ |
| I-98 | NH | methyl | —O-R2′ |
| I-99 | NH | ethyl | —O-R2′ |
| I-100 | NH | —O-methyl | —O-R2′ |
| I-101 | NH | —C(═O)—OH | —O-R2′ |
| I-102 | NH | —C(═O)—CH3 | —O-R2′ |
| I-103 | NH | —C(═O)—C3H7 | —O-R2′ |
| I-104 | NH | —C(═O)—O-methyl | —O-R2′ |
| I-105 | NH | H | amide′ |
| I-106 | NH | methyl | amide′ |
| I-107 | NH | ethyl | amide′ |
| I-108 | NH | —O-methyl | amide′ |
| I-109 | NH | —C(═O)—OH | amide′ |
| I-110 | NH | —C(═O)—CH3 | amide′ |
| I-111 | NH | —C(═O)—C3H7 | amide′ |
| I-112 | NH | —C(═O)—O-methyl | amide′ |
| I-113 | N(alkyl) | H | —O-R2′ |
| I-114 | N(alkyl) | methyl | —O-R2′ |
| I-115 | N(alkyl) | ethyl | —O-R2′ |
| I-116 | N(alkyl) | —O-methyl | —O-R2′ |
| I-117 | N(alkyl) | —C(═O)—OH | —O-R2′ |
| I-118 | N(alkyl) | —C(═O)—CH3 | —O-R2′ |
| I-119 | N(alkyl) | —C(═O)—C3H7 | —O-R2′ |
| I-120 | N(alkyl) | —C(═O)—O-methyl | —O-R2′ |
| I-121 | N(alkyl) | H | amide′ |
| I-122 | N(alkyl) | methyl | amide′ |
| I-123 | N(alkyl) | ethyl | amide′ |
| I-124 | N(alkyl) | —O-methyl | amide′ |
| I-125 | N(alkyl) | —C(═O)—OH | amide′ |
| I-126 | N(alkyl) | —C(═O)—CH3 | amide′ |
| I-127 | N(alkyl) | —C(═O)—C3H7 | amide′ |
| I-128 | N(alkyl) | —C(═O)—O-methyl | amide′ |
| 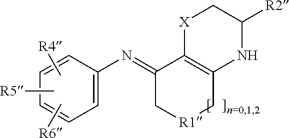 | R1″ | X | R2″ |
| II-1 | CH2 | CH2 | —CH2—OH |
| II-2 | CH2 | C═O | —CH2—OH |
| II-3 | CH2 | O | —CH2—OH |
| II-4 | CH2 | S | —CH2—OH |
| II-5 | CH2 | SO | —CH2—OH |
| II-6 | CH2 | SO2 | —CH2—OH |
| II-7 | CH2 | NH | —CH2—OH |
| II-8 | CH2 | N(alkyl) | —CH2—OH |
| II-9 | CH2 | CH2 | —C(═O)—O—Y |
| II-10 | CH2 | C═O | —C(═O)—O—Y |
| II-11 | CH2 | O | —C(═O)—O—Y |
| II-12 | CH2 | S | —C(═O)—O—Y |
| II-13 | CH2 | SO | —C(═O)—O—Y |
| II-14 | CH2 | SO2 | —C(═O)—O—Y |
| II-15 | CH2 | NH | —C(═O)—O—Y |
| II-16 | CH2 | N(alkyl) | —C(═O)—O—Y |
| II-17 | CH2 | CH2 | —C(═O)-amide″ |
| II-18 | CH2 | C═O | —C(═O)-amide″ |
| II-19 | CH2 | O | —C(═O)-amide″ |
| II-20 | CH2 | S | —C(═O)-amide″ |
| II-21 | CH2 | SO | —C(═O)-amide″ |
| II-22 | CH2 | SO2 | —C(═O)-amide″ |
| II-23 | CH2 | NH | —C(═O)-amide″ |
| II-24 | CH2 | N(alkyl) | —C(═O)-amide″ |
| II-25 | C(methyl)2 | CH2 | —CH2—OH |
| II-26 | C(methyl)2 | C═O | —CH2—OH |
| II-27 | C(methyl)2 | O | —CH2—OH |
| II-28 | C(methyl)2 | S | —CH2—OH |
| II-29 | C(methyl)2 | SO | —CH2—OH |
| II-30 | C(methyl)2 | SO2 | —CH2—OH |
| II-31 | C(methyl)2 | NH | —CH2—OH |
| II-32 | C(methyl)2 | N(alkyl) | —CH2—OH |
| II-33 | C(methyl)2 | CH2 | —C(═O)—O—Y |
| II-34 | C(methyl)2 | C═O | —C(═O)—O—Y |
| II-35 | C(methyl)2 | O | —C(═O)—O—Y |
| II-36 | C(methyl)2 | S | —C(═O)—O—Y |
| II-37 | C(methyl)2 | SO | —C(═O)—O—Y |
| II-38 | C(methyl)2 | SO2 | —C(═O)—O—Y |
| II-39 | C(methyl)2 | NH | —C(═O)—O—Y |
| II-40 | C(methyl)2 | N(alkyl) | —C(═O)—O—Y |
| II-41 | C(methyl)2 | CH2 | —C(═O)-amide″ |
| II-42 | C(methyl)2 | C═O | —C(═O)-amide″ |
| II-43 | C(methyl)2 | O | —C(═O)-amide″ |
| II-44 | C(methyl)2 | S | —C(═O)-amide″ |
| II-45 | C(methyl)2 | SO | —C(═O)-amide″ |
| II-46 | C(methyl)2 | SO2 | —C(═O)-amide″ |
| II-47 | C(methyl)2 | NH | —C(═O)-amide″ |
| II-48 | C(methyl)2 | N(alkyl) | —C(═O)-amide″ |
| II-49 | O | CH2 | —CH2—OH |
| II-50 | O | C═O | —CH2—OH |
| II-51 | O | O | —CH2—OH |
| II-52 | O | S | —CH2—OH |
| II-53 | O | SO | —CH2—OH |
| II-54 | O | SO2 | —CH2—OH |
| II-55 | O | NH | —CH2—OH |
| II-56 | O | N(alkyl) | —CH2—OH |
| II-57 | O | CH2 | —C(═O)—O—Y |
| II-58 | O | C═O | —C(═O)—O—Y |
| II-59 | O | O | —C(═O)—O—Y |
| II-60 | O | S | —C(═O)—O—Y |
| II-61 | O | SO | —C(═O)—O—Y |
| II-62 | O | SO2 | —C(═O)—O—Y |
| II-63 | O | NH | —C(═O)—O—Y |
| II-64 | O | N(alkyl) | —C(═O)—O—Y |
| II-65 | O | CH2 | —C(═O)-amide″ |
| II-66 | O | C═O | —C(═O)-amide″ |
| II-67 | O | O | —C(═O)-amide″ |
| II-68 | O | S | —C(═O)-amide″ |
| II-69 | O | SO | —C(═O)-amide″ |
| II-70 | O | SO2 | —C(═O)-amide″ |
| II-71 | O | NH | —C(═O)-amide″ |
| II-72 | O | N(alkyl) | —C(═O)-amide″ |
| II-73 | S | CH2 | —CH2—OH |
| II-74 | S | C═O | —CH2—OH |
| II-75 | S | O | —CH2—OH |
| II-76 | S | S | —CH2—OH |
| II-77 | S | SO | —CH2—OH |
| II-78 | S | SO2 | —CH2—OH |
| II-79 | S | NH | —CH2—OH |
| II-80 | S | N(alkyl) | —CH2—OH |
| II-81 | S | CH2 | —C(═O)—O—Y |
| II-82 | S | C═O | —C(═O)—O—Y |
| II-83 | S | O | —C(═O)—O—Y |
| II-84 | S | S | —C(═O)—O—Y |
| II-85 | S | SO | —C(═O)—O—Y |
| II-86 | S | SO2 | —C(═O)—O—Y |
| II-87 | S | NH | —C(═O)—O—Y |
| II-88 | S | N(alkyl) | —C(═O)—O—Y |
| II-89 | S | CH2 | —C(═O)-amide″ |
| II-90 | S | C═O | —C(═O)-amide″ |
| II-91 | S | O | —C(═O)-amide″ |
| II-92 | S | S | —C(═O)-amide″ |
| II-93 | S | SO | —C(═O)-amide″ |
| II-94 | S | SO2 | —C(═O)-amide″ |
| II-95 | S | NH | —C(═O)-amide″ |
| II-96 | S | N(alkyl) | —C(═O)-amide″ |
| II-97 | SO | CH2 | —CH2—OH |
| II-98 | SO | C═O | —CH2—OH |
| II-99 | SO | O | —CH2—OH |
| II-100 | SO | S | —CH2—OH |
| II-101 | SO | SO | —CH2—OH |
| II-102 | SO | SO2 | —CH2—OH |
| II-103 | SO | NH | —CH2—OH |
| II-104 | SO | N(alkyl) | —CH2—OH |
| II-105 | SO | CH2 | —C(═O)—O—Y |
| II-106 | SO | C═O | —C(═O)—O—Y |
| II-107 | SO | O | —C(═O)—O—Y |
| II-108 | SO | S | —C(═O)—O—Y |
| II-109 | SO | SO | —C(═O)—O—Y |
| II-110 | SO | SO2 | —C(═O)—O—Y |
| II-111 | SO | NH | —C(═O)—O—Y |
| II-112 | SO | N(alkyl) | —C(═O)—O—Y |
| II-113 | SO | CH2 | —C(═O)-amide″ |
| II-114 | SO | C═O | —C(═O)-amide″ |
| II-115 | SO | O | —C(═O)-amide″ |
| II-116 | SO | S | —C(═O)-amide″ |
| II-117 | SO | SO | —C(═O)-amide″ |
| II-118 | SO | SO2 | —C(═O)-amide″ |
| II-119 | SO | NH | —C(═O)-amide″ |
| II-120 | SO | N(alkyl) | —C(═O)-amide″ |
| II-121 | SO2 | CH2 | —CH2—OH |
| II-122 | SO2 | C═O | —CH2—OH |
| II-123 | SO2 | O | —CH2—OH |
| II-124 | SO2 | S | —CH2—OH |
| II-125 | SO2 | SO | —CH2—OH |
| II-126 | SO2 | SO2 | —CH2—OH |
| II-127 | SO2 | NH | —CH2—OH |
| II-128 | SO2 | N(alkyl) | —CH2—OH |
| II-129 | SO2 | CH2 | —C(═O)—O—Y |
| II-130 | SO2 | C═O | —C(═O)—O—Y |
| II-131 | SO2 | O | —C(═O)—O—Y |
| II-132 | SO2 | S | —C(═O)—O—Y |
| II-133 | SO2 | SO | —C(═O)—O—Y |
| II-134 | SO2 | SO2 | —C(═O)—O—Y |
| II-135 | SO2 | NH | —C(═O)—O—Y |
| II-136 | SO2 | N(alkyl) | —C(═O)—O—Y |
| II-137 | SO2 | CH2 | —C(═O)-amide″ |
| II-138 | SO2 | C═O | —C(═O)-amide″ |
| II-139 | SO2 | O | —C(═O)-amide″ |
| II-140 | SO2 | S | —C(═O)-amide″ |
| II-141 | SO2 | SO | —C(═O)-amide″ |
| II-142 | SO2 | SO2 | —C(═O)-amide″ |
| II-143 | SO2 | NH | —C(═O)-amide″ |
| II-144 | SO2 | N(alkyl) | —C(═O)-amide″ |
| II-145 | NH | CH2 | —CH2—OH |
| II-146 | NH | C═O | —CH2—OH |
| II-147 | NH | O | —CH2—OH |
| II-148 | NH | S | —CH2—OH |
| II-149 | NH | SO | —CH2—OH |
| II-150 | NH | SO2 | —CH2—OH |
| II-151 | NH | NH | —CH2—OH |
| II-152 | NH | N(alkyl) | —CH2—OH |
| II-153 | NH | CH2 | —C(═O)—O—Y |
| II-154 | NH | C═O | —C(═O)—O—Y |
| II-155 | NH | O | —C(═O)—O—Y |
| II-156 | NH | S | —C(═O)—O—Y |
| II-157 | NH | SO | —C(═O)—O—Y |
| II-158 | NH | SO2 | —C(═O)—O—Y |
| II-159 | NH | NH | —C(═O)—O—Y |
| II-160 | NH | N(alkyl) | —C(═O)—O—Y |
| II-161 | NH | CH2 | —C(═O)-amide″ |
| II-162 | NH | C═O | —C(═O)-amide″ |
| II-163 | NH | O | —C(═O)-amide″ |
| II-164 | NH | S | —C(═O)-amide′′ |
| II-165 | NH | SO | —C(═O)-amide″ |
| II-166 | NH | SO2 | —C(═O)-amide″ |
| II-167 | NH | NH | —C(═O)-amide″ |
| II-168 | NH | N(alkyl) | —C(═O)-amide″ |
| II-169 | N(alkyl) | CH2 | —CH2—OH |
| II-170 | N(alkyl) | C═O | —CH2—OH |
| II-171 | N(alkyl) | O | —CH2—OH |
| II-172 | N(alkyl) | S | —CH2—OH |
| II-173 | N(alkyl) | SO | —CH2—OH |
| II-174 | N(alkyl) | SO2 | —CH2—OH |
| II-175 | N(alkyl) | NH | —CH2—OH |
| II-176 | N(alkyl) | N(alkyl) | —CH2—OH |
| II-177 | N(alkyl) | CH2 | —C(═O)—O—Y |
| II-178 | N(alkyl) | C═O | —C(═O)—O—Y |
| II-179 | N(alkyl) | O | —C(═O)—O—Y |
| II-180 | N(alkyl) | S | —C(═O)—O—Y |
| II-181 | N(alkyl) | SO | —C(═O)—O—Y |
| II-182 | N(alkyl) | SO2 | —C(═O)—O—Y |
| II-183 | N(alkyl) | NH | —C(═O)—O—Y |
| II-184 | N(alkyl) | N(alkyl) | —C(═O)—O—Y |
| II-185 | N(alkyl) | CH2 | —C(═O)-amide″ |
| II-186 | N(alkyl) | C═O | —C(═O)-amide″ |
| II-187 | N(alkyl) | O | —C(═O)-amide″ |
| II-188 | N(alkyl) | S | —C(═O)-amide″ |
| II-189 | N(alkyl) | SO | —C(═O)-amide″ |
| II-190 | N(alkyl) | SO2 | —C(═O)-amide″ |
| II-191 | N(alkyl) | NH | —C(═O)-amide″ |
| II-192 | N(alkyl) | N(alkyl) | —C(═O)-amide″ |

wherein in the above mycosporine-like amino acid compounds the phenyl ring may be unsubstituted or substituted, and R4′, R5′, R6′, R4″, R5″, R6″, amide′, Y, amide″ and alkyl have the same meaning as defined in any one of the preceding claims, or a tautomer or a stereoisomer or a salt of the afore-mentioned compounds;
or wherein the mycosporine-like amino acid is selected from the group consisting of 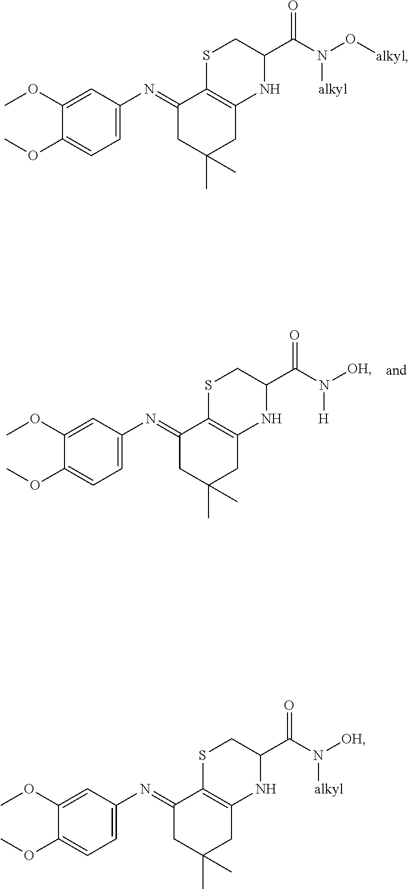
wherein alkyl is either methyl or ethyl or propyl or butyl,
or a tautomer or a salt of the afore-mentioned compounds;
or any mixture of the afore-mentioned compounds.

7. The sunscreen product, cosmetic or pharmaceutical preparation or homecare product according to claim 1, wherein in the general formulae (VI), X is S, SO or SO₂, and/or wherein in the general formulae (V) to (VIII) n=1.

8. The sunscreen product, cosmetic or pharmaceutical preparation or homecare product according to claim 1, wherein in the general formulae (V), (VII) and (VIII) the substituents R4′, R5′ and R6′ which may be identical or different, are independently from each other selected from the group consisting of H, OH, alkyl, alkoxy, —S(═O)₂OH, and sulfonyl;
or
wherein in the general formulae (VI), the substituents R4″, R5″ and R6″ which may be identical or different, are independently from each other selected from the group consisting of H, OH, alkyl, alkoxy, —S(═O)₂OH, and sulfonyl.

9. The sunscreen product, cosmetic or pharmaceutical preparation or homecare product according to claim 1, wherein in the general formulae (V) to (VIII) the substituted phenyl bonded to the imino functionality is selected from the group consisting of 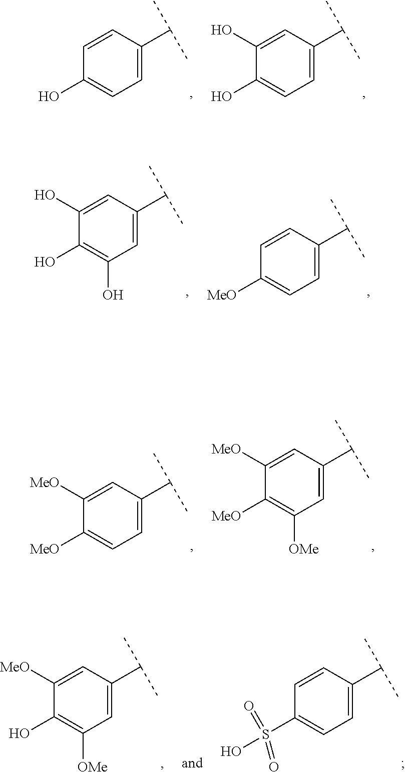
wherein the dotted line designates the binding site.

10. The sunscreen product, cosmetic or pharmaceutical preparation or homecare product according to claim 1, wherein in the general formulae (V), (VII) and (VIII) the substituents R4′, R5′, R6′, which may be identical or different, are independently from each other selected from the group consisting of H, OH, alkyl, alkoxy, —S(═O)₂OH and sulfonyl; with the proviso, that if the substituted phenyl ring is monosubstituted, the substituent is either in the ortho or meta-position to the imino-functionality; or wherein in the general formulae (VI), the substituents R4″, R5″, R6″, which may be identical or different, are independently from each other selected from the group consisting of H, OH, alkyl, alkoxy, —S(═O)₂OH and sulfonyl; with the proviso, that if the substituted phenyl ring is monosubstituted, the substituent is either in the ortho or meta-position to the imino-functionality.

11. The sunscreen product, cosmetic or pharmaceutical preparation or homecare product according to claim 1, wherein the mycosporine-like amino acid compound is selected from the group consisting of

| Com- | | |
|---|---|---|
| pound | Structure | INCI/chemical name |
| MAA-1 | 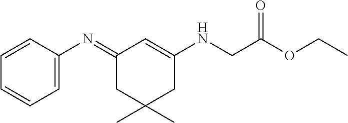 [SMILES: CCOC(=O)CNC1=C/C(=N/c2ccccc2)CC(C)(C)C1] | ethyl 2-[[5,5- dimethyl-3- phenylimino- cyclohexen-1- yl]amino]acetate |
| MAA-2 | 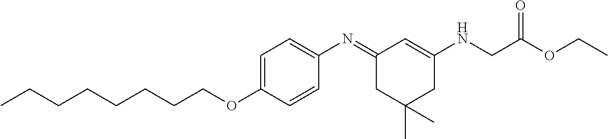 [SMILES: CCCCCCCCOc1ccc(/N=C2/C=C(NCC(=O)OCC)CC(C)(C)C2)cc1] | ethyl 2-((5,5- dimethyl-3-((4- octyoxy)phenyl) imino)cyclohex-1- en-1-yl)amino)acetate |
| MAA-3 | 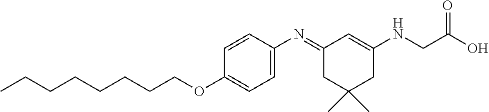 [SMILES: CCCCCCCCOc1ccc(/N=C2/C=C(NCC(=O)O)CC(C)(C)C2)cc1] | 5,5-dimethyl-3-((4- (octyloxy)phenyl) imino)cyclohex-1- en-1-yl-glycine |
| MAA-4 | 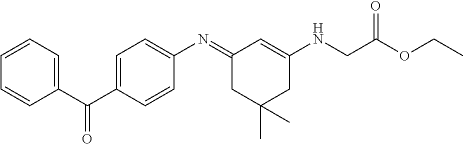 [SMILES: CCOC(=O)CNC1=C/C(=N/c2ccc(C(=O)c3ccccc3)cc2)CC(C)(C)C1] | ethyl 2-((3-((4- benzoylphenyl) imino)-5,5- dimethylcyclohex-1-en- 1-yl)amino)acetate |
| MAA-5 | 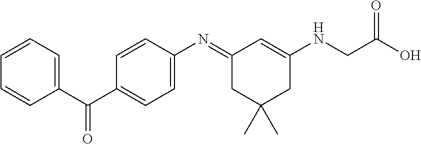 [SMILES: CC1(C)CC(NCC(=O)O)=C/C(=N/c2ccc(C(=O)c3ccccc3)cc2)C1] | 3-((4-benzoylphenyl) imino)-5,5- dimethylcyclohex- 1-en-1-yl-glycine |
| MAA-6 | 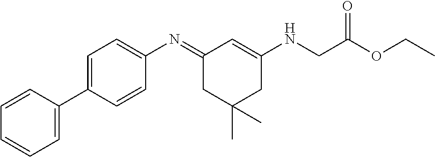 [SMILES: CCOC(=O)CNC1=C/C(=N/c2ccc(-c3ccccc3)cc2)CC(C)(C)C1] | ethyl 2-((3-([1,1′- biphenyl]-4- ylimino)-5,5- dimethylcyclohex-1-en- 1.yl)amino)acetate |
| MAA-7 | 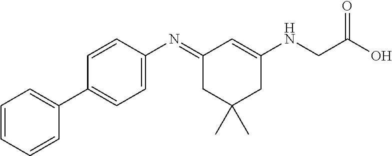 [SMILES: CC1(C)CC(NCC(=O)O)=C/C(=N/c2ccc(-c3ccccc3)cc2)C1] | 3-([1,1′-biphenyl]- 4-ylimino)-5,5- dimethylcyclohex-1- en-1-yl-glycine |
| MAA-8 | 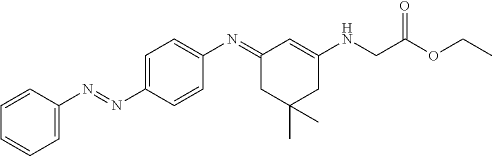 [SMILES: CCOC(=O)CNC1=C/C(=N/c2ccc(/N=N/c3ccccc3)cc2)CC(C)(C)C1] | ethyl 2-((5,5- dimethyl-3-((4- (phenyldiazenyl) phenyl)inino (cyclohex-1-en-1- yl)amino)acetate |
| MAA-9 | 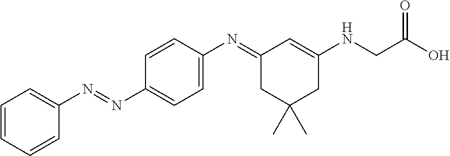 [SMILES: CC1(C)CC(NCC(=O)O)=C/C(=N/c2ccc(/N=N/c3ccccc3)cc2)C1] | 5,5-dimethyl-3-((4-((E)- phenyldiazenyl) phenyl)imino) cyclohex-1-en-1- yl-glycine |
| MAA-11 | 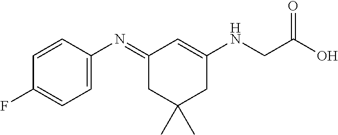 [SMILES: CC1(C)CC(NCC(=O)O)=C/C(=N/c2ccc(F)cc2)C1] | 3-((4-fluorophenyl) imino)-5,5- dimethylcyclohex- 1-en-1-yl-glycine |
| MAA-12 | 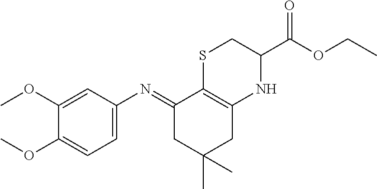 [SMILES: CCOC(=O)C1CSC2=C(CC(C)(C)C/C2=N\c2ccc(OC)c(OC)c2)N1] | ethyl 8-((3,4- dimethoyxphenyl) imino)-6,6- dimethyl-3,4,5,6,7,8- hexahydro-2-H- benzol[b][1,4]thiazine- 3-carboxylate |
| MAA-13 | 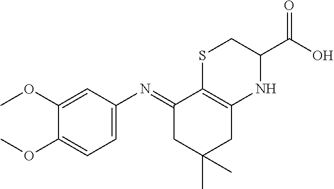 [SMILES: COc1ccc(/N=C2\CC(C)(C)CC3=C2SCC(C(=O)O)N3)cc1OC] | 8-((3,4-dimethoyxphenyl) imino)-6,6- dimethyl-3,4,5,6,7,8- hexahydro-2-H- benzol[b][1,4]thiazine- 3-carboxylic acid |
| MAA-14 | 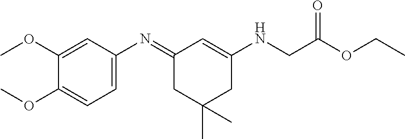 [SMILES: CCOC(=O)CNC1=C/C(=N/c2ccc(OC)c(OC)c2)CC(C)(C)C1] | ethyl 2-((3-((3,4- dimethoxyphenyl) imino)-5,5- dimethylcyclohex-1-en- 1-yl)amino)acetate |
| MAA-16 | 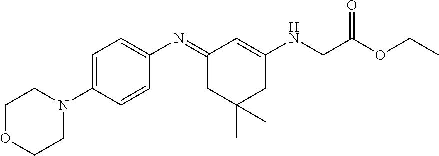 [SMILES: CCOC(=O)CNC1=C/C(=N/c2ccc(N3CCOCC3)cc2)CC(C)(C)C1] | ethyl 2-((5,5- dimethyl-3-((4- morpholinophenyl) imino)cyclohex- 1-en-1-yl)amino)acetate |
| MAA-17 | 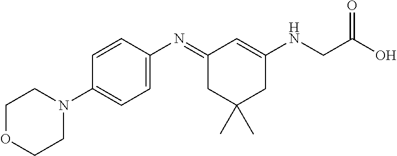 [SMILES: CC1(C)CC(NCC(=O)O)=C/C(=N/c2ccc(N3CCOCC3)cc2)C1] | 5,5-dimethyl-3-((4- morpholinophenyl) imino)cyclohex- 1-en-1-yl-glycine |
| MAA-20 | 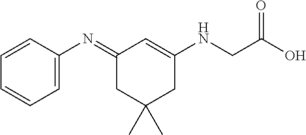 [SMILES: CC1(C)CC(NCC(=O)O)=C/C(=N/c2ccccc2)C1] | 2-[(5,5-dimethyl- 3-phenylimino- cyclohexen-1-yl) amino]acetic acid |
| MAA-21 | 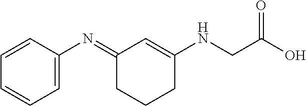 [SMILES: O=C(O)CNC1=C/C(=N/c2ccccc2)CCC1] | 2-[(3- phenyliminocyclohexen-1- yl)amino]acetic acid |
| MAA-22 | 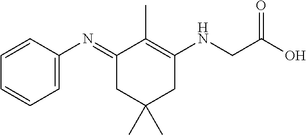 [SMILES: CC1=C(NCC(=O)O)CC(C)(C)C/C1=N\c1ccccc1] | 2,5,5-trimethyl-3- (phenylimino) cyclohex-1-en-1-yl- glycine |
| MAA-23 | 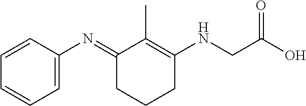 [SMILES: CC1=C(NCC(=O)O)CCC/C1=N\c1ccccc1] | 2-[(2-methyl-3- phenylimino- cyclohexen-1-yl) amino]acetic acid |
| MAA-24 | 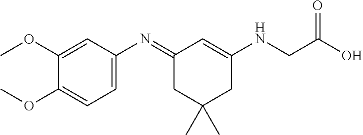 [SMILES: COc1ccc(/N=C2/C=C(NCC(=O)O)CC(C)(C)C2)cc1OC] | 3-((3,4-dimethoxyphenyl) imino)-5,5- dimethylcyclohex- 1-en-1-yl-glycine |
| MAA-25 | 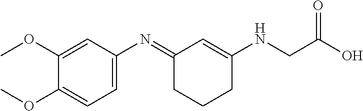 [SMILES: COc1ccc(/N=C2/C=C(NCC(=O)O)CCC2)cc1OC] | 3-((3,4- dimethoxyphenyl) imino)cyclohex-1- en-1-yl-glycine |
| MAA-26 | 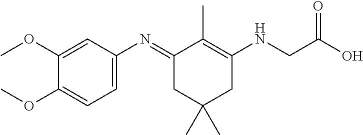 [SMILES: COc1ccc(/N=C2\CC(C)(C)CC(NCC(=O)O)=C2C)cc1OC] | 3-((3,4- dimethoxyphenyl)imino)- 2,5,5-trimethylcyclohex- 1-en-1-yl- glycine |
| MAA-27 | 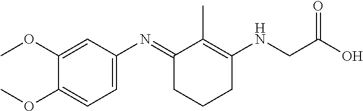 [SMILES: COc1ccc(/N=C2\CCCC(NCC(=O)O)=C2C)cc1OC] | 2-[(3-(3,4- dimethoxyphenyl) imino-2- methyl-cyclohexen- 1-yl)amino]acetic acid |
| MAA-28 | 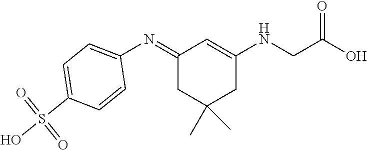 [SMILES: CC1(C)CC(NCC(=O)O)=C/C(=N/c2ccc(S(=O)(=O)O)cc2)C1] | 2-[(5,5-dimethyl-3-(4- sulfophenyl)imino- cyclohexen-1- yl)amino]acetic acid |
| MAA-29 | 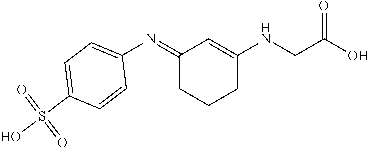 [SMILES: O=C(O)CNC1=C/C(=N/c2ccc(S(=O)(=O)O)cc2)CCC1] | 2-[(3-(4- sulfophenyl) iminocyclohexen-1- yl)amino]acetic acid |
| MAA-30 | 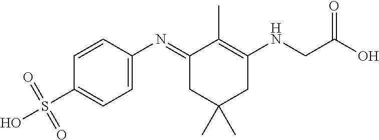 [SMILES: CC1=C(NCC(=O)O)CC(C)(C)C/C1=N\c1ccc(S(=O)(=O)O)cc1] | 2,5,5-trimethyl-3-((4- sulfophenyl)imino) cyclohex-1-en-1- yl-glycine |
| MAA-31 | 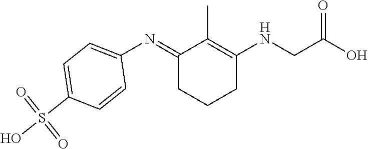 [SMILES: CC1=C(NCC(=O)O)CCC/C1=N\c1ccc(S(=O)(=O)O)cc1] | 2-methyl-3-((4- sulfophenyl)imino) cyclohex-1-en-1- yl-glycine |
| MAA-32 | 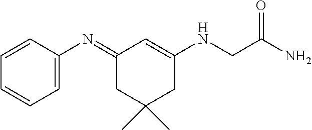 [SMILES: CC1(C)CC(NCC(N)=O)=C/C(=N/c2ccccc2)C1] | 2-[(5,5-dimethyl- 3-phenylimino- cyclohexen-1-yl) amino]acetamide |
| MAA-33 | 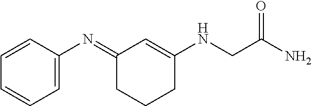 [SMILES: NC(=O)CNC1=C/C(=N/c2ccccc2)CCC1] | 2-[(3- phenyliminocyclohexen- 1-yl)amino]acetamide |
| MAA-34 | 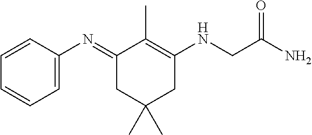 [SMILES: CC1=C(NCC(N)=O)CC(C)(C)C/C1=N\c1ccccc1] | 2-((2,5,5-trimethyl-3- (phenylimino) cyclohex-1-en-1- yl)amino)acetamide |
| MAA-35 | 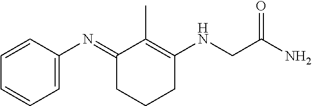 [SMILES: CC1=C(NCC(N)=O)CCC/C1=N\c1ccccc1] | 2-((2-methyl-3- (phenylimino) cyclohex-1-en-1- yl)amino)acetamide |
| MAA-36 | 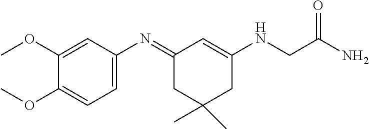 [SMILES: COc1ccc(/N=C2/C=C(NCC(N)=O)CC(C)(C)C2)cc1OC] | 2-((3-((3,4- dimethoxyphenyl)imino)- 5,5-dimethylcyclohex- 1-en-1- yl)amino)acetamide |
| MAA-37 | 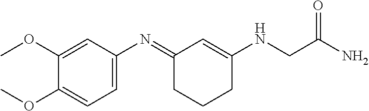 [SMILES: COc1ccc(/N=C2/C=C(NCC(N)=O)CCC2)cc1OC] | 2-[(3-(3,4- dimethoxyphenyl) iminocyclohexen- 1-yl)amino]acetamide |
| MAA-38 | 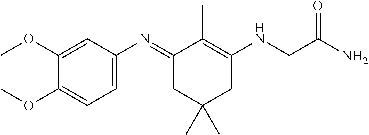 [SMILES: COc1ccc(/N=C2\CC(C)(C)CC(NCC(N)=O)=C2C)cc1OC] | 2-((3-((3,4- dimethoxyphenyl)imino)- 2,5,5-trimethylcyclohex- 1-en-1- yl)amino)acetamide |
| MAA-39 | 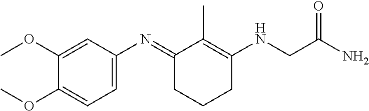 [SMILES: COc1ccc(/N=C2\CCCC(NCC(N)=O)=C2C)cc1OC] | 2-((3-((3,4- dimethoxyphenyl) imino)- 2-methylcyclohex-1-en-1- yl)amino)acetamide |

or a tautomer or a stereoisomer or a salt of the afore-mentioned compounds;
or any mixture of the afore-mentioned compounds.

12. The sunscreen product, cosmetic or pharmaceutical preparation or homecare product according to claim 1, wherein the at least one primary organic and/or inorganic UV-filter is selected from the group consisting of Camphor Benzalkonium Methosulfate, Homosalate, Benzophenone-3, Phenylbenzimidazole Sulfonic Acid, Terephthalylidene Dicamphor Sulfonic Acid, Butyl Methoxydibenzoylmethane, Benzylidene Camphor Sulfonic Acid, Octocrylene, Polyacrylamidomethyl Benzylidene Camphor, Ethylhexyl Methoxycinnamate, PEG-25 PABA, Isoamyl p-Methoxycinnamate, Ethylhexyl Triazone, Drometrizole Trisiloxane, Diethylhexyl Butamido Triazone, 4-Methylbenzylidene Camphor, Ethylhexyl Salicylate, Ethylhexyl Dimethyl PABA, Benzophenone-4, Benzophenone-5, Methylene Bis-benzotriazolyl Tetramethylbutylphenol (nano), Disodium Phenyl Dibenzimidazole Tetrasulfonate, Bis-Ethylhexyloxyphenol Methoxyphenyl Triazine, Polysilicone-15, Titanium Dioxide, Titanium Dioxide (nano), Diethylamino Hydroxy benzoyl Hexyl Benzoate, Tris-biphenyl triazine (nano), Zinc Oxide, Zinc Oxide (nano), Phenylene Bis-Diphenyltriazine, Methoxypropylamino Cyclohexenylidene Ethoxyethylcyanoacetate, Bis-(Diethylaminohydroxybenzoyl Benzoyl) Piperazine, TEA-Salicylate, Benzophenone-8, PABA, Ethylhexyl Dimethyl PABA, Menthyl Anthranilate, and any mixture thereof.

13. The sunscreen product, cosmetic or pharmaceutical preparation or homecare product according to claim 1, wherein the at least one film forming agent is selected from the group consisting of Polyamide-8, Polyamide-3, Trimethylpentanediol/Adipic Acid/Glycerin Crosspolymer, Octyldodecyl Citrate Crosspolymer, Polyglyceryl-3 Methyl Glucose Distearate, C28-52 Olefin/Undecylenic Acid Copolymer, Hydrolyzed Jojoba esters, Jojoba Esters, Aqua, Cyclopentasiloxane Acrylates/Dimethicone copolymer, Acrylates/Beheneth-25 Methacylate Copolymer, Styrene/Acrylates Copolymer, Polyurethane, Polyurethane-2 and Polymethyl Methacrylate, Acrylates/Octylacrylamide Copolymer, Beeswax (Cera Alba)(and) Sodium Stearoyl Lactylate, Pullulan, Xanthan Gum, Hydrogenated Palm Oil, Saccharum officinarum Extract (SymEffect™ UV), Microcrystalline cellulose (and) Sphingomonas ferment extract (and) Cellulose gum, Acacia Senegal Gum and Xanthan Gum, Microcrystalline Cellulose (and) Cellulose Gum, Sodium Stearoyl Glutamate, Hydrogenated Polycyclopentadiene (and) Caprylic/Capric Triglyceride, Glyceryl Hydrogenated Rosinate (and) Caprylic/Capric Triglyceride (and) Tocopherol, Galactoarabinan, C26-28 Alkyl Dimethicone, Caprylyl Methicone, Stearyl Dimethicone (and) Octadecene, VP/Hexadecene Copolymer, Triacontanyl PVP, Capryloyl Glycerin/Sebacic Acid Copolymer, Poly C10-30 Alkyl Acrylate, Microcrystalline Cellulose, and any mixture thereof.

14. The sunscreen product, cosmetic or pharmaceutical preparation or homecare product according to claim 1, comprising the at least one primary organic and/or inorganic UV-filter or a mixture thereof in an amount of 0.01 to 80.0% by weight, based on the total weight of the product or preparation.

15. The sunscreen product, cosmetic or pharmaceutical preparation or homecare product according to claim 1, comprising the at least one mycosporine-like amino acid compound or a mixture thereof in an amount of 0.001 to 15.0% by weight, based on the total weight of the product or preparation.

16. The sunscreen product, cosmetic or pharmaceutical preparation or homecare product according to claim 1, comprising the at least one film forming agent or a mixture thereof in an amount of 0.01 to 10.0% by weight, based on the total weight of the product or preparation.

17. The sunscreen product, cosmetic or pharmaceutical preparation or homecare product according to claim 1, further comprising at least one 1,2-alkanediol or 2,3-alkanediol with 5 to 12 carbon atoms-.

18-19. (canceled)

20. The sunscreen product, cosmetic or pharmaceutical preparation or homecare product according to claim 1 in the form of
(i) a dispersion;
(ii) a water free formulation; or
(iii) an aqueous aqueous/alcoholic, aqueous/glycolic, or alcoholic/glycolic solution.

21. (canceled)

22. The sunscreen product, cosmetic or pharmaceutical preparation or homecare product according to claim 1, wherein the water resistance after immersion in water is increased by at least 5%, compared to a sunscreen product, cosmetic or pharmaceutical preparation or homecare product without the mycosporine-like amino acid compound.

23. (canceled)

24. Method of increasing the water resistance of a sunscreen product, cosmetic or pharmaceutical preparation or homecare product comprising at least one primary organic and/or inorganic UV-filter and optionally at least one film forming agent, said method comprising adding of an effective amount of a mycosporine-like amino acid compound or a mixture thereof to said sunscreen product, cosmetic or pharmaceutical preparation or homecare product.
